SEQUENCE LISTING (382 sequences)

SEQ ID NO: 1 - protein, 257 aa, Homo sapiens
  REGION 1..257: note = misc_feature - Human folate receptor alpha (hFOLR1)
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 2 - protein, 257 aa, Macaca fascicularis
  REGION 1..257: note = misc_feature - Cynomolgus folate receptor alpha
MAQRMTTQLLLLLVWVAVVGEAQTRTARARTELLNVCMNAKHHKEKPGPEDKLHEQCRPWKKNACCSTNTSQEAHKDVSYLYRFNWNHCGEMAPACKRHFIQDTCLYECSPNLGPWIQQVDQSWRKERVLNVPLCKEDCERWWEDCRTSYTCKSNWHKGWNWTSGFNKCPVGAACQPFHFYFPTPTVLCNEIWTYSYKVSNYSRGSGRCIQMWFDPAQGNPNEEVARFYAAAMSGAGPWAAWPLLLSLALTLLWLLS

SEQ ID NO: 3 - protein, 255 aa, Mus musculus
  REGION 1..255: note = misc_feature - Murine folate receptor alpha
MAHLMTVQLLLLVMWMAECAQSRATRARTELLNVCMDAKHHKEKPGPEDNLHDQCSPWKTNSCCSTNTSQEAHKDISYLYRFNWNHCGTMTSECKRHFIQDTCLYECSPNLGPWIQQVDQSWRKERILDVPLCKEDCQQWWEDCQSSFTCKSNWHKGWNWSSGHNECPVGASCHPFTFYFPTSAALCEEIWSHSYKLSNYSRGSGRCIQMWFDPAQGNPNEEVARFYAEAMSGAGFHGTWPLLCSLSLVLLWVIS

SEQ ID NO: 4 - protein, 7 aa, synthetic construct
  REGION 1..7: note = Synthetic: SRP1848-A01
GFNITRY

SEQ ID NO: 5 - protein, 7 aa, synthetic construct
  REGION 1..7: note = Synthetic: SRP1848-A02
GFNISGF

SEQ ID NO: 6 - protein, 7 aa, synthetic construct
  REGION 1..7: note = Synthetic: SRP1848-A04
GFNIDQS

SEQ ID NO: 7 - protein, 7 aa, synthetic construct
  REGION 1..7: note = Synthetic: SRP1848-A06
GFNIGNS

SEQ ID NO: 8 - protein, 7 aa, synthetic construct
  REGION 1..7: note = Synthetic: SRP1848-A07
GFNIGYH

SEQ ID NO: 9 - protein, 7 aa, synthetic construct
  REGION 1..7: note = Synthetic: SRP1848-A08
GSNIRKH

SEQ ID NO: 10 - protein, 7 aa, synthetic construct
  REGION 1..7: note = Synthetic: SRP1848-A09
GFNIRKQ

SEQ ID NO: 11 - protein, 7 aa, synthetic construct
  REGION 1..7: note = Synthetic: SRP1848-A10
GFNIRKY

SEQ ID NO: 12 - protein, 7 aa, synthetic construct
  REGION 1..7: note = Synthetic: SRP1848-B01
GFNIRNY

SEQ ID NO: 13 - protein, 7 aa, synthetic construct
  REGION 1..7: note = Synthetic: SRP1848-B03
GFNISMK

SEQ ID NO: 14 - protein, 7 aa, synthetic construct
  REGION 1..7: note = Synthetic: SRP1848-B04
SFNISNH

SEQ ID NO: 15 - protein, 7 aa, synthetic construct
  REGION 1..7: note = Synthetic: SRP1848-B05
GFNISNY

SEQ ID NO: 16 - protein, 7 aa, synthetic construct
  REGION 1..7: note = Synthetic: SRP1848-B06
GFNISNY

SEQ ID NO: 17 - protein, 7 aa, synthetic construct
  REGION 1..7: note = Synthetic: SRP1848-B07
GFNISRF

SEQ ID NO: 18 - protein, 7 aa, synthetic construct
  REGION 1..7: note = Synthetic: SRP1848-B09
GFNITNY

SEQ ID NO: 19 - protein, 7 aa, synthetic construct
  REGION 1..7: note = Synthetic: SRP1848-B10
GFNTTTK

SEQ ID NO: 20 - protein, 7 aa, synthetic construct
  REGION 1..7: note = Synthetic: SRP1848-B11
GFNIGNN

SEQ ID NO: 21 - protein, 7 aa, synthetic construct
  REGION 1..7: note = Synthetic: SRP1848-C01
GFNIGNS

SEQ ID NO: 22 - protein, 7 aa, synthetic construct
  REGION 1..7: note = Synthetic: SRP1848-C03
GFNIGVY

SEQ ID NO: 23 - protein, 7 aa, synthetic construct
  REGION 1..7: note = Synthetic: SRP1848-C04
GFNIRHY

SEQ ID NO: 24 - protein, 7 aa, synthetic construct
  REGION 1..7: note = Synthetic: SRP1848-C05
GFNIRKY

SEQ ID NO: 25 - protein, 7 aa, synthetic construct
  REGION 1..7: note = Synthetic: SRP1848-C07
GFNIRKY

SEQ ID NO: 26 - protein, 7 aa, synthetic construct
  REGION 1..7: note = Synthetic: SRP1848-C10
GFNIRTY

SEQ ID NO: 27 - protein, 7 aa, synthetic construct
  REGION 1..7: note = Synthetic: SRP1848-D02
GFNISHN

SEQ ID NO: 28 - protein, 7 aa, synthetic construct
  REGION 1..7: note = Synthetic: SRP1848-D03
GFNIRYF

SEQ ID NO: 29 - protein, 7 aa, synthetic construct
  REGION 1..7: note = Synthetic: SRP1848-D04
GFNISHY

SEQ ID NO: 30 - protein, 7 aa, synthetic construct
  REGION 1..7: note = Synthetic: SRP1848-D05
GFNISIS

SEQ ID NO: 31 - protein, 7 aa, synthetic construct
  REGION 1..7: note = Synthetic: SRP1848-D07
GFNISKY

SEQ ID NO: 32 - protein, 7 aa, synthetic construct
  REGION 1..7: note = Synthetic: SRP1848-D09
GFNISNY

SEQ ID NO: 33 - protein, 7 aa, synthetic construct
  REGION 1..7: note = Synthetic: SRP1848-D10
GFNISRN

SEQ ID NO: 34 - protein, 7 aa, synthetic construct
  REGION 1..7: note = Synthetic: SRP1848-E01
GFNITNK

SEQ ID NO: 35 - protein, 7 aa, synthetic construct
  REGION 1..7: note = Synthetic: SRP1848-E02
GFNIGKY

SEQ ID NO: 36 - protein, 7 aa, synthetic construct
  REGION 1..7: note = Synthetic: SRP1848-E03
GFNIGNY

SEQ ID NO: 37 - protein, 7 aa, synthetic construct
  REGION 1..7: note = Synthetic: SRP1848-E05
GFNIGVY

SEQ ID NO: 38 - protein, 7 aa, synthetic construct
  REGION 1..7: note = Synthetic: SRP1848-E06
GFNINRY

SEQ ID NO: 39 - protein, 7 aa, synthetic construct
  REGION 1..7: note = Synthetic: SRP1848-E07
GFNIRKS

SEQ ID NO: 40 - protein, 7 aa, synthetic construct
  REGION 1..7: note = Synthetic: SRP1848-F01
GFNIRTY

SEQ ID NO: 41 - protein, 7 aa, synthetic construct
  REGION 1..7: note = Synthetic: SRP1848-F02
GFNIRTY

SEQ ID NO: 42 - protein, 7 aa, synthetic construct
  REGION 1..7: note = Synthetic: SRP1848-F04
GFNISNY

SEQ ID NO: 43 - protein, 7 aa, synthetic construct
  REGION 1..7: note = Synthetic: SRP1848-F05
GFNISKS

SEQ ID NO: 44 - protein, 7 aa, synthetic construct
  REGION 1..7: note = Synthetic: SRP1848-F06
GFNISLS

SEQ ID NO: 45 - protein, 7 aa, synthetic construct
  REGION 1..7: note = Synthetic: SRP1848-F07
GFNISNH

SEQ ID NO: 46 - protein, 7 aa, synthetic construct
  REGION 1..7: note = Synthetic: SRP1848-F08
GFNISNH

SEQ ID NO: 47 - protein, 7 aa, synthetic construct
  REGION 1..7: note = Synthetic: SRP1848-F09
GFNISNH

SEQ ID NO: 48 - protein, 7 aa, synthetic construct
  REGION 1..7: note = Synthetic: SRP1848-F10
GFNISNN

SEQ ID NO: 49 - protein, 7 aa, synthetic construct
  REGION 1..7: note = Synthetic: SRP1848-F11
GFNISNN

SEQ ID NO: 50 - protein, 7 aa, synthetic construct
  REGION 1..7: note = Synthetic: SRP1848-G01
GFNISRH

SEQ ID NO: 51 - protein, 7 aa, synthetic construct
  REGION 1..7: note = Synthetic: SRP1848-G03
GFNISTY

SEQ ID NO: 52 - protein, 7 aa, synthetic construct
  REGION 1..7: note = Synthetic: SRP1848-G04
GFNIHST

SEQ ID NO: 53 - protein, 7 aa, synthetic construct
  REGION 1..7: note = Synthetic: SRP1848-G06
GFNIRST

SEQ ID NO: 54 - protein, 7 aa, synthetic construct
  REGION 1..7: note = Synthetic: SRP1848-G07
GFNIHST

SEQ ID NO: 55 - protein, 7 aa, synthetic construct
  REGION 1..7: note = Synthetic: SRP1848-G09
GFNIRGT

SEQ ID NO: 56 - protein, 7 aa, synthetic construct
  REGION 1..7: note = Synthetic: SRP1848-G10
GFNIRST

SEQ ID NO: 57 - protein, 7 aa, synthetic construct
  REGION 1..7: note = Synthetic: SRP1848-G11
GFNISST

SEQ ID NO: 58 - protein, 7 aa, synthetic construct
  REGION 1..7: note = Synthetic: SRP1848-H01
GFNIRTQ

SEQ ID NO: 59 - protein, 9 aa, synthetic construct
  REGION 1..9: note = Synthetic: SRP2060-E10
GFSLSTFGM

SEQ ID NO: 60 - protein, 9 aa, synthetic construct
  REGION 1..9: note = Synthetic: SRP2060-E05
GFSLSTFGM

SEQ ID NO: 61 - protein, 9 aa, synthetic construct
  REGION 1..9: note = Synthetic: SRP2060-B01
GFSLSTFGM

SEQ ID NO: 62 - protein, 9 aa, synthetic construct
  REGION 1..9: note = Synthetic: SRP2060-A06
GFSLSTFGM

SEQ ID NO: 63 - protein, 5 aa, synthetic construct
  REGION 1..5: note = Synthetic: SRP1848-A01
RYSIH

SEQ ID NO: 64 - protein, 5 aa, synthetic construct
  REGION 1..5: note = Synthetic: SRP1848-A02
GFRIH

SEQ ID NO: 65 - protein, 5 aa, synthetic construct
  REGION 1..5: note = Synthetic: SRP1848-A04
QSSIH

SEQ ID NO: 66 - protein, 5 aa, synthetic construct
  REGION 1..5: note = Synthetic: SRP1848-A06
NSYIH

SEQ ID NO: 67 - protein, 5 aa, synthetic construct
  REGION 1..5: note = Synthetic: SRP1848-A07
YHSIH

SEQ ID NO: 68 - protein, 5 aa, synthetic construct
  REGION 1..5: note = Synthetic: SRP1848-A08
KHSIH

SEQ ID NO: 69 - protein, 5 aa, synthetic construct
  REGION 1..5: note = Synthetic: SRP1848-A09
KQSIH

SEQ ID NO: 70 - protein, 5 aa, synthetic construct
  REGION 1..5: note = Synthetic: SRP1848-A10
KYSIH

SEQ ID NO: 71 - protein, 5 aa, synthetic construct
  REGION 1..5: note = Synthetic: SRP1848-B01
NYSIH

SEQ ID NO: 72 - protein, 5 aa, synthetic construct
  REGION 1..5: note = Synthetic: SRP1848-B03
MKYIH

SEQ ID NO: 73 - protein, 5 aa, synthetic construct
  REGION 1..5: note = Synthetic: SRP1848-B04
NHSIH

SEQ ID NO: 74 - protein, 5 aa, synthetic construct
  REGION 1..5: note = Synthetic: SRP1848-B05
NYYIH

SEQ ID NO: 75 - protein, 5 aa, synthetic construct
  REGION 1..5: note = Synthetic: SRP1848-B06
NYYIH

SEQ ID NO: 76 - protein, 5 aa, synthetic construct
  REGION 1..5: note = Synthetic: SRP1848-B07
RFYIH

SEQ ID NO: 77 - protein, 5 aa, synthetic construct
  REGION 1..5: note = Synthetic: SRP1848-B09
NYYIH

SEQ ID NO: 78 - protein, 5 aa, synthetic construct
  REGION 1..5: note = Synthetic: SRP1848-B10
TKSIH

SEQ ID NO: 79 - protein, 5 aa, synthetic construct
  REGION 1..5: note = Synthetic: SRP1848-B11
NNSIH

SEQ ID NO: 80 - protein, 5 aa, synthetic construct
  REGION 1..5: note = Synthetic: SRP1848-C01
NSYIH

SEQ ID NO: 81 - protein, 5 aa, synthetic construct
  REGION 1..5: note = Synthetic: SRP1848-C03
VYSIH

SEQ ID NO: 82 - protein, 5 aa, synthetic construct
  REGION 1..5: note = Synthetic: SRP1848-C04
HYSIH

SEQ ID NO: 83 - protein, 5 aa, synthetic construct
  REGION 1..5: note = Synthetic: SRP1848-C05
KYSIH

SEQ ID NO: 84 - protein, 5 aa, synthetic construct
  REGION 1..5: note = Synthetic: SRP1848-C07
KYSIH

SEQ ID NO: 85 - protein, 5 aa, synthetic construct
  REGION 1..5: note = Synthetic: SRP1848-C10
TYYIH

SEQ ID NO: 86 - protein, 5 aa, synthetic construct
  REGION 1..5: note = Synthetic: SRP1848-D02
HNYIH

SEQ ID NO: 87 - protein, 5 aa, synthetic construct
  REGION 1..5: note = Synthetic: SRP1848-D03
YFSIH

SEQ ID NO: 88 - protein, 5 aa, synthetic construct
  REGION 1..5: note = Synthetic: SRP1848-D04
HYSIH

SEQ ID NO: 89 - protein, 5 aa, synthetic construct
  REGION 1..5: note = Synthetic: SRP1848-D05
ISYIH

SEQ ID NO: 90 - protein, 5 aa, synthetic construct
  REGION 1..5: note = Synthetic: SRP1848-D07
KYYIH

SEQ ID NO: 91 - protein, 5 aa, synthetic construct
  REGION 1..5: note = Synthetic: SRP1848-D09
NYYIH

SEQ ID NO: 92 - protein, 5 aa, synthetic construct
  REGION 1..5: note = Synthetic: SRP1848-D10
RNSIH

SEQ ID NO: 93 - protein, 5 aa, synthetic construct
  REGION 1..5: note = Synthetic: SRP1848-E01
NKYIH

SEQ ID NO: 94 - protein, 5 aa, synthetic construct
  REGION 1..5: note = Synthetic: SRP1848-E02
KYSIH

SEQ ID NO: 95 - protein, 5 aa, synthetic construct
  REGION 1..5: note = Synthetic: SRP1848-E03
NYYIH

SEQ ID NO: 96 - protein, 5 aa, synthetic construct
  REGION 1..5: note = Synthetic: SRP1848-E05
VYYIH

SEQ ID NO: 97 - protein, 5 aa, synthetic construct
  REGION 1..5: note = Synthetic: SRP1848-E06
RYYIH

SEQ ID NO: 98 - protein, 5 aa, synthetic construct
  REGION 1..5: note = Synthetic: SRP1848-E07
KSSIH

SEQ ID NO: 99 - protein, 5 aa, synthetic construct
  REGION 1..5: note = Synthetic: SRP1848-F01
TYSIH

SEQ ID NO: 100 - protein, 5 aa, synthetic construct
  REGION 1..5: note = Synthetic: SRP1848-F02
TYSIH

SEQ ID NO: 101 - protein, 5 aa, synthetic construct
  REGION 1..5: note = Synthetic: SRP1848-F04
NYSIH

SEQ ID NO: 102 - protein, 5 aa, synthetic construct
  REGION 1..5: note = Synthetic: SRP1848-F05
KSSIH

SEQ ID NO: 103 - protein, 5 aa, synthetic construct
  REGION 1..5: note = Synthetic: SRP1848-F06
LSYIH

SEQ ID NO: 104 - protein, 5 aa, synthetic construct
  REGION 1..5: note = Synthetic: SRP1848-F07
NHSIH

SEQ ID NO: 105 - protein, 5 aa, synthetic construct
  REGION 1..5: note = Synthetic: SRP1848-F08
NHSIH

SEQ ID NO: 106 - protein, 5 aa, synthetic construct
  REGION 1..5: note = Synthetic: SRP1848-F09
NHYIH

SEQ ID NO: 107 - protein, 5 aa, synthetic construct
  REGION 1..5: note = Synthetic: SRP1848-F10
NNSIH

SEQ ID NO: 108 - protein, 5 aa, synthetic construct
  REGION 1..5: note = Synthetic: SRP1848-F11
NNYIH

SEQ ID NO: 109 - protein, 5 aa, synthetic construct
  REGION 1..5: note = Synthetic: SRP1848-G01
RHSIH

SEQ ID NO: 110 - protein, 5 aa, synthetic construct
  REGION 1..5: note = Synthetic: SRP1848-G03
TYYIH

SEQ ID NO: 111 - protein, 5 aa, synthetic construct
  REGION 1..5: note = Synthetic: SRP1848-G04
STDIH

SEQ ID NO: 112 - protein, 5 aa, synthetic construct
  REGION 1..5: note = Synthetic: SRP1848-G06
STDIH

SEQ ID NO: 113 - protein, 5 aa, synthetic construct
  REGION 1..5: note = Synthetic: SRP1848-G07
STDIH

SEQ ID NO: 114 - protein, 5 aa, synthetic construct
  REGION 1..5: note = Synthetic: SRP1848-G09
GTDIH

SEQ ID NO: 115 - protein, 5 aa, synthetic construct
  REGION 1..5: note = Synthetic: SRP1848-G10
STDIH

SEQ ID NO: 116 - protein, 5 aa, synthetic construct
  REGION 1..5: note = Synthetic: SRP1848-G11
STDIH

SEQ ID NO: 117 - protein, 5 aa, synthetic construct
  REGION 1..5: note = Synthetic: SRP1848-H01
TQSIH

SEQ ID NO: 118 - protein, 7 aa, synthetic construct
  REGION 1..7: note = Synthetic: SRP2060-E10
TFGMGVG

SEQ ID NO: 119 - protein, 7 aa, synthetic construct
  REGION 1..7: note = Synthetic: SRP2060-E05
TFGMGVG

SEQ ID NO: 120 - protein, 7 aa, synthetic construct
  REGION 1..7: note = Synthetic: SRP2060-B01
TFGMGVG

SEQ ID NO: 121 - protein, 7 aa, synthetic construct
  REGION 1..7: note = Synthetic: SRP2060-A06
TFGMGVG

SEQ ID NO: 122 - protein, 6 aa, synthetic construct
  REGION 1..6: note = Synthetic: SRP1848-A01
LPESGG

SEQ ID NO: 123 - protein, 6 aa, synthetic construct
  REGION 1..6: note = Synthetic: SRP1848-A02
YPESGA

SEQ ID NO: 124 - protein, 6 aa, synthetic construct
  REGION 1..6: note = Synthetic: SRP1848-A04
YPVDGT

SEQ ID NO: 125 - protein, 6 aa, synthetic construct
  REGION 1..6: note = Synthetic: SRP1848-A06
TPIDGN

SEQ ID NO: 126 - protein, 6 aa, synthetic construct
  REGION 1..6: note = Synthetic: SRP1848-A07
FPVDGT

SEQ ID NO: 127 - protein, 6 aa, synthetic construct
  REGION 1..6: note = Synthetic: SRP1848-A08
YPNDGT

SEQ ID NO: 128 - protein, 6 aa, synthetic construct
  REGION 1..6: note = Synthetic: SRP1848-A09
FPNDGT

SEQ ID NO: 129 - protein, 6 aa, synthetic construct
  REGION 1..6: note = Synthetic: SRP1848-A10
FPIDDI

SEQ ID NO: 130 - protein, 6 aa, synthetic construct
  REGION 1..6: note = Synthetic: SRP1848-B01
YPVDGI

SEQ ID NO: 131 - protein, 6 aa, synthetic construct
  REGION 1..6: note = Synthetic: SRP1848-B03
TPIDGM

SEQ ID NO: 132 - protein, 6 aa, synthetic construct
  REGION 1..6: note = Synthetic: SRP1848-B04
YPVDGI

SEQ ID NO: 133 - protein, 6 aa, synthetic construct
  REGION 1..6: note = Synthetic: SRP1848-B05
SPIDGY

SEQ ID NO: 134 - protein, 6 aa, synthetic construct
  REGION 1..6: note = Synthetic: SRP1848-B06
TPIDGY

SEQ ID NO: 135 - protein, 6 aa, synthetic construct
  REGION 1..6: note = Synthetic: SRP1848-B07
SPYDGF

SEQ ID NO: 136 - protein, 6 aa, synthetic construct
  REGION 1..6: note = Synthetic: SRP1848-B09
TPVDGY

SEQ ID NO: 137 - protein, 6 aa, synthetic construct
  REGION 1..6: note = Synthetic: SRP1848-B10
YPRDGI

SEQ ID NO: 138 - protein, 6 aa, synthetic construct
  REGION 1..6: note = Synthetic: SRP1848-B11
SPIDGF

SEQ ID NO: 139 - protein, 6 aa, synthetic construct
  REGION 1..6: note = Synthetic: SRP1848-C01
TPNDGY

SEQ ID NO: 140 - protein, 6 aa, synthetic construct
  REGION 1..6: note = Synthetic: SRP1848-C03
YPIDGN

SEQ ID NO: 141 - protein, 6 aa, synthetic construct
  REGION 1..6: note = Synthetic: SRP1848-C04
YPGPGN

SEQ ID NO: 142 - protein, 6 aa, synthetic construct
  REGION 1..6: note = Synthetic: SRP1848-C05
FPIDGI

SEQ ID NO: 143 - protein, 6 aa, synthetic construct
  REGION 1..6: note = Synthetic: SRP1848-C07
FPIDGI

SEQ ID NO: 144 - protein, 6 aa, synthetic construct
  REGION 1..6: note = Synthetic: SRP1848-C10
SPIDGY

SEQ ID NO: 145 - protein, 6 aa, synthetic construct
  REGION 1..6: note = Synthetic: SRP1848-D02
TPQDGY

SEQ ID NO: 146 - protein, 6 aa, synthetic construct
  REGION 1..6: note = Synthetic: SRP1848-D03
FPNDGS

SEQ ID NO: 147 - protein, 6 aa, synthetic construct
  REGION 1..6: note = Synthetic: SRP1848-D04
YPRDGI

SEQ ID NO: 148 - protein, 6 aa, synthetic construct
  REGION 1..6: note = Synthetic: SRP1848-D05
SPIDGY

SEQ ID NO: 149 - protein, 6 aa, synthetic construct
  REGION 1..6: note = Synthetic: SRP1848-D07
SPNDGY

SEQ ID NO: 150 - protein, 6 aa, synthetic construct
  REGION 1..6: note = Synthetic: SRP1848-D09
SPNDGY

SEQ ID NO: 151 - protein, 6 aa, synthetic construct
  REGION 1..6: note = Synthetic: SRP1848-D10
SPNDGT

SEQ ID NO: 152 - protein, 6 aa, synthetic construct
  REGION 1..6: note = Synthetic: SRP1848-E01
TPFDGF

SEQ ID NO: 153 - protein, 6 aa, synthetic construct
  REGION 1..6: note = Synthetic: SRP1848-E02
YPNDGN

SEQ ID NO: 154 - protein, 6 aa, synthetic construct
  REGION 1..6: note = Synthetic: SRP1848-E03
TPRDGF

SEQ ID NO: 155 - protein, 6 aa, synthetic construct
  REGION 1..6: note = Synthetic: SRP1848-E05
TPNDGY

SEQ ID NO: 156 - protein, 6 aa, synthetic construct
  REGION 1..6: note = Synthetic: SRP1848-E06
TPNDGY

SEQ ID NO: 157 - protein, 6 aa, synthetic construct
  REGION 1..6: note = Synthetic: SRP1848-E07
FPYDGS

SEQ ID NO: 158 - protein, 6 aa, synthetic construct
  REGION 1..6: note = Synthetic: SRP1848-F01
FPNDGT

SEQ ID NO: 159 - protein, 6 aa, synthetic construct
  REGION 1..6: note = Synthetic: SRP1848-F02
FPNDGT

SEQ ID NO: 160 - protein, 6 aa, synthetic construct
  REGION 1..6: note = Synthetic: SRP1848-F04
YPIDGI

SEQ ID NO: 161 - protein, 6 aa, synthetic construct
  REGION 1..6: note = Synthetic: SRP1848-F05
YPNDGS

SEQ ID NO: 162 - protein, 6 aa, synthetic construct
  REGION 1..6: note = Synthetic: SRP1848-F06
SPIDGN

SEQ ID NO: 163 - protein, 6 aa, synthetic construct
  REGION 1..6: note = Synthetic: SRP1848-F07
YPNDGI

SEQ ID NO: 164 - protein, 6 aa, synthetic construct
  REGION 1..6: note = Synthetic: SRP1848-F08
YPVDGI

SEQ ID NO: 165 - protein, 6 aa, synthetic construct
  REGION 1..6: note = Synthetic: SRP1848-F09
SPLDGY

SEQ ID NO: 166 - protein, 6 aa, synthetic construct
  REGION 1..6: note = Synthetic: SRP1848-F10
FPNDGY

SEQ ID NO: 167 - protein, 6 aa, synthetic construct
  REGION 1..6: note = Synthetic: SRP1848-F11
TPIDGN

SEQ ID NO: 168 - protein, 6 aa, synthetic construct
  REGION 1..6: note = Synthetic: SRP1848-G01
APNDGS

SEQ ID NO: 169 - protein, 6 aa, synthetic construct
  REGION 1..6: note = Synthetic: SRP1848-G03
TPSDGF

SEQ ID NO: 170 - protein, 6 aa, synthetic construct
  REGION 1..6: note = Synthetic: SRP1848-G04
TPAGGA

SEQ ID NO: 171 - protein, 6 aa, synthetic construct
  REGION 1..6: note = Synthetic: SRP1848-G06
TPAGGA

SEQ ID NO: 172 - protein, 6 aa, synthetic construct
  REGION 1..6: note = Synthetic: SRP1848-G07
TPAGGA

SEQ ID NO: 173 - protein, 6 aa, synthetic construct
  REGION 1..6: note = Synthetic: SRP1848-G09
TPAGGA

SEQ ID NO: 174 - protein, 6 aa, synthetic construct
  REGION 1..6: note = Synthetic: SRP1848-G10
TPAGGA

SEQ ID NO: 175 - protein, 6 aa, synthetic construct
  REGION 1..6: note = Synthetic: SRP1848-G11
TPAGGA

SEQ ID NO: 176 - protein, 6 aa, synthetic construct
  REGION 1..6: note = Synthetic: SRP1848-H01
FPIDGI

SEQ ID NO: 177 - protein, 5 aa, synthetic construct
  REGION 1..5: note = Synthetic: SRP2060-E10
WWDDD

SEQ ID NO: 178 - protein, 5 aa, synthetic construct
  REGION 1..5: note = Synthetic: SRP2060-E05
WWDDD

SEQ ID NO: 179 - protein, 5 aa, synthetic construct
  REGION 1..5: note = Synthetic: SRP2060-B01
WWDDD

SEQ ID NO: 180 - protein, 5 aa, synthetic construct
  REGION 1..5: note = Synthetic: SRP2060-A06
WWDDD

SEQ ID NO: 181 - protein, 17 aa, synthetic construct
  REGION 1..17: note = Synthetic: SRP1848-A01
GILPESGGTSYADSVKG

SEQ ID NO: 182 - protein, 17 aa, synthetic construct
  REGION 1..17: note = Synthetic: SRP1848-A02
GIYPESGATYYADSVKG

SEQ ID NO: 183 - protein, 17 aa, synthetic construct
  REGION 1..17: note = Synthetic: SRP1848-A04
VIYPVDGTTDYADSVKG

SEQ ID NO: 184 - protein, 17 aa, synthetic construct
  REGION 1..17: note = Synthetic: SRP1848-A06
GITPIDGNTDYADSVKG

SEQ ID NO: 185 - protein, 17 aa, synthetic construct
  REGION 1..17: note = Synthetic: SRP1848-A07
EIFPVDGTTDYADSVKG

SEQ ID NO: 186 - protein, 17 aa, synthetic construct
  REGION 1..17: note = Synthetic: SRP1848-A08
SIYPNDGTTDYADSVKG

SEQ ID NO: 187 - protein, 17 aa, synthetic construct
  REGION 1..17: note = Synthetic: SRP1848-A09
SIFPNDGTTDYADSVKG

SEQ ID NO: 188 - protein, 17 aa, synthetic construct
  REGION 1..17: note = Synthetic: SRP1848-A10
DIFPIDDITDYADSVKG

SEQ ID NO: 189 - protein, 17 aa, synthetic construct
  REGION 1..17: note = Synthetic: SRP1848-B01
EIYPVDGITDYADSVKG

SEQ ID NO: 190 - protein, 17 aa, synthetic construct
  REGION 1..17: note = Synthetic: SRP1848-B03
GITPIDGMTDYADSVKG

SEQ ID NO: 191 - protein, 17 aa, synthetic construct
  REGION 1..17: note = Synthetic: SRP1848-B04
EIYPVDGITDYADSVKG

SEQ ID NO: 192 - protein, 17 aa, synthetic construct
  REGION 1..17: note = Synthetic: SRP1848-B05
GISPIDGYTDYADSMKG

SEQ ID NO: 193 - protein, 17 aa, synthetic construct
  REGION 1..17: note = Synthetic: SRP1848-B06
GITPIDGYTDYADSVKG

SEQ ID NO: 194 - protein, 17 aa, synthetic construct
  REGION 1..17: note = Synthetic: SRP1848-B07
GISPYDGFTDYADSVKG

SEQ ID NO: 195 - protein, 17 aa, synthetic construct
  REGION 1..17: note = Synthetic: SRP1848-B09
GITPVDGYTDYADRVKG

SEQ ID NO: 196 - protein, 17 aa, synthetic construct
  REGION 1..17: note = Synthetic: SRP1848-B10
EIYPRDGITDYADSVKG

SEQ ID NO: 197 - protein, 17 aa, synthetic construct
  REGION 1..17: note = Synthetic: SRP1848-B11
DISPIDGFTDYADSVKG

SEQ ID NO: 198 - protein, 17 aa, synthetic construct
  REGION 1..17: note = Synthetic: SRP1848-C01
GVTPNDGYTDYADSVKG

SEQ ID NO: 199 - protein, 17 aa, synthetic construct
  REGION 1..17: note = Synthetic: SRP1848-C03
EIYPIDGNTDYADSVKG

SEQ ID NO: 200 - protein, 17 aa, synthetic construct
  REGION 1..17: note = Synthetic: SRP1848-C04
EIYPGPGNTDYADSVKG

SEQ ID NO: 201 - protein, 17 aa, synthetic construct
  REGION 1..17: note = Synthetic: SRP1848-C05
DIFPIDGINDYADSVKG

SEQ ID NO: 202 - protein, 17 aa, synthetic construct
  REGION 1..17: note = Synthetic: SRP1848-C07
DIFPIDGITDYADSVKG

SEQ ID NO: 203 - protein, 17 aa, synthetic construct
  REGION 1..17: note = Synthetic: SRP1848-C10
GISPIDGYTDYADSMKG

SEQ ID NO: 204 - protein, 17 aa, synthetic construct
  REGION 1..17: note = Synthetic: SRP1848-D02
GITPQDGYTDYADSVKG

SEQ ID NO: 205 - protein, 17 aa, synthetic construct
  REGION 1..17: note = Synthetic: SRP1848-D03
DIFPNDGSTDYADSVKG

SEQ ID NO: 206 - protein, 17 aa, synthetic construct
  REGION 1..17: note = Synthetic: SRP1848-D04
EIYPRDGITDYADSVKG

SEQ ID NO: 207 - protein, 17 aa, synthetic construct
  REGION 1..17: note = Synthetic: SRP1848-D05
GISPIDGYTDYADSVKG

SEQ ID NO: 208 - protein, 17 aa, synthetic construct
  REGION 1..17: note = Synthetic: SRP1848-D07
GISPNDGYTDYADSVKG

SEQ ID NO: 209 - protein, 17 aa, synthetic construct
  REGION 1..17: note = Synthetic: SRP1848-D09
GISPNDGYTDYADSVKG

SEQ ID NO: 210 - protein, 17 aa, synthetic construct
  REGION 1..17: note = Synthetic: SRP1848-D10
WISPNDGTTDYADSVKG

SEQ ID NO: 211 - protein, 17 aa, synthetic construct
  REGION 1..17: note = Synthetic: SRP1848-E01
GITPFDGFTDYADSVKG

SEQ ID NO: 212 - protein, 17 aa, synthetic construct
  REGION 1..17: note = Synthetic: SRP1848-E02
EIYPNDGNTDYADSVKG

SEQ ID NO: 213 - protein, 17 aa, synthetic construct
  REGION 1..17: note = Synthetic: SRP1848-E03
GITPRDGFTDYADSVKG

SEQ ID NO: 214 - protein, 17 aa, synthetic construct
  REGION 1..17: note = Synthetic: SRP1848-E05
GITPNDGYTDYADSVKG

SEQ ID NO: 215 - protein, 17 aa, synthetic construct
  REGION 1..17: note = Synthetic: SRP1848-E06
GITPNDGYTDYADSVEG

SEQ ID NO: 216 - protein, 17 aa, synthetic construct
  REGION 1..17: note = Synthetic: SRP1848-E07
EIFPYDGSTDYADNVKG

SEQ ID NO: 217 - protein, 17 aa, synthetic construct
  REGION 1..17: note = Synthetic: SRP1848-F01
SIFPNDGTTDYADSVKG

SEQ ID NO: 218 - protein, 17 aa, synthetic construct
  REGION 1..17: note = Synthetic: SRP1848-F02
SIFPNDGTTDYADSVKG

SEQ ID NO: 219 - protein, 17 aa, synthetic construct
  REGION 1..17: note = Synthetic: SRP1848-F04
EIYPIDGITDYADSVKG

SEQ ID NO: 220 - protein, 17 aa, synthetic construct
  REGION 1..17: note = Synthetic: SRP1848-F05
EIYPNDGSTDYADSVKG

SEQ ID NO: 221 - protein, 17 aa, synthetic construct
  REGION 1..17: note = Synthetic: SRP1848-F06
GISPIDGNTDYADSVKG

SEQ ID NO: 222 - protein, 17 aa, synthetic construct
  REGION 1..17: note = Synthetic: SRP1848-F07
EIYPNDGITDYADSVKG

SEQ ID NO: 223 - protein, 17 aa, synthetic construct
  REGION 1..17: note = Synthetic: SRP1848-F08
EIYPVDGITDYADSVKG

SEQ ID NO: 224 - protein, 17 aa, synthetic construct
  REGION 1..17: note = Synthetic: SRP1848-F09
GISPLDGYTDYADSVKG

SEQ ID NO: 225 - protein, 17 aa, synthetic construct
  REGION 1..17: note = Synthetic: SRP1848-F10
SIFPNDGYTDYADSVKG

SEQ ID NO: 226 - protein, 17 aa, synthetic construct
  REGION 1..17: note = Synthetic: SRP1848-F11
GITPIDGNTDYADSVKG

SEQ ID NO: 227 - protein, 17 aa, synthetic construct
  REGION 1..17: note = Synthetic: SRP1848-G01
WIAPNDGSTDYADSVKG

SEQ ID NO: 228 - protein, 17 aa, synthetic construct
  REGION 1..17: note = Synthetic: SRP1848-G03
GITPSDGFTDYADSVKG

SEQ ID NO: 229 - protein, 17 aa, synthetic construct
  REGION 1..17: note = Synthetic: SRP1848-G04
YITPAGGATFYADSVKG

SEQ ID NO: 230 - protein, 17 aa, synthetic construct
  REGION 1..17: note = Synthetic: SRP1848-G06
YITPAGGATYYADNVKG

SEQ ID NO: 231 - protein, 17 aa, synthetic construct
  REGION 1..17: note = Synthetic: SRP1848-G07
YITPAGGATWYADSVKG

SEQ ID NO: 232 - protein, 17 aa, synthetic construct
  REGION 1..17: note = Synthetic: SRP1848-G09
YITPAGGATFYADSVKG

SEQ ID NO: 233 - protein, 17 aa, synthetic construct
  REGION 1..17: note = Synthetic: SRP1848-G10
YITPAGGATYYADSVKG

SEQ ID NO: 234 - protein, 17 aa, synthetic construct
  REGION 1..17: note = Synthetic: SRP1848-G11
YITPAGGATWYADSVKG

SEQ ID NO: 235 - protein, 17 aa, synthetic construct
  REGION 1..17: note = Synthetic: SRP1848-H01
DIFPIDGITDYADSVKG

SEQ ID NO: 236 - protein, 16 aa, synthetic construct
  REGION 1..16: note = Synthetic: SRP2060-E10
HIWWDDDKYYHPALKG

SEQ ID NO: 237 - protein, 16 aa, synthetic construct
  REGION 1..16: note = Synthetic: SRP2060-E05
HIWWDDDKYYHPALKG

SEQ ID NO: 238 - protein, 16 aa, synthetic construct
  REGION 1..16: note = Synthetic: SRP2060-B01
HIWWDDDKYYHPALKG

SEQ ID NO: 239 - protein, 16 aa, synthetic construct
  REGION 1..16: note = Synthetic: SRP2060-A06
HIWWDDDKYYYPALKG

SEQ ID NO: 240 - protein, 15 aa, synthetic construct
  REGION 1..15: note = Synthetic: SRP1848-A01
HIYPWDWFSNYVLDY

SEQ ID NO: 241 - protein, 15 aa, synthetic construct
  REGION 1..15: note = Synthetic: SRP1848-A02
HLYVWDWVLDHVLDY

SEQ ID NO: 242 - protein, 15 aa, synthetic construct
  REGION 1..15: note = Synthetic: SRP1848-A04
GAWSWRSGYGYYIDY

SEQ ID NO: 243 - protein, 15 aa, synthetic construct
  REGION 1..15: note = Synthetic: SRP1848-A06
GAWSWRSGYGYYIDY

SEQ ID NO: 244 - protein, 15 aa, synthetic construct
  REGION 1..15: note = Synthetic: SRP1848-A07
GFWAWRSGYGYYLDY

SEQ ID NO: 245 - protein, 15 aa, synthetic construct
  REGION 1..15: note = Synthetic: SRP1848-A08
GSWFWRAGYGYYLDY

SEQ ID NO: 246 - protein, 15 aa, synthetic construct
  REGION 1..15: note = Synthetic: SRP1848-A09
GSWFWRSGYGYFLEY

SEQ ID NO: 247 - protein, 15 aa, synthetic construct
  REGION 1..15: note = Synthetic: SRP1848-A10
GSWSWPSGHSYYLDY

SEQ ID NO: 248 - protein, 15 aa, synthetic construct
  REGION 1..15: note = Synthetic: SRP1848-B01
GFWSWPSGYSYFLDY

SEQ ID NO: 249 - protein, 15 aa, synthetic construct
  REGION 1..15: note = Synthetic: SRP1848-B03
GSWSWPSGYSYYLDY

SEQ ID NO: 250 - protein, 15 aa, synthetic construct
  REGION 1..15: note = Synthetic: SRP1848-B04
GRYSWRAGYSYYLDY

SEQ ID NO: 251 - protein, 15 aa, synthetic construct
  REGION 1..15: note = Synthetic: SRP1848-B05
GSWFWQSGYGYYLDY

SEQ ID NO: 252 - protein, 15 aa, synthetic construct
  REGION 1..15: note = Synthetic: SRP1848-B06
GFWSWPSGYGYYQDY

SEQ ID NO: 253 - protein, 15 aa, synthetic construct
  REGION 1..15: note = Synthetic: SRP1848-B07
GSWSWPAGYGYYQDY

SEQ ID NO: 254 - protein, 15 aa, synthetic construct
  REGION 1..15: note = Synthetic: SRP1848-B09
GAWSWRSGYGYYMDY

SEQ ID NO: 255 - protein, 15 aa, synthetic construct
  REGION 1..15: note = Synthetic: SRP1848-B10
GGWHWRSGYSYYLDY

SEQ ID NO: 256 - protein, 15 aa, synthetic construct
  REGION 1..15: note = Synthetic: SRP1848-B11
GSWSWRAGYGYYLDY

SEQ ID NO: 257 - protein, 15 aa, synthetic construct
  REGION 1..15: note = Synthetic: SRP1848-C01
GSWFWRAGYGYYLDY

SEQ ID NO: 258 - protein, 15 aa, synthetic construct
  REGION 1..15: note = Synthetic: SRP1848-C03
GSWAWRSGYSYYLDY

SEQ ID NO: 259 - protein, 15 aa, synthetic construct
  REGION 1..15: note = Synthetic: SRP1848-C04
GSLSWRAGYGYYLDY

SEQ ID NO: 260 - protein, 15 aa, synthetic construct
  REGION 1..15: note = Synthetic: SRP1848-C05
GSWSWKAGYGYYLDY

SEQ ID NO: 261 - protein, 15 aa, synthetic construct
  REGION 1..15: note = Synthetic: SRP1848-C07
GSWSWPAGYGYYQDY

SEQ ID NO: 262 - protein, 15 aa, synthetic construct
  REGION 1..15: note = Synthetic: SRP1848-C10
GSWSWPAGYGYYLDY

SEQ ID NO: 263 - protein, 15 aa, synthetic construct
  REGION 1..15: note = Synthetic: SRP1848-D02
GAWSWRAGYGYYLDY

SEQ ID NO: 264 - protein, 15 aa, synthetic construct
  REGION 1..15: note = Synthetic: SRP1848-D03
GHWSWPSGYWYYLDY

SEQ ID NO: 265 - protein, 15 aa, synthetic construct
  REGION 1..15: note = Synthetic: SRP1848-D04
GYWFWRSGYGYYLDY

SEQ ID NO: 266 - protein, 15 aa, synthetic construct
  REGION 1..15: note = Synthetic: SRP1848-D05
GSWSWRAGYGYYLDY

SEQ ID NO: 267 - protein, 15 aa, synthetic construct
  REGION 1..15: note = Synthetic: SRP1848-D07
GFWAWRSGYGYYLDY

SEQ ID NO: 268 - protein, 15 aa, synthetic construct
  REGION 1..15: note = Synthetic: SRP1848-D09
GSWSWRHGYGYYLDY

SEQ ID NO: 269 - protein, 15 aa, synthetic construct
  REGION 1..15: note = Synthetic: SRP1848-D10
GAWSWRSGYGYYIDY

SEQ ID NO: 270 - protein, 15 aa, synthetic construct
  REGION 1..15: note = Synthetic: SRP1848-E01
GSWSWPAGYGYYQDY

SEQ ID NO: 271 - protein, 15 aa, synthetic construct
  REGION 1..15: note = Synthetic: SRP1848-E02
GSWSWRSGYGYYLDY

SEQ ID NO: 272 - protein, 15 aa, synthetic construct
  REGION 1..15: note = Synthetic: SRP1848-E03
GSWSWPAGHSYYLDY

SEQ ID NO: 273 - protein, 15 aa, synthetic construct
  REGION 1..15: note = Synthetic: SRP1848-E05
GFWAWRSGYGYYLDY

SEQ ID NO: 274 - protein, 15 aa, synthetic construct
  REGION 1..15: note = Synthetic: SRP1848-E06
GTWSWPSGHSYYLDY

SEQ ID NO: 275 - protein, 15 aa, synthetic construct
  REGION 1..15: note = Synthetic: SRP1848-E07
GAWSWRSGYGYYIDY

SEQ ID NO: 276 - protein, 15 aa, synthetic construct
  REGION 1..15: note = Synthetic: SRP1848-F01
GSWAWRAGYSYYLDY

SEQ ID NO: 277 - protein, 15 aa, synthetic construct
  REGION 1..15: note = Synthetic: SRP1848-F02
GSWSWQAGYGYYLDY

SEQ ID NO: 278 - protein, 15 aa, synthetic construct
  REGION 1..15: note = Synthetic: SRP1848-F04
GSWFWRSGYGYYLDY

SEQ ID NO: 279 - protein, 15 aa, synthetic construct
  REGION 1..15: note = Synthetic: SRP1848-F05
GSWAWRSGYSYFLDY

SEQ ID NO: 280 - protein, 15 aa, synthetic construct
  REGION 1..15: note = Synthetic: SRP1848-F06
GFWAWRSGYGYYLDY

SEQ ID NO: 281 - protein, 15 aa, synthetic construct
  REGION 1..15: note = Synthetic: SRP1848-F07
GSWDWRSGYSYYLDY

SEQ ID NO: 282 - protein, 15 aa, synthetic construct
  REGION 1..15: note = Synthetic: SRP1848-F08
GSWYWQSGYSYYLDY

SEQ ID NO: 283 - protein, 15 aa, synthetic construct
  REGION 1..15: note = Synthetic: SRP1848-F09
GAWSWRSGYGYYIDY

SEQ ID NO: 284 - protein, 15 aa, synthetic construct
  REGION 1..15: note = Synthetic: SRP1848-F10
GSWFWRSGYGYYLDY

SEQ ID NO: 285 - protein, 15 aa, synthetic construct
  REGION 1..15: note = Synthetic: SRP1848-F11
GSWYWRAGYGYYLDY

SEQ ID NO: 286 - protein, 15 aa, synthetic construct
  REGION 1..15: note = Synthetic: SRP1848-G01
GSWAWRSGYSYFLDY

SEQ ID NO: 287 - protein, 15 aa, synthetic construct
  REGION 1..15: note = Synthetic: SRP1848-G03
GSWSWPSGHGYFLDY

SEQ ID NO: 288 - protein, 11 aa, synthetic construct
  REGION 1..11: note = Synthetic: SRP1848-G04
YPYWFAGYMDY

SEQ ID NO: 289 - protein, 11 aa, synthetic construct
  REGION 1..11: note = Synthetic: SRP1848-G06
QPYWFAGYMDY

SEQ ID NO: 290 - protein, 11 aa, synthetic construct
  REGION 1..11: note = Synthetic: SRP1848-G07
YPFWFAGYMDY

SEQ ID NO: 291 - protein, 11 aa, synthetic construct
  REGION 1..11: note = Synthetic: SRP1848-G09
HEYWFSGYMDY

SEQ ID NO: 292 - protein, 11 aa, synthetic construct
  REGION 1..11: note = Synthetic: SRP1848-G10
YPYWFAGYIDY

SEQ ID NO: 293 - protein, 11 aa, synthetic construct
  REGION 1..11: note = Synthetic: SRP1848-G11
YPYWFSGYMDY

SEQ ID NO: 294 - protein, 15 aa, synthetic construct
  REGION 1..15: note = Synthetic: SRP1848-H01
GSWSWPSGMDYYLDY

SEQ ID NO: 295 - protein, 16 aa, synthetic construct
  REGION 1..16: note = Synthetic: SRP2060-E10
NHFPHYYGSSHWYFNV

SEQ ID NO: 296 - protein, 16 aa, synthetic construct
  REGION 1..16: note = Synthetic: SRP2060-E05
NHFPHYYGSSHWYFNV

SEQ ID NO: 297 - protein, 16 aa, synthetic construct
  REGION 1..16: note = Synthetic: SRP2060-B01
NHFPHYYGSSHWYFNV

SEQ ID NO: 298 - protein, 16 aa, synthetic construct
  REGION 1..16: note = Synthetic: SRP2060-A06
NHFPHYYGSSHWYFDV

SEQ ID NO: 299 - protein, 11 aa, synthetic construct
  REGION 1..11: note = Synthetic: trastuzumab
RASQDVNTAVA

SEQ ID NO: 300 - protein, 11 aa, synthetic construct
  REGION 1..11: note = Synthetic: H6D1-LC4
KASQDINSYLS

SEQ ID NO: 301 - protein, 11 aa, synthetic construct
  REGION 1..11: note = Synthetic: H6D1-LC5
KASQDINSYLS

SEQ ID NO: 302 - protein, 7 aa, synthetic construct
  REGION 1..7: note = Synthetic: trastuzumab
SASFLYS

SEQ ID NO: 303 - protein, 7 aa, synthetic construct
  REGION 1..7: note = Synthetic: H6D1-LC4
RANRLVD

SEQ ID NO: 304 - protein, 7 aa, synthetic construct
  REGION 1..7: note = Synthetic: H6D1-LC5
RANRLVD

SEQ ID NO: 305 - protein, 9 aa, synthetic construct
  REGION 1..9: note = Synthetic: trastuzumab
QQHYTTPPT

SEQ ID NO: 306 - protein, 9 aa, synthetic construct
  REGION 1..9: note = Synthetic: H6D1-LC4
LQYDEFPYT

SEQ ID NO: 307 - protein, 9 aa, synthetic construct
  REGION 1..9: note = Synthetic: H6D1-LC5
LQYDEFPYT

SEQ ID NO: 308 - protein, 124 aa, synthetic construct
  REGION 1..124: note = Synthetic: SRP1848-A01
EVQLVESGGGLVQPGGSLRLSCAASGFNITRYSIHWVRQAPGKGLEWVAGILPESGGTSYADSVKGRFTISADTSKNTAYLQMNSLRAEDTAVYYCARHIYPWDWFSNYVLDYWGQGTLVTVSS

SEQ ID NO: 309 - protein, 124 aa, synthetic construct
  REGION 1..124: note = Synthetic: SRP1848-A02
EVQLVESGGGLVQPGGSLRLSCAASGFNISGFRIHWVRQAPGKGLEWVAGIYPESGATYYADSVKGRFTISADTSKNTAYLQMNSLRAEDTAVYYCARHLYVWDWVLDHVLDYWGQGTLVTVSS

SEQ ID NO: 310 - protein, 124 aa, synthetic construct
  REGION 1..124: note = Synthetic: SRP1848-A04
EVQLVESGGGLVQPGGSLRLSCAASGFNIDQSSIHWVRQAPGKGLEWVGVIYPVDGTTDYADSVKGRFTISADTSKNTAYLQMNSLRAEDTAVYYCARGAWSWRSGYGYYIDYWGQGTLVTVSS

SEQ ID NO: 311 - protein, 124 aa, synthetic construct
  REGION 1..124: note = Synthetic: SRP1848-A06
EVQLVESGGGLVQPGGSLRLSCAASGFNIGNSYIHWVRQAPGKGLEWVGGITPIDGNTDYADSVKGRFTISADTSKNTAYLQMNSLRAEDTAVYYCARGAWSWRSGYGYYIDYWGQGTLVTVSS

SEQ ID NO: 312 - protein, 124 aa, synthetic construct
  REGION 1..124: note = Synthetic: SRP1848-A07
EVQLVESGGGLVQPGGSLRLSCAASGFNIGYHSIHWVRQAPGKGLEWVGEIFPVDGTTDYADSVKGRFTISADTSKNTAYLHMNSLRAEDTAVYYCARGFWAWRSGYGYYLDYWGQGTLVTVSS

SEQ ID NO: 313 - protein, 124 aa, synthetic construct
  REGION 1..124: note = Synthetic: SRP1848-A08
EVQLVESGGGLVQPGGSLRLSCAASGSNIRKHSIHWVRQAPGKGLEWVGSIYPNDGTTDYADSVKGRFTISADTSKNTAYLQMNSLRAEDTAVYYCARGSWFWRAGYGYYLDYWGQGTLVTVSS

SEQ ID NO: 314 - protein, 124 aa, synthetic construct
  REGION 1..124: note = Synthetic: SRP1848-A09
EVQLVESGGGLVQPGGSLRLSCAASGFNIRKQSIHWVRQAPGKGLEWVGSIFPNDGTTDYADSVKGRFTISADTSKNTAYLQVNSLRAEDTAVYYCARGSWFWRSGYGYFLEYWGQGTLVTVSS

SEQ ID NO: 315 - protein, 124 aa, synthetic construct
  REGION 1..124: note = Synthetic: SRP1848-A10
EVQLVESGGGLVQPGGSLRLSCAASGFNIRKYSIHWARQAPGKGLEWVGDIFPIDDITDYADSVKGRFTISADTSKNTAYLQMNSLRAEDTAVYYCARGSWSWPSGHSYYLDYWGQGTLVTVSS

SEQ ID NO: 316 - protein, 124 aa, synthetic construct
  REGION 1..124: note = Synthetic: SRP1848-B01
EVQLVESGGGLVQPGGSLRLSCAASGFNIRNYSIHWVRQAPGKGLEWVGEIYPVDGITDYADSVKGRFTISADTSKNTAYLQMNSLRAEDTAVYYCARGFWSWPSGYSYFLDYWGQGTLVTVSS

SEQ ID NO: 317 - protein, 124 aa, synthetic construct
  REGION 1..124: note = Synthetic: SRP1848-B03
EVQLVESGGGLVQPGGSLRLSCAASGFNISMKYIHWVRQAPGKGLEWVGGITPIDGMTDYADSVKGRFTISADTSKNTAYLQMNSLRAEDTAVYYCARGSWSWPSGYSYYLDYWGQGTLVTVSS

SEQ ID NO: 318 - protein, 124 aa, synthetic construct
  REGION 1..124: note = Synthetic: SRP1848-B04
EVQLVESGGGLVQPGGSLRLSCAASSFNISNHSIHWVRQAPGKGLEWVGEIYPVDGITDYADSVKGRFTISADTSKNTAYLQMNSLRAEDTAVYYCARGRYSWRAGYSYYLDYWGQGTLVTVSS

SEQ ID NO: 319 - protein, 124 aa, synthetic construct
  REGION 1..124: note = Synthetic: SRP1848-B05
EVQLVESGGGLVQPGGSLRLSCAASGFNISNYYIHWVRQAPGKGLEWVGGISPIDGYTDYADSMKGRFTISADTSKNTAYLQMSSLRAEDTAVYYCARGSWFWQSGYGYYLDYWGQGTLVTVSS

SEQ ID NO: 320 - protein, 124 aa, synthetic construct
  REGION 1..124: note = Synthetic: SRP1848-B06
EVQLVESGGGLVQPGGSLRLSCAASGFNISNYYIHWVRQAPGKGLEWVGGITPIDGYTDYADSVKGRFTISADTSKNTAYLQMNSLRAEDTAVYYCARGFWSWPSGYGYYQDYWGQGTLVTVSS

SEQ ID NO: 321 - protein, 124 aa, synthetic construct
  REGION 1..124: note = Synthetic: SRP1848-B07
EVQLVESGGGLVQPGGSLRLSCAASGFNISRFYIHWVRQAPGKGLEWVGGISPYDGFTDYADSVKGRFTISADTSKNTAYLQMNSLRAEDTAVYYCARGSWSWPAGYGYYQDYWGQGTLVTVSS

SEQ ID NO: 322 - protein, 124 aa, synthetic construct
  REGION 1..124: note = Synthetic: SRP1848-B09
EVQLVESGGGLVQPGGSLRLSCAAGGFNITNYYIHWVRQAPGKGLEWVGGITPVDGYTDYADRVKGRFTISADTSKNTAYLQMNSLRAEDTAVYYCARGAWSWRSGYGYYMDYWGQGTLVTVSS

SEQ ID NO: 323 - protein, 124 aa, synthetic construct
  REGION 1..124: note = Synthetic: SRP1848-B10
EVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSS

SEQ ID NO: 324 - protein, 124 aa, synthetic construct
  REGION 1..124: note = Synthetic: SRP1848-B11
EVQLVESGGGLVQPGGSLRLSCAASGFNIGNNSIHWVRQAPGKGLEWVGDISPIDGFTDYADSVKGRFTISADTSKNTAYLQMNSLRAEDTAVYYCARGSWSWRAGYGYYLDYWGQGTLVTVSS

SEQ ID NO: 325 - protein, 124 aa, synthetic construct
  REGION 1..124: note = Synthetic: SRP1848-C01
EVQLVESGGGLVQPGGSLRLSCAASGFNIGNSYIHWVRQAPGKGLEWVGGVTPNDGYTDYADSVKGRFTISADTSKNTTYLQMNSLRAEDTAVYYCARGSWFWRAGYGYYLDYWGQGALVTVSS

SEQ ID NO: 326 - protein, 124 aa, synthetic construct
  REGION 1..124: note = Synthetic: SRP1848-C03
EVQLVESGGGLVQPGGSLRLSCAASGFNIGVYSIHWVRQAPGKGLEWVGEIYPIDGNTDYADSVKGRFTISADTSKNTAYLQMNSLRAEDTAVYYCARGSWAWRSGYSYYLDYWGQGTLVTVSS

SEQ ID NO: 327 - protein, 124 aa, synthetic construct
  REGION 1..124: note = Synthetic: SRP1848-C04
EVQLVESGGGLVQPGGSLRLSCAASGFNIRHYSIHWVRQAPGKGLEWVGEIYPGPGNTDYADSVKGRFTISADTSKNTAYLQMNSLRAEDTAVYYCARGSLSWRAGYGYYLDYWGQGTLVTVSS

SEQ ID NO: 328 - protein, 124 aa, synthetic construct
  REGION 1..124: note = Synthetic: SRP1848-C05
EVQLVESGGGLVQPGGSLRLSCAASGFNIRKYSIHWVRQAPGKGLEWVGDIFPIDGINDYADSVKGRFTISADTSKNTAYLQMNSLRAEDTAVYYCARGSWSWKAGYGYYLDYWGQGTLVTVSS

SEQ ID NO: 329 - protein, 124 aa, synthetic construct
  REGION 1..124: note = Synthetic: SRP1848-C07
EVQLVESGGGLVQPGGSLRLSCAASGFNIRKYSIHWVRQAPGKGLEWVGDIFPIDGITDYADSMKGRFTISADTSKNTAYLQMNSLRAEDTAVYYCARGSWSWPAGYGYYQDYWGQGTLVTVSS

SEQ ID NO: 330 - protein, 124 aa, synthetic construct
  REGION 1..124: note = Synthetic: SRP1848-C10
EVQLVESGGGLVQPGGSLRLSCAASGFNIRTYYIHWVRQAPGKGLEWVGGISPIDGYTDYADSVKGRFTISADTSKNTAYLQMNSLRAEDTAVYYCARGSWSWPAGYGYYLDYWGQGTLVTVSS

SEQ ID NO: 331 - protein, 124 aa, synthetic construct
  REGION 1..124: note = Synthetic: SRP1848-D02
EVQLVESGGGLVQPGGSLRLSCAASGFNISHNYIHWVRQAPGKGLEWVGGITPQDGYTDYADSVKGRFTISADTSKNTAYLQMNRLRAEDTAVYYCARGAWSWRAGYGYYLDYWGQGTLVTVSS

SEQ ID NO: 332 - protein, 124 aa, synthetic construct
  REGION 1..124: note = Synthetic: SRP1848-D03
EVQLVESGGGVVQPGGSLRLSCAASGFNIRYFSIHWVRQAPGKGLEWVGDIFPNDGSTDYADSVKGRFTISADTSKNTAYLQMNSLRAEETAVYYCARGHWSWPSGYWYYLDYWGQGTLVTVSS

SEQ ID NO: 333 - protein, 124 aa, synthetic construct
  REGION 1..124: note = Synthetic: SRP1848-D04
EVQLVESGGGLVQPGGSLRLSCAASGFNISHYSIHWVRQAPGKGLEWVGEIYPRDGITDYADSVKGRFTISADTSKNTAYLQMNSLSAEDTAVYYCARGYWFWRSGYGYYLDYWGQGTLVTVSS

SEQ ID NO: 334 - protein, 124 aa, synthetic construct
  REGION 1..124: note = Synthetic: SRP1848-D05
EVQLVESGGGLVQPGGSLRLSCAASGFNISISYIHWVRQAPGKGLEWVGGISPIDGYTDYADSVKGRFTISADTSKNTAYLQMNSLRAEDTAVYYCARGSWSWRAGYGYYLDYWGQGTLVTVSS

SEQ ID NO: 335 - protein, 124 aa, synthetic construct
  REGION 1..124: note = Synthetic: SRP1848-D07
EVQLVESGGGLVQPGGSLRLSCAASGFNISKYYIHWVRQAPGKGLEWVGGISPNDGYTDYADSVKGRFAISADTSKNTAYLQMNSLRAEDTAVYYCARGFWAWRSGYGYYLDYWGQGTLVTVSS

SEQ ID NO: 336 - protein, 124 aa, synthetic construct
  REGION 1..124: note = Synthetic: SRP1848-D09
EVQLVESGGGLVQPGGSLRLSCAASGFNISNYYIHWVRQAPGKGLEWVGGISPNDGYTDYADSVKGRFTISADTSKNTAYLQMNSLRAEDTAVYYCARGSWSWRHGYGYYLDYWGQGTLVTVSS

SEQ ID NO: 337 - protein, 124 aa, synthetic construct
  REGION 1..124: note = Synthetic: SRP1848-D10
EVQLVESGGGLVQPGGSLRLSCAASGFNISRNSIHWVRQAPGKGLEWVGWISPNDGTTDYADSVKGRFTISADGSKNTAYLQMNSLRAEDTAVYYCARGAWSWRSGYGYYIDYWGQGTLVTVSS

SEQ ID NO: 338 - protein, 124 aa, synthetic construct
  REGION 1..124: note = Synthetic: SRP1848-E01
EVQLVESGGGLVQPGGSLRLSCAASGFNITNKYIHWVRQAPGKGLEWVGGITPFDGFTDYADSVKGRFTISADTSKNTAYLQMNSLRAEDTAVYYCARGSWSWPAGYGYYQDYWGQGTLVTVSS

SEQ ID NO: 339 - protein, 124 aa, synthetic construct
  REGION 1..124: note = Synthetic: SRP1848-E02
EVQLVESGGGLVQPGGSLRLSCAASGFNIGKYSIHWVRQAPGKGLEWVGEIYPNDGNTDYADSVKGRFTISADTSKNTAYLQMNSLRAEDTAVYYCARGSWSWRSGYGYYLDYWGQGTLVTVSS

SEQ ID NO: 340 - protein, 124 aa, synthetic construct
  REGION 1..124: note = Synthetic: SRP1848-E03
EVQLVESGGGLAQPGGSLRLSCAASGFNIGNYYIHWVRQAPGKGLEWVGGITPRDGFTDYADSVKGRFTISADTSKNTAYLQVNSLRAEDTAVYYCARGSWSWPAGHSYYLDYWGQGTLVTVSS

SEQ ID NO: 341 - protein, 124 aa, synthetic construct
  REGION 1..124: note = Synthetic: SRP1848-E05
EVQLVESGGGLVQPGGSLRVSCAASGFNIGVYYIHWVRQAPGKGLEWVGGITPNDGYTDYADSVKGRFTISADTSKNTAYLQMNSLRAEDTAVYYCARGFWAWRSGYGYYLDYWGQGTLVTVSS

SEQ ID NO: 342 - protein, 124 aa, synthetic construct
  REGION 1..124: note = Synthetic: SRP1848-E06
EVQLVESGGGLVQPSGSLRLSCAASGFNINRYYIHWVRQAPGKGLEWVGGITPNDGYTDYADSVEGRFTTSADTSKNTAYLQMNSLRAEDTAVYYCARGTWSWPSGHSYYLDYWGQGTLVTVSS

SEQ ID NO: 343 - protein, 124 aa, synthetic construct
  REGION 1..124: note = Synthetic: SRP1848-E07
EVQLVESGGGLVQPGGSLRLSCAASGFNIRKSSIHWVRQAPGKGLEWVGEIFPYDGSTDYADNVKGRFTISADTSKNTAYLQMNSLRAEDTAVYYCARGAWSWRSGYGYYIDYWGQGTLVTVSS

SEQ ID NO: 344 - protein, 124 aa, synthetic construct
  REGION 1..124: note = Synthetic: SRP1848-F01
EVQLVESGGGLVQPGGSLRLSCAASGFNIRTYSIHWVRQAPGKGLEWVGSIFPNDGTTDYADSVKGRFTISADTSKNTAYLQMNSLRAEDTAVYYCARGSWAWRAGYSYYLDYWGQGTLVTVSS

SEQ ID NO: 345 - protein, 124 aa, synthetic construct
  REGION 1..124: note = Synthetic: SRP1848-F02
EVQLVESGGGLVQPGGSLRLSCAASGFNIRTYSIHWVRQAPGKGLEWVGSIFPNDGTTDYADSVKGRLTISADTSKNTAYLQMNSLRAEDTAVYYCARGSWSWQAGYGYYLDYWGQGTLVTVSS

SEQ ID NO: 346 - protein, 124 aa, synthetic construct
  REGION 1..124: note = Synthetic: SRP1848-F04
EVQLVESGGGLVQPGGSLRLSCAASGFNISNYSIHWVRQAPGKGLEWVGEIYPIDGITDYADSVKGRFTISADTSKNTAYLQMNSLRAEDTAVYYCARGSWFWRSGYGYYLDYWGQGTLVTVSS

SEQ ID NO: 347 - protein, 124 aa, synthetic construct
  REGION 1..124: note = Synthetic: SRP1848-F05
EVQLVESGGGLVQPGGSLRLSCAASGFNISKSSIHWVRQAPGKGLEWVGEIYPNDGSTDYADSVKGRFTISADTSKNTAYLQMNSLRAEDTAVYYCARGSWAWRSGYSYFLDYWGQGTLVTVSS

SEQ ID NO: 348 - protein, 124 aa, synthetic construct
  REGION 1..124: note = Synthetic: SRP1848-F06
EVQLVESGGGLVQPGGSLRLSCAASGFNISLSYIHWVRQAPGKGLEWVGGISPIDGNTDYADSVKGRFTISADTSKNTAYLQMNSLRAEDTAVYYCARGFWAWRSGYGYYLDYWGQGTLVTVSS

SEQ ID NO: 349 - protein, 124 aa, synthetic construct
  REGION 1..124: note = Synthetic: SRP1848-F07
EVQLVESGGGLVQPGGSLRLSCAASGFNISNHSIHWVRQAPGKGLEWVGEIYPNDGITDYADSVKGRFTISADTSKNTAYLQMNSLSAEDTAVYYCARGSWDWRSGYSYYLDYWGQGTLVTVSS

SEQ ID NO: 350 - protein, 124 aa, synthetic construct
  REGION 1..124: note = Synthetic: SRP1848-F08
EVQLVESGGGLVQPGGSLRLSCAAGGFNISNHSIHWVRQAPGKGVEWVGEIYPVDGITDYADSVKGRFTISADTSKNTAYLRMNSLRAEDTAVYYCARGSWYWQSGYSYYLDYWGQGTLVTVSS

SEQ ID NO: 351 - protein, 124 aa, synthetic construct
  REGION 1..124: note = Synthetic: SRP1848-F09
EVQLVESGGGLVQPGGSLRLSCAASGFNISNHYIHWVRQAPGKGLEWVGGISPLDGYTDYADSVKGRFTISADTSKNTAYLQMNSLRAEDTAVYYCARGAWSWRSGYGYYIDYWGQGTLVTVSS

SEQ ID NO: 352 - protein, 124 aa, synthetic construct
  REGION 1..124: note = Synthetic: SRP1848-F10
EVQLVESGGGLVQPGGSLRLSCAASGFNISNNSIHWVRQAPGKGLEWVGSIFPNDGYTDYADSVKGRFTISADTSKNTAYLQMNSLRAEDTAVYYCARGSWFWRSGYGYYLDYWGQGTLVTVSS

SEQ ID NO: 353 - protein, 124 aa, synthetic construct
  REGION 1..124: note = Synthetic: SRP1848-F11
EVQLVESGGGLVQPGGSLRLSCAASGFNISNNYIHWVRQAPGKGLEWVGGITPIDGNTDYADSVKGRFTISADTSMNTAYLQMNSLRAEDTAVYYCARGSWYWRAGYGYYLDYWGQGALVTVSS

SEQ ID NO: 354 - protein, 124 aa, synthetic construct
  REGION 1..124: note = Synthetic: SRP1848-G01
EVQLVESGGGLVQPGGSLRLSCAASGFNISRHSIHWVRQAPGKGLEWVGWIAPNDGSTDYADSVKGRFTISADTSKNTAYLQMNSLRAEDTAVYYCARGSWAWRSGYSYFLDYWGQGTLVTVSS

SEQ ID NO: 355 - protein, 124 aa, synthetic construct
  REGION 1..124: note = Synthetic: SRP1848-G03
EVQLVESGGGLVQPGGSLRLSCAASGFNISTYYIHWVRQAPGKGLEWVGGITPSDGFTDYADSVKGRSTISADTSKNTAYLQMNSLRAEDTAVYYCARGSWSWPSGHGYFLDYWGQGTLVTVSS

SEQ ID NO: 356 - protein, 120 aa, synthetic construct
  REGION 1..120: note = Synthetic: SRP1848-G04
EVQLVESGGGLVQPGGSLRLSCAASGFNIHSTDIHWVRQAPGKGLEWVAYITPAGGATFYADSVKGRFTISADTSKNTAYLQMNSLRAEDTAVYYCARYPYWFAGYMDYWGQGTLVTVSS

SEQ ID NO: 357 - protein, 120 aa, synthetic construct
  REGION 1..120: note = Synthetic: SRP1848-G06
EVQLVESGGGLVQPGGSLRLSCAASGFNIRSTDIHWVRQAPGKGLEWVAYITPAGGATYYADNVKGRFTISADTSKNTAYLQMNSLRAEDTAVYYCARQPYWFAGYMDYWGQGTLVTVSS

SEQ ID NO: 358 - protein, 120 aa, synthetic construct
  REGION 1..120: note = Synthetic: SRP1848-G07
EVQLVESGGGLVQPGGSLRLSCAASGFNIHSTDIHWVRQAPGKGLEWVAYITPAGGATWYADSVKGRFTISADTSKNTAYLQMNSLRAEDTAVYYCARYPFWFAGYMDYWGQGTLVTVSS

SEQ ID NO: 359 - protein, 120 aa, synthetic construct
  REGION 1..120: note = Synthetic: SRP1848-G09
EVQLVESGGGLVQPGGSLRLSCAASGFNIRGTDIHWVRQAPGKGLEWVAYITPAGGATFYADSVKGRFTISADTSKNTAYLQMNSLRAEDTAVYYCARHEYWFSGYMDYWGQGTLVTVSS

SEQ ID NO: 360 - protein, 120 aa, synthetic construct
  REGION 1..120: note = Synthetic: SRP1848-G10
EVQLVESGGGLVQPGSSLRLSCAASGFNIRSTDIHWVRQAPGKGLEWVAYITPAGGATYYADSVKGRFTISADTSKNTAYLQMNSLRAEDTAVYYCARYPYWFAGYIDYWGQGTLVTVSS

SEQ ID NO: 361 - protein, 120 aa, synthetic construct
  REGION 1..120: note = Synthetic: SRP1848-G11
EVQLVESGGGLVQPGGSLRLSCAASGFNISSTDIHWVRQAPGKGLEWVAYITPAGGATWYADSVKGRFTISADTSKNTAYLQMNSLRAEDTAVYYCARYPYWFSGYMDYWGQGTLVTVSS

SEQ ID NO: 362 - protein, 124 aa, synthetic construct
  REGION 1..124: note = Synthetic: SRP1848-H01
EVQLVESGGGLVQPGGSLRLSCAASGFNIRTQSIHWVRQAPGKGLEWIGDIFPIDGITDYADSVKGRFTISADTSKNTAYLQMNSLRAEDTAVYYCARGSWSWPSGMDYYLDYWGQGTLVTVSS

SEQ ID NO: 363 - protein, 126 aa, synthetic construct
  REGION 1..126: note = Synthetic: SRP2060-E10
EVQLLESGGGLVQPGGSLRLSCAFSGFSLSTFGMGVGWVRQAPGKGLEWVSHIWWDDDKYYHPALKGRFTISKDNSKNTVYLQMNSLRAEDTAVYYCGRNHFPHYYGSSHWYFNVWGQGTTVTVSS

SEQ ID NO: 364 - protein, 126 aa, synthetic construct
  REGION 1..126: note = Synthetic: SRP2060-E05
EVQLLESGGGLVQPGGSLRLSCAFSGFSLSTFGMGVGWVRQAPGKGLEWVSHIWWDDDKYYHPALKGRFTVSKDNSKNTVYLQMNSLRAEDTAVYYCGRNHFPHYYGSSHWYFNVWGQGTTVTVSS

SEQ ID NO: 365 - protein, 126 aa, synthetic construct
  REGION 1..126: note = Synthetic: SRP2060-B01
EVQLLESGGGLVQPGGSLRLSCALSGFSLSTFGMGVGWVRQATGKGLEWVSHIWWDDDKYYHPALKGRFTISKDNSKNTVHLQMNSLRAEDTAVYYCGRNHFPHYYGSSHWYFNVWGQGTTVTVSS

SEQ ID NO: 366 - protein, 126 aa, synthetic construct
  REGION 1..126: note = Synthetic: SRP2060-A06
EVQLLESGGGLVQPGGSLRLSCAFSGFSLSTFGMGVGWVRQAPGKGLEWVGHIWWDDDKYYYPALKGRFTISKDNSKNTVYLQMNSLRAEDTAVYYCGRNHFPHYYGSSHWYFDVWGQGTTVTVSS

SEQ ID NO: 367 - protein, 107 aa, synthetic construct [sequence omitted]

SEQ ID NO: 368 - protein, 107 aa, synthetic construct
  REGION 1..107: note = Synthetic: H6D1-LC4
EIVMTQSPATLSLSPGERATLSCKASQDINSYLSWYQQKPGQAPRLLIYRANRLVDGIPARFSGSGSGTDYTLTISSLEPEDFAVYYCLQYDEFPYTFGGGTKVEIK

SEQ ID NO: 369 - protein, 107 aa, synthetic construct
  REGION 1..107: note = Synthetic: H6D1-LC5
DIQMTQSPSTLSASVGDRVTITCKASQDINSYLSWYQQKPGKAPKLLIYRANRLVDGVPSRFSGSGSGTEFTLTISSLQPDDFATYYCLQYDEFPYTFGGGTKVEIK

SEQ ID NO: 370 - protein, 330 aa, Homo sapiens
  REGION 1..330: note = misc_feature - Human IgG1 HC Constant
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371 - protein, 107 aa, Homo sapiens [sequence omitted]

SEQ ID NO: 372 - protein, 323 aa, Mus musculus
  REGION 1..323: note = misc_feature - Mouse IgG1 HC Constant
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 373 - protein, 107 aa, Mus musculus
  REGION 1..107: note = misc_feature - Mouse IgG LC Constant Ckappa
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 374 - protein, 108 aa, synthetic construct
  REGION 1..108: note = Synthetic: Kappa LC
HMTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 375 - protein, 106 aa, synthetic construct
  REGION 1..106: note = Synthetic: Lambda LD
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 376 - protein, 20 aa, synthetic construct
  REGION 1..20: note = Synthetic: FlagHis Tag
GSGDYKDDDDKGSGHHHHHH

SEQ ID NO: 377 - protein, 15 aa, synthetic construct
  REGION 1..15: note = Synthetic: Linker
GGGGSGGGGSGGGGS

SEQ ID NO: 378 - protein, 11 aa, synthetic construct
  REGION 1..11: note = Synthetic: Linker
AAGSDQEPKSS

SEQ ID NO: 379 - protein, 247 aa, synthetic construct
  REGION 1..247: note = Synthetic: 1848-B10-VH-(G4S)3-VL
MEVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 380 - protein, 247 aa, synthetic construct
  REGION 1..247: note = Synthetic: 1848-B10-VL-(G4S)3-VH
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSS

SEQ ID NO: 381 - protein, 485 aa, synthetic construct
  REGION 1..485: note = Synthetic: 1848-B10-VH-(G4S)3-VL
MEVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKAAGSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 382 - protein, 485 aa, synthetic construct
  REGION 1..485: note = Synthetic: 1848-B10-VL-(G4S)3-VH
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSSAAGSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0408228 A1
(43) Pub. Date: Dec. 12, 2024

(54) ANTI-FOLATE RECEPTOR CONJUGATE CANCER THERAPY

(71) Applicant: SUTRO BIOPHARMA, INC., South San Francisco, CA (US)

(72) Inventors: Hatem DOKAINISH, South San Francisco, CA (US); Lin LU, South San Francisco, CA (US); Craig Jerome BERMAN, South San Francisco, CA (US); Arturo MOLINA, South San Francisco, CA (US)

(21) Appl. No.: 18/715,623

(22) PCT Filed: Nov. 30, 2022
(86) PCT No.: PCT/US2022/051449

§ 371 Date: May 31, 2024

Related U.S. Application Data
(60) Provisional application No. 63/264,784, filed on Dec. 1, 2021.
(60) Provisional application No. 63/332,973, filed on Apr. 20, 2022.

(51) Int. Cl. A61K 47/68; A61P 35/00; C07K 16/28

(52) U.S. Cl. CPC A61K 47/6849; A61K 47/68031; A61P 35/00; C07K 16/28; C07K 2317/24

(57) ABSTRACT

The present disclosure relates to therapies with antibody conjugates with binding specificity for folate receptor alpha (FOLR1) and its isoforms and homologs. Also provided are methods of using the combinations with antibody conjugates and compositions thereof, such as in therapeutic and diagnostic methods.

### FIELD OF THE INVENTION

The present application claims the benefit of US provisional application nos. 63/264,784, filed Dec. 1, 2021, and 63/332,973, filed Apr. 20, 2022, the contents of which are hereby incorporated by reference in their entireties.

### FIELD OF THE INVENTION

Provided herein are cancer therapies with antibody conjugates having binding specificity for folate receptor alpha (FolRα or FOLR1) and compositions for administering the same, including pharmaceutical compositions. The cancer therapies are useful in methods of treatment and prevention of cell proliferation and cancer. The cancer therapies are also useful in methods of treatment and prevention, of autoimmune diseases, infectious diseases, and inflammatory conditions.

### BACKGROUND

Folate receptors, or folate binding proteins (FBPs), include single chain glycoproteins that bind and contribute to the update of folates and other compounds in vivo. Elwood, 1989, J. Biol. Chem. 264:14893-14901. Certain folate receptors are single-chain glycoproteins with a high affinity binding site for folate and other compounds such as methotrexate. Elwood, p. 14893. The human FOLR1 gene encodes the adult folate receptor, a 30 kDa polypeptide with about 257 amino acids with three potential N-linked glycosylation sites. Elwood, p. 14893; Lacey et al., 1989, J. Clin. Invest. 84:715-720. Homologous genes and polypeptides have been identified in dozens of species.

The mature folate receptor glycoprotein has a size of about 42 kDa and has been observed to participate in the internalization of folates and antifolates into cells. Elwood et al., 1997, Biochemistry 36:1467-1478. Expression has been observed in human cerebellum and kidney cells, along with human cancer cell lines. Elwood et al., 1997, p. 1467. In addition to internalization of folate, a folate receptor has been shown to be a significant cofactor for cellular entry of viruses, particularly Marburg and Ebola viruses. Chan et al., 2001, Cell 106:117-126. Due to these internalization properties, the folate receptor has been proposed as a target for diagnostic and therapeutic agents. For instance, diagnostic and therapeutic agents have been linked to folate for internalization into cells expressing the folate receptor. See, e.g., Leamon, 2008, Curr. Opin. Investig. Drugs 9:1277-1286; Paulos et al., 2004, Adv. Drug Del. Rev. 56:1205-1217.

Folate receptor alpha (FolRα or FOLR1) is a glycosylphosphatidylinositol linked cell-surface glycoprotein that has high affinity for folates. Except for low levels in kidney and lung, most normal tissues do not express FOLR1, but high levels of FOLR1 have been found in serous and endometrioid epithelial ovarian cancer, endometrial adenocarcinoma, non-small cell lung carcinoma (NSCLC) of the adenocarcinoma subtype, and triple-negative breast cancer (TNBC). FOLR1 expression is maintained in metastatic foci and recurrent carcinomas in ovarian cancer patients, and FOLR1 expression has been observed after chemotherapy in epithelial ovarian and endometrial cancers. These properties, together with the highly restricted expression of FOLR1 on normal tissues, make FOLR1 a highly promising target for cancer therapy. As such, the folate receptor provides a potential target for diagnostics and therapeutics for cancers and inflammatory conditions.

There is a need for improved therapeutic methods of modulating the immune regulation of folate receptor alpha (FolRα or FOLR1) and the downstream signaling processes activated by folate receptor alpha (FolRα or FOLR1) for treatment, particularly improved methods that provide safe and effective doses of therapeutic conjugates directed to FOLR1.

### SUMMARY

Provided herein are methods of treating cancer in human patients with antibody conjugates that selectively bind folate receptor alpha (FolRα or FOLR1), at particular doses and on particular dosing schedules. The antibody conjugates comprise an antibody that binds folate receptor alpha (FolRα or FOLR1) linked to one or more payload moieties. The antibody can be linked to the payload directly by a covalent bond or indirectly by way of a linker. Folate receptor alpha (FolRα or FOLR1) antibodies are described in detail herein, as are useful payload moieties, and useful linkers.

In one aspect, provided herein are methods of using the anti-FOLR1 antibody conjugates and compositions. In certain embodiments, the methods are methods of treatment. In certain embodiments, the conjugates are administered on a dosing schedule. In certain embodiments, the conjugates are administered at a first dose for 1-3 doses and then at a second dose for at least 1-3 doses. In certain embodiments, the first dose is higher than the second dose. In certain embodiments, the first dose is about 5.2 mg/kg. In certain embodiments, the second dose is about 4.3 mg/kg. In certain embodiments, the first dose is about 4.3 mg/kg. In certain embodiments, the second dose is about 3.5 mg/kg. In certain embodiments, the second dose is maintained throughout the remainder of therapy. In certain embodiments, there is a third dose of about 3.5 mg/kg or about 2.9 mg/kg. In certain embodiments, there is a fourth dose of about 2.9 mg/kg. In particular embodiments, each new dose is lower than the previous dose. In certain embodiments, the method is used to treat a disease or condition. In certain embodiments, the disease or condition is a cancer.

In certain embodiments, provided herein are methods of treating cancer in a subject in need thereof, comprising the steps of administering to the subject a first dose of a folate receptor alpha (FOLR1) antibody conjugate, wherein the FOLR1 antibody conjugate comprises an antibody linked to at least one payload moiety, and wherein the first dose is about 5.2 mg/kg; and administering to the subject a second dose of the FOLR1 antibody conjugate, wherein the second dose is administered after the first dose, wherein the amount of the second dose is less than the amount of the first dose, and wherein the first dose and second dose are administered at least about three weeks apart.

In certain embodiments, provided herein are methods of treating cancer in a subject in need thereof, comprising the steps of administering to the subject a first dose of a folate receptor alpha (FOLR1) antibody conjugate, wherein the FOLR1 antibody conjugate comprises an antibody linked to at least one payload moiety, and wherein the first dose is about 4.3 mg/kg; and administering to the subject a second dose of the FOLR1 antibody conjugate, wherein the second dose is administered after the first dose, wherein the amount of the second dose is less than the amount of the first dose, and wherein the first dose and second dose are administered at least about three weeks apart.

In certain embodiments, provided herein are methods of treating cancer in a subject in need thereof, comprising: administering to the subject a first dose of a folate receptor alpha (FOLR1) antibody conjugate, wherein the FOLR1 antibody conjugate comprises an antibody linked to at least one payload moiety; and administering to the subject a second dose of the FOLR1 antibody conjugate, wherein the second dose is about 4.3 mg/kg or less, wherein the second dose is administered after the first dose, and wherein the amount of the first dose is greater than the amount of the second dose, and wherein the first dose is administered once every three weeks or longer for one to six cycles.

In certain embodiments, the first dose is about 5.2 mg/kg and the second dose is selected from about 4.3 mg/kg, about 3.5 mg/kg, and about 2.9 mg/kg. In certain embodiments, there is a third dose, and the third dose is selected from about 3.5 mg/kg and about 2.9 mg/kg. In certain embodiments, there is a fourth dose, and the fourth dose is about 2.9 mg/kg. In certain embodiments, the doses are about 5.2 mg/kg and about 4.3 mg/kg in sequence. In certain embodiments, the doses are about 5.2 mg/kg, about 4.3 mg/kg, and about 3.5 mg/kg in sequence. In certain embodiments, the doses are about 5.2 mg/kg, about 4.3 mg/kg, about 3.5 mg/kg, and about 2.9 mg/kg in sequence. In certain embodiments, the doses are about 5.2 mg/kg, about 4.3 mg/kg, and about 2.9 mg/kg in sequence. In certain embodiments, the doses are about 5.2 mg/kg and about 3.5 mg/kg in sequence. In certain embodiments, the doses are about 5.2 mg/kg, about 3.5 mg/kg, and about 2.9 mg/kg in sequence. In certain embodiments, the doses are about 5.2 mg/kg and about 2.9 mg/kg in sequence. The dosing cycles are as described herein for each first, second, third, or fourth dose. In particular embodiments, each dose is administered about three weeks from the previous and subsequent doses.

In certain embodiments, the first dose is about 4.3 mg/kg and the second dose is selected from about 3.5 mg/kg and about 2.9 mg/kg. In certain embodiments, the first dose is about 4.3 mg/kg and the second dose is about 3.5 mg/kg. the first dose is about 4.3 mg/kg and the second dose is about 2.9 mg/kg. In certain embodiments, there is a third dose, and the third dose is about 2.9 mg/kg, following a second dose of 3.5 mg/kg and a first dose of about 4.3 mg/kg. In certain embodiments, the doses are about 4.3 mg/kg and about 3.5 mg/kg in sequence. In certain embodiments, the doses are about 4.3 mg/kg, about 3.5 mg/kg, and about 2.9 mg/kg in sequence. In certain embodiments, the doses are about 4.3 mg/kg, and about 2.9 mg/kg in sequence. The dosing cycles are as described herein for each first, second, or third dose. In particular embodiments, each dose is administered about three weeks from the previous and subsequent doses.

In certain embodiments, provided herein are methods of treating cancer in a subject in need thereof, comprising: administering to the subject a first dose of a folate receptor alpha (FOLR1) antibody conjugate, wherein the FOLR1 antibody conjugate comprises an antibody linked to at least one payload moiety; and administering to the subject a second dose of the FOLR1 antibody conjugate, wherein the second dose is administered after the first dose, wherein the amount of the first dose is the same or greater than the amount of the second dose, wherein a tumor tissue sample from the subject has a tumor proportion score (TPS) of about 25% or more, and wherein the subject has a tumor reduction of at least 30% by volume after three weeks from administering the first dose.

In certain embodiments, the FOLR1 antibody conjugate is administered in combination with a second agent. In certain embodiments, the second agent is a granulocyte colony stimulating factor (GCSF). In certain embodiments, the second agent is pegfilgrastim. In certain embodiments, the second agent is filgrastim. The second agent is administered as described herein, for instance according to labelled instructions. In certain embodiments, the second agent lowers the rate of neutropenia in subjects. In certain embodiments, the dose of pegfilgrastim is selected in view of the half-life of the FOLR1 antibody conjugate, for instance on starting on day 8±2 of a therapeutic cycle. While not intending to be bound by any particular theory of operation, administration of pegfilgrastim on cycle day 8±2 rather than cycle day 1 or 2 (with conventional chemotherapy) was discovered to avoid high levels of exposure of FOLR1 antibody conjugate at the same time as the pegfilgrastim. The half-life of FOLR1 antibody conjugate is longer than conventional small molecule chemotherapy agents. Stimulation of granulopoieses in the presence of high concentrations of a potentially cytotoxic agent (or radiation) can have a deleterious effect on granulocyte production (Viswanathan et al., 2014, Cancer 120(24):3870-83). Further, FOLR1 antibody conjugate-related neutropenia was discovered to occur primarily between cycle day 15 and 21, delayed compared to small molecule cytotoxic agents.

In some embodiments, the antibody conjugates bind human folate receptor alpha. In some embodiments, the antibody conjugates also bind homologs of human folate receptor alpha. In some aspects, the antibody conjugates also bind homologs of cynomolgus monkey and/or mouse folate receptor alpha. In certain embodiments, the compositions are pharmaceutical compositions. Any suitable pharmaceutical composition may be used. In certain embodiments, the pharmaceutical composition for the anti-FOLR1 antibody conjugates is a composition for intravenous (IV) administration.

The methods, kits, and compositions disclosed herein are useful for treating a disease or disorder. In certain embodiments, the disease or disorder is a cancer. In certain embodiments, the cancer is an endometrial or ovarian cancer. In certain embodiments, the conjugates and compositions provided herein are for use in therapy. In certain embodiments, provided herein are the conjugates and compositions for use in the treatment of a cancer.

### BRIEF DESCRIPTION OF THE FIGURES

FIG. 1 provides tumor reduction in patients following administration of a conjugate described herein at initial doses of 4.3 mg/kg or 5.2 mg/kg.

FIG. 2 provides objective patient responses to administration of a conjugate described herein at initial doses of 4.3 mg/kg or 5.2 mg/kg. RECIST is response evaluation criteria in solid tumors, CR is complete response, PR is partial response, PRu is unverified partial response, SD is stable disease, and PD is progressive disease.

FIG. 3 provides change in tumor diameters over time since treatment in patients with administration of a conjugate described herein at initial doses of 4.3 mg/kg or 5.2 mg/kg.

FIG. 4 provides treatment duration in patients with administration of a conjugate described herein at initial doses of 4.3 mg/kg or 5.2 mg/kg.

FIG. 5 provides the percent of patients with positive histochemistry with administration of a conjugate described herein at initial doses of 4.3 mg/kg or 5.2 mg/kg. PR is partial response, PRu is unverified partial response, SD is stable disease, and PD is progressive disease.

### DETAILED DESCRIPTION OF THE EMBODIMENTS

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

It is understood that aspects and embodiments described herein as “comprising” include “consisting of” and “consisting essentially of” embodiments.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value ±10%, ±5%, or 1%. In certain embodiments, the term “about” indicates the designated value ±one standard deviation of that value.

The term “combinations thereof” includes every possible combination of elements to which the term refers to. For example, a sentence stating that “if α₂ is A, then α₃ is not D; α₅ is not S; or α₆ is not S; or combinations thereof” includes the following combinations when α₂ is A: (1) α₃ is not D; (2) α₅ is not S; (3) α₆ is not S; (4) α₃ is not D; Us is not S; and α₆ is not S; (5) α3 is not D and α₅ is not S; (6) α₃ is not D and α₆ is not S; and (7) α₅ is not S and α₆ is not S.

The terms “folate receptor alpha” and “folate receptor 1” are used interchangeably herein. Folate receptor alpha is also known by synonyms, including FOLR1, FolRα, folate binding protein, FBP, adult folate binding protein, Folbp1, FR-alpha, FRα, KB cells FBP, and ovarian tumor-associated antigen MOv18, among others. Unless specified otherwise, the terms include any variants, isoforms and species homologs of human folate receptor alpha that are naturally expressed by cells, or that are expressed by cells transfected with a folate receptor alpha or FOLR1 gene. Folate receptor alpha proteins include, for example, human folate receptor alpha (SEQ ID NO: 1). In some embodiments, folate receptor alpha proteins include cynomolgus monkey folate receptor alpha (SEQ ID NO: 2). In some embodiments, folate receptor alpha proteins include murine folate receptor alpha (SEQ ID NO: 3).

The term “angiogenesis inhibitor” as used herein refers to a substance that inhibits the formation of new blood vessels.

The term “biologic” as used herein, refers to a drug substance is made by a living organism or derived from a living organism or made through recombinant DNA or controlled gene expression methodologies.

The term “biosimilar” or “follow-on-biologic” as used herein, refers to products that have similar structures and properties to existing biologic products. Thus, the term “biosimilar” is generally used to describe subsequent versions (generally from a different source) of “innovator biopharmaceutical products” that have previously been approved and officially granted marketing authorization. Because biologics have a high degree of molecular complexity, and are generally sensitive to changes in manufacturing processes (e.g. if different cell lines are used in their production), and since subsequent follow-on manufacturers generally do not have access to the originators molecular clone, cell bank, know-how regarding the production process, nor to the active drug substance itself (only the innovator's commercialized drug product), a “biosimilar” may not be exactly the same as the innovator drug product. However, biosimilars must demonstrate that they have no clinically meaningful differences from their reference products in terms of safety and effectiveness. Therefore, because biosimilars are subsequent versions of a known product and must demonstrate that they have no clinically meaningful differences from their reference products, the term “biosimilar” as used herein includes currently known and approved “biosimilars” as well as any “biosimilars” developed in the future.

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H}) and a heavy chain constant region (C_{H}). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated C_{L}.

The term “antibody” describes a type of immunoglobulin molecule and is used herein in its broadest sense. An antibody specifically includes intact antibodies (e.g., intact immunoglobulins), and antibody fragments. Antibodies comprise at least one antigen-binding domain. One example of an antigen-binding domain is an antigen binding domain formed by a V_{H}-V_{L} dimer. A “folate receptor alpha antibody,” “anti-folate receptor alpha antibody,” “folate receptor alpha Ab,” “folate receptor alpha-specific antibody,” “anti-folate receptor alpha Ab,” “FOLR1 antibody,” “FolRα antibody,” “anti-FOLR1 antibody,” “anti-FolRα antibody,” “FOLR1 Ab,” “FolRα Ab,” “FOLR1-specific antibody,” “FolRα-specific antibody,” “anti-FolRα Ab,” or “anti-FOLR1 Ab” is an antibody, as described herein, which binds specifically to folate receptor alpha or FOLR1. In some embodiments, the antibody binds the extracellular domain of folate receptor alpha (FOLR1).

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, MD, incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plickthun, J. Mol. Biol., 2001, 309:657-70 (“AHo” numbering scheme), each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

TABLE 1
Residues in CDRs according to
Kabat and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 | H31-H35B | H26-H32 or H34* |
| (Kabat Numbering) | | |
| H1 | H31-H35 | H26-H32 |
| (Chothia Numbering) | | |
| H2 | H50-H65 | H52-H56 |
| H3 | H95-H102 | H95-H102 |

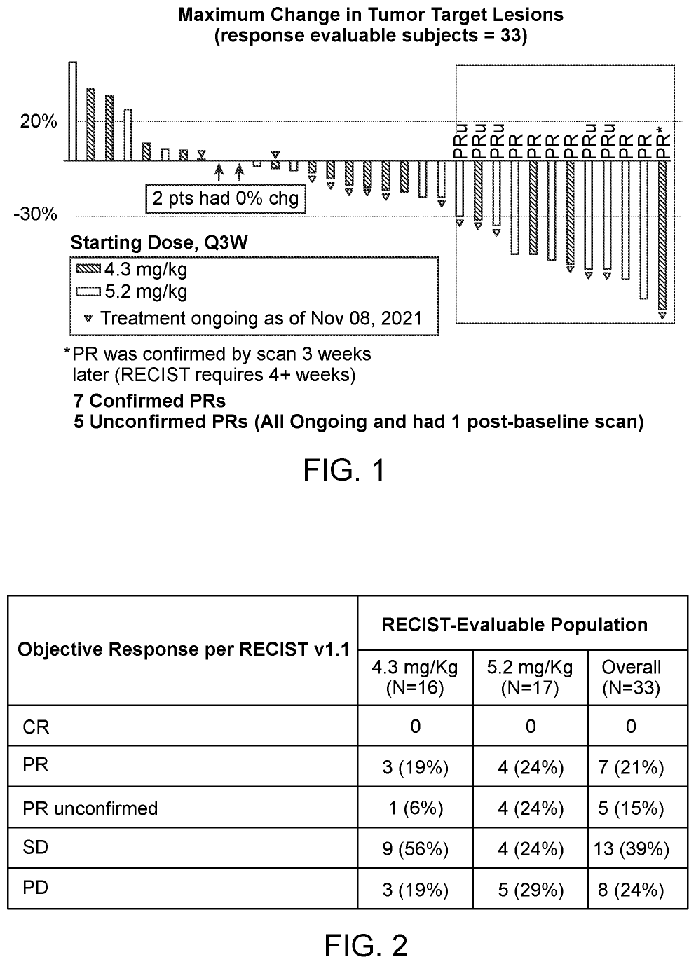

*The C-terminus of CDR-H1, when numbered using the Kabat numbering convention, varies between H32 and H34, depending on the length of the CDR, as illustrated in FIG. 1.

Unless otherwise specified, the numbering scheme used for identification of a particular CDR herein is the Kabat/Chothia numbering scheme. Where the residues encompassed by these two numbering schemes diverge (e.g., CDR-H1 and/or CDR-H2), the numbering scheme is specified as either Kabat or Chothia. For convenience, CDR-H3 is sometimes referred to herein as either Kabat or Chothia. However, this is not intended to imply differences in sequence where they do not exist, and one of skill in the art can readily confirm whether the sequences are the same or different by examining the sequences.

CDRs may be assigned, for example, using antibody numbering software, such as Abnum, available at www.bioinf.org.uk/abs/abnum/, and described in Abhinandan and Martin, Immunology, 2008, 45:3832-3839, and abYsis.org, Swindells et al. 2017, J. Mol. Biol. 429:356-364, each incorporated by reference in its entirety.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen binding or variable region of an intact antibody. Antibody fragments include, for example, Fv fragments, Fab fragments, F(ab′)2 fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length antibody.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with ß-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). In some embodiments, the linker is SEQ ID NO: 377. In some embodiments, the linker is SEQ ID NO: 378. Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminus of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used. In some cases, the Fc domain comprises an IgG1 Fc domain. In some embodiments, the IgG1 Fc domain comprises SEQ ID NO: 370, or a portion thereof. SEQ ID NO: 370 provides the sequence of C_{H1}, C_{H2}, and C_{H3} of the human IgG1 constant region.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from anon-human source that utilizes a human antibody repertoire or human antibody-encoding sequences (e.g., obtained from human sources or designed de novo). Human antibodies specifically exclude humanized antibodies.

An “isolated antibody” is one that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. An isolated antibody includes an antibody in situ within recombinant cells, since at least one component of the antibody's natural environment is not present. In some aspects, an isolated antibody is prepared by at least one purification step.

In some embodiments, an isolated antibody is purified to at least 80%, 85%, 90%, 95%, or 99% by weight. In some embodiments, an isolated antibody is purified to at least 80%, 85%, 90%, 95%, or 99% by volume. In some embodiments, an isolated antibody is provided as a solution comprising at least 85%, 90%, 95%, 98%, 99% to 100% by weight. In some embodiments, an isolated antibody is provided as a solution comprising at least 85%, 90%, 95%, 98%, 99% to 100% by volume.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can be represented by the dissociation constant (K_{D}). Affinity can be measured by common methods known in the art, including those described herein. Affinity can be determined, for example, using surface plasmon resonance (SPR) technology, such as a Biacore® instrument. In some embodiments, the affinity is determined at 25° C.

With regard to the binding of an antibody to a target molecule, the terms “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction. Specific binding can be measured, for example, by determining binding of a molecule compared to binding of a control molecule. Specific binding can also be determined by competition with a control molecule that mimics the antibody binding site on the target. In that case, specific binding is indicated if the binding of the antibody to the target is competitively inhibited by the control molecule.

The term “k_{d}” (sec⁻¹), as used herein, refers to the dissociation rate constant of a particular antibody-antigen interaction. This value is also referred to as the k_{off} value.

The term “kₐ” (M⁻¹×sec⁻¹), as used herein, refers to the association rate constant of a particular antibody-antigen interaction. This value is also referred to as the kₒₙ value.

The term “K_{D}” (M), as used herein, refers to the dissociation equilibrium constant of a particular antibody-antigen interaction. K_{D}=k_{d}/kₐ.

The term “K_{A}” (M⁻¹), as used herein, refers to the association equilibrium constant of a particular antibody-antigen interaction. K_{A}=kₐ/k_{d}.

An “affinity matured” antibody is one with one or more alterations in one or more CDRs or FRs that result in an improvement in the affinity of the antibody for its antigen, compared to a parent antibody which does not possess the alteration(s). In one embodiment, an affinity matured antibody has nanomolar or picomolar affinity for the target antigen. Affinity matured antibodies may be produced using a variety of methods known in the art. For example, Marks et al. (Bio Technology, 1992, 10:779-783, incorporated by reference in its entirety) describes affinity maturation by V_{H} and V_{L} domain shuffling. Random mutagenesis of CDR and/or framework residues is described by, for example, Barbas et al. (Proc. Nat. Acad. Sci. U.S.A., 1994, 91:3809-3813); Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896, each of which is incorporated by reference in its entirety.

When used herein in the context of two or more antibodies, the term “competes with” or “cross-competes with” indicates that the two or more antibodies compete for binding to an antigen (e.g., folate receptor alpha, or FOLR1). In one exemplary assay, FOLR1 is coated on a plate and allowed to bind a first antibody, after which a second, labeled antibody is added. If the presence of the first antibody reduces binding of the second antibody, then the antibodies compete. In another exemplary assay, a first antibody is coated on a plate and allowed to bind the antigen, and then the second antibody is added. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 50%, at least 60%, at least 70%, at least 80%, or at least 90%.

The term “epitope” means a portion of an antigen capable of specific binding to an antibody. Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter is lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to variants of folate receptor alpha (FOLR1) with different point-mutations.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution of an amino acid with a chemically or functionally similar amino acid. Conservative substitution tables providing similar amino acids are well known in the art. Polypeptide sequences having such substitutions are known as “conservatively modified variants.” Such conservatively modified variants are in addition to and do not exclude polymorphic variants, interspecies homologs, and alleles. By way of example, the groups of amino acids provided in Tables 2-4 are, in some embodiments, considered conservative substitutions for one another.

TABLE 2
Selected groups of amino acids that are considered
conservative substitutions for one another,
in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |
| Alcohol Group-Containing Residues | S and T |
| Aliphatic Residues | I, L, V, and M |
| Cycloalkenyl-associated Residues | F, H, W, and Y |
| Hydrophobic Residues | A, C, F, G, H, I, L, |
| | M, R, T, V, W, and Y |
| Negatively Charged Residues | D and E |
| Polar Residues | C, D, E, H, K, |
| | N, Q, R, S, and T |
| Positively Charged Residues | H, K, and R |
| Small Residues | A, C, D, G, N, |
| | P, S, T, and V |
| Very Small Residues | A, G, and S |
| Residues Involved in Turn Formation | A, C, D, E, G, H, K, |
| | N, Q, R, S, P, and T |
| Flexible Residues | Q, T, K, S, G, |
| | P, D, E, and R |

TABLE 3
Additional selected groups of amino acids that
are considered conservative substitutions for
one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that are
considered conservative substitutions for one
another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, NY. An antibody generated by making one or more conservative substitutions of amino acid residues in a parent antibody is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Naturally encoded amino acids are the proteinogenic amino acids known to those of skill in the art. They include the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and the less common pyrrolysine and selenocysteine. Naturally encoded amino acids include post-translational variants of the 22 naturally occurring amino acids such as prenylated amino acids, isoprenylated amino acids, myrisoylated amino acids, palmitoylated amino acids, N-linked glycosylated amino acids, O-linked glycosylated amino acids, phosphorylated amino acids and acylated amino acids.

The term “non-natural amino acid” refers to an amino acid that is not a proteinogenic amino acid, or a post-translationally modified variant thereof. In particular, the term refers to an amino acid that is not one of the 20 common amino acids or pyrrolysine or selenocysteine, or post-translationally modified variants thereof.

The term “conjugate” or “antibody conjugate” refers to an antibody linked to one or more payload moieties. The antibody can be any antibody described herein. The payload can be any payload described herein. The antibody can be directly linked to the payload via a covalent bond, or the antibody can be linked to the payload indirectly via a linker. Typically, the linker is covalently bonded to the antibody and also covalently bonded to the payload. The term “antibody drug conjugate” or “ADC” refers to a conjugate wherein at least one payload is a therapeutic moiety such as a drug.

The term “payload” refers to a molecular moiety that can be conjugated to an antibody. In particular embodiments, payloads are selected from the group consisting of therapeutic moieties and labelling moieties.

The term “linker” refers to a molecular moiety that is capable of forming at least two covalent bonds. Typically, a linker is capable of forming at least one covalent bond to an antibody and at least another covalent bond to a payload. In certain embodiments, a linker can form more than one covalent bond to an antibody. In certain embodiments, a linker can form more than one covalent bond to a payload or can form covalent bonds to more than one payload. After a linker forms a bond to an antibody, or a payload, or both, the remaining structure, i.e. the residue of the linker after one or more covalent bonds are formed, may still be referred to as a “linker” herein. The term “linker precursor” refers to a linker having one or more reactive groups capable of forming a covalent bond with an antibody or payload, or both. In some embodiments, the linker is a cleavable linker. For example, a cleavable linker can be one that is released by an bio-labile function, which may or may not be engineered. In some embodiments, the linker is a non-cleavable linker. For example, a non-cleavable linker can be one that is released upon degradation of the antibody.

The terms “pharmaceutical formulation” and “pharmaceutical composition” refer to preparations that are in such form as to permit the biological activity of the active ingredient to be effective, and that contain no additional components that are unacceptably toxic to an individual to which the formulation or composition would be administered. Such formulations or compositions may be sterile.

“Excipients” as used herein include pharmaceutically acceptable excipients, carriers, vehicles, or stabilizers that are nontoxic to the cell or mammal being exposed thereto at the dosages and concentrations employed. In some embodiments, the physiologically acceptable excipient is an aqueous pH buffered solution.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “therapeutically effective amount” or “effective amount” refers to an amount of an antibody or composition that when administered to a subject is effective to treat a disease or disorder. In some embodiments, a therapeutically effective amount or effective amount refers to an amount of an antibody or composition that when administered to a subject is effective to prevent or ameliorate a disease or the progression of the disease, or result in amelioration of symptoms.

As used herein, the term “inhibits growth” (e.g. referring to cells, such as tumor cells) is intended to include any measurable decrease in cell growth (e.g., tumor cell growth) when contacted with a folate receptor alpha (FOLR1) antibody, as compared to the growth of the same cells not in contact with a FOLR1 antibody. In some embodiments, growth may be inhibited by at least about 10%, 20%, 30%, 40%, 50%, 60%, 70%, 80%, 90%, 99%, or 100%. The decrease in cell growth can occur by a variety of mechanisms, including but not limited to antibody internalization, apoptosis, necrosis, and/or effector function-mediated activity.

As used herein, the term “subject” means a mammalian subject. Exemplary subjects include, but are not limited to humans, monkeys, dogs, cats, mice, rats, cows, horses, camels, avians, goats, and sheep. In certain embodiments, the subject is a human. In some embodiments, the subject has a disease that can be treated or diagnosed with an antibody provided herein. In some embodiments, the disease is epithelial ovarian, fallopian tube, or primary peritoneal cancer. In some embodiments, the disease is gastric carcinoma, colorectal carcinoma, renal cell carcinoma, cervical carcinoma, non-small cell lung carcinoma, ovarian cancer, breast cancer, triple-negative breast cancer, endometrial cancer, prostate cancer, and/or a cancer of epithelial origin.

As used herein, the term “cycle” indicates one period of dosing on a dosing schedule. The cycle includes the day of the dose and the following days up to the next dose. Exemplary cycles have lengths of 7 days, 10 days, 14 days, and 21 days.

In some chemical structures illustrated herein, certain substituents, chemical groups, and atoms are depicted with a curvy/wavy line (e.g.,

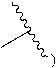

that intersects a bond or bonds to indicate the atom through which the substituents, chemical groups, and atoms are bonded. For example, in some structures, such as but not limited to

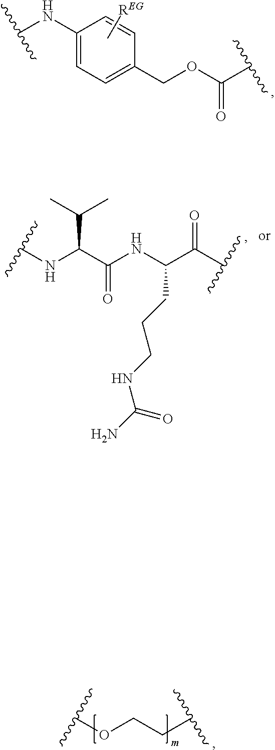

this curvy/wavy line indicates the atoms in the backbone of a conjugate or linker-payload structure to which the illustrated chemical entity is bonded. In some structures, such as but not limited to

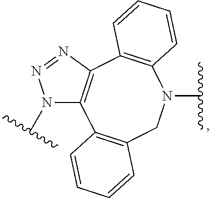 [SMILES: *N1Cc2ccccc2-c2c(nnn2*)-c2ccccc21 ; ","]

this curvy/wavy line indicates the atoms in the antibody or antibody fragment as well as the atoms in the backbone of a conjugate or linker-payload structure to which the illustrated chemical entity is bonded.

The term “site-specific” refers to a modification of a polypeptide at a predetermined sequence location in the polypeptide. The modification is at a single, predictable residue of the polypeptide with little or no variation. In particular embodiments, a modified amino acid is introduced at that sequence location, for instance recombinantly or synthetically. Similarly, a moiety can be “site-specifically” linked to a residue at a particular sequence location in the polypeptide. In certain embodiments, a polypeptide can comprise more than one site-specific modification.

### 1. Dosing

Provided herein are methods of treating cancer in a patient in need thereof by administering an anti-FOLR1 antibody drug conjugate according to a dosing schedule. The patient can be any patient deemed suitable for treatment by the practitioner of skill. In certain embodiments, the patient has cancer. In certain embodiments, the patient has endometrial cancer. In certain embodiments, the patient has ovarian cancer. In certain embodiments, the subject previously received cancer treatment. In certain embodiments, the subject did not previously receive cancer treatment.

The anti-FOLR1 antibody conjugate or composition thereof is suitably administered to the patient over a series of treatments. In certain embodiments, an anti-FOLR1 antibody conjugate is administered at two different doses. In certain embodiments, an anti-FOLR1 antibody conjugate described herein is administered to an individual at a fixed dose based on the individual's weight (e.g., mg/kg).

In certain embodiments, the anti-FOLR1 antibody conjugate is administered at a first dose one to three times and then at a second dose at least one to three times. In certain embodiments, the second dose is lower than the first dose. In certain embodiments, the first dose is administered for one cycle. In certain embodiments, the first dose is administered for two cycles. In certain embodiments, the first dose is administered for three cycles. In certain embodiments, the second dose is administered for at least one cycle. In certain embodiments, the second dose is administered for at least two cycles. In certain embodiments, the second dose is administered for at least three cycles. In certain embodiments, the second dose is administered for the remainder of the treating. In certain embodiments, each cycle is one week after the previous cycle.

Each dose can be administered according to a suitable schedule, for example, once, two times, three times, or for times weekly. In certain embodiments, each dose is administered about once every three, four, or five weeks. In certain embodiments, each dose is administered about once every three weeks. In certain embodiments, each dose is administered about once every four weeks. In certain embodiments, each dose is administered about once every five weeks. In certain embodiments, each dose is administered daily. In certain embodiments, each dose is separated by a cycle length from the previous dose. In certain embodiments, the cycle length is one day, two days, three days, four days, five days, six days, weekly, ten days, or two weeks, three weeks, four weeks, or more. In particular embodiments the cycle length is 21 days. The cycle length may be 21, 22, 23, 24, 25, 26, 27, 28, 29, 30, 31, 32, 33, 34 or 35 days. In certain embodiments, the clinician or practicing physician will adjust the cycle length. In certain embodiments, the clinician or treating physician will switch from the first dose to the second dose as described herein. Furthermore, it is noted that the clinician or treating physician will know how and when to interrupt, adjust, or terminate therapy in conjunction with subject response. For example, the clinician or treating physician may further adjust dose level in conjunction with subject side effects from treatment or underlying disease for which the subject is receiving treatment.

In certain embodiments, the first dose is about 5.2 mg/kg. In certain embodiments, the first dose is 5.2 mg/kg. In certain embodiments, the second dose is about 4.3 mg/kg. In certain embodiments, the second dose is 4.3 mg/kg. In certain embodiments, the first dose is about 5.2 mg/kg and the second dose is about 4.3 mg/kg. In certain embodiments, the first dose is 5.2 mg/kg and the second dose is 4.3 mg/kg. In certain embodiments, the administering is by intravenous (IV) administration.

In certain embodiments, the first dose is about 4.3 mg/kg. In certain embodiments, the first dose is 4.3 mg/kg. In certain embodiments, the first dose is about 4.3 mg/kg and the second dose is about 3.5 mg/kg. In certain embodiments, the first dose is 4.3 mg/kg and the second dose is 3.5 mg/kg.

In certain embodiments, the second dose is about 4.3 mg/kg. In certain embodiments, the second dose is 4.3 mg/kg. In certain embodiments, the second dose is about 3.5 mg/kg. In certain embodiments, the second dose is 3.5 mg/kg. In certain embodiments, the second dose is about 2.9 mg/kg. In certain embodiments, the second dose is 2.9 mg/kg.

In certain embodiments, first dose is about 5.2 mg/kg; the second dose is administered after the first dose, the amount of the second dose is less than the amount of the first dose, and the first dose and second dose are administered at least about three weeks apart. In certain embodiments, the second dose is about 4.3 mg/kg or less, the second dose is administered after the first dose, the amount of the first dose is greater than the amount of the second dose, and wherein the first dose is administered once every three weeks or longer for one to six cycles. In certain embodiments, the second dose is administered after the first dose, the amount of the first dose is the same or greater than the amount of the second dose, a tumor tissue sample from the subject has a tumor proportion score (TPS) of about 25% or more, and the subject has a tumor reduction of at least 30% by volume after three weeks from administering the first dose.

In certain embodiments, the first dose is about 5.2 mg/kg. In certain embodiments, the second dose is about 4.3 mg/kg or less. In certain embodiments, the second dose is about 4.3 mg/kg. In certain embodiments, the first dose is about 5.2 mg/kg, and the second dose is about 4.3 mg/kg.

In certain embodiments, the methods further comprise administering a third dose of the FOLR1 antibody conjugate. In certain embodiments, the amount of the third dose is less than the amount of the second dose. In certain embodiments, the amount of the third dose is about 3.5 mg/kg or less. In certain embodiments, the amount of the third dose is about 3.5 mg/kg. In certain embodiments, the amount of the third dose is 3.5 mg/kg. In certain embodiments, the amount of the third dose is about 2.9 mg/kg. In certain embodiments, the amount of the third dose is 2.9 mg/kg. In certain embodiments, the first dose is about 5.2 mg/kg, the second dose is about 4.3 mg/kg, and the third dose is about 3.5 mg/kg. In certain embodiments, the first dose is about 4.3 mg/kg, the second dose is about 3.5 mg/kg, and the third dose is about 2.9 mg/kg.

In certain embodiments, a dose of an antibody conjugate or composition provided herein can be administered to achieve a steady-state concentration of the antibody in blood or serum of the subject. The steady-state concentration can be determined by measurement according to techniques available to those of skill or can be based on the physical characteristics of the subject such as height, weight and age.

In certain embodiments, the therapy continues until the patient reaches remission or partial remission. In certain embodiments, the therapy continues until the clinician or treating physician decides to stop. In certain embodiments, the therapy continues for one, two, three, four, five, six, seven, eight, nine, ten, fifteen, twenty, twenty-five, or thirty weeks, or more.

In certain embodiments, the first dose is administered about once every three weeks or longer for one to five cycles. In certain embodiments, the first dose is administered once every three weeks or longer for one to five cycles. For example, the first dose may be administered once about every three, four or five weeks for one, two, three, four or five cycles. In certain embodiments, the first dose is administered for one to three cycles. In certain embodiments, the first dose is administered for two to four cycles. In certain embodiments, the second dose is administered at least about three weeks after the first dose is administered. In certain embodiments, the second dose is administered at least about six weeks after the first dose is administered. In certain embodiments, the second dose is administered at least about six weeks after the first dose is administered. In certain embodiments, the second dose is administered once every three weeks. In certain embodiments, the second dose is administered once every three weeks for at least about 3 weeks. In certain embodiments, the second dose is administered once every three weeks for at least about 2 months. In certain embodiments, the second dose is administered once every three weeks for at least about 3 months. In certain embodiments, the second dose is administered once every three weeks for at least about 4 months. In certain embodiments, the second dose is administered once every three weeks for the remainder of the treating. In certain embodiments, the first dose and the second dose are administered at least about three weeks apart. For example, the first dose and the second dose may be administered about three, four, five, or six weeks apart.

### 2. Conjugates

In the methods, anti-FOLR1 antibody conjugate can be any anti-FOLR1 antibody conjugate provided herein. The conjugates comprise an antibody to FOLR1 covalently linked directly or indirectly, via a linker, to a payload. In certain embodiments, the antibody is linked to one payload. In further embodiments, the antibody is linked to more than one payload. In certain embodiments, the antibody is linked to two, three, four, five, six, seven, eight, or more payloads. In certain embodiments, the anti-FOLR1 antibody conjugate is an anti-FOLR1 antibody conjugate described in U.S. Pat. No. 10,596,270, the content of which is hereby incorporated by reference.

In certain embodiments, the anti-FOLR1 antibody conjugate is according to the formula of Conjugate P, described herein, wherein the antibody is 1848-H01 conjugated through p-azidomethylphenylalanine residues at heavy chain positions Y180 and F404. In certain embodiments, the antibody of the anti-FOLR1 antibody conjugate comprises three heavy chain CDRs of heavy chain SEQ ID NO:362 and three light chain CDRs of light chain SEQ ID NO:367. In certain embodiments, the antibody of the anti-FOLR1 antibody conjugate comprises the three heavy chain CDRs of SEQ ID NOS: 58, 176, and 294 and three light chain CDRs of light chain SEQ ID NO:367. the antibody of the anti-FOLR1 antibody conjugate comprises the three heavy chain CDRs of SEQ ID NOS: 117, 235, and 294 and three light chain CDRs of light chain SEQ ID NO:367. In certain embodiments, the antibody of the anti-FOLR1 antibody conjugate comprises the VH region of heavy chain SEQ ID NO:362 and the VL region of light chain SEQ ID NO:367. In certain embodiments, the antibody of the anti-FOLR1 antibody conjugate comprises heavy chain SEQ ID NO:362 and light chain SEQ ID NO:367. In each of these embodiments, the antibody may comprise Y180 and F404 mutations to p-azidomethylphenylalanine.

The payload can be any payload deemed useful by the practitioner of skill. In certain embodiments, the payload is a therapeutic moiety. In certain embodiments, the payload is a diagnostic moiety, e.g., a label. Useful payloads are described in the sections and examples below.

The linker can be any linker capable of forming at least one bond to the antibody and at least one bond to a payload. Useful linkers are described the sections and examples below.

In the conjugates provided herein, the antibody can be any antibody with binding specificity for folate receptor alpha (FOLR1 or FolRα). The FOLR1 can be from any species. In certain embodiments, the FOLR1 is a vertebrate FOLR1. In certain embodiments, the FOLR1 is a mammalian FOLR1. In certain embodiments, the FOLR1 is human FOLR1. In certain embodiments, the FOLR1 is mouse FOLR1. In certain embodiments, the FOLR1 is cynomolgus FOLR1.

In certain embodiments, the antibody to folate receptor alpha (FOLR1 or FolRα) competes with an antibody described herein for binding. In certain embodiments, the antibody to FOLR1 binds to the same epitope as an antibody described herein.

The antibody is typically a protein comprising multiple polypeptide chains. In certain embodiments, the antibody is a heterotetramer comprising two identical light (L) chains and two identical heavy (H) chains. Each light chain can be linked to a heavy chain by one covalent disulfide bond. Each heavy chain can be linked to the other heavy chain by one or more covalent disulfide bonds. Each heavy chain and each light chain can also have one or more intrachain disulfide bonds. As is known to those of skill in the art, each heavy chain typically comprises a variable domain (V_{H}) followed by a number of constant domains. Each light chain typically comprises a variable domain at one end (V_{L}) and a constant domain. As is known to those of skill in the art, antibodies typically have selective affinity for their target molecules, i.e. antigens.

The antibodies provided herein can have any antibody form known to those of skill in the art. They can be full-length, or fragments. Exemplary full-length antibodies include IgA, IgA1, IgA2, IgD, IgE, IgG, IgG1, IgG2, IgG3, IgG4, IgM, etc. Exemplary fragments include Fv, Fab, Fc, scFv, scFv-Fc, etc.

In certain embodiments, the antibody of the conjugate comprises one, two, three, four, five, or six of the CDR sequences described herein. In certain embodiments, the antibody of the conjugate comprises a heavy chain variable domain (V_{H}) described herein. In certain embodiments, the antibody of the conjugate comprises a light chain variable domain (V_{L}) described herein. In certain embodiments, the antibody of the conjugate comprises a heavy chain variable domain (V_{H}) described herein and a light chain variable domain (V_{L}) described herein. In certain embodiments, the antibody of the conjugate comprises a paired heavy chain variable domain and a light chain variable domain described herein (V_{H}-V_{L} pair).

In certain embodiments, the antibody of the conjugate comprises any of the amino acid sequences of the antibodies described above. In certain embodiments, the antibody comprises any of the amino acid sequences above with up to 10 amino acid substitutions. In certain embodiments, the antibody comprises any of the amino acid sequences above with up to 9 amino acid substitutions. In certain embodiments, the antibody comprises any of the amino acid sequences above with up to 8 amino acid substitutions. In certain embodiments, the antibody comprises any of the amino acid sequences above with up to 7 amino acid substitutions. In certain embodiments, the antibody comprises any of the amino acid sequences above with up to 6 amino acid substitutions. In certain embodiments, the antibody comprises any of the amino acid sequences above with up to 5 amino acid substitutions. In certain embodiments, the antibody comprises any of the amino acid sequences above with up to 4 amino acid substitutions. In certain embodiments, the antibody comprises any of the amino acid sequences above with up to 3 amino acid substitutions. In certain embodiments, the antibody comprises any of the amino acid sequences above with up to 2 amino acid substitutions. In certain embodiments, the antibody comprises any of the amino acid sequences above with up to 1 conservative amino acid substitution. In some embodiments, the amino acid substitutions are conservative amino acid substitutions. For example, in certain embodiments, the antibody comprises any of the amino acid sequences above with up to 10 conservative amino acid substitutions. In certain embodiments, the antibody comprises any of the amino acid sequences above with up to 9 conservative amino acid substitutions. In certain embodiments, the antibody comprises any of the amino acid sequences above with up to 8 conservative amino acid substitutions. In certain embodiments, the antibody comprises any of the amino acid sequences above with up to 7 conservative amino acid substitutions. In certain embodiments, the antibody comprises any of the amino acid sequences above with up to 6 conservative amino acid substitutions. In certain embodiments, the antibody comprises any of the amino acid sequences above with up to 5 conservative amino acid substitutions. In certain embodiments, the antibody comprises any of the amino acid sequences above with up to 4 conservative amino acid substitutions. In certain embodiments, the antibody comprises any of the amino acid sequences above with up to 3 conservative amino acid substitutions. In certain embodiments, the antibody comprises any of the amino acid sequences above with up to 2 conservative amino acid substitutions. In certain embodiments, the antibody comprises any of the amino acid sequences above with up to 1 conservative amino acid substitution.

In certain embodiments, the antibody conjugate can be formed from an antibody that comprises one or more reactive groups. In certain embodiments, the antibody conjugate can be formed from an antibody comprising all naturally encoded amino acids. Those of skill in the art will recognize that several naturally encoded amino acids include reactive groups capable of conjugation to a payload or to a linker. These reactive groups include cysteine side chains, lysine side chains, and amino-terminal groups. In these embodiments, the antibody conjugate can comprise a payload or linker linked to the residue of an antibody reactive group. In these embodiments, the payload precursor or linker precursor comprises a reactive group capable of forming a bond with an antibody reactive group. Typical reactive groups include maleimide groups, activated carbonates (including but not limited to, p-nitrophenyl ester), activated esters (including but not limited to, N-hydroxysuccinimide, p-nitrophenyl ester, and aldehydes). Particularly useful reactive groups include maleimide and succinimide, for instance N-hydroxysuccinimide, for forming bonds to cysteine and lysine side chains. Further reactive groups are described in the sections and examples below.

In further embodiments, the antibody comprises one or more modified amino acids having a reactive group, as described herein. Typically, the modified amino acid is not a naturally encoded amino acid. These modified amino acids can comprise a reactive group useful for forming a covalent bond to a linker precursor or to a payload precursor. One of skill in the art can use the reactive group to link the polypeptide to any molecular entity capable of forming a covalent bond to the modified amino acid. Thus, provided herein are conjugates comprising an antibody comprising a modified amino acid residue linked to a payload directly or indirectly via a linker. Exemplary modified amino acids are described in the sections below. Generally, the modified amino acids have reactive groups capable of forming bonds to linkers or payloads with complementary reactive groups.

The non-natural amino acids are positioned at select locations in a polypeptide chain of the antibody. These locations were identified as providing optimum sites for substitution with the non-natural amino acids. Each site is capable of bearing a non-natural amino acid with optimum structure, function and/or methods for producing the antibody.

In certain embodiments, a site-specific position for substitution provides an antibody that is stable. Stability can be measured by any technique apparent to those of skill in the art.

In certain embodiments, a site-specific position for substitution provides an antibody that has optimal functional properties. For instance, the antibody can show little or no loss of binding affinity for its target antigen compared to an antibody without the site-specific non-natural amino acid. In certain embodiments, the antibody can show enhanced binding compared to an antibody without the site-specific non-natural amino acid.

In certain embodiments, a site-specific position for substitution provides an antibody that can be made advantageously. For instance, in certain embodiments, the antibody shows advantageous properties in its methods of synthesis, discussed below. In certain embodiments, the antibody can show little or no loss in yield in production compared to an antibody without the site-specific non-natural amino acid. In certain embodiments, the antibody can show enhanced yield in production compared to an antibody without the site-specific non-natural amino acid. In certain embodiments, the antibody can show little or no loss of tRNA suppression compared to an antibody without the site-specific non-natural amino acid. In certain embodiments, the antibody can show enhanced tRNA suppression in production compared to an antibody without the site-specific non-natural amino acid.

In certain embodiments, a site-specific position for substitution provides an antibody that has advantageous solubility. In certain embodiments, the antibody can show little or no loss in solubility compared to an antibody without the site-specific non-natural amino acid. In certain embodiments, the antibody can show enhanced solubility compared to an antibody without the site-specific non-natural amino acid.

In certain embodiments, a site-specific position for substitution provides an antibody that has advantageous expression. In certain embodiments, the antibody can show little or no loss in expression compared to an antibody without the site-specific non-natural amino acid. In certain embodiments, the antibody can show enhanced expression compared to an antibody without the site-specific non-natural amino acid.

In certain embodiments, a site-specific position for substitution provides an antibody that has advantageous folding. In certain embodiments, the antibody can show little or no loss in proper folding compared to an antibody without the site-specific non-natural amino acid. In certain embodiments, the antibody can show enhanced folding compared to an antibody without the site-specific non-natural amino acid.

In certain embodiments, a site-specific position for substitution provides an antibody that is capable of advantageous conjugation. As described below, several non-natural amino acids have side chains or functional groups that facilitate conjugation of the antibody to a second agent, either directly or via a linker. In certain embodiments, the antibody can show enhanced conjugation efficiency compared to an antibody without the same or other non-natural amino acids at other positions. In certain embodiments, the antibody can show enhanced conjugation yield compared to an antibody without the same or other non-natural amino acids at other positions. In certain embodiments, the antibody can show enhanced conjugation specificity compared to an antibody without the same or other non-natural amino acids at other positions.

The one or more non-natural amino acids are located at selected site-specific positions in at least one polypeptide chain of the antibody. The polypeptide chain can be any polypeptide chain of the antibody without limitation, including either light chain or either heavy chain. The site-specific position can be in any domain of the antibody, including any variable domain and any constant domain.

In certain embodiments, the antibodies provided herein comprise one non-natural amino acid at a site-specific position. In certain embodiments, the antibodies provided herein comprise two non-natural amino acids at site-specific positions. In certain embodiments, the antibodies provided herein comprise three non-natural amino acids at site-specific positions. In certain embodiments, the antibodies provided herein comprise more than three non-natural amino acids at site-specific positions.

In certain embodiments, the antibodies provided herein comprise one or more non-natural amino acids each at a position selected from the group consisting of heavy chain or light chain residues HC-F404, HC-K121, HC-Y180, HC-F241, HC-221, LC-T22, LC-S7, LC-N152, LC-K42, LC-E161, LC-D170, HC-S136, HC-S25, HC-A40, HC-S119, HC-S190, HC-K222, HC-R19, HC-Y52, or HC-S70 according to the Kabat or Chothia or EU numbering scheme, or a post-translationally modified variant thereof. In these designations, HC indicates a heavy chain residue, and LC indicates a light chain residue.

In certain embodiments, the antibody is linked site-specifically to at least one payload. In certain embodiments, the antibody comprises one or more non-natural amino acids at sites selected from the group consisting of: HC F404, HC-K121, HC-Y180, HC-F241, HC-221, LC-T22, LC-S7, LC-N152, LC-K42, LC-E161, LC-D170, HC-S136, HC S25, HC-A40, HC-S119, HC-S190, HC-K222, HC-R19, HC-Y52, or HC-S70, according to the Kabat, Chothia, or EU numbering scheme In certain embodiments, the antibody comprises one or more non-natural amino acids at sites selected from the group consisting of: HC F404, HC-Y180, and LC-K42, according to the Kabat, Chothia, or EU numbering scheme. In certain embodiments, the antibody comprises a non-natural amino acid at site HC-F404. In certain embodiments, the antibody comprises a non-natural amino acid at site HC-F404 In certain embodiments, the antibody comprises non-natural amino acids at sites HC-F404 and HC-Y180. In certain embodiments, a residue of the one or more non-natural amino acids is linked to the payload moiety via a linker that is hydrolytically stable. In certain embodiments, a residue of the one or more non-natural amino acids is linked to the payload moiety via a linker that is cleavable.

In certain embodiments, the one or more non-natural amino acids is selected from the group consisting of p-acetyl-L-phenylalanine, O-methyl-L-tyrosine, an -3-(2-naphthyl)alanine, 3-methyl-phenylalanine, O-4-allyl-L-tyrosine, 4-propyl-L-tyrosine, a tri-O-acetyl-GlcNAcP-seine, L-Dopa, fluorinated phenylalanine, isopropyl-L-phenylalanine, p-azido-L-phenylalanine, p-azido-methyl-L-phenylalanine, compound 56, p-acyl-L-phenylalanine, p-benzoyl-L-phenylalanine, L-phosphoserine, phosphonoserine, phosphonotyrosine, p-iodo-phenylalanine, p-bromophenylalanine, p-amino-L-phenylalanine, isopropyl-L-phenylalanine, and p-propargyloxy-phenylalanine. In certain embodiments, the non-natural amino acid residue is a residue of compound (30) or compound (56).

In particular embodiments, provided herein are anti-FOLR1 conjugates according to any of Formulas 101a-104b wherein COMP indicates a residue of the non-natural amino acid according to Formula (30), below. In particular embodiments, provided herein are anti-FOLR1 conjugates according to any of Formulas 101a-104b wherein COMP indicates a residue of the non-natural amino acid according to Formula (30), below, at heavy chain position 404 according to the EU numbering system. In particular embodiments, provided herein are anti-FOLR1 conjugates according to any of Formulas 101a-104b wherein COMP indicates a residue of the non-natural amino acid according to Formula (30), below, at heavy chain position 180 according to the EU numbering system. In particular embodiments, provided herein are anti-FOLR1 conjugates according to any of Formulas 101a-104b wherein COMP indicates a residue of the non-natural amino acid according to Formula (30), below, at heavy chain position 241 according to the EU numbering system. In particular embodiments, provided herein are anti-FOLR1 conjugates according to any of Formulas 101a-104b wherein COMP indicates a residue of the non-natural amino acid according to Formula (30), below, at heavy chain position 222 according to the EU numbering system. In particular embodiments, provided herein are anti-FOLR1 conjugates according to any of Formulas 101a-104b wherein COMP indicates a residue of the non-natural amino acid according to Formula (30), below, at light chain position 7 according to the Kabat or Chothia numbering system. In particular embodiments, provided herein are anti-FOLR1 conjugates according to any of Formulas 101a-104b wherein COMP indicates a residue of the non-natural amino acid according to Formula (30), below, at light chain position 42 according to the Kabat or Chothia numbering system. In certain embodiments, PAY is selected from the group consisting of maytansine, hemiasterlin, amanitin, monomethyl auristatin F (MMAF), and monomethyl auristatin E (MMAE). In certain embodiments, the PAY is maytansine. In certain embodiments, PAY is hemiasterlin. In certain embodiments, PAY is amanitin. In certain embodiments, PAY is MMAF. In certain embodiments, PAY is MMAE.

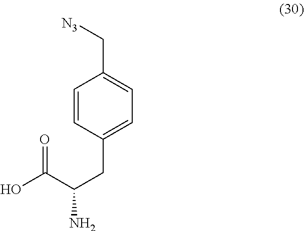 [SMILES: [N-]=[N+]=NCc1ccc(C[C@H](N)C(=O)O)cc1]

In particular embodiments, provided herein are anti-FOLR1 conjugates according to any of Formulas 101a-104b wherein COMP indicates a residue of the non-natural amino acid according to Formula (56), below. In particular embodiments, provided herein are anti-FOLR1 conjugates according to any of Formulas 101a-104b wherein COMP indicates a residue of the non-natural amino acid according to Formula (56), below, at heavy chain position 404 according to the EU numbering system. In particular embodiments, provided herein are anti-FOLR1 conjugates according to any of Formulas 101a-104b wherein COMP indicates a residue of the non-natural amino acid according to Formula (56), below, at heavy chain position 180 according to the EU numbering system. In particular embodiments, provided herein are anti-FOLR1 conjugates according to any of Formulas 101a-104b wherein COMP indicates a residue of the non-natural amino acid according to Formula (56), below, at heavy chain position 241 according to the EU numbering system. In particular embodiments, provided herein are anti-FOLR1 conjugates according to any of Formulas 101a-104b wherein COMP indicates a residue of the non-natural amino acid according to Formula (56), below, at heavy chain position 222 according to the EU numbering system. In particular embodiments, provided herein are anti-FOLR1 conjugates according to any of Formulas 101a-104b wherein COMP indicates a residue of the non-natural amino acid according to Formula (56), below, at light chain position 7 according to the Kabat or Chothia numbering system. In particular embodiments, provided herein are anti-FOLR1 conjugates according to any of Formulas 101a-104b wherein COMP indicates a residue of the non-natural amino acid according to Formula (56), below, at light chain position 42 according to the Kabat or Chothia numbering system. In certain embodiments, PAY is selected from the group consisting of maytansine, hemiasterlin, amanitin, MMAF, and MMAE. In certain embodiments, the PAY is maytansine. In certain embodiments, PAY is hemiasterlin. In certain embodiments, PAY is amanitin. In certain embodiments, PAY is MMAF. In certain embodiments, PAY is MMAE.

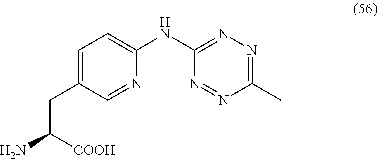 [SMILES: Cc1nnc(Nc2ccc(C[C@H](N)C(=O)O)cn2)nn1]

In particular embodiments, provided herein are anti-FOLR1 conjugates according to any of Formulas 101a-104b wherein COMP indicates a non-natural amino acid residue of para-azido-L-phenylalanine. In particular embodiments, provided herein are anti-FOLR1 conjugates according to any of Formulas 101a-104b wherein COMP indicates the non-natural amino acid residue para-azido-phenylalanine at heavy chain position 404 according to the EU numbering system. In particular embodiments, provided herein are anti-FOLR1 conjugates according to any of Formulas 101a-104b wherein COMP indicates a non-natural amino acid residue of para-azido-L-phenylalanine at heavy chain position 180 according to the EU numbering system. In particular embodiments, provided herein are anti-FOLR1 conjugates according to any of Formulas 101a-104b wherein COMP indicates a non-natural amino acid residue para-azido-L-phenylalanine at heavy chain position 241 according to the EU numbering system. In particular embodiments, provided herein are anti-FOLR1 conjugates according to any of Formulas 101a-104b wherein COMP indicates a non-natural amino acid residue para-azido-L-phenylalanine at heavy chain position 222 according to the EU numbering system. In particular embodiments, provided herein are anti-FOLR1 conjugates according to any of Formulas 101a-104b wherein COMP indicates a non-natural amino acid residue para-azido-L-phenylalanine at light chain position 7 according to the Kabat or Chothia numbering system. In particular embodiments, provided herein are anti-FOLR1 conjugates according to any of Formulas 101a-104b wherein COMP indicates a non-natural amino acid residue para-azido-L-phenylalanine at light chain position 42 according to the Kabat or Chothia numbering system. In certain embodiments, PAY is selected from the group consisting of maytansine, hemiasterlin, amanitin, MMAF, and MMAE. In certain embodiments, the PAY is maytansine. In certain embodiments, PAY is hemiasterlin. In certain embodiments, PAY is amanitin. In certain embodiments, PAY is MMAF. In certain embodiments, PAY is MMAE.

In certain embodiments, the at least one payload moiety is selected from the group consisting of maytansines, hemiasterlins, amanitins, and auristatins. In certain embodiments, the at least one payload moiety is selected from the group consisting of DM1, hemiasterlin, amanitin, MMAF, and MMAE. In certain embodiments, the at least one payload moiety is a hemiasterlin derivative. In certain embodiments, the at least one payload moiety is:

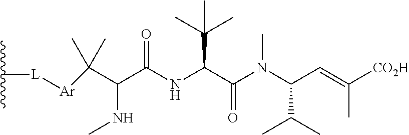

wherein Ar is aryl or heteroaryl, L is a linker, and the wiggly line indicates a bond to the antibody. In certain embodiments, the at least one payload moiety is:

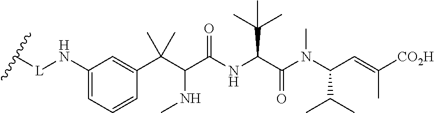

wherein L is a linker, and the wiggly line indicates a bond to the antibody.

In some embodiments, provided herein are anti-FOLR1 conjugates comprising a modified hemiasterlin and linker as described, for example, in PCT Publication No. WO 2016/123582. For example, the conjugate can have a structure comprising any of Formulas 1000-1000b, 1001-1001b, 1002-1002b, and I-XIXb-2, 101-111b, or 1-8b as described in PCT Publication No. WO 2016/2016/123582. Examples of conjugates comprising a modified hemiasterlin and linker are provided below.

In some embodiments, provided herein are anti-FOLR1 conjugates having the structure of Conjugate M:

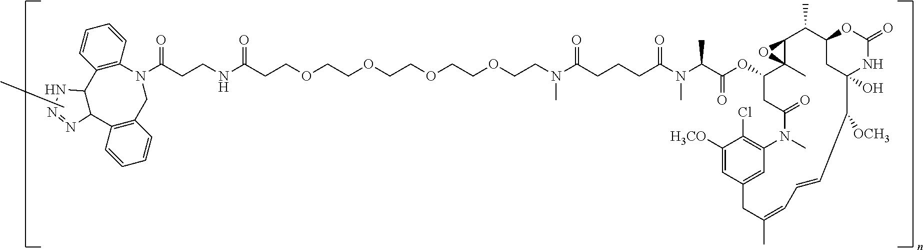

where n is an integer from 1 to 6. In some embodiments, n is an integer from 1 to 4. In some embodiments, n is 2. For example, in some embodiments, the anti-FOLR1 conjugate has the structure:

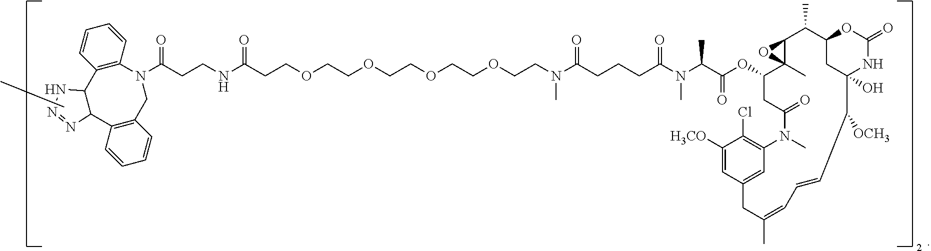

In some embodiments, n is 4. For example, in some embodiments, the anti-FLOR1 conjugate has the structure:

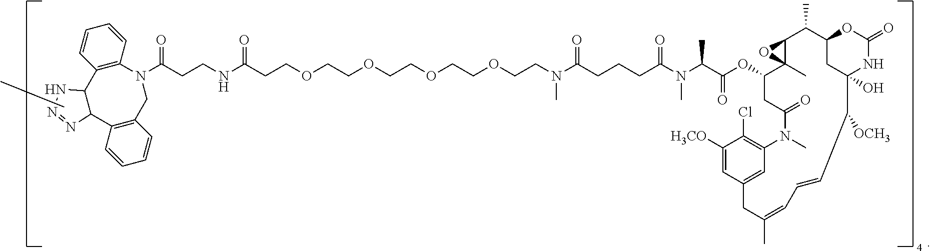

In some embodiments, provided herein are anti-FOLR1 conjugates having the structure of Conjugate P:

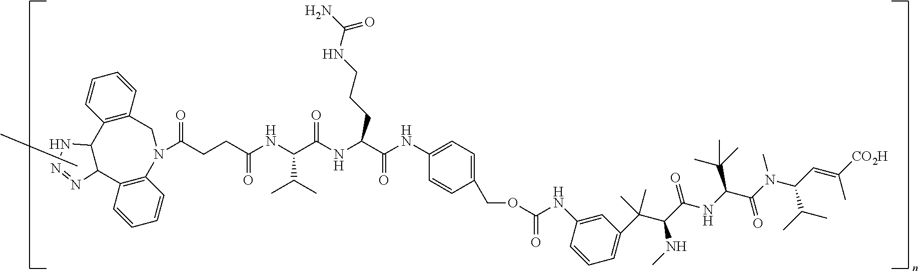

where n is an integer from 1 to 6. In some embodiments, n is an integer from 1 to 4. In some embodiments, n is 2. For example, in some embodiments, the anti-FOLR1 conjugate has the structure:

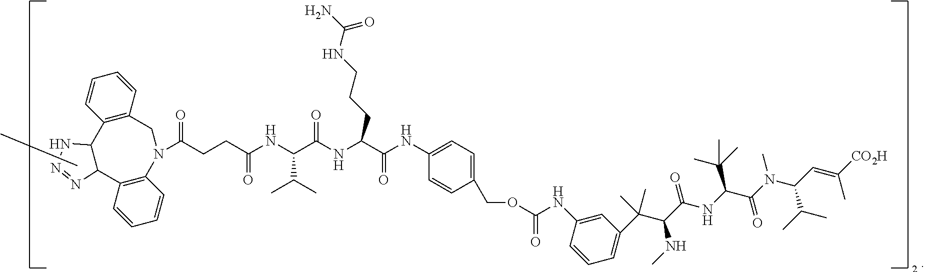

In some embodiments, n is 4. For example, in some embodiments, the anti-FOLR1 conjugate has the structure:

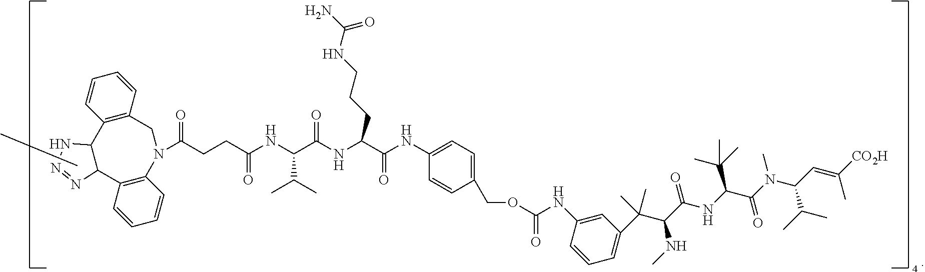

In some embodiments, provided herein are anti-FOLR1 conjugates having the structure of Conjugate Q:

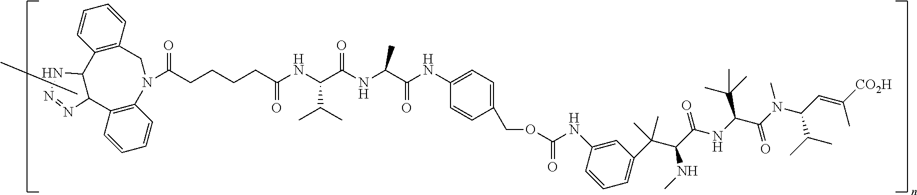

where n is an integer from 1 to 6. In some embodiments, n is an integer from 1 to 4. In some embodiments, n is 2. For example, in some embodiments, the anti-FOLR1 conjugate has the structure:

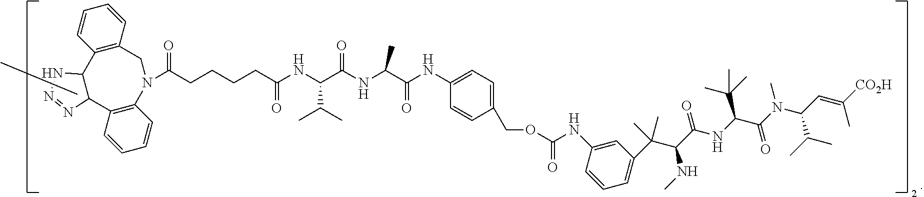

In some embodiments, n is 4. For example, in some embodiments, the anti-FOLR1 conjugate has the structure:

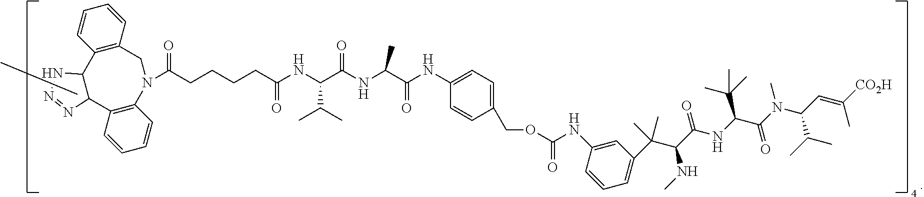

In any of the foregoing embodiments wherein the anti-FOLR1 conjugate has a structure according to Conjugate M, Conjugate P, or Conjugate Q, the bracketed structure can be covalently bonded to one or more non-natural amino acids of the antibody, wherein the one or more non-natural amino acids are located at sites selected from the group consisting of: HC-F404, HC-Y180, and LC-K42 according to the Kabat or EU numbering scheme of Kabat. In some embodiments, the bracketed structure is covalently bonded to one or more non-natural amino acids at site HC-F404 of the antibody. In some embodiments, the bracketed structure is covalently bonded to one or more non-natural amino acids at site HC-Y180 of the antibody. In some embodiments, the bracketed structure is covalently bonded to one or more non-natural amino acids at site LC-K42 of the antibody. In some embodiments, the bracketed structure is covalently bonded to one or more non-natural amino acids at sites HC-F404 and HC-Y180 of the antibody. In some embodiments, at least one bracketed structure is covalently bonded to a non-natural amino acid at site HC-F404 of the antibody, and at least one bracketed structure is covalently bonded a non-natural amino acid at site HC-Y180 of the antibody. In some embodiments, the bracketed structure is covalently bonded to one or more non-natural amino acids at sites HC-Y180 and LC-K42 of the antibody. In some embodiments, at least one bracketed structure is covalently bonded to a non-natural amino acid at site HC-Y180 of the antibody, and at least one bracketed structure is covalently bonded a non-natural amino acid at site LC-K32 of the antibody.

### 5. Antibody Specificity

The conjugates comprise antibodies that selectively bind human folate receptor alpha. In some aspects, the antibody selectively binds to the extracellular domain of human folate receptor alpha (human FOLR1).

In some embodiments, the antibody binds to a homolog of human FOLR1. In some aspects, the antibody binds to a homolog of human FOLR1 from a species selected from monkeys, mice, dogs, cats, rats, cows, horses, goats and sheep. In some aspects, the homolog is a cynomolgus monkey homolog. In some aspects, the homolog is a mouse or murine analog.

In some embodiments, the antibodies comprise at least one CDR sequence defined by a consensus sequence provided in this disclosure. In some embodiments, the antibodies comprise an illustrative CDR, V_{H}, or V_{L} sequence provided in this disclosure, or a variant thereof. In some aspects, the variant is a variant with a conservative amino acid substitution.

In some embodiments, the antibody has one or more CDRs having particular lengths, in terms of the number of amino acid residues. In some embodiments, the Chothia CDR-H1 of the antibody is 6, 7, or 8 residues in length. In some embodiments, the Kabat CDR-H1 of the antibody is 4, 5, or 6 residues in length. In some embodiments, the Chothia CDR-H2 of the antibody is 5, 6, or 7 residues in length. In some embodiments, the Kabat CDR-H2 of the antibody is 16, 17, or 18 residues in length. In some embodiments, the Kabat/Chothia CDR-H3 of the antibody is 13, 14, 15, 16, or 17 residues in length.

In some aspects, the Kabat/Chothia CDR-L1 of the antibody is 11, 12, 13, 14, 15, 16, 17, or 18 residues in length. In some aspects, the Kabat/Chothia CDR-L2 of the antibody is 6, 7, or 8 residues in length. In some aspects, the Kabat/Chothia CDR-L3 of the antibody is 8, 9, or 10 residues in length.

In some embodiments, the antibody comprises a light chain. In some aspects, the light chain is a kappa light chain. In some aspects, the light chain is a lambda light chain.

In some embodiments, the antibody comprises a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the antibody is an antibody fragment. In some aspects, the antibody fragment is an Fv fragment. In some aspects, the antibody fragment is a Fab fragment. In some aspects, the antibody fragment is a F(ab′)₂ fragment. In some aspects, the antibody fragment is a Fab′ fragment. In some aspects, the antibody fragment is an scFv (sFv) fragment. In some aspects, the antibody fragment is an scFv-Fc fragment.

In some embodiments, the antibody is a monoclonal antibody. In some embodiments, the antibody is a polyclonal antibody.

In some embodiments, the antibody is a chimeric antibody. In some embodiments, the antibody is a humanized antibody. In some embodiments, the antibody is a human antibody.

In some embodiments, the antibody is an affinity matured antibody. In some aspects, the antibody is an affinity matured antibody derived from an illustrative sequence provided in this disclosure.

The antibodies provided herein may be useful for the treatment of a variety of diseases and conditions including cancers. In some embodiments, the antibodies provided herein may be useful for the treatment of cancers of solid tumors. For example, the antibodies provided herein can be useful for the treatment of colorectal cancer.

In certain embodiments, the antibody comprises: three heavy chain CDRs and three light chain CDRs of a V_{H} region provided herein, and a V_{L} region provided herein. In certain embodiments, the V_{H} region is selected from SEQ ID NOs:308-366. In certain embodiments, the V_{L} region is selected from SEQ ID NOs: 367-369. In particular embodiments, the V_{H} region is according to SEQ ID NO:362, and a V_{L} region is according to SEQ ID NO:367. CDR sequences can be identified by routine techniques well known to those of skill in the art. In certain embodiments, the CDRs are identified according to Kabat numbering. In certain embodiments, the CDRs are identified according to Chothia numbering. In certain embodiments, the CDRs are identified according to Martin numbering. In certain embodiments, the CDRs are identified according to contact numbering. In certain embodiments, the CDRs identified according to AHo numbering. In certain embodiments, the CDRs identified according to IMGT numbering. Tools for identifying CDR sequences are available, for example, abYsis or abnum, supra.

### 5.1 CDR-H3 Sequences

In some embodiments, the antibody comprises a CDR-H3 sequence comprising, consisting of, or consisting essentially of a CDR-H3 sequence of an illustrative antibody or V_{H} sequence provided herein. In some aspects, the CDR-H3 sequence is a CDR-H3 sequence of a V_{H} sequence provided in SEQ ID NOs: 308-366.

In some embodiments, the antibody comprises a CDR-H3 sequence comprising, consisting of, or consisting essentially of a sequence selected from SEQ ID NOs: 240-298. In some aspects, the antibody comprises a CDR-H3 sequence comprising, consisting of, or consisting essentially of SEQ ID NO: 240. In some aspects, the antibody comprises a CDR-H3 sequence comprising, consisting of, or consisting essentially of SEQ ID NO: 241. In some aspects, the antibody comprises a CDR-H3 sequence comprising, consisting of, or consisting essentially of SEQ ID NO: 242. In some aspects, the antibody comprises a CDR-H3 sequence comprising, consisting of, or consisting essentially of SEQ ID NO: 243. In some aspects, the antibody comprises a CDR-H3 sequence comprising, consisting of, or consisting essentially of SEQ ID NO: 244. In some aspects, the antibody comprises a CDR-H3 sequence comprising, consisting of, or consisting essentially of SEQ ID NO: 245. In some aspects, the antibody comprises a CDR-H3 sequence comprising, consisting of, or consisting essentially of SEQ ID NO: 246. In some aspects, the antibody comprises a CDR-H3 sequence comprising, consisting of, or consisting essentially of SEQ ID NO: 247. In some aspects, the antibody comprises a CDR-H3 sequence comprising, consisting of, or consisting essentially of SEQ ID NO: 248. In some aspects, the antibody comprises a CDR-H3 sequence comprising, consisting of, or consisting essentially of SEQ ID NO: 249. In some aspects, the antibody comprises a CDR-H3 sequence comprising, consisting of, or consisting essentially of SEQ ID NO: 250. In some aspects, the antibody comprises a CDR-H3 sequence comprising, consisting of, or consisting essentially of SEQ ID NO: 251. In some aspects, the antibody comprises a CDR-H3 sequence comprising, consisting of, or consisting essentially of SEQ ID NO: 252. In some aspects, the antibody comprises a CDR-H3 sequence comprising, consisting of, or consisting essentially of SEQ ID NO: 253. In some aspects, the antibody comprises a CDR-H3 sequence comprising, consisting of, or consisting essentially of SEQ ID NO: 254. In some aspects, the antibody comprises a CDR-H3 sequence comprising, consisting of, or consisting essentially of SEQ ID NO: 255. In some aspects, the antibody comprises a CDR-H3 sequence comprising, consisting of, or consisting essentially of SEQ ID NO: 256. In some aspects, the antibody comprises a CDR-H3 sequence comprising, consisting of, or consisting essentially of SEQ ID NO: 257. In some aspects, the antibody comprises a CDR-H3 sequence comprising, consisting of, or consisting essentially of SEQ ID NO: 258. In some aspects, the antibody comprises a CDR-H3 sequence comprising, consisting of, or consisting essentially of SEQ ID NO: 259. In some aspects, the antibody comprises a CDR-H3 sequence comprising, consisting of, or consisting essentially of SEQ ID NO: 260. In some aspects, the antibody comprises a CDR-H3 sequence comprising, consisting of, or consisting essentially of SEQ ID NO: 261. In some aspects, the antibody comprises a CDR-H3 sequence comprising, consisting of, or consisting essentially of SEQ ID NO: 262. In some aspects, the antibody comprises a CDR-H3 sequence comprising, consisting of, or consisting essentially of SEQ ID NO: 263. In some aspects, the antibody comprises a CDR-H3 sequence comprising, consisting of, or consisting essentially of SEQ ID NO: 264. In some aspects, the antibody comprises a CDR-H3 sequence comprising, consisting of, or consisting essentially of SEQ ID NO: 265. In some aspects, the antibody comprises a CDR-H3 sequence comprising, consisting of, or consisting essentially of SEQ ID NO: 266. In some aspects, the antibody comprises a CDR-H3 sequence comprising, consisting of, or consisting essentially of SEQ ID NO: 267. In some aspects, the antibody comprises a CDR-H3 sequence comprising, consisting of, or consisting essentially of SEQ ID NO: 268. In some aspects, the antibody comprises a CDR-H3 sequence comprising, consisting of, or consisting essentially of SEQ ID NO: 269. In some aspects, the antibody comprises a CDR-H3 sequence comprising, consisting of, or consisting essentially of SEQ ID NO: 270. In some aspects, the antibody comprises a CDR-H3 sequence comprising, consisting of, or consisting essentially of SEQ ID NO: 271. In some aspects, the antibody comprises a CDR-H3 sequence comprising, consisting of, or consisting essentially of SEQ ID NO: 272. In some aspects, the antibody comprises a CDR-H3 sequence comprising, consisting of, or consisting essentially of SEQ ID NO: 273. In some aspects, the antibody comprises a CDR-H3 sequence comprising, consisting of, or consisting essentially of SEQ ID NO: 274. In some aspects, the antibody comprises a CDR-H3 sequence comprising, consisting of, or consisting essentially of SEQ ID NO: 275. In some aspects, the antibody comprises a CDR-H3 sequence comprising, consisting of, or consisting essentially of SEQ ID NO: 276. In some aspects, the antibody comprises a CDR-H3 sequence comprising, consisting of, or consisting essentially of SEQ ID NO: 277. In some aspects, the antibody comprises a CDR-H3 sequence comprising, consisting of, or consisting essentially of SEQ ID NO: 278. In some aspects, the antibody comprises a CDR-H3 sequence comprising, consisting of, or consisting essentially of SEQ ID NO: 279. In some aspects, the antibody comprises a CDR-H3 sequence comprising, consisting of, or consisting essentially of SEQ ID NO: 280. In some aspects, the antibody comprises a CDR-H3 sequence comprising, consisting of, or consisting essentially of SEQ ID NO: 281. In some aspects, the antibody comprises a CDR-H3 sequence comprising, consisting of, or consisting essentially of SEQ ID NO: 282. In some aspects, the antibody comprises a CDR-H3 sequence comprising, consisting of, or consisting essentially of SEQ ID NO: 283. In some aspects, the antibody comprises a CDR-H3 sequence comprising, consisting of, or consisting essentially of SEQ ID NO: 284. In some aspects, the antibody comprises a CDR-H3 sequence comprising, consisting of, or consisting essentially of SEQ ID NO: 285. In some aspects, the antibody comprises a CDR-H3 sequence comprising, consisting of, or consisting essentially of SEQ ID NO: 286. In some aspects, the antibody comprises a CDR-H3 sequence comprising, consisting of, or consisting essentially of SEQ ID NO: 287. In some aspects, the antibody comprises a CDR-H3 sequence comprising, consisting of, or consisting essentially of SEQ ID NO: 288. In some aspects, the antibody comprises a CDR-H3 sequence comprising, consisting of, or consisting essentially of SEQ ID NO: 289. In some aspects, the antibody comprises a CDR-H3 sequence comprising, consisting of, or consisting essentially of SEQ ID NO: 290. In some aspects, the antibody comprises a CDR-H3 sequence comprising, consisting of, or consisting essentially of SEQ ID NO: 291. In some aspects, the antibody comprises a CDR-H3 sequence comprising, consisting of, or consisting essentially of SEQ ID NO: 292. In some aspects, the antibody comprises a CDR-H3 sequence comprising, consisting of, or consisting essentially of SEQ ID NO: 293. In some aspects, the antibody comprises a CDR-H3 sequence comprising, consisting of, or consisting essentially of SEQ ID NO: 294. In some aspects, the antibody comprises a CDR-H3 sequence comprising, consisting of, or consisting essentially of SEQ ID NO: 295. In some aspects, the antibody comprises a CDR-H3 sequence comprising, consisting of, or consisting essentially of SEQ ID NO: 296. In some aspects, the antibody comprises a CDR-H3 sequence comprising, consisting of, or consisting essentially of SEQ ID NO: 297. In some aspects, the antibody comprises a CDR-H3 sequence comprising, consisting of, or consisting essentially of SEQ ID NO: 298.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

### 5.2 V_{H} Sequences Comprising Illustrative CDRs

In some embodiments, the antibody comprises a V_{H} sequence comprising one or more CDR-H sequences comprising, consisting of, or consisting essentially of one or more illustrative CDR-H sequences provided in this disclosure, and variants thereof. In some embodiments, the CDR-H sequences comprise, consist of, or consist essentially of one or more CDR-H sequences provided in a V_{H} sequence selected from SEQ ID NOs: 308-366.

### 5.2.1. V_{H} Sequences Comprising Illustrative Kabat CDRs

In some embodiments, the antibody comprises a V_{H} sequence comprising one or more Kabat CDR-H sequences comprising, consisting of, or consisting essentially of one or more illustrative Kabat CDR-H sequences provided in this disclosure, and variants thereof.

### 5.2.1.1. Kabat CDR-H3

In some embodiments, the antibody comprises a V_{H} sequence comprising a CDR-H3 sequence, wherein the CDR-H3 sequence comprises, consists of, or consists essentially of a Kabat CDR-H3 sequence of an illustrative antibody or V_{H} sequence provided herein. In some aspects, the Kabat CDR-H3 sequence is a Kabat CDR-H3 sequence of a V_{H} sequence provided in SEQ ID NOs: 308-366.

In some embodiments, the antibody comprises a V_{H} sequence comprising a Kabat CDR-H3 sequence comprising, consisting of, or consisting essentially of a sequence selected from SEQ ID NOs: 240-298. In some aspects, the antibody comprises a V_{H} sequence comprising a Kabat CDR-H3 sequence comprising, consisting of, or consisting essentially of SEQ ID NO: 240. In some aspects, the antibody comprises a V_{H} sequence comprising a Kabat CDR-H3 sequence comprising, consisting of, or consisting essentially of SEQ ID NO: 241. In some aspects, the antibody comprises a V_{H} sequence comprising a Kabat CDR-H3 sequence comprising, consisting of, or consisting essentially of SEQ ID NO: 242. In some aspects, the antibody comprises a V_{H} sequence comprising a Kabat CDR-H3 sequence comprising, consisting of, or consisting essentially of SEQ ID NO: 243. In some aspects, the antibody comprises a V_{H} sequence comprising a Kabat CDR-H3 sequence comprising, consisting of, or consisting essentially of SEQ ID NO: 244. In some aspects, the antibody comprises a V_{H}sequence comprising a Kabat CDR-H3 sequence comprising, consisting of, or consisting essentially of SEQ ID NO: 245. In some aspects, the antibody comprises a V_{H} sequence comprising a Kabat CDR-H3 sequence comprising, consisting of, or consisting essentially of SEQ ID NO: 246. In some aspects, the antibody comprises a V_{H} sequence comprising a Kabat CDR-H3 sequence comprising, consisting of, or consisting essentially of SEQ ID NO: 247. In some aspects, the antibody comprises a V_{H} sequence comprising a Kabat CDR-H3 sequence comprising, consisting of, or consisting essentially of SEQ ID NO: 248. In some aspects, the antibody comprises a V_{H} sequence comprising a Kabat CDR-H3 sequence comprising, consisting of, or consisting essentially of SEQ ID NO: 249. In some aspects, the antibody comprises a V_{H} sequence comprising a Kabat CDR-H3 sequence comprising, consisting of, or consisting essentially of SEQ ID NO: 250. In some aspects, the antibody comprises a V_{H}sequence comprising a Kabat CDR-H3 sequence comprising, consisting of, or consisting essentially of SEQ ID NO: 251. In some aspects, the antibody comprises a V_{H} sequence comprising a Kabat CDR-H3 sequence comprising, consisting of, or consisting essentially of SEQ ID NO: 252. In some aspects, the antibody comprises a V_{H} sequence comprising a Kabat CDR-H3 sequence comprising, consisting of, or consisting essentially of SEQ ID NO: 253. In some aspects, the antibody comprises a V_{H} sequence comprising a Kabat CDR-H3 sequence comprising, consisting of, or consisting essentially of SEQ ID NO: 254. In some aspects, the antibody comprises a V_{H} sequence comprising a Kabat CDR-H3 sequence comprising, consisting of, or consisting essentially of SEQ ID NO: 255. In some aspects, the antibody comprises a V_{H} sequence comprising a Kabat CDR-H3 sequence comprising, consisting of, or consisting essentially of SEQ ID NO: 256. In some aspects, the antibody comprises a V_{H} sequence comprising a Kabat CDR-H3 sequence comprising, consisting of, or consisting essentially of SEQ ID NO: 257. In some aspects, the antibody comprises a V_{H} sequence comprising a Kabat CDR-H3 sequence comprising, consisting of, or consisting essentially of SEQ ID NO: 258. In some aspects, the antibody comprises a V_{H} sequence comprising a Kabat CDR-H3 sequence comprising, consisting of, or consisting essentially of SEQ ID NO: 259. In some aspects, the antibody comprises a V_{H} sequence comprising a Kabat CDR-H3 sequence comprising, consisting of, or consisting essentially of SEQ ID NO: 260. In some aspects, the antibody comprises a V_{H} sequence comprising a Kabat CDR-H3 sequence comprising, consisting of, or consisting essentially of SEQ ID NO: 261. In some aspects, the antibody comprises a V_{H} sequence comprising a Kabat CDR-H3 sequence comprising, consisting of, or consisting essentially of SEQ ID NO: 262. In some aspects, the antibody comprises a V_{H}sequence comprising a Kabat CDR-H3 sequence comprising, consisting of, or consisting essentially of SEQ ID NO: 263. In some aspects, the antibody comprises a V_{H} sequence comprising a Kabat CDR-H3 sequence comprising, consisting of, or consisting essentially of SEQ ID NO: 264. In some aspects, the antibody comprises a V_{H} sequence comprising a Kabat CDR-H3 sequence comprising, consisting of, or consisting essentially of SEQ ID NO: 265. In some aspects, the antibody comprises a V_{H} sequence comprising a Kabat CDR-H3 sequence comprising, consisting of, or consisting essentially of SEQ ID NO: 266. In some aspects, the antibody comprises a V_{H} sequence comprising a Kabat CDR-H3 sequence comprising, consisting of, or consisting essentially of SEQ ID NO: 267. In some aspects, the antibody comprises a V_{H} sequence comprising a Kabat CDR-H3 sequence comprising, consisting of, or consisting essentially of SEQ ID NO: 268. In some aspects, the antibody comprises a V_{H}sequence comprising a Kabat CDR-H3 sequence comprising, consisting of, or consisting essentially of SEQ ID NO: 269. In some aspects, the antibody comprises a V_{H} sequence comprising a Kabat CDR-H3 sequence comprising, consisting of, or consisting essentially of SEQ ID NO: 270. In some aspects, the antibody comprises a V_{H} sequence comprising a Kabat CDR-H3 sequence comprising, consisting of, or consisting essentially of SEQ ID NO: 271. In some aspects, the antibody comprises a V_{H} sequence comprising a Kabat CDR-H3 sequence comprising, consisting of, or consisting essentially of SEQ ID NO: 272. In some aspects, the antibody comprises a V_{H} sequence comprising a Kabat CDR-H3 sequence comprising, consisting of, or consisting essentially of SEQ ID NO: 273. In some aspects, the antibody comprises a V_{H} sequence comprising a Kabat CDR-H3 sequence comprising, consisting of, or consisting essentially of SEQ ID NO: 274. In some aspects, the antibody comprises a V_{H}sequence comprising a Kabat CDR-H3 sequence comprising, consisting of, or consisting essentially of SEQ ID NO: 275. In some aspects, the antibody comprises a V_{H} sequence comprising a Kabat CDR-H3 sequence comprising, consisting of, or consisting essentially of SEQ ID NO: 276. In some aspects, the antibody comprises a V_{H} sequence comprising a Kabat CDR-H3 sequence comprising, consisting of, or consisting essentially of SEQ ID NO: 277. In some aspects, the antibody comprises a V_{H} sequence comprising a Kabat CDR-H3 sequence comprising, consisting of, or consisting essentially of SEQ ID NO: 278. In some aspects, the antibody comprises a V_{H} sequence comprising a Kabat CDR-H3 sequence comprising, consisting of, or consisting essentially of SEQ ID NO: 279. In some aspects, the antibody comprises a V_{H} sequence comprising a Kabat CDR-H3 sequence comprising, consisting of, or consisting essentially of SEQ ID NO: 280. In some aspects, the antibody comprises a V_{H}sequence comprising a Kabat CDR-H3 sequence comprising, consisting of, or consisting essentially of SEQ ID NO: 281. In some aspects, the antibody comprises a V_{H} sequence comprising a Kabat CDR-H3 sequence comprising, consisting of, or consisting essentially of SEQ ID NO: 282. In some aspects, the antibody comprises a V_{H} sequence comprising a Kabat CDR-H3 sequence comprising, consisting of, or consisting essentially of SEQ ID NO: 283. In some aspects, the antibody comprises a V_{H} sequence comprising a Kabat CDR-H3 sequence comprising, consisting of, or consisting essentially of SEQ ID NO: 284. In some aspects, the antibody comprises a V_{H} sequence comprising a Kabat CDR-H3 sequence comprising, consisting of, or consisting essentially of SEQ ID NO: 285. In some aspects, the antibody comprises a V_{H} sequence comprising a Kabat CDR-H3 sequence comprising, consisting of, or consisting essentially of SEQ ID NO: 286. In some aspects, the antibody comprises a V_{H}sequence comprising a Kabat CDR-H3 sequence comprising, consisting of, or consisting essentially of SEQ ID NO: 287. In some aspects, the antibody comprises a V_{H} sequence comprising a Kabat CDR-H3 sequence comprising, consisting of, or consisting essentially of SEQ ID NO: 288. In some aspects, the antibody comprises a V_{H} sequence comprising a Kabat CDR-H3 sequence comprising, consisting of, or consisting essentially of SEQ ID NO: 289. In some aspects, the antibody comprises a V_{H} sequence comprising a Kabat CDR-H3 sequence comprising, consisting of, or consisting essentially of SEQ ID NO: 290. In some aspects, the antibody comprises a V_{H} sequence comprising a Kabat CDR-H3 sequence comprising, consisting of, or consisting essentially of SEQ ID NO: 291. In some aspects, the antibody comprises a V_{H} sequence comprising a Kabat CDR-H3 sequence comprising, consisting of, or consisting essentially of SEQ ID NO: 292. In some aspects, the antibody comprises a V_{H} sequence comprising a Kabat CDR-H3 sequence comprising, consisting of, or consisting essentially of SEQ ID NO: 293. In some aspects, the antibody comprises a V_{H} sequence comprising a Kabat CDR-H3 sequence comprising, consisting of, or consisting essentially of SEQ ID NO: 294. In some aspects, the antibody comprises a V_{H} sequence comprising a Kabat CDR-H3 sequence comprising, consisting of, or consisting essentially of SEQ ID NO: 295. In some aspects, the antibody comprises a V_{H} sequence comprising a Kabat CDR-H3 sequence comprising, consisting of, or consisting essentially of SEQ ID NO: 296. In some aspects, the antibody comprises a V_{H} sequence comprising a Kabat CDR-H3 sequence comprising, consisting of, or consisting essentially of SEQ ID NO: 297. In some aspects, the antibody comprises a V_{H} sequence comprising a Kabat CDR-H3 sequence comprising, consisting of, or consisting essentially of SEQ ID NO: 298.

### 5.2.1.2. Kabat CDR-H2

In some embodiments, the antibody comprises a V_{H} sequence comprising a CDR-H2 sequence, wherein the CDR-H2 sequence comprises, consists of, or consists essentially of a Kabat CDR-H2 sequence of an illustrative antibody or V_{H} sequence provided herein. In some aspects, the Kabat CDR-H2 sequence is a Kabat CDR-H2 sequence of a V_{H} sequence provided in SEQ ID NOs: 308-366.

In some embodiments, the antibody comprises a V_{H} sequence comprising a Kabat CDR-H2 sequence comprising, consisting of, or consisting essentially of a sequence selected from SEQ ID NOs: 181-239. In some aspects, the antibody comprises a V_{H} sequence comprising a Kabat CDR-H2 sequence comprising, consisting of, or consisting essentially of SEQ ID NO: 181. In some aspects, the antibody comprises a V_{H} sequence comprising a Kabat CDR-H2 sequence comprising, consisting of, or consisting essentially of SEQ ID NO: 182. In some aspects, the antibody comprises a V_{H} sequence comprising a Kabat CDR-H2 sequence comprising, consisting of, or consisting essentially of SEQ ID NO: 183. In some aspects, the antibody comprises a V_{H} sequence comprising a Kabat CDR-H2 sequence comprising, consisting of, or consisting essentially of SEQ ID NO: 184. In some aspects, the antibody comprises a V_{H} sequence comprising a Kabat CDR-H2 sequence comprising, consisting of, or consisting essentially of SEQ ID NO: 185. In some aspects, the antibody comprises a V_{H}sequence comprising a Kabat CDR-H2 sequence comprising, consisting of, or consisting essentially of SEQ ID NO: 186. In some aspects, the antibody comprises a V_{H} sequence comprising a Kabat CDR-H2 sequence comprising, consisting of, or consisting essentially of SEQ ID NO: 187. In some aspects, the antibody comprises a V_{H} sequence comprising a Kabat CDR-H2 sequence comprising, consisting of, or consisting essentially of SEQ ID NO: 188. In some aspects, the antibody comprises a V_{H} sequence comprising a Kabat CDR-H2 sequence comprising, consisting of, or consisting essentially of SEQ ID NO: 189. In some aspects, the antibody comprises a V_{H} sequence comprising a Kabat CDR-H2 sequence comprising, consisting of, or consisting essentially of SEQ ID NO: 190. In some aspects, the antibody comprises a V_{H} sequence comprising a Kabat CDR-H2 sequence comprising, consisting of, or consisting essentially of SEQ ID NO: 191. In some aspects, the antibody comprises a V_{H}sequence comprising a Kabat CDR-H2 sequence comprising, consisting of, or consisting essentially of SEQ ID NO: 192. In some aspects, the antibody comprises a V_{H} sequence comprising a Kabat CDR-H2 sequence comprising, consisting of, or consisting essentially of SEQ ID NO: 193. In some aspects, the antibody comprises a V_{H} sequence comprising a Kabat CDR-H2 sequence comprising, consisting of, or consisting essentially of SEQ ID NO: 194. In some aspects, the antibody comprises a V_{H} sequence comprising a Kabat CDR-H2 sequence comprising, consisting of, or consisting essentially of SEQ ID NO: 195. In some aspects, the antibody comprises a V_{H} sequence comprising a Kabat CDR-H2 sequence comprising, consisting of, or consisting essentially of SEQ ID NO: 196. In some aspects, the antibody comprises a V_{H} sequence comprising a Kabat CDR-H2 sequence comprising, consisting of, or consisting essentially of SEQ ID NO: 197. In some aspects, the antibody comprises a V_{H}sequence comprising a Kabat CDR-H2 sequence comprising, consisting of, or consisting essentially of SEQ ID NO: 198. In some aspects, the antibody comprises a V_{H} sequence comprising a Kabat CDR-H2 sequence comprising, consisting of, or consisting essentially of SEQ ID NO: 199. In some aspects, the antibody comprises a V_{H} sequence comprising a Kabat CDR-H2 sequence comprising, consisting of, or consisting essentially of SEQ ID NO: 200. In some aspects, the antibody comprises a V_{H} sequence comprising a Kabat CDR-H2 sequence comprising, consisting of, or consisting essentially of SEQ ID NO: 201. In some aspects, the antibody comprises a V_{H} sequence comprising a Kabat CDR-H2 sequence comprising, consisting of, or consisting essentially of SEQ ID NO: 202. In some aspects, the antibody comprises a V_{H} sequence comprising a Kabat CDR-H2 sequence comprising, consisting of, or consisting essentially of SEQ ID NO: 203. In some aspects, the antibody comprises a V_{H}sequence comprising a Kabat CDR-H2 sequence comprising, consisting of, or consisting essentially of SEQ ID NO: 204. In some aspects, the antibody comprises a V_{H} sequence comprising a Kabat CDR-H2 sequence comprising, consisting of, or consisting essentially of SEQ ID NO: 205. In some aspects, the antibody comprises a V_{H} sequence comprising a Kabat CDR-H2 sequence comprising, consisting of, or consisting essentially of SEQ ID NO: 206. In some aspects, the antibody comprises a V_{H} sequence comprising a Kabat CDR-H2 sequence comprising, consisting of, or consisting essentially of SEQ ID NO: 207. In some aspects, the antibody comprises a V_{H} sequence comprising a Kabat CDR-H2 sequence comprising, consisting of, or consisting essentially of SEQ ID NO: 208. In some aspects, the antibody comprises a V_{H} sequence comprising a Kabat CDR-H2 sequence comprising, consisting of, or consisting essentially of SEQ ID NO: 209. In some aspects, the antibody comprises a V_{H}sequence comprising a Kabat CDR-H2 sequence comprising, consisting of, or consisting essentially of SEQ ID NO: 210. In some aspects, the antibody comprises a V_{H} sequence comprising a Kabat CDR-H2 sequence comprising, consisting of, or consisting essentially of SEQ ID NO: 211. In some aspects, the antibody comprises a V_{H} sequence comprising a Kabat CDR-H2 sequence comprising, consisting of, or consisting essentially of SEQ ID NO: 212. In some aspects, the antibody comprises a V_{H} sequence comprising a Kabat CDR-H2 sequence comprising, consisting of, or consisting essentially of SEQ ID NO: 213. In some aspects, the antibody comprises a V_{H} sequence comprising a Kabat CDR-H2 sequence comprising, consisting of, or consisting essentially of SEQ ID NO: 214. In some aspects, the antibody comprises a V_{H} sequence comprising a Kabat CDR-H2 sequence comprising, consisting of, or consisting essentially of SEQ ID NO: 215. In some aspects, the antibody comprises a V_{H}sequence comprising a Kabat CDR-H2 sequence comprising, consisting of, or consisting essentially of SEQ ID NO: 216. In some aspects, the antibody comprises a V_{H} sequence comprising a Kabat CDR-H2 sequence comprising, consisting of, or consisting essentially of SEQ ID NO: 217. In some aspects, the antibody comprises a V_{H} sequence comprising a Kabat CDR-H2 sequence comprising, consisting of, or consisting essentially of SEQ ID NO: 218. In some aspects, the antibody comprises a V_{H} sequence comprising a Kabat CDR-H2 sequence comprising, consisting of, or consisting essentially of SEQ ID NO: 219. In some aspects, the antibody comprises a V_{H} sequence comprising a Kabat CDR-H2 sequence comprising, consisting of, or consisting essentially of SEQ ID NO: 220. In some aspects, the antibody comprises a V_{H} sequence comprising a Kabat CDR-H2 sequence comprising, consisting of, or consisting essentially of SEQ ID NO: 221. In some aspects, the antibody comprises a V_{H}sequence comprising a Kabat CDR-H2 sequence comprising, consisting of, or consisting essentially of SEQ ID NO: 222. In some aspects, the antibody comprises a V_{H} sequence comprising a Kabat CDR-H2 sequence comprising, consisting of, or consisting essentially of SEQ ID NO: 223. In some aspects, the antibody comprises a V_{H} sequence comprising a Kabat CDR-H2 sequence comprising, consisting of, or consisting essentially of SEQ ID NO: 224. In some aspects, the antibody comprises a V_{H} sequence comprising a Kabat CDR-H2 sequence comprising, consisting of, or consisting essentially of SEQ ID NO: 225. In some aspects, the antibody comprises a V_{H} sequence comprising a Kabat CDR-H2 sequence comprising, consisting of, or consisting essentially of SEQ ID NO: 226. In some aspects, the antibody comprises a V_{H} sequence comprising a Kabat CDR-H2 sequence comprising, consisting of, or consisting essentially of SEQ ID NO: 227. In some aspects, the antibody comprises a V_{H}sequence comprising a Kabat CDR-H2 sequence comprising, consisting of, or consisting essentially of SEQ ID NO: 228. In some aspects, the antibody comprises a V_{H} sequence comprising a Kabat CDR-H2 sequence comprising, consisting of, or consisting essentially of SEQ ID NO: 229. In some aspects, the antibody comprises a V_{H} sequence comprising a Kabat CDR-H2 sequence comprising, consisting of, or consisting essentially of SEQ ID NO: 230. In some aspects, the antibody comprises a V_{H} sequence comprising a Kabat CDR-H2 sequence comprising, consisting of, or consisting essentially of SEQ ID NO: 231. In some aspects, the antibody comprises a V_{H} sequence comprising a Kabat CDR-H2 sequence comprising, consisting of, or consisting essentially of SEQ ID NO: 232. In some aspects, the antibody comprises a V_{H} sequence comprising a Kabat CDR-H2 sequence comprising, consisting of, or consisting essentially of SEQ ID NO: 233. In some aspects, the antibody comprises a V_{H}sequence comprising a Kabat CDR-H2 sequence comprising, consisting of, or consisting essentially of SEQ ID NO: 234. In some aspects, the antibody comprises a V_{H} sequence comprising a Kabat CDR-H2 sequence comprising, consisting of, or consisting essentially of SEQ ID NO: 235. In some aspects, the antibody comprises a V_{H} sequence comprising a Kabat CDR-H2 sequence comprising, consisting of, or consisting essentially of SEQ ID NO: 236. In some aspects, the antibody comprises a V_{H} sequence comprising a Kabat CDR-H2 sequence comprising, consisting of, or consisting essentially of SEQ ID NO: 237. In some aspects, the antibody comprises a V_{H} sequence comprising a Kabat CDR-H2 sequence comprising, consisting of, or consisting essentially of SEQ ID NO: 238. In some aspects, the antibody comprises a V_{H} sequence comprising a Kabat CDR-H2 sequence comprising, consisting of, or consisting essentially of SEQ ID NO: 239.

### 5.2.1.3. Kabat CDR-H1

In some embodiments, the antibody comprises a V_{H} sequence comprising a CDR-H1 sequence, wherein the CDR-H1 sequence comprises, consists of, or consists essentially of a Kabat CDR-H1 sequence of an illustrative antibody or V_{H} sequence provided herein. In some aspects, the Kabat CDR-H1 sequence is a Kabat CDR-H1 sequence of a V_{H} sequence provided in SEQ ID NOs: 308-366.

In some embodiments, the antibody comprises a V_{H} sequence comprising a Kabat CDR-H1 sequence comprising, consisting of, or consisting essentially of a sequence selected from SEQ ID NOs: 63-121. In some aspects, the antibody comprises a V_{H} sequence comprising a Kabat CDR-H1 sequence comprising, consisting of, or consisting essentially of SEQ ID NO: 63. In some aspects, the antibody comprises a V_{H} sequence comprising a Kabat CDR-H1 sequence comprising, consisting of, or consisting essentially of SEQ ID NO: 64. In some aspects, the antibody comprises a V_{H} sequence comprising a Kabat CDR-H1 sequence comprising, consisting of, or consisting essentially of SEQ ID NO: 65. In some aspects, the antibody comprises a V_{H} sequence comprising a Kabat CDR-H1 sequence comprising, consisting of, or consisting essentially of SEQ ID NO: 66. In some aspects, the antibody comprises a V_{H} sequence comprising a Kabat CDR-H1 sequence comprising, consisting of, or consisting essentially of SEQ ID NO: 67. In some aspects, the antibody comprises a V_{H} sequence comprising a Kabat CDR-H1 sequence comprising, consisting of, or consisting essentially of SEQ ID NO: 68. In some aspects, the antibody comprises a V_{H} sequence comprising a Kabat CDR-H1 sequence comprising, consisting of, or consisting essentially of SEQ ID NO: 69. In some aspects, the antibody comprises a V_{H} sequence comprising a Kabat CDR-H1 sequence comprising, consisting of, or consisting essentially of SEQ ID NO: 70. In some aspects, the antibody comprises a V_{H} sequence comprising a Kabat CDR-H1 sequence comprising, consisting of, or consisting essentially of SEQ ID NO: 71. In some aspects, the antibody comprises a V_{H} sequence comprising a Kabat CDR-H1 sequence comprising, consisting of, or consisting essentially of SEQ ID NO: 72. In some aspects, the antibody comprises a V_{H} sequence comprising a Kabat CDR-H1 sequence comprising, consisting of, or consisting essentially of SEQ ID NO: 73. In some aspects, the antibody comprises a V_{H} sequence comprising a Kabat CDR-H1 sequence comprising, consisting of, or consisting essentially of SEQ ID NO: 74. In some aspects, the antibody comprises a V_{H} sequence comprising a Kabat CDR-H1 sequence comprising, consisting of, or consisting essentially of SEQ ID NO: 75. In some aspects, the antibody comprises a V_{H} sequence comprising a Kabat CDR-H1 sequence comprising, consisting of, or consisting essentially of SEQ ID NO: 76. In some aspects, the antibody comprises a V_{H} sequence comprising a Kabat CDR-H1 sequence comprising, consisting of, or consisting essentially of SEQ ID NO: 77. In some aspects, the antibody comprises a V_{H} sequence comprising a Kabat CDR-H1 sequence comprising, consisting of, or consisting essentially of SEQ ID NO: 78. In some aspects, the antibody comprises a V_{H} sequence comprising a Kabat CDR-H1 sequence comprising, consisting of, or consisting essentially of SEQ ID NO: 79. In some aspects, the antibody comprises a V_{H} sequence comprising a Kabat CDR-H1 sequence comprising, consisting of, or consisting essentially of SEQ ID NO: 80. In some aspects, the antibody comprises a V_{H} sequence comprising a Kabat CDR-H1 sequence comprising, consisting of, or consisting essentially of SEQ ID NO: 81. In some aspects, the antibody comprises a V_{H} sequence comprising a Kabat CDR-H1 sequence comprising, consisting of, or consisting essentially of SEQ ID NO: 82. In some aspects, the antibody comprises a V_{H} sequence comprising a Kabat CDR-H1 sequence comprising, consisting of, or consisting essentially of SEQ ID NO: 83. In some aspects, the antibody comprises a V_{H} sequence comprising a Kabat CDR-H1 sequence comprising, consisting of, or consisting essentially of SEQ ID NO: 84. In some aspects, the antibody comprises a V_{H} sequence comprising a Kabat CDR-H1 sequence comprising, consisting of, or consisting essentially of SEQ ID NO: 85. In some aspects, the antibody comprises a V_{H} sequence comprising a Kabat CDR-H1 sequence comprising, consisting of, or consisting essentially of SEQ ID NO: 86. In some aspects, the antibody comprises a V_{H} sequence comprising a Kabat CDR-H1 sequence comprising, consisting of, or consisting essentially of SEQ ID NO: 87. In some aspects, the antibody comprises a V_{H} sequence comprising a Kabat CDR-H1 sequence comprising, consisting of, or consisting essentially of SEQ ID NO: 88. In some aspects, the antibody comprises a V_{H} sequence comprising a Kabat CDR-H1 sequence comprising, consisting of, or consisting essentially of SEQ ID NO: 89. In some aspects, the antibody comprises a V_{H} sequence comprising a Kabat CDR-H1 sequence comprising, consisting of, or consisting essentially of SEQ ID NO: 90. In some aspects, the antibody comprises a V_{H} sequence comprising a Kabat CDR-H1 sequence comprising, consisting of, or consisting essentially of SEQ ID NO: 91. In some aspects, the antibody comprises a V_{H} sequence comprising a Kabat CDR-H1 sequence comprising, consisting of, or consisting essentially of SEQ ID NO: 92. In some aspects, the antibody comprises a V_{H} sequence comprising a Kabat CDR-H1 sequence comprising, consisting of, or consisting essentially of SEQ ID NO: 93. In some aspects, the antibody comprises a V_{H} sequence comprising a Kabat CDR-H1 sequence comprising, consisting of, or consisting essentially of SEQ ID NO: 94. In some aspects, the antibody comprises a V_{H} sequence comprising a Kabat CDR-H1 sequence comprising, consisting of, or consisting essentially of SEQ ID NO: 95. In some aspects, the antibody comprises a V_{H} sequence comprising a Kabat CDR-H1 sequence comprising, consisting of, or consisting essentially of SEQ ID NO: 96. In some aspects, the antibody comprises a V_{H} sequence comprising a Kabat CDR-H1 sequence comprising, consisting of, or consisting essentially of SEQ ID NO: 97. In some aspects, the antibody comprises a V_{H} sequence comprising a Kabat CDR-H1 sequence comprising, consisting of, or consisting essentially of SEQ ID NO: 98. In some aspects, the antibody comprises a V_{H} sequence comprising a Kabat CDR-H1 sequence comprising, consisting of, or consisting essentially of SEQ ID NO: 99. In some aspects, the antibody comprises a V_{H} sequence comprising a Kabat CDR-H1 sequence comprising, consisting of, or consisting essentially of SEQ ID NO: 100. In some aspects, the antibody comprises a V_{H} sequence comprising a Kabat CDR-H1 sequence comprising, consisting of, or consisting essentially of SEQ ID NO: 101. In some aspects, the antibody comprises a V_{H}sequence comprising a Kabat CDR-H1 sequence comprising, consisting of, or consisting essentially of SEQ ID NO: 102. In some aspects, the antibody comprises a V_{H} sequence comprising a Kabat CDR-H1 sequence comprising, consisting of, or consisting essentially of SEQ ID NO: 103. In some aspects, the antibody comprises a V_{H} sequence comprising a Kabat CDR-H1 sequence comprising, consisting of, or consisting essentially of SEQ ID NO: 104. In some aspects, the antibody comprises a V_{H} sequence comprising a Kabat CDR-H1 sequence comprising, consisting of, or consisting essentially of SEQ ID NO: 105. In some aspects, the antibody comprises a V_{H} sequence comprising a Kabat CDR-H1 sequence comprising, consisting of, or consisting essentially of SEQ ID NO: 106. In some aspects, the antibody comprises a V_{H} sequence comprising a Kabat CDR-H1 sequence comprising, consisting of, or consisting essentially of SEQ ID NO: 107. In some aspects, the antibody comprises a V_{H}sequence comprising a Kabat CDR-H1 sequence comprising, consisting of, or consisting essentially of SEQ ID NO: 108. In some aspects, the antibody comprises a V_{H} sequence comprising a Kabat CDR-H1 sequence comprising, consisting of, or consisting essentially of SEQ ID NO: 109. In some aspects, the antibody comprises a V_{H} sequence comprising a Kabat CDR-H1 sequence comprising, consisting of, or consisting essentially of SEQ ID NO: 110. In some aspects, the antibody comprises a V_{H} sequence comprising a Kabat CDR-H1 sequence comprising, consisting of, or consisting essentially of SEQ ID NO: 111. In some aspects, the antibody comprises a V_{H} sequence comprising a Kabat CDR-H1 sequence comprising, consisting of, or consisting essentially of SEQ ID NO: 112. In some aspects, the antibody comprises a V_{H} sequence comprising a Kabat CDR-H1 sequence comprising, consisting of, or consisting essentially of SEQ ID NO: 113. In some aspects, the antibody comprises a V_{H}sequence comprising a Kabat CDR-H1 sequence comprising, consisting of, or consisting essentially of SEQ ID NO: 114. In some aspects, the antibody comprises a V_{H} sequence comprising a Kabat CDR-H1 sequence comprising, consisting of, or consisting essentially of SEQ ID NO: 115. In some aspects, the antibody comprises a V_{H} sequence comprising a Kabat CDR-H1 sequence comprising, consisting of, or consisting essentially of SEQ ID NO: 116. In some aspects, the antibody comprises a V_{H} sequence comprising a Kabat CDR-H1 sequence comprising, consisting of, or consisting essentially of SEQ ID NO: 117. In some aspects, the antibody comprises a V_{H} sequence comprising a Kabat CDR-H1 sequence comprising, consisting of, or consisting essentially of SEQ ID NO: 118. In some aspects, the antibody comprises a V_{H} sequence comprising a Kabat CDR-H1 sequence comprising, consisting of, or consisting essentially of SEQ ID NO: 119. In some aspects, the antibody comprises a V_{H}sequence comprising a Kabat CDR-H1 sequence comprising, consisting of, or consisting essentially of SEQ ID NO: 120. In some aspects, the antibody comprises a V_{H} sequence comprising a Kabat CDR-H1 sequence comprising, consisting of, or consisting essentially of SEQ ID NO: 121.

### 5.2.1.4. Kabat CDR-H3+Kabat CDR-H2

In some embodiments, the antibody comprises a V_{H} sequence comprising a Kabat CDR-H3 sequence comprising, consisting of, or consisting essentially of a sequence selected from SEQ ID NOs: 240-298, and a Kabat CDR-H2 sequence comprising, consisting of, or consisting essentially of a sequence selected from SEQ ID NOs: 181-239. In some aspects, the Kabat CDR-H3 sequence and the Kabat CDR-H2 sequence are both from a single illustrative V_{H} sequence provided in this disclosure. For example, in some aspects, the Kabat CDR-H3 and Kabat CDR-H2 are both from a single illustrative V_{H} sequence selected from SEQ ID NOs: 308-366.

### 5.2.1.5. Kabat CDR-H3+Kabat CDR-H1

In some embodiments, the antibody comprises a V_{H} sequence comprising a Kabat CDR-H3 sequence comprising, consisting of, or consisting essentially of a sequence selected from SEQ ID NOs: 240-298, and a Kabat CDR-H1 sequence comprising, consisting of, or consisting essentially of a sequence selected from SEQ ID NOs: 63-121. In some aspects, the Kabat CDR-H3 sequence and the Kabat CDR-H1 sequence are both from a single illustrative V_{H} sequence provided in this disclosure. For example, in some aspects, the Kabat CDR-H3 and Kabat CDR-H1 are both from a single illustrative V_{H} sequence selected from SEQ ID NOs: 308-366.

### 5.2.1.6 Kabat CDR-H1+Kabat CDR-H2

In some embodiments, the antibody comprises a V_{H} sequence comprising a Kabat CDR-H1 sequence comprising, consisting of, or consisting essentially of a sequence selected from SEQ ID NOs: 63-121 and a Kabat CDR-H2 sequence comprising, consisting of, or consisting essentially of a sequence selected from SEQ ID NOs: 181-239. In some aspects, the Kabat CDR-H1 sequence and the Kabat CDR-H2 sequence are both from a single illustrative V_{H} sequence provided in this disclosure. For example, in some aspects, the Kabat CDR-H1 and Kabat CDR-H2 are both from a single illustrative V_{H} sequence selected from SEQ ID NOs: 308-366.

### 5.2.1.7. Kabat CDR-H1+Kabat CDR-H2+Kabat CDR-H3

In some embodiments, the antibody comprises a V_{H} sequence comprising a Kabat CDR-H1 sequence comprising, consisting of, or consisting essentially of a sequence selected from SEQ ID NOs: 63-121, a Kabat CDR-H2 sequence comprising, consisting of, or consisting essentially of a sequence selected from SEQ ID NOs: 181-239, and a Kabat CDR-H3 sequence comprising, consisting of, or consisting essentially of a sequence selected from SEQ ID NOs: 240-298. In some aspects, the Kabat CDR-H1 sequence, Kabat CDR-H2 sequence, and Kabat CDR-H3 sequence are all from a single illustrative V_{H} sequence provided in this disclosure. For example, in some aspects, the Kabat CDR-H1, Kabat CDR-H2, and Kabat CDR-H3 are all from a single illustrative V_{H} sequence selected from SEQ ID NOs: 308-366.

### 5.2.1.8. Variants of V_{H} Sequences Comprising Illustrative Kabat CDRs

In some embodiments, the V_{H} sequences provided herein comprise a variant of an illustrative Kabat CDR-H3, CDR-H2, and/or CDR-H1 sequence provided in this disclosure.

In some aspects, the Kabat CDR-H3 sequence comprises, consists of, or consists essentially of a variant of an illustrative Kabat CDR-H3 sequence provided in this disclosure. In some aspects, the Kabat CDR-H3 sequence comprises, consists of, or consists essentially of a sequence having at least 70%, 75%, 80%, 85%, 90%, or 95% identity with any of the illustrative Kabat CDR-H3 sequences provided in this disclosure. In some aspects, the Kabat CDR-H3 sequence comprises, consists of, or consists essentially of any of the illustrative Kabat CDR-H3 sequences provided in this disclosure, with 1, 2, or 3 amino acid substitutions. In some aspects, the amino acid substitutions are conservative amino acid substitutions.

In some aspects, the Kabat CDR-H2 sequence comprises, consists of, or consists essentially of a variant of an illustrative Kabat CDR-H2 sequence provided in this disclosure. In some aspects, the Kabat CDR-H2 sequence comprises, consists of, or consists essentially of a sequence having at least 70%, 75%, 80%, 85%, 90%, or 95% identity with any of the illustrative Kabat CDR-H2 sequences provided in this disclosure. In some aspects, the Kabat CDR-H2 sequence comprises, consists of, or consists essentially of any of the illustrative Kabat CDR-H2 sequences provided in this disclosure, with 1, 2, or 3 amino acid substitutions. In some aspects, the amino acid substitutions are conservative amino acid substitutions.

In some aspects, the Kabat CDR-H1 sequence comprises, consists of, or consists essentially of a variant of an illustrative Kabat CDR-H1 sequence provided in this disclosure. In some aspects, the Kabat CDR-H1 sequence comprises, consists of, or consists essentially of a sequence having at least 70%, 75%, 80%, 85%, 90%, or 95% identity with any of the illustrative Kabat CDR-H1 sequences provided in this disclosure. In some aspects, the Kabat CDR-H1 sequence comprises, consists of, or consists essentially of any of the illustrative Kabat CDR-H1 sequences provided in this disclosure, with 1, 2, or 3 amino acid substitutions. In some aspects, the amino acid substitutions are conservative amino acid substitutions.

### 5.2.2. V_{H} Sequences Comprising Illustrative Chothia CDRs

In some embodiments, the antibody comprises a V_{H} sequence comprising one or more Chothia CDR-H sequences comprising, consisting of, or consisting essentially of one or more illustrative Chothia CDR-H sequences provided in this disclosure, and variants thereof.

### 5.2.2.1. Chothia CDR-H3

In some embodiments, the antibody comprises a V_{H} sequence comprising a CDR-H3 sequence, wherein the CDR-H3 sequence comprises, consists of, or consists essentially of a Chothia CDR-H3 sequence of an illustrative antibody or V_{H} sequence provided herein. In some aspects, the Chothia CDR-H3 sequence is a Chothia CDR-H3 sequence of a V_{H} sequence provided in SEQ ID NOs: 308-366.

In some embodiments, the antibody comprises a V_{H} sequence comprising a Chothia CDR-H3 sequence comprising, consisting of, or consisting essentially of a sequence selected from SEQ ID NOs: 240-298. In some aspects, the antibody comprises a V_{H} sequence comprising a Chothia CDR-H3 sequence comprising, consisting of, or consisting essentially of SEQ ID NO: 240. In some aspects, the antibody comprises a V_{H} sequence comprising a Chothia CDR-H3 sequence comprising, consisting of, or consisting essentially of SEQ ID NO: 241. In some aspects, the antibody comprises a V_{H} sequence comprising a Chothia CDR-H3 sequence comprising, consisting of, or consisting essentially of SEQ ID NO: 242. In some aspects, the antibody comprises a V_{H} sequence comprising a Chothia CDR-H3 sequence comprising, consisting of, or consisting essentially of SEQ ID NO: 243. In some aspects, the antibody comprises a V_{H} sequence comprising a Chothia CDR-H3 sequence comprising, consisting of, or consisting essentially of SEQ ID NO: 244. In some aspects, the antibody comprises a V_{H} sequence comprising a Chothia CDR-H3 sequence comprising, consisting of, or consisting essentially of SEQ ID NO: 245. In some aspects, the antibody comprises a V_{H} sequence comprising a Chothia CDR-H3 sequence comprising, consisting of, or consisting essentially of SEQ ID NO: 246. In some aspects, the antibody comprises a V_{H} sequence comprising a Chothia CDR-H3 sequence comprising, consisting of, or consisting essentially of SEQ ID NO: 247. In some aspects, the antibody comprises a V_{H} sequence comprising a Chothia CDR-H3 sequence comprising, consisting of, or consisting essentially of SEQ ID NO: 248. In some aspects, the antibody comprises a V_{H} sequence comprising a Chothia CDR-H3 sequence comprising, consisting of, or consisting essentially of SEQ ID NO: 249. In some aspects, the antibody comprises a V_{H} sequence comprising a Chothia CDR-H3 sequence comprising, consisting of, or consisting essentially of SEQ ID NO: 250. In some aspects, the antibody comprises a V_{H}sequence comprising a Chothia CDR-H3 sequence comprising, consisting of, or consisting essentially of SEQ ID NO: 251. In some aspects, the antibody comprises a V_{H} sequence comprising a Chothia CDR-H3 sequence comprising, consisting of, or consisting essentially of SEQ ID NO: 252. In some aspects, the antibody comprises a V_{H} sequence comprising a Chothia CDR-H3 sequence comprising, consisting of, or consisting essentially of SEQ ID NO: 253. In some aspects, the antibody comprises a V_{H} sequence comprising a Chothia CDR-H3 sequence comprising, consisting of, or consisting essentially of SEQ ID NO: 254. In some aspects, the antibody comprises a V_{H} sequence comprising a Chothia CDR-H3 sequence comprising, consisting of, or consisting essentially of SEQ ID NO: 255. In some aspects, the antibody comprises a V_{H} sequence comprising a Chothia CDR-H3 sequence comprising, consisting of, or consisting essentially of SEQ ID NO: 256. In some aspects, the antibody comprises a V_{H}sequence comprising a Chothia CDR-H3 sequence comprising, consisting of, or consisting essentially of SEQ ID NO: 257. In some aspects, the antibody comprises a V_{H} sequence comprising a Chothia CDR-H3 sequence comprising, consisting of, or consisting essentially of SEQ ID NO: 258. In some aspects, the antibody comprises a V_{H} sequence comprising a Chothia CDR-H3 sequence comprising, consisting of, or consisting essentially of SEQ ID NO: 259. In some aspects, the antibody comprises a V_{H} sequence comprising a Chothia CDR-H3 sequence comprising, consisting of, or consisting essentially of SEQ ID NO: 260. In some aspects, the antibody comprises a V_{H} sequence comprising a Chothia CDR-H3 sequence comprising, consisting of, or consisting essentially of SEQ ID NO: 261. In some aspects, the antibody comprises a V_{H} sequence comprising a Chothia CDR-H3 sequence comprising, consisting of, or consisting essentially of SEQ ID NO: 262. In some aspects, the antibody comprises a V_{H}sequence comprising a Chothia CDR-H3 sequence comprising, consisting of, or consisting essentially of SEQ ID NO: 263. In some aspects, the antibody comprises a V_{H} sequence comprising a Chothia CDR-H3 sequence comprising, consisting of, or consisting essentially of SEQ ID NO: 264. In some aspects, the antibody comprises a V_{H} sequence comprising a Chothia CDR-H3 sequence comprising, consisting of, or consisting essentially of SEQ ID NO: 265. In some aspects, the antibody comprises a V_{H} sequence comprising a Chothia CDR-H3 sequence comprising, consisting of, or consisting essentially of SEQ ID NO: 266. In some aspects, the antibody comprises a V_{H} sequence comprising a Chothia CDR-H3 sequence comprising, consisting of, or consisting essentially of SEQ ID NO: 267. In some aspects, the antibody comprises a V_{H} sequence comprising a Chothia CDR-H3 sequence comprising, consisting of, or consisting essentially of SEQ ID NO: 268. In some aspects, the antibody comprises a V_{H}sequence comprising a Chothia CDR-H3 sequence comprising, consisting of, or consisting essentially of SEQ ID NO: 269. In some aspects, the antibody comprises a V_{H} sequence comprising a Chothia CDR-H3 sequence comprising, consisting of, or consisting essentially of SEQ ID NO: 270. In some aspects, the antibody comprises a V_{H} sequence comprising a Chothia CDR-H3 sequence comprising, consisting of, or consisting essentially of SEQ ID NO: 271. In some aspects, the antibody comprises a V_{H} sequence comprising a Chothia CDR-H3 sequence comprising, consisting of, or consisting essentially of SEQ ID NO: 272. In some aspects, the antibody comprises a V_{H} sequence comprising a Chothia CDR-H3 sequence comprising, consisting of, or consisting essentially of SEQ ID NO: 273. In some aspects, the antibody comprises a V_{H} sequence comprising a Chothia CDR-H3 sequence comprising, consisting of, or consisting essentially of SEQ ID NO: 274. In some aspects, the antibody comprises a V_{H}sequence comprising a Chothia CDR-H3 sequence comprising, consisting of, or consisting essentially of SEQ ID NO: 275. In some aspects, the antibody comprises a V_{H} sequence comprising a Chothia CDR-H3 sequence comprising, consisting of, or consisting essentially of SEQ ID NO: 276. In some aspects, the antibody comprises a V_{H} sequence comprising a Chothia CDR-H3 sequence comprising, consisting of, or consisting essentially of SEQ ID NO: 277. In some aspects, the antibody comprises a V_{H} sequence comprising a Chothia CDR-H3 sequence comprising, consisting of, or consisting essentially of SEQ ID NO: 278. In some aspects, the antibody comprises a V_{H} sequence comprising a Chothia CDR-H3 sequence comprising, consisting of, or consisting essentially of SEQ ID NO: 279. In some aspects, the antibody comprises a V_{H} sequence comprising a Chothia CDR-H3 sequence comprising, consisting of, or consisting essentially of SEQ ID NO: 280. In some aspects, the antibody comprises a V_{H}sequence comprising a Chothia CDR-H3 sequence comprising, consisting of, or consisting essentially of SEQ ID NO: 281. In some aspects, the antibody comprises a V_{H} sequence comprising a Chothia CDR-H3 sequence comprising, consisting of, or consisting essentially of SEQ ID NO: 282. In some aspects, the antibody comprises a V_{H} sequence comprising a Chothia CDR-H3 sequence comprising, consisting of, or consisting essentially of SEQ ID NO: 283. In some aspects, the antibody comprises a V_{H} sequence comprising a Chothia CDR-H3 sequence comprising, consisting of, or consisting essentially of SEQ ID NO: 284. In some aspects, the antibody comprises a V_{H} sequence comprising a Chothia CDR-H3 sequence comprising, consisting of, or consisting essentially of SEQ ID NO: 285. In some aspects, the antibody comprises a V_{H} sequence comprising a Chothia CDR-H3 sequence comprising, consisting of, or consisting essentially of SEQ ID NO: 286. In some aspects, the antibody comprises a V_{H}sequence comprising a Chothia CDR-H3 sequence comprising, consisting of, or consisting essentially of SEQ ID NO: 287. In some aspects, the antibody comprises a V_{H} sequence comprising a Chothia CDR-H3 sequence comprising, consisting of, or consisting essentially of SEQ ID NO: 288. In some aspects, the antibody comprises a V_{H} sequence comprising a Chothia CDR-H3 sequence comprising, consisting of, or consisting essentially of SEQ ID NO: 289. In some aspects, the antibody comprises a V_{H} sequence comprising a Chothia CDR-H3 sequence comprising, consisting of, or consisting essentially of SEQ ID NO: 290. In some aspects, the antibody comprises a V_{H} sequence comprising a Chothia CDR-H3 sequence comprising, consisting of, or consisting essentially of SEQ ID NO: 291. In some aspects, the antibody comprises a V_{H} sequence comprising a Chothia CDR-H3 sequence comprising, consisting of, or consisting essentially of SEQ ID NO: 292. In some aspects, the antibody comprises a V_{H}sequence comprising a Chothia CDR-H3 sequence comprising, consisting of, or consisting essentially of SEQ ID NO: 293. In some aspects, the antibody comprises a V_{H} sequence comprising a Chothia CDR-H3 sequence comprising, consisting of, or consisting essentially of SEQ ID NO: 294. In some aspects, the antibody comprises a V_{H} sequence comprising a Chothia CDR-H3 sequence comprising, consisting of, or consisting essentially of SEQ ID NO: 295. In some aspects, the antibody comprises a V_{H} sequence comprising a Chothia CDR-H3 sequence comprising, consisting of, or consisting essentially of SEQ ID NO: 296. In some aspects, the antibody comprises a V_{H} sequence comprising a Chothia CDR-H3 sequence comprising, consisting of, or consisting essentially of SEQ ID NO: 297. In some aspects, the antibody comprises a V_{H} sequence comprising a Chothia CDR-H3 sequence comprising, consisting of, or consisting essentially of SEQ ID NO: 298.

### 5.2.2.2. Chothia CDR-H2

In some embodiments, the antibody comprises a V_{H} sequence comprising a CDR-H2 sequence, wherein the CDR-H2 sequence comprises, consists of, or consists essentially of a Chothia CDR-H2 sequence of an illustrative antibody or V_{H} sequence provided herein. In some aspects, the Chothia CDR-H2 sequence is a Chothia CDR-H2 sequence of a V_{H} sequence provided in SEQ ID NOs: 308-366.

In some embodiments, the antibody comprises a V_{H} sequence comprising a Chothia CDR-H2 sequence comprising, consisting of, or consisting essentially of a sequence selected from SEQ ID NOs: 122-180. In some aspects, the antibody comprises a V_{H} sequence comprising a Chothia CDR-H2 sequence comprising, consisting of, or consisting essentially of SEQ ID NO: 122. In some aspects, the antibody comprises a V_{H} sequence comprising a Chothia CDR-H2 sequence comprising, consisting of, or consisting essentially of SEQ ID NO: 123. In some aspects, the antibody comprises a V_{H} sequence comprising a Chothia CDR-H2 sequence comprising, consisting of, or consisting essentially of SEQ ID NO: 124. In some aspects, the antibody comprises a V_{H} sequence comprising a Chothia CDR-H2 sequence comprising, consisting of, or consisting essentially of SEQ ID NO: 125. In some aspects, the antibody comprises a V_{H} sequence comprising a Chothia CDR-H2 sequence comprising, consisting of, or consisting essentially of SEQ ID NO: 126. In some aspects, the antibody comprises a V_{H}sequence comprising a Chothia CDR-H2 sequence comprising, consisting of, or consisting essentially of SEQ ID NO: 127. In some aspects, the antibody comprises a V_{H} sequence comprising a Chothia CDR-H2 sequence comprising, consisting of, or consisting essentially of SEQ ID NO: 128. In some aspects, the antibody comprises a V_{H} sequence comprising a Chothia CDR-H2 sequence comprising, consisting of, or consisting essentially of SEQ ID NO: 129. In some aspects, the antibody comprises a V_{H} sequence comprising a Chothia CDR-H2 sequence comprising, consisting of, or consisting essentially of SEQ ID NO: 130. In some aspects, the antibody comprises a V_{H} sequence comprising a Chothia CDR-H2 sequence comprising, consisting of, or consisting essentially of SEQ ID NO: 131. In some aspects, the antibody comprises a V_{H} sequence comprising a Chothia CDR-H2 sequence comprising, consisting of, or consisting essentially of SEQ ID NO: 132. In some aspects, the antibody comprises a V_{H}sequence comprising a Chothia CDR-H2 sequence comprising, consisting of, or consisting essentially of SEQ ID NO: 133. In some aspects, the antibody comprises a V_{H} sequence comprising a Chothia CDR-H2 sequence comprising, consisting of, or consisting essentially of SEQ ID NO: 134. In some aspects, the antibody comprises a V_{H} sequence comprising a Chothia CDR-H2 sequence comprising, consisting of, or consisting essentially of SEQ ID NO: 135. In some aspects, the antibody comprises a V_{H} sequence comprising a Chothia CDR-H2 sequence comprising, consisting of, or consisting essentially of SEQ ID NO: 136. In some aspects, the antibody comprises a V_{H} sequence comprising a Chothia CDR-H2 sequence comprising, consisting of, or consisting essentially of SEQ ID NO: 137. In some aspects, the antibody comprises a V_{H} sequence comprising a Chothia CDR-H2 sequence comprising, consisting of, or consisting essentially of SEQ ID NO: 138. In some aspects, the antibody comprises a V_{H}sequence comprising a Chothia CDR-H2 sequence comprising, consisting of, or consisting essentially of SEQ ID NO: 139. In some aspects, the antibody comprises a V_{H} sequence comprising a Chothia CDR-H2 sequence comprising, consisting of, or consisting essentially of SEQ ID NO: 140. In some aspects, the antibody comprises a V_{H} sequence comprising a Chothia CDR-H2 sequence comprising, consisting of, or consisting essentially of SEQ ID NO: 141. In some aspects, the antibody comprises a V_{H} sequence comprising a Chothia CDR-H2 sequence comprising, consisting of, or consisting essentially of SEQ ID NO: 142. In some aspects, the antibody comprises a V_{H} sequence comprising a Chothia CDR-H2 sequence comprising, consisting of, or consisting essentially of SEQ ID NO: 143. In some aspects, the antibody comprises a V_{H} sequence comprising a Chothia CDR-H2 sequence comprising, consisting of, or consisting essentially of SEQ ID NO: 144. In some aspects, the antibody comprises a V_{H}sequence comprising a Chothia CDR-H2 sequence comprising, consisting of, or consisting essentially of SEQ ID NO: 145. In some aspects, the antibody comprises a V_{H} sequence comprising a Chothia CDR-H2 sequence comprising, consisting of, or consisting essentially of SEQ ID NO: 146. In some aspects, the antibody comprises a V_{H} sequence comprising a Chothia CDR-H2 sequence comprising, consisting of, or consisting essentially of SEQ ID NO: 147. In some aspects, the antibody comprises a V_{H} sequence comprising a Chothia CDR-H2 sequence comprising, consisting of, or consisting essentially of SEQ ID NO: 148. In some aspects, the antibody comprises a V_{H} sequence comprising a Chothia CDR-H2 sequence comprising, consisting of, or consisting essentially of SEQ ID NO: 149. In some aspects, the antibody comprises a V_{H} sequence comprising a Chothia CDR-H2 sequence comprising, consisting of, or consisting essentially of SEQ ID NO: 150. In some aspects, the antibody comprises a V_{H}sequence comprising a Chothia CDR-H2 sequence comprising, consisting of, or consisting essentially of SEQ ID NO: 151. In some aspects, the antibody comprises a V_{H} sequence comprising a Chothia CDR-H2 sequence comprising, consisting of, or consisting essentially of SEQ ID NO: 152. In some aspects, the antibody comprises a V_{H} sequence comprising a Chothia CDR-H2 sequence comprising, consisting of, or consisting essentially of SEQ ID NO: 153. In some aspects, the antibody comprises a V_{H} sequence comprising a Chothia CDR-H2 sequence comprising, consisting of, or consisting essentially of SEQ ID NO: 154. In some aspects, the antibody comprises a V_{H} sequence comprising a Chothia CDR-H2 sequence comprising, consisting of, or consisting essentially of SEQ ID NO: 155. In some aspects, the antibody comprises a V_{H} sequence comprising a Chothia CDR-H2 sequence comprising, consisting of, or consisting essentially of SEQ ID NO: 156. In some aspects, the antibody comprises a V_{H} sequence comprising a Chothia CDR-H2 sequence comprising, consisting of, or consisting essentially of SEQ ID NO: 157. In some aspects, the antibody comprises a V_{H} sequence comprising a Chothia CDR-H2 sequence comprising, consisting of, or consisting essentially of SEQ ID NO: 158. In some aspects, the antibody comprises a V_{H} sequence comprising a Chothia CDR-H2 sequence comprising, consisting of, or consisting essentially of SEQ ID NO: 159. In some aspects, the antibody comprises a V_{H} sequence comprising a Chothia CDR-H2 sequence comprising, consisting of, or consisting essentially of SEQ ID NO: 160. In some aspects, the antibody comprises a V_{H} sequence comprising a Chothia CDR-H2 sequence comprising, consisting of, or consisting essentially of SEQ ID NO: 161. In some aspects, the antibody comprises a V_{H} sequence comprising a Chothia CDR-H2 sequence comprising, consisting of, or consisting essentially of SEQ ID NO: 162. In some aspects, the antibody comprises a V_{H} sequence comprising a Chothia CDR-H2 sequence comprising, consisting of, or consisting essentially of SEQ ID NO: 163. In some aspects, the antibody comprises a V_{H} sequence comprising a Chothia CDR-H2 sequence comprising, consisting of, or consisting essentially of SEQ ID NO: 164. In some aspects, the antibody comprises a V_{H} sequence comprising a Chothia CDR-H2 sequence comprising, consisting of, or consisting essentially of SEQ ID NO: 165. In some aspects, the antibody comprises a V_{H} sequence comprising a Chothia CDR-H2 sequence comprising, consisting of, or consisting essentially of SEQ ID NO: 166. In some aspects, the antibody comprises a V_{H} sequence comprising a Chothia CDR-H2 sequence comprising, consisting of, or consisting essentially of SEQ ID NO: 167. In some aspects, the antibody comprises a V_{H} sequence comprising a Chothia CDR-H2 sequence comprising, consisting of, or consisting essentially of SEQ ID NO: 168. In some aspects, the antibody comprises a V_{H}sequence comprising a Chothia CDR-H2 sequence comprising, consisting of, or consisting essentially of SEQ ID NO: 169. In some aspects, the antibody comprises a V_{H} sequence comprising a Chothia CDR-H2 sequence comprising, consisting of, or consisting essentially of SEQ ID NO: 170. In some aspects, the antibody comprises a V_{H} sequence comprising a Chothia CDR-H2 sequence comprising, consisting of, or consisting essentially of SEQ ID NO: 171. In some aspects, the antibody comprises a V_{H} sequence comprising a Chothia CDR-H2 sequence comprising, consisting of, or consisting essentially of SEQ ID NO: 172. In some aspects, the antibody comprises a V_{H} sequence comprising a Chothia CDR-H2 sequence comprising, consisting of, or consisting essentially of SEQ ID NO: 173. In some aspects, the antibody comprises a V_{H} sequence comprising a Chothia CDR-H2 sequence comprising, consisting of, or consisting essentially of SEQ ID NO: 174. In some aspects, the antibody comprises a V_{H} sequence comprising a Chothia CDR-H2 sequence comprising, consisting of, or consisting essentially of SEQ ID NO: 175. In some aspects, the antibody comprises a V_{H} sequence comprising a Chothia CDR-H2 sequence comprising, consisting of, or consisting essentially of SEQ ID NO: 176. In some aspects, the antibody comprises a V_{H} sequence comprising a Chothia CDR-H2 sequence comprising, consisting of, or consisting essentially of SEQ ID NO: 177. In some aspects, the antibody comprises a V_{H} sequence comprising a Chothia CDR-H2 sequence comprising, consisting of, or consisting essentially of SEQ ID NO: 178. In some aspects, the antibody comprises a V_{H} sequence comprising a Chothia CDR-H2 sequence comprising, consisting of, or consisting essentially of SEQ ID NO: 179. In some aspects, the antibody comprises a V_{H} sequence comprising a Chothia CDR-H2 sequence comprising, consisting of, or consisting essentially of SEQ ID NO: 180.

### 5.2.2.3. Chothia CDR-H1

In some embodiments, the antibody comprises a V_{H} sequence comprising a CDR-H1 sequence, wherein the CDR-H1 sequence comprises, consists of, or consists essentially of a Chothia CDR-H1 sequence of an illustrative antibody or V_{H} sequence provided herein. In some aspects, the Chothia CDR-H1 sequence is a Chothia CDR-H1 sequence of a V_{H} sequence provided in SEQ ID NOs: 308-366.

In some embodiments, the antibody comprises a V_{H} sequence comprising a Chothia CDR-H1 sequence comprising, consisting of, or consisting essentially of a sequence selected from SEQ ID NOs: 4-62. In some aspects, the antibody comprises a V_{H} sequence comprising a Chothia CDR-H1 sequence comprising, consisting of, or consisting essentially of SEQ ID NO: 4. In some aspects, the antibody comprises a V_{H} sequence comprising a Chothia CDR-H1 sequence comprising, consisting of, or consisting essentially of SEQ ID NO: 5. In some aspects, the antibody comprises a V_{H} sequence comprising a Chothia CDR-H1 sequence comprising, consisting of, or consisting essentially of SEQ ID NO: 6. In some aspects, the antibody comprises a V_{H} sequence comprising a Chothia CDR-H1 sequence comprising, consisting of, or consisting essentially of SEQ ID NO: 7. In some aspects, the antibody comprises a V_{H} sequence comprising a Chothia CDR-H1 sequence comprising, consisting of, or consisting essentially of SEQ ID NO: 8. In some aspects, the antibody comprises a V_{H} sequence comprising a Chothia CDR-H1 sequence comprising, consisting of, or consisting essentially of SEQ ID NO: 9. In some aspects, the antibody comprises a V_{H} sequence comprising a Chothia CDR-H1 sequence comprising, consisting of, or consisting essentially of SEQ ID NO: 10. In some aspects, the antibody comprises a V_{H} sequence comprising a Chothia CDR-H1 sequence comprising, consisting of, or consisting essentially of SEQ ID NO: 11. In some aspects, the antibody comprises a V_{H} sequence comprising a Chothia CDR-H1 sequence comprising, consisting of, or consisting essentially of SEQ ID NO: 12. In some aspects, the antibody comprises a V_{H} sequence comprising a Chothia CDR-H1 sequence comprising, consisting of, or consisting essentially of SEQ ID NO: 13. In some aspects, the antibody comprises a V_{H} sequence comprising a Chothia CDR-H1 sequence comprising, consisting of, or consisting essentially of SEQ ID NO: 14. In some aspects, the antibody comprises a V_{H} sequence comprising a Chothia CDR-H1 sequence comprising, consisting of, or consisting essentially of SEQ ID NO: 15. In some aspects, the antibody comprises a V_{H} sequence comprising a Chothia CDR-H1 sequence comprising, consisting of, or consisting essentially of SEQ ID NO: 16. In some aspects, the antibody comprises a V_{H} sequence comprising a Chothia CDR-H1 sequence comprising, consisting of, or consisting essentially of SEQ ID NO: 17. In some aspects, the antibody comprises a V_{H} sequence comprising a Chothia CDR-H1 sequence comprising, consisting of, or consisting essentially of SEQ ID NO: 18. In some aspects, the antibody comprises a V_{H} sequence comprising a Chothia CDR-H1 sequence comprising, consisting of, or consisting essentially of SEQ ID NO: 19. In some aspects, the antibody comprises a V_{H} sequence comprising a Chothia CDR-H1 sequence comprising, consisting of, or consisting essentially of SEQ ID NO: 20. In some aspects, the antibody comprises a V_{H} sequence comprising a Chothia CDR-H1 sequence comprising, consisting of, or consisting essentially of SEQ ID NO: 21. In some aspects, the antibody comprises a V_{H} sequence comprising a Chothia CDR-H1 sequence comprising, consisting of, or consisting essentially of SEQ ID NO: 22. In some aspects, the antibody comprises a V_{H} sequence comprising a Chothia CDR-H1 sequence comprising, consisting of, or consisting essentially of SEQ ID NO: 23. In some aspects, the antibody comprises a V_{H} sequence comprising a Chothia CDR-H1 sequence comprising, consisting of, or consisting essentially of SEQ ID NO: 24. In some aspects, the antibody comprises a V_{H} sequence comprising a Chothia CDR-H1 sequence comprising, consisting of, or consisting essentially of SEQ ID NO: 25. In some aspects, the antibody comprises a V_{H} sequence comprising a Chothia CDR-H1 sequence comprising, consisting of, or consisting essentially of SEQ ID NO: 26. In some aspects, the antibody comprises a V_{H} sequence comprising a Chothia CDR-H1 sequence comprising, consisting of, or consisting essentially of SEQ ID NO: 27. In some aspects, the antibody comprises a V_{H} sequence comprising a Chothia CDR-H1 sequence comprising, consisting of, or consisting essentially of SEQ ID NO: 28. In some aspects, the antibody comprises a V_{H} sequence comprising a Chothia CDR-H1 sequence comprising, consisting of, or consisting essentially of SEQ ID NO: 29. In some aspects, the antibody comprises a V_{H} sequence comprising a Chothia CDR-H1 sequence comprising, consisting of, or consisting essentially of SEQ ID NO: 30. In some aspects, the antibody comprises a V_{H} sequence comprising a Chothia CDR-H1 sequence comprising, consisting of, or consisting essentially of SEQ ID NO: 31. In some aspects, the antibody comprises a V_{H} sequence comprising a Chothia CDR-H1 sequence comprising, consisting of, or consisting essentially of SEQ ID NO: 32. In some aspects, the antibody comprises a V_{H} sequence comprising a Chothia CDR-H1 sequence comprising, consisting of, or consisting essentially of SEQ ID NO: 33. In some aspects, the antibody comprises a V_{H} sequence comprising a Chothia CDR-H1 sequence comprising, consisting of, or consisting essentially of SEQ ID NO: 34. In some aspects, the antibody comprises a V_{H} sequence comprising a Chothia CDR-H1 sequence comprising, consisting of, or consisting essentially of SEQ ID NO: 35. In some aspects, the antibody comprises a V_{H} sequence comprising a Chothia CDR-H1 sequence comprising, consisting of, or consisting essentially of SEQ ID NO: 36. In some aspects, the antibody comprises a V_{H} sequence comprising a Chothia CDR-H1 sequence comprising, consisting of, or consisting essentially of SEQ ID NO: 37. In some aspects, the antibody comprises a V_{H} sequence comprising a Chothia CDR-H1 sequence comprising, consisting of, or consisting essentially of SEQ ID NO: 38. In some aspects, the antibody comprises a V_{H} sequence comprising a Chothia CDR-H1 sequence comprising, consisting of, or consisting essentially of SEQ ID NO: 39. In some aspects, the antibody comprises a V_{H} sequence comprising a Chothia CDR-H1 sequence comprising, consisting of, or consisting essentially of SEQ ID NO: 40. In some aspects, the antibody comprises a V_{H} sequence comprising a Chothia CDR-H1 sequence comprising, consisting of, or consisting essentially of SEQ ID NO: 41. In some aspects, the antibody comprises a V_{H} sequence comprising a Chothia CDR-H1 sequence comprising, consisting of, or consisting essentially of SEQ ID NO: 42. In some aspects, the antibody comprises a V_{H} sequence comprising a Chothia CDR-H1 sequence comprising, consisting of, or consisting essentially of SEQ ID NO: 43. In some aspects, the antibody comprises a V_{H} sequence comprising a Chothia CDR-H1 sequence comprising, consisting of, or consisting essentially of SEQ ID NO: 44. In some aspects, the antibody comprises a V_{H} sequence comprising a Chothia CDR-H1 sequence comprising, consisting of, or consisting essentially of SEQ ID NO: 45. In some aspects, the antibody comprises a V_{H} sequence comprising a Chothia CDR-H1 sequence comprising, consisting of, or consisting essentially of SEQ ID NO: 46. In some aspects, the antibody comprises a V_{H} sequence comprising a Chothia CDR-H1 sequence comprising, consisting of, or consisting essentially of SEQ ID NO: 47. In some aspects, the antibody comprises a V_{H} sequence comprising a Chothia CDR-H1 sequence comprising, consisting of, or consisting essentially of SEQ ID NO: 48. In some aspects, the antibody comprises a V_{H} sequence comprising a Chothia CDR-H1 sequence comprising, consisting of, or consisting essentially of SEQ ID NO: 49. In some aspects, the antibody comprises a V_{H} sequence comprising a Chothia CDR-H1 sequence comprising, consisting of, or consisting essentially of SEQ ID NO: 50. In some aspects, the antibody comprises a V_{H} sequence comprising a Chothia CDR-H1 sequence comprising, consisting of, or consisting essentially of SEQ ID NO: 51. In some aspects, the antibody comprises a V_{H} sequence comprising a Chothia CDR-H1 sequence comprising, consisting of, or consisting essentially of SEQ ID NO: 52. In some aspects, the antibody comprises a V_{H} sequence comprising a Chothia CDR-H1 sequence comprising, consisting of, or consisting essentially of SEQ ID NO: 53. In some aspects, the antibody comprises a V_{H} sequence comprising a Chothia CDR-H1 sequence comprising, consisting of, or consisting essentially of SEQ ID NO: 54. In some aspects, the antibody comprises a V_{H} sequence comprising a Chothia CDR-H1 sequence comprising, consisting of, or consisting essentially of SEQ ID NO: 55. In some aspects, the antibody comprises a V_{H} sequence comprising a Chothia CDR-H1 sequence comprising, consisting of, or consisting essentially of SEQ ID NO: 56. In some aspects, the antibody comprises a V_{H} sequence comprising a Chothia CDR-H1 sequence comprising, consisting of, or consisting essentially of SEQ ID NO: 57. In some aspects, the antibody comprises a V_{H} sequence comprising a Chothia CDR-H1 sequence comprising, consisting of, or consisting essentially of SEQ ID NO: 58. In some aspects, the antibody comprises a V_{H} sequence comprising a Chothia CDR-H1 sequence comprising, consisting of, or consisting essentially of SEQ ID NO: 59. In some aspects, the antibody comprises a V_{H} sequence comprising a Chothia CDR-H1 sequence comprising, consisting of, or consisting essentially of SEQ ID NO: 60. In some aspects, the antibody comprises a V_{H} sequence comprising a Chothia CDR-H1 sequence comprising, consisting of, or consisting essentially of SEQ ID NO: 61. In some aspects, the antibody comprises a V_{H} sequence comprising a Chothia CDR-H1 sequence comprising, consisting of, or consisting essentially of SEQ ID NO: 62.

### 5.2.2.4. Chothia CDR-H3+Chothia CDR-H2

In some embodiments, the antibody comprises a V_{H} sequence comprising a Chothia CDR-H3 sequence comprising, consisting of, or consisting essentially of a sequence selected from SEQ ID NOs: 240-298, and a Chothia CDR-H2 sequence comprising, consisting of, or consisting essentially of a sequence selected from SEQ ID NOs: 122-180. In some aspects, the Chothia CDR-H3 sequence and the Chothia CDR-H2 sequence are both from a single illustrative V_{H} sequence provided in this disclosure. For example, in some aspects, the Chothia CDR-H3 and Chothia CDR-H2 are both from a single illustrative V_{H} sequence selected from SEQ ID NOs: 308-366.

### 5.2.2.5. Chothia CDR-H3+Chothia CDR-H1

In some embodiments, the antibody comprises a V_{H} sequence comprising a Chothia CDR-H3 sequence comprising, consisting of, or consisting essentially of a sequence selected from SEQ ID NOs: 240-298, and a Chothia CDR-H1 sequence comprising, consisting of, or consisting essentially of a sequence selected from SEQ ID NOs: 4-62. In some aspects, the Chothia CDR-H3 sequence and the Chothia CDR-H1 sequence are both from a single illustrative V_{H} sequence provided in this disclosure. For example, in some aspects, the Chothia CDR-H3 and Chothia CDR-H1 are both from a single illustrative V_{H} sequence selected from SEQ ID NOs: 308-366.

### 5.2.2.6 Chothia CDR-H1+Chothia CDR-H2

In some embodiments, the antibody comprises a V_{H} sequence comprising a Chothia CDR-H1 sequence comprising, consisting of, or consisting essentially of a sequence selected from SEQ ID NOs: 4-62 and a Chothia CDR-H2 sequence comprising, consisting of, or consisting essentially of a sequence selected from SEQ ID NOs: 122-180. In some aspects, the Chothia CDR-H1 sequence and the Chothia CDR-H2 sequence are both from a single illustrative V_{H} sequence provided in this disclosure. For example, in some aspects, the Chothia CDR-H1 and Chothia CDR-H2 are both from a single illustrative V_{H} sequence selected from SEQ ID NOs: 308-366.

### 5.2.2.7. Chothia CDR-H1+Chothia CDR-H2+Chothia CDR-H3

In some embodiments, the antibody comprises a V_{H} sequence comprising a Chothia CDR-H1 sequence comprising, consisting of, or consisting essentially of a sequence selected from SEQ ID NOs: 4-62, a Chothia CDR-H2 sequence comprising, consisting of, or consisting essentially of a sequence selected from SEQ ID NOs: 122-180, and a Chothia CDR-H3 sequence comprising, consisting of, or consisting essentially of a sequence selected from SEQ ID NOs: 240-298. In some aspects, the Chothia CDR-H1 sequence, Chothia CDR-H2 sequence, and Chothia CDR-H3 sequence are all from a single illustrative V_{H} sequence provided in this disclosure. For example, in some aspects, the Chothia CDR-H1, Chothia CDR-H2, and Chothia CDR-H3 are all from a single illustrative V_{H} sequence selected from SEQ ID NOs: 308-366.

### 5.2.2.8. Variants of V_{H} Sequences Comprising Illustrative Chothia CDRs

In some embodiments, the V_{H} sequences provided herein comprise a variant of an illustrative Chothia CDR-H3, CDR-H2, and/or CDR-H1 sequence provided in this disclosure.

In some aspects, the Chothia CDR-H3 sequence comprises, consists of, or consists essentially of a variant of an illustrative Chothia CDR-H3 sequence provided in this disclosure. In some aspects, the Chothia CDR-H3 sequence comprises, consists of, or consists essentially of a sequence having at least 70%, 75%, 80%, 85%, 90%, or 95% identity with any of the illustrative Chothia CDR-H3 sequences provided in this disclosure. In some aspects, the Chothia CDR-H3 sequence comprises, consists of, or consists essentially of any of the illustrative Chothia CDR-H3 sequences provided in this disclosure, with 1, 2, or 3 amino acid substitutions. In some aspects, the amino acid substitutions are conservative amino acid substitutions.

In some aspects, the Chothia CDR-H2 sequence comprises, consists of, or consists essentially of a variant of an illustrative Chothia CDR-H2 sequence provided in this disclosure. In some aspects, the Chothia CDR-H2 sequence comprises, consists of, or consists essentially of a sequence having at least 70%, 75%, 80%, 85%, 90%, or 95% identity with any of the illustrative Chothia CDR-H2 sequences provided in this disclosure. In some aspects, the Chothia CDR-H2 sequence comprises, consists of, or consists essentially of any of the illustrative Chothia CDR-H2 sequences provided in this disclosure, with 1, 2, or 3 amino acid substitutions. In some aspects, the amino acid substitutions are conservative amino acid substitutions.

In some aspects, the Chothia CDR-H1 sequence comprises, consists of, or consists essentially of a variant of an illustrative Chothia CDR-H1 sequence provided in this disclosure. In some aspects, the Chothia CDR-H1 sequence comprises, consists of, or consists essentially of a sequence having at least 70%, 75%, 80%, 85%, 90%, or 95% identity with any of the illustrative Chothia CDR-H1 sequences provided in this disclosure. In some aspects, the Chothia CDR-H1 sequence comprises, consists of, or consists essentially of any of the illustrative Chothia CDR-H1 sequences provided in this disclosure, with 1, 2, or 3 amino acid substitutions. In some aspects, the amino acid substitutions are conservative amino acid substitutions.

### 5.3. V_{H} Sequences

In some embodiments, the antibody comprises, consists of, or consists essentially of a V_{H} sequence provided in SEQ ID NOs: 308-366.

In some embodiments, the antibody comprises a V_{H} sequence comprising, consisting of, or consisting essentially of a sequence selected from SEQ ID NOs: 308-366. In some aspects, the antibody comprises a V_{H} sequence comprising, consisting of, or consisting essentially of SEQ ID NO: 308. In some aspects, the antibody comprises a V_{H} sequence comprising, consisting of, or consisting essentially of SEQ ID NO: 309. In some aspects, the antibody comprises a V_{H}sequence comprising, consisting of, or consisting essentially of SEQ ID NO: 310. In some aspects, the antibody comprises a V_{H} sequence comprising, consisting of, or consisting essentially of SEQ ID NO: 311. In some aspects, the antibody comprises a V_{H} sequence comprising, consisting of, or consisting essentially of SEQ ID NO: 312. In some aspects, the antibody comprises a V_{H} sequence comprising, consisting of, or consisting essentially of SEQ ID NO: 313. In some aspects, the antibody comprises a V_{H} sequence comprising, consisting of, or consisting essentially of SEQ ID NO: 314. In some aspects, the antibody comprises a V_{H} sequence comprising, consisting of, or consisting essentially of SEQ ID NO: 315. In some aspects, the antibody comprises a V_{H} sequence comprising, consisting of, or consisting essentially of SEQ ID NO: 316. In some aspects, the antibody comprises a V_{H} sequence comprising, consisting of, or consisting essentially of SEQ ID NO: 317. In some aspects, the antibody comprises a V_{H} sequence comprising, consisting of, or consisting essentially of SEQ ID NO: 318. In some aspects, the antibody comprises a V_{H} sequence comprising, consisting of, or consisting essentially of SEQ ID NO: 319. In some aspects, the antibody comprises a V_{H}sequence comprising, consisting of, or consisting essentially of SEQ ID NO: 320. In some aspects, the antibody comprises a V_{H} sequence comprising, consisting of, or consisting essentially of SEQ ID NO: 321. In some aspects, the antibody comprises a V_{H} sequence comprising, consisting of, or consisting essentially of SEQ ID NO: 322. In some aspects, the antibody comprises a V_{H} sequence comprising, consisting of, or consisting essentially of SEQ ID NO: 323. In some aspects, the antibody comprises a V_{H} sequence comprising, consisting of, or consisting essentially of SEQ ID NO: 324. In some aspects, the antibody comprises a V_{H} sequence comprising, consisting of, or consisting essentially of SEQ ID NO: 325. In some aspects, the antibody comprises a V_{H} sequence comprising, consisting of, or consisting essentially of SEQ ID NO: 326. In some aspects, the antibody comprises a V_{H} sequence comprising, consisting of, or consisting essentially of SEQ ID NO: 327. In some aspects, the antibody comprises a V_{H} sequence comprising, consisting of, or consisting essentially of SEQ ID NO: 328. In some aspects, the antibody comprises a V_{H} sequence comprising, consisting of, or consisting essentially of SEQ ID NO: 329. In some aspects, the antibody comprises a V_{H}sequence comprising, consisting of, or consisting essentially of SEQ ID NO: 330. In some aspects, the antibody comprises a V_{H} sequence comprising, consisting of, or consisting essentially of SEQ ID NO: 331. In some aspects, the antibody comprises a V_{H} sequence comprising, consisting of, or consisting essentially of SEQ ID NO: 332. In some aspects, the antibody comprises a V_{H} sequence comprising, consisting of, or consisting essentially of SEQ ID NO: 333. In some aspects, the antibody comprises a V_{H} sequence comprising, consisting of, or consisting essentially of SEQ ID NO: 334. In some aspects, the antibody comprises a V_{H} sequence comprising, consisting of, or consisting essentially of SEQ ID NO: 335. In some aspects, the antibody comprises a V_{H} sequence comprising, consisting of, or consisting essentially of SEQ ID NO: 336. In some aspects, the antibody comprises a V_{H} sequence comprising, consisting of, or consisting essentially of SEQ ID NO: 337. In some aspects, the antibody comprises a V_{H} sequence comprising, consisting of, or consisting essentially of SEQ ID NO: 338. In some aspects, the antibody comprises a V_{H} sequence comprising, consisting of, or consisting essentially of SEQ ID NO: 339. In some aspects, the antibody comprises a V_{H}sequence comprising, consisting of, or consisting essentially of SEQ ID NO: 340. In some aspects, the antibody comprises a V_{H} sequence comprising, consisting of, or consisting essentially of SEQ ID NO: 341. In some aspects, the antibody comprises a V_{H} sequence comprising, consisting of, or consisting essentially of SEQ ID NO: 342. In some aspects, the antibody comprises a V_{H} sequence comprising, consisting of, or consisting essentially of SEQ ID NO: 343. In some aspects, the antibody comprises a V_{H} sequence comprising, consisting of, or consisting essentially of SEQ ID NO: 344. In some aspects, the antibody comprises a V_{H} sequence comprising, consisting of, or consisting essentially of SEQ ID NO: 345. In some aspects, the antibody comprises a V_{H} sequence comprising, consisting of, or consisting essentially of SEQ ID NO: 346. In some aspects, the antibody comprises a V_{H} sequence comprising, consisting of, or consisting essentially of SEQ ID NO: 347. In some aspects, the antibody comprises a V_{H} sequence comprising, consisting of, or consisting essentially of SEQ ID NO: 348. In some aspects, the antibody comprises a V_{H} sequence comprising, consisting of, or consisting essentially of SEQ ID NO: 349. In some aspects, the antibody comprises a V_{H}sequence comprising, consisting of, or consisting essentially of SEQ ID NO: 350. In some aspects, the antibody comprises a V_{H} sequence comprising, consisting of, or consisting essentially of SEQ ID NO: 351. In some aspects, the antibody comprises a V_{H} sequence comprising, consisting of, or consisting essentially of SEQ ID NO: 352. In some aspects, the antibody comprises a V_{H} sequence comprising, consisting of, or consisting essentially of SEQ ID NO: 353. In some aspects, the antibody comprises a V_{H} sequence comprising, consisting of, or consisting essentially of SEQ ID NO: 354. In some aspects, the antibody comprises a V_{H}sequence comprising, consisting of, or consisting essentially of SEQ ID NO: 355. In some aspects, the antibody comprises a V_{H} sequence comprising, consisting of, or consisting essentially of SEQ ID NO: 356. In some aspects, the antibody comprises a V_{H} sequence comprising, consisting of, or consisting essentially of SEQ ID NO: 357. In some aspects, the antibody comprises a V_{H} sequence comprising, consisting of, or consisting essentially of SEQ ID NO: 358. In some aspects, the antibody comprises a V_{H} sequence comprising, consisting of, or consisting essentially of SEQ ID NO: 359. In some aspects, the antibody comprises a V_{H}sequence comprising, consisting of, or consisting essentially of SEQ ID NO: 360. In some aspects, the antibody comprises a V_{H} sequence comprising, consisting of, or consisting essentially of SEQ ID NO: 361. In some aspects, the antibody comprises a V_{H} sequence comprising, consisting of, or consisting essentially of SEQ ID NO: 362. In some aspects, the antibody comprises a V_{H} sequence comprising, consisting of, or consisting essentially of SEQ ID NO: 363. In some aspects, the antibody comprises a V_{H} sequence comprising, consisting of, or consisting essentially of SEQ ID NO: 364. In some aspects, the antibody comprises a V_{H}sequence comprising, consisting of, or consisting essentially of SEQ ID NO: 365. In some aspects, the antibody comprises a V_{H} sequence comprising, consisting of, or consisting essentially of SEQ ID NO: 366.

### 5.3.1. Variants of V_{H} Sequences

In some embodiments, the V_{H} sequences provided herein comprise, consist of, or consist essentially of a variant of an illustrative V_{H} sequence provided in this disclosure.

In some aspects, the V_{H} sequence comprises, consists of, or consists essentially of a variant of an illustrative V_{H} sequence provided in this disclosure. In some aspects, the V_{H}sequence comprises, consists of, or consists essentially of a sequence having at least 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 5.4. CDR-L3 Sequences

In some embodiments, the antibody comprises a CDR-L3 sequence comprising, consisting of, or consisting essentially of a CDR-L3 sequence of an illustrative antibody or V_{L}sequence provided herein. In some aspects, the CDR-L3 sequence is a CDR-L3 sequence of a V_{L} sequence provided in SEQ ID NOs: 367-369.

In some embodiments, the antibody comprises a CDR-L3 sequence comprising, consisting of, or consisting essentially of a sequence selected from SEQ ID NOs: 305-307. In some aspects, the antibody comprises a CDR-L3 sequence comprising, consisting of, or consisting essentially of SEQ ID NO: 305. In some aspects, the antibody comprises a CDR-L3 sequence comprising, consisting of, or consisting essentially of SEQ ID NO: 306. In some aspects, the antibody comprises a CDR-L3 sequence comprising, consisting of, or consisting essentially of SEQ ID NO: 307.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

### 5.5. V_{L} Sequences Comprising Illustrative CDRs

In some embodiments, the antibody comprises a V_{L} sequence comprising one or more CDR-L sequences comprising, consisting of, or consisting essentially of one or more illustrative CDR-L sequences provided in this disclosure, and variants thereof.

### 5.5.1. CDR-L3

In some embodiments, the antibody comprises a V_{L} sequence comprising a CDR-L3 sequence, wherein the CDR-L3 sequence comprises, consists of, or consists essentially of a CDR-L3 sequence of an illustrative antibody or V_{L} sequence provided herein. In some aspects, the CDR-L3 sequence is a CDR-L3 sequence of a V_{L} sequence provided in SEQ ID NOs: 367-369.

In some embodiments, the antibody comprises a V_{L} sequence comprising a CDR-L3 sequence comprising, consisting of, or consisting essentially of a sequence selected from SEQ ID NOs: 305-307. In some aspects, the antibody comprises a V_{L} sequence comprising a CDR-L3 sequence comprising, consisting of, or consisting essentially of SEQ ID NO: 305. In some aspects, the antibody comprises a V_{L} sequence comprising a CDR-L3 sequence comprising, consisting of, or consisting essentially of SEQ ID NO: 306. In some aspects, the antibody comprises a V_{L} sequence comprising a CDR-L3 sequence comprising, consisting of, or consisting essentially of SEQ ID NO: 307.

### 5.5.2. CDR-L2

In some embodiments, the antibody comprises a V_{L} sequence comprising a CDR-L2 sequence, wherein the CDR-L2 sequence comprises, consists of, or consists essentially of a CDR-L2 sequence of an illustrative antibody or V_{L} sequence provided herein. In some aspects, the CDR-L2 sequence is a CDR-L2 sequence of a V_{L} sequence provided in SEQ ID NOs: 367-369.

In some embodiments, the antibody comprises a V_{L} sequence comprising a CDR-L2 sequence comprising, consisting of, or consisting essentially of a sequence selected from SEQ ID NOs: 302-304. In some aspects, the antibody comprises a V_{L} sequence comprising a CDR-L2 sequence comprising, consisting of, or consisting essentially of SEQ ID NO: 302. In some aspects, the antibody comprises a V_{L} sequence comprising a CDR-L2 sequence comprising, consisting of, or consisting essentially of SEQ ID NO: 303. In some aspects, the antibody comprises a V_{L} sequence comprising a CDR-L2 sequence comprising, consisting of, or consisting essentially of SEQ ID NO: 304.

### 5.5.3. CDR-L1

In some embodiments, the antibody comprises a V_{L} sequence comprising a CDR-L1 sequence, wherein the CDR-L1 sequence comprises, consists of, or consists essentially of a CDR-L1 sequence of an illustrative antibody or V_{L} sequence provided herein. In some aspects, the CDR-L1 sequence is a CDR-L1 sequence of a V_{L} sequence provided in SEQ ID NOs: 367-369.

In some embodiments, the antibody comprises a V_{L} sequence comprising a CDR-L1 sequence comprising, consisting of, or consisting essentially of a sequence selected from SEQ ID NOs: 299-301. In some aspects, the antibody comprises a V_{L} sequence comprising a CDR-L1 sequence comprising, consisting of, or consisting essentially of SEQ ID NO: 299. In some aspects, the antibody comprises a V_{L} sequence comprising a CDR-L1 sequence comprising, consisting of, or consisting essentially of SEQ ID NO: 300. In some aspects, the antibody comprises a V_{L} sequence comprising a CDR-L1 sequence comprising, consisting of, or consisting essentially of SEQ ID NO: 301.

### 5.5.4. CDR-L3+CDR-L2

In some embodiments, the antibody comprises a V_{L} sequence comprising a CDR-L3 sequence comprising, consisting of, or consisting essentially of a sequence selected from SEQ ID NOs: 305-307 and a CDR-L2 sequence comprising, consisting of, or consisting essentially of a sequence selected from SEQ ID NOs: 302-304. In some aspects, the CDR-L3 sequence and the CDR-L2 sequence are both from a single illustrative V_{L} sequence provided in this disclosure. For example, in some aspects, the CDR-L3 and CDR-L2 are both from a single illustrative V_{L}sequence selected from SEQ ID NOs: 367-369.

### 5.5.5. CDR-L3+CDR-L1

In some embodiments, the antibody comprises a V_{L} sequence comprising a CDR-L3 sequence comprising, consisting of, or consisting essentially of a sequence selected from SEQ ID NOs: 305-307 and a CDR-L1 sequence comprising, consisting of, or consisting essentially of a sequence selected from SEQ ID NOs: 299-301. In some aspects, the CDR-L3 sequence and the CDR-L1 sequence are both from a single illustrative V_{L} sequence provided in this disclosure. For example, in some aspects, the CDR-L3 and CDR-L1 are both from a single illustrative V_{L}sequence selected from SEQ ID NOs: 367-369.

### 5.5.6. CDR-L1+CDR-L2

In some embodiments, the antibody comprises a V_{L} sequence comprising a CDR-L1 sequence comprising, consisting of, or consisting essentially of a sequence selected from SEQ ID NOs: 299-301 and a CDR-L2 sequence comprising, consisting of, or consisting essentially of a sequence selected from SEQ ID NOs: 302-304. In some aspects, the CDR-L1 sequence and the CDR-L2 sequence are both from a single illustrative V_{L} sequence provided in this disclosure. For example, in some aspects, the CDR-L1 and CDR-L2 are both from a single illustrative V_{L}sequence selected from SEQ ID NOs: 367-369.

### 5.5.7. CDR-L1+CDR-L2+CDR-L3

In some embodiments, the antibody comprises a V_{L} sequence comprising a CDR-L1 sequence comprising, consisting of, or consisting essentially of a sequence selected from SEQ ID NOs: 299-301, a CDR-L2 sequence comprising, consisting of, or consisting essentially of a sequence selected from SEQ ID NOs: 302-304, and a CDR-L3 sequence comprising, consisting of, or consisting essentially of a sequence selected from SEQ ID NOs: 305-307. In some aspects, the CDR-L1 sequence, CDR-L2 sequence, and CDR-L3 sequence are all from a single illustrative V_{L} sequence provided in this disclosure. For example, in some aspects, the CDR-L1, CDR-L2, and CDR-L3 are all from a single illustrative V_{L} sequence selected from SEQ ID NOs: 367-369.

### 5.5.8. Variants of V_{L} Sequences Comprising Illustrative CDR-Ls

In some embodiments, the V_{L} sequences provided herein comprise a variant of an illustrative CDR-L3, CDR-L2, and/or CDR-L1 sequence provided in this disclosure.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

### 5.6. V_{L} Sequences

In some embodiments, the antibody comprises, consists of, or consists essentially of a V_{L} sequence provided in SEQ ID NOs: 367-369.

In some embodiments, the antibody comprises a V_{L} sequence comprising, consisting of, or consisting essentially of a sequence selected from SEQ ID NOs: 367-369. In some aspects, the antibody comprises a V_{L} sequence comprising, consisting of, or consisting essentially of SEQ ID NO: 367. In some aspects, the antibody comprises a V_{L} sequence comprising, consisting of, or consisting essentially of SEQ ID NO: 368. In some aspects, the antibody comprises a V_{L}sequence comprising, consisting of, or consisting essentially of SEQ ID NO: 369.

### 5.61. Variants of V_{L} Sequences

In some embodiments, the V_{L} sequences provided herein comprise, consist of, or consist essentially of a variant of an illustrative V_{L} sequence provided in this disclosure.

In some aspects, the V_{L} sequence comprises, consists of, or consists essentially of a variant of an illustrative V_{L} sequence provided in this disclosure. In some aspects, the V_{L}sequence comprises, consists of, or consists essentially of a sequence having at least 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 5.7. Pairs

### 5.7.1. CDR-H3 CDR-L3 Pairs

In some embodiments, the antibody comprises a CDR-H3 sequence and a CDR-L3 sequence. In some aspects, the CDR-H3 sequence is part of a V_{H} and the CDR-L3 sequence is part of a V_{L}.

In some aspects, the CDR-H3 sequence is a CDR-H3 sequence comprising, consisting of, or consisting essentially of SEQ ID NOs: 240-298, and the CDR-L3 sequence is a CDR-L3 sequence comprising, consisting of, or consisting essentially of SEQ ID NOs: 305-307.

In some aspects, the CDR-H3-CDR-L3 pairs are selected from SEQ ID NO: 305 and SEQ ID NO: 240; SEQ ID NO: 305 and SEQ ID NO: 241; SEQ ID NO: 305 and SEQ ID NO: 242; SEQ ID NO: 305 and SEQ ID NO: 243; SEQ ID NO: 305 and SEQ ID NO: 244; SEQ ID NO: 305 and SEQ ID NO: 245; SEQ ID NO: 305 and SEQ ID NO: 246; SEQ ID NO: 305 and SEQ ID NO: 247; SEQ ID NO: 305 and SEQ ID NO: 248; SEQ ID NO: 305 and SEQ ID NO: 249; SEQ ID NO: 305 and SEQ ID NO: 250; SEQ ID NO: 305 and SEQ ID NO: 251; SEQ ID NO: 305 and SEQ ID NO: 252; SEQ ID NO: 305 and SEQ ID NO: 253; SEQ ID NO: 305 and SEQ ID NO: 254; SEQ ID NO: 305 and SEQ ID NO: 255; SEQ ID NO: 305 and SEQ ID NO: 256; SEQ ID NO: 305 and SEQ ID NO: 257; SEQ ID NO: 305 and SEQ ID NO: 258; SEQ ID NO: 305 and SEQ ID NO: 259; SEQ ID NO: 305 and SEQ ID NO: 260; SEQ ID NO: 305 and SEQ ID NO: 261; SEQ ID NO: 305 and SEQ ID NO: 262; SEQ ID NO: 305 and SEQ ID NO: 263; SEQ ID NO: 305 and SEQ ID NO: 264; SEQ ID NO: 305 and SEQ ID NO: 265; SEQ ID NO: 305 and SEQ ID NO: 266; SEQ ID NO: 305 and SEQ ID NO: 267; SEQ ID NO: 305 and SEQ ID NO: 268; SEQ ID NO: 305 and SEQ ID NO: 269; SEQ ID NO: 305 and SEQ ID NO: 270; SEQ ID NO: 305 and SEQ ID NO: 271; SEQ ID NO: 305 and SEQ ID NO: 272; SEQ ID NO: 305 and SEQ ID NO: 273; SEQ ID NO: 305 and SEQ ID NO: 274; SEQ ID NO: 305 and SEQ ID NO: 275; SEQ ID NO: 305 and SEQ ID NO: 276; SEQ ID NO: 305 and SEQ ID NO: 277; SEQ ID NO: 305 and SEQ ID NO: 278; SEQ ID NO: 305 and SEQ ID NO: 279; SEQ ID NO: 305 and SEQ ID NO: 280; SEQ ID NO: 305 and SEQ ID NO: 281; SEQ ID NO: 305 and SEQ ID NO: 282; SEQ ID NO: 305 and SEQ ID NO: 283; SEQ ID NO: 305 and SEQ ID NO: 284; SEQ ID NO: 305 and SEQ ID NO: 285; SEQ ID NO: 305 and SEQ ID NO: 286; SEQ ID NO: 305 and SEQ ID NO: 287; SEQ ID NO: 305 and SEQ ID NO: 288; SEQ ID NO: 305 and SEQ ID NO: 289; SEQ ID NO: 305 and SEQ ID NO: 290; SEQ ID NO: 305 and SEQ ID NO: 291; SEQ ID NO: 305 and SEQ ID NO: 292; SEQ ID NO: 305 and SEQ ID NO: 293; SEQ ID NO: 305 and SEQ ID NO: 294; SEQ ID NO: 305 and SEQ ID NO: 295; SEQ ID NO: 305 and SEQ ID NO: 296; SEQ ID NO: 305 and SEQ ID NO: 297; and SEQ ID NO: 305 and SEQ ID NO: 298.

In some aspects, the CDR-H3-CDR-L3 pairs are selected from SEQ ID NO: 306 and SEQ ID NO: 240; SEQ ID NO: 306 and SEQ ID NO: 241; SEQ ID NO: 306 and SEQ ID NO: 242; SEQ ID NO: 306 and SEQ ID NO: 243; SEQ ID NO: 306 and SEQ ID NO: 244; SEQ ID NO: 306 and SEQ ID NO: 245; SEQ ID NO: 306 and SEQ ID NO: 246; SEQ ID NO: 306 and SEQ ID NO: 247; SEQ ID NO: 306 and SEQ ID NO: 248; SEQ ID NO: 306 and SEQ ID NO: 249; SEQ ID NO: 306 and SEQ ID NO: 250; SEQ ID NO: 306 and SEQ ID NO: 251; SEQ ID NO: 306 and SEQ ID NO: 252; SEQ ID NO: 306 and SEQ ID NO: 253; SEQ ID NO: 306 and SEQ ID NO: 254; SEQ ID NO: 306 and SEQ ID NO: 255; SEQ ID NO: 306 and SEQ ID NO: 256; SEQ ID NO: 306 and SEQ ID NO: 257; SEQ ID NO: 306 and SEQ ID NO: 258; SEQ ID NO: 306 and SEQ ID NO: 259; SEQ ID NO: 306 and SEQ ID NO: 260; SEQ ID NO: 306 and SEQ ID NO: 261; SEQ ID NO: 306 and SEQ ID NO: 262; SEQ ID NO: 306 and SEQ ID NO: 263; SEQ ID NO: 306 and SEQ ID NO: 264; SEQ ID NO: 306 and SEQ ID NO: 265; SEQ ID NO: 306 and SEQ ID NO: 266; SEQ ID NO: 306 and SEQ ID NO: 267; SEQ ID NO: 306 and SEQ ID NO: 268; SEQ ID NO: 306 and SEQ ID NO: 269; SEQ ID NO: 306 and SEQ ID NO: 270; SEQ ID NO: 306 and SEQ ID NO: 271; SEQ ID NO: 306 and SEQ ID NO: 272; SEQ ID NO: 306 and SEQ ID NO: 273; SEQ ID NO: 306 and SEQ ID NO: 274; SEQ ID NO: 306 and SEQ ID NO: 275; SEQ ID NO: 306 and SEQ ID NO: 276; SEQ ID NO: 306 and SEQ ID NO: 277; SEQ ID NO: 306 and SEQ ID NO: 278; SEQ ID NO: 306 and SEQ ID NO: 279; SEQ ID NO: 306 and SEQ ID NO: 280; SEQ ID NO: 306 and SEQ ID NO: 281; SEQ ID NO: 306 and SEQ ID NO: 282; SEQ ID NO: 306 and SEQ ID NO: 283; SEQ ID NO: 306 and SEQ ID NO: 284; SEQ ID NO: 306 and SEQ ID NO: 285; SEQ ID NO: 306 and SEQ ID NO: 286; SEQ ID NO: 306 and SEQ ID NO: 287; SEQ ID NO: 306 and SEQ ID NO: 288; SEQ ID NO: 306 and SEQ ID NO: 289; SEQ ID NO: 306 and SEQ ID NO: 290; SEQ ID NO: 306 and SEQ ID NO: 291; SEQ ID NO: 306 and SEQ ID NO: 292; SEQ ID NO: 306 and SEQ ID NO: 293; SEQ ID NO: 306 and SEQ ID NO: 294; SEQ ID NO: 306 and SEQ ID NO: 295; SEQ ID NO: 306 and SEQ ID NO: 296; SEQ ID NO: 306 and SEQ ID NO: 297; and SEQ ID NO: 306 and SEQ ID NO: 298.

In some aspects, the CDR-H3-CDR-L3 pairs are selected from SEQ ID NO: 307 and SEQ ID NO: 240; SEQ ID NO: 307 and SEQ ID NO: 241; SEQ ID NO: 307 and SEQ ID NO: 242; SEQ ID NO: 307 and SEQ ID NO: 243; SEQ ID NO: 307 and SEQ ID NO: 244; SEQ ID NO: 307 and SEQ ID NO: 245; SEQ ID NO: 307 and SEQ ID NO: 246; SEQ ID NO: 307 and SEQ ID NO: 247; SEQ ID NO: 307 and SEQ ID NO: 248; SEQ ID NO: 307 and SEQ ID NO: 249; SEQ ID NO: 307 and SEQ ID NO: 250; SEQ ID NO: 307 and SEQ ID NO: 251; SEQ ID NO: 307 and SEQ ID NO: 252; SEQ ID NO: 307 and SEQ ID NO: 253; SEQ ID NO: 307 and SEQ ID NO: 254; SEQ ID NO: 307 and SEQ ID NO: 255; SEQ ID NO: 307 and SEQ ID NO: 256; SEQ ID NO: 307 and SEQ ID NO: 257; SEQ ID NO: 307 and SEQ ID NO: 258; SEQ ID NO: 307 and SEQ ID NO: 259; SEQ ID NO: 307 and SEQ ID NO: 260; SEQ ID NO: 307 and SEQ ID NO: 261; SEQ ID NO: 307 and SEQ ID NO: 262; SEQ ID NO: 307 and SEQ ID NO: 263; SEQ ID NO: 307 and SEQ ID NO: 264; SEQ ID NO: 307 and SEQ ID NO: 265; SEQ ID NO: 307 and SEQ ID NO: 266; SEQ ID NO: 307 and SEQ ID NO: 267; SEQ ID NO: 307 and SEQ ID NO: 268; SEQ ID NO: 307 and SEQ ID NO: 269; SEQ ID NO: 307 and SEQ ID NO: 270; SEQ ID NO: 307 and SEQ ID NO: 271; SEQ ID NO: 307 and SEQ ID NO: 272; SEQ ID NO: 307 and SEQ ID NO: 273; SEQ ID NO: 307 and SEQ ID NO: 274; SEQ ID NO: 307 and SEQ ID NO: 275; SEQ ID NO: 307 and SEQ ID NO: 276; SEQ ID NO: 307 and SEQ ID NO: 277; SEQ ID NO: 307 and SEQ ID NO: 278; SEQ ID NO: 307 and SEQ ID NO: 279; SEQ ID NO: 307 and SEQ ID NO: 280; SEQ ID NO: 307 and SEQ ID NO: 281; SEQ ID NO: 307 and SEQ ID NO: 282; SEQ ID NO: 307 and SEQ ID NO: 283; SEQ ID NO: 307 and SEQ ID NO: 284; SEQ ID NO: 307 and SEQ ID NO: 285; SEQ ID NO: 307 and SEQ ID NO: 286; SEQ ID NO: 307 and SEQ ID NO: 287; SEQ ID NO: 307 and SEQ ID NO: 288; SEQ ID NO: 307 and SEQ ID NO: 289; SEQ ID NO: 307 and SEQ ID NO: 290; SEQ ID NO: 307 and SEQ ID NO: 291; SEQ ID NO: 307 and SEQ ID NO: 292; SEQ ID NO: 307 and SEQ ID NO: 293; SEQ ID NO: 307 and SEQ ID NO: 294; SEQ ID NO: 307 and SEQ ID NO: 295; SEQ ID NO: 307 and SEQ ID NO: 296; SEQ ID NO: 307 and SEQ ID NO: 297; and SEQ ID NO: 307 and SEQ ID NO: 298.

### 5.7.1.1. Variants of CDR-H3-CDR-L3 Pairs

In some embodiments, the CDR-H3-CDR-L3 pairs provided herein comprise a variant of an illustrative CDR-H3 and/or CDR-L1 sequence provided in this disclosure.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

### 5.7.2. CDR-H1-CDR-L1 Pairs

In some embodiments, the antibody comprises a CDR-H1 sequence and a CDR-L1 sequence. In some aspects, the CDR-H1 sequence is part of a V_{H} and the CDR-L1 sequence is part of a V_{L}.

In some aspects, the CDR-H1 sequence is a Chothia CDR-H1 sequence comprising, consisting of, or consisting essentially of SEQ ID NOs: 4-62, and the CDR-L1 sequence is a CDR-L1 sequence comprising, consisting of, or consisting essentially of SEQ ID NOs: 299-301.

In some aspects, the CDR-H1 sequence is a Kabat CDR-H1 sequence comprising, consisting of, or consisting essentially of SEQ ID NOs: 63-121, and the CDR-L1 sequence is a CDR-L1 sequence comprising, consisting of, or consisting essentially of SEQ ID NOs: 299-301.

### 5.7.2.1. Variants of CDR-H1-CDR-L1 Pairs

In some embodiments, the CDR-H1-CDR-L1 pairs provided herein comprise a variant of an illustrative CDR-H1 and/or CDR-L1 sequence provided in this disclosure.

In some aspects, the CDR-H1 sequence comprises, consists of, or consists essentially of a variant of an illustrative CDR-H1 sequence provided in this disclosure. In some aspects, the CDR-H1 sequence comprises, consists of, or consists essentially of a sequence having at least 70%, 75%, 80%, 85%, 90%, or 95% identity with any of the illustrative CDR-H1 sequences provided in this disclosure. In some aspects, the CDR-H1 sequence comprises, consists of, or consists essentially of any of the illustrative CDR-H1 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

### 5.7.3. CDR-H2 CDR-L2 Pairs

In some embodiments, the antibody comprises a CDR-H2 sequence and a CDR-L2 sequence. In some aspects, the CDR-H2 sequence is part of a V_{H} and the CDR-L2 sequence is part of a V_{L}.

In some aspects, the CDR-H2 sequence is a Chothia CDR-H2 sequence comprising, consisting of, or consisting essentially of SEQ ID NOs: 122-180, and the CDR-L2 sequence is a CDR-L2 sequence comprising, consisting of, or consisting essentially of SEQ ID NOs: 302-304.

In some aspects, the CDR-H1 sequence is a Kabat CDR-H2 sequence comprising, consisting of, or consisting essentially of SEQ ID NOs: 181-239, and the CDR-L2 sequence is a CDR-L2 sequence comprising, consisting of, or consisting essentially of SEQ ID NOs: 302-304.

### 5.7.3.1. Variants of CDR-H2-CDR-L2 Pairs

In some embodiments, the CDR-H2-CDR-L2 pairs provided herein comprise a variant of an illustrative CDR-H2 and/or CDR-L2 sequence provided in this disclosure.

In some aspects, the CDR-H2 sequence comprises, consists of, or consists essentially of a variant of an illustrative CDR-H2 sequence provided in this disclosure. In some aspects, the CDR-H2 sequence comprises, consists of, or consists essentially of a sequence having at least 70%, 75%, 80%, 85%, 90%, or 95% identity with any of the illustrative CDR-H2 sequences provided in this disclosure. In some aspects, the CDR-H2 sequence comprises, consists of, or consists essentially of any of the illustrative CDR-H2 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

5.7.4. V_{H}-V_{L} PairS

In some embodiments, the antibody comprises a V_{H} sequence and a V_{L} sequence.

In some aspects, the V_{H} sequence is a V_{H} sequence comprising, consisting of, or consisting essentially of SEQ ID NOs: 308-366, and the V_{L} sequence is a V_{L} sequence comprising, consisting of, or consisting essentially of SEQ ID NOs: 367-369.

In some aspects, the V_{H}-V_{L} pairs are selected from SEQ ID NO: 367 and SEQ ID NO: 308; SEQ ID NO: 367 and SEQ ID NO: 309; SEQ ID NO: 367 and SEQ ID NO: 310; SEQ ID NO: 367 and SEQ ID NO: 311; SEQ ID NO: 367 and SEQ ID NO: 312; SEQ ID NO: 367 and SEQ ID NO: 313; SEQ ID NO: 367 and SEQ ID NO: 314; SEQ ID NO: 367 and SEQ ID NO: 315; SEQ ID NO: 367 and SEQ ID NO: 316; SEQ ID NO: 367 and SEQ ID NO: 317; SEQ ID NO: 367 and SEQ ID NO: 318; SEQ ID NO: 367 and SEQ ID NO: 319; SEQ ID NO: 367 and SEQ ID NO: 320; SEQ ID NO: 367 and SEQ ID NO: 321; SEQ ID NO: 367 and SEQ ID NO: 322; SEQ ID NO: 367 and SEQ ID NO: 323; SEQ ID NO: 367 and SEQ ID NO: 324; SEQ ID NO: 367 and SEQ ID NO: 325; SEQ ID NO: 367 and SEQ ID NO: 326; SEQ ID NO: 367 and SEQ ID NO: 327; SEQ ID NO: 367 and SEQ ID NO: 328; SEQ ID NO: 367 and SEQ ID NO: 329; SEQ ID NO: 367 and SEQ ID NO: 330; SEQ ID NO: 367 and SEQ ID NO: 331; SEQ ID NO: 367 and SEQ ID NO: 332; SEQ ID NO: 367 and SEQ ID NO: 333; SEQ ID NO: 367 and SEQ ID NO: 334; SEQ ID NO: 367 and SEQ ID NO: 335; SEQ ID NO: 367 and SEQ ID NO: 336; SEQ ID NO: 367 and SEQ ID NO: 337; SEQ ID NO: 367 and SEQ ID NO: 338; SEQ ID NO: 367 and SEQ ID NO: 339; SEQ ID NO: 367 and SEQ ID NO: 340; SEQ ID NO: 367 and SEQ ID NO: 341; SEQ ID NO: 367 and SEQ ID NO: 342; SEQ ID NO: 367 and SEQ ID NO: 343; SEQ ID NO: 367 and SEQ ID NO: 344; SEQ ID NO: 367 and SEQ ID NO: 345; SEQ ID NO: 367 and SEQ ID NO: 346; SEQ ID NO: 367 and SEQ ID NO: 347; SEQ ID NO: 367 and SEQ ID NO: 348; SEQ ID NO: 367 and SEQ ID NO: 349; SEQ ID NO: 367 and SEQ ID NO: 350; SEQ ID NO: 367 and SEQ ID NO: 351; SEQ ID NO: 367 and SEQ ID NO: 352; SEQ ID NO: 367 and SEQ ID NO: 353; SEQ ID NO: 367 and SEQ ID NO: 354; SEQ ID NO: 367 and SEQ ID NO: 355; SEQ ID NO: 367 and SEQ ID NO: 356; SEQ ID NO: 367 and SEQ ID NO: 357; SEQ ID NO: 367 and SEQ ID NO: 358; SEQ ID NO: 367 and SEQ ID NO: 359; SEQ ID NO: 367 and SEQ ID NO: 360; SEQ ID NO: 367 and SEQ ID NO: 361; SEQ ID NO: 367 and SEQ ID NO: 362; SEQ ID NO: 367 and SEQ ID NO: 363; SEQ ID NO: 367 and SEQ ID NO: 364; SEQ ID NO: 367 and SEQ ID NO: 365; and SEQ ID NO: 367 and SEQ ID NO: 366.

In some aspects, the V_{H}-V_{L} pairs are selected from SEQ ID NO: 368 and SEQ ID NO: 308; SEQ ID NO: 368 and SEQ ID NO: 309; SEQ ID NO: 368 and SEQ ID NO: 310; SEQ ID NO: 368 and SEQ ID NO: 311; SEQ ID NO: 368 and SEQ ID NO: 312; SEQ ID NO: 368 and SEQ ID NO: 313; SEQ ID NO: 368 and SEQ ID NO: 314; SEQ ID NO: 368 and SEQ ID NO: 315; SEQ ID NO: 368 and SEQ ID NO: 316; SEQ ID NO: 368 and SEQ ID NO: 317; SEQ ID NO: 368 and SEQ ID NO: 318; SEQ ID NO: 368 and SEQ ID NO: 319; SEQ ID NO: 368 and SEQ ID NO: 320; SEQ ID NO: 368 and SEQ ID NO: 321; SEQ ID NO: 368 and SEQ ID NO: 322; SEQ ID NO: 368 and SEQ ID NO: 323; SEQ ID NO: 368 and SEQ ID NO: 324; SEQ ID NO: 368 and SEQ ID NO: 325; SEQ ID NO: 368 and SEQ ID NO: 326; SEQ ID NO: 368 and SEQ ID NO: 327; SEQ ID NO: 368 and SEQ ID NO: 328; SEQ ID NO: 368 and SEQ ID NO: 329; SEQ ID NO: 368 and SEQ ID NO: 330; SEQ ID NO: 368 and SEQ ID NO: 331; SEQ ID NO: 368 and SEQ ID NO: 332; SEQ ID NO: 368 and SEQ ID NO: 333; SEQ ID NO: 368 and SEQ ID NO: 334; SEQ ID NO: 368 and SEQ ID NO: 335; SEQ ID NO: 368 and SEQ ID NO: 336; SEQ ID NO: 368 and SEQ ID NO: 337; SEQ ID NO: 368 and SEQ ID NO: 338; SEQ ID NO: 368 and SEQ ID NO: 339; SEQ ID NO: 368 and SEQ ID NO: 340; SEQ ID NO: 368 and SEQ ID NO: 341; SEQ ID NO: 368 and SEQ ID NO: 342; SEQ ID NO: 368 and SEQ ID NO: 343; SEQ ID NO: 368 and SEQ ID NO: 344; SEQ ID NO: 368 and SEQ ID NO: 345; SEQ ID NO: 368 and SEQ ID NO: 346; SEQ ID NO: 368 and SEQ ID NO: 347; SEQ ID NO: 368 and SEQ ID NO: 348; SEQ ID NO: 368 and SEQ ID NO: 349; SEQ ID NO: 368 and SEQ ID NO: 350; SEQ ID NO: 368 and SEQ ID NO: 351; SEQ ID NO: 368 and SEQ ID NO: 352; SEQ ID NO: 368 and SEQ ID NO: 353; SEQ ID NO: 368 and SEQ ID NO: 354; SEQ ID NO: 368 and SEQ ID NO: 355; SEQ ID NO: 368 and SEQ ID NO: 356; SEQ ID NO: 368 and SEQ ID NO: 357; SEQ ID NO: 368 and SEQ ID NO: 358; SEQ ID NO: 368 and SEQ ID NO: 359; SEQ ID NO: 368 and SEQ ID NO: 360; SEQ ID NO: 368 and SEQ ID NO: 361; SEQ ID NO: 368 and SEQ ID NO: 362; SEQ ID NO: 368 and SEQ ID NO: 363; SEQ ID NO: 368 and SEQ ID NO: 364; SEQ ID NO: 368 and SEQ ID NO: 365; and SEQ ID NO: 368 and SEQ ID NO: 366.

In some aspects, the V_{H}-V_{L} pairs are selected from SEQ ID NO: 369 and SEQ ID NO: 308; SEQ ID NO: 369 and SEQ ID NO: 309; SEQ ID NO: 369 and SEQ ID NO: 310; SEQ ID NO: 369 and SEQ ID NO: 311; SEQ ID NO: 369 and SEQ ID NO: 312; SEQ ID NO: 369 and SEQ ID NO: 313; SEQ ID NO: 369 and SEQ ID NO: 314; SEQ ID NO: 369 and SEQ ID NO: 315; SEQ ID NO: 369 and SEQ ID NO: 316; SEQ ID NO: 369 and SEQ ID NO: 317; SEQ ID NO: 369 and SEQ ID NO: 318; SEQ ID NO: 369 and SEQ ID NO: 319; SEQ ID NO: 369 and SEQ ID NO: 320; SEQ ID NO: 369 and SEQ ID NO: 321; SEQ ID NO: 369 and SEQ ID NO: 322; SEQ ID NO: 369 and SEQ ID NO: 323; SEQ ID NO: 369 and SEQ ID NO: 324; SEQ ID NO: 369 and SEQ ID NO: 325; SEQ ID NO: 369 and SEQ ID NO: 326; SEQ ID NO: 369 and SEQ ID NO: 327; SEQ ID NO: 369 and SEQ ID NO: 328; SEQ ID NO: 369 and SEQ ID NO: 329; SEQ ID NO: 369 and SEQ ID NO: 330; SEQ ID NO: 369 and SEQ ID NO: 331; SEQ ID NO: 369 and SEQ ID NO: 332; SEQ ID NO: 369 and SEQ ID NO: 333; SEQ ID NO: 369 and SEQ ID NO: 334; SEQ ID NO: 369 and SEQ ID NO: 335; SEQ ID NO: 369 and SEQ ID NO: 336; SEQ ID NO: 369 and SEQ ID NO: 337; SEQ ID NO: 369 and SEQ ID NO: 338; SEQ ID NO: 369 and SEQ ID NO: 339; SEQ ID NO: 369 and SEQ ID NO: 340; SEQ ID NO: 369 and SEQ ID NO: 341; SEQ ID NO: 369 and SEQ ID NO: 342; SEQ ID NO: 369 and SEQ ID NO: 343; SEQ ID NO: 369 and SEQ ID NO: 344; SEQ ID NO: 369 and SEQ ID NO: 345; SEQ ID NO: 369 and SEQ ID NO: 346; SEQ ID NO: 369 and SEQ ID NO: 347; SEQ ID NO: 369 and SEQ ID NO: 348; SEQ ID NO: 369 and SEQ ID NO: 349; SEQ ID NO: 369 and SEQ ID NO: 350; SEQ ID NO: 369 and SEQ ID NO: 351; SEQ ID NO: 369 and SEQ ID NO: 352; SEQ ID NO: 369 and SEQ ID NO: 353; SEQ ID NO: 369 and SEQ ID NO: 354; SEQ ID NO: 369 and SEQ ID NO: 355; SEQ ID NO: 369 and SEQ ID NO: 356; SEQ ID NO: 369 and SEQ ID NO: 357; SEQ ID NO: 369 and SEQ ID NO: 358; SEQ ID NO: 369 and SEQ ID NO: 359; SEQ ID NO: 369 and SEQ ID NO: 360; SEQ ID NO: 369 and SEQ ID NO: 361; SEQ ID NO: 369 and SEQ ID NO: 362; SEQ ID NO: 369 and SEQ ID NO: 363; SEQ ID NO: 369 and SEQ ID NO: 364; SEQ ID NO: 369 and SEQ ID NO: 365; and SEQ ID NO: 369 and SEQ ID NO: 366.

5.7.4.1. Variants of V_{H}-V_{L} Pairs

In some embodiments, the V_{H}-V_{L} pairs provided herein comprise a variant of an illustrative V_{H} and/or V_{L} sequence provided in this disclosure.

In some aspects, the V_{H} sequence comprises, consists of, or consists essentially of a variant of an illustrative V_{H} sequence provided in this disclosure. In some aspects, the V_{H}sequence comprises, consists of, or consists essentially of a sequence having at least 85%, 90%, 95%, 96%, 97%, 98%, 99%, or 99.10% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

In some aspects, the V_{L} sequence comprises, consists of, or consists essentially of a variant of an illustrative V_{L} sequence provided in this disclosure. In some aspects, the V_{L}sequence comprises, consists of, or consists essentially of a sequence having at least 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 5.8. Antibodies Comprising All Six CDRs

In some embodiments, the antibody comprises a CDR-H1 sequence, a CDR-H2 sequence, a CDR-H3 sequence, a CDR-L1 sequence, and a CDR-L3 sequence. In some aspects, the CDR sequences are part of a V_{H} (for CDR-H) or V_{L} (for CDR-L).

In some aspects, the CDR-H1 sequence is a Chothia CDR-H1 sequence comprising, consisting of, or consisting essentially of SEQ ID NOs: 4-62; the CDR-H2 sequence is a Chothia CDR-H2 sequence comprising, consisting of, or consisting essentially of SEQ ID NOs: 122-180; the CDR-H3 sequence is a CDR-H3 sequence comprising, consisting of, or consisting essentially of SEQ ID NOs: 240-298; the CDR-L1 sequence is a CDR-L1 sequence comprising, consisting of, or consisting essentially of SEQ ID NOs: 299-301; the CDR-L2 sequence is a CDR-L2 sequence comprising, consisting of, or consisting essentially of SEQ ID NOs: 302-304; and the CDR-L3 sequence is a CDR-L3 sequence comprising, consisting of, or consisting essentially of SEQ ID NOs: 305-307.

In some aspects, the CDR-H1 sequence is a Chothia CDR-H1 sequence comprising, consisting of, or consisting essentially of SEQ ID NO: 19; the CDR-H2 sequence is a Chothia CDR-H2 sequence comprising, consisting of, or consisting essentially of SEQ ID NO: 137; the CDR-H3 sequence is a CDR-H3 sequence comprising, consisting of, or consisting essentially of SEQ ID NO: 255; the CDR-L1 sequence is a CDR-L1 sequence comprising, consisting of, or consisting essentially of SEQ ID NOs: 299-301; the CDR-L2 sequence is a CDR-L2 sequence comprising, consisting of, or consisting essentially of SEQ ID NOs: 302-304; and the CDR-L3 sequence is a CDR-L3 sequence comprising, consisting of, or consisting essentially of SEQ ID NOs: 305-307.

In some aspects, the CDR-H1 sequence is a Chothia CDR-H1 sequence comprising, consisting of, or consisting essentially of SEQ ID NO: 58; the CDR-H2 sequence is a Chothia CDR-H2 sequence comprising, consisting of, or consisting essentially of SEQ ID NO: 176; the CDR-H3 sequence is a CDR-H3 sequence comprising, consisting of, or consisting essentially of SEQ ID NO: 294; the CDR-L1 sequence is a CDR-L1 sequence comprising, consisting of, or consisting essentially of SEQ ID NOs: 299-301; the CDR-L2 sequence is a CDR-L2 sequence comprising, consisting of, or consisting essentially of SEQ ID NOs: 302-304; and the CDR-L3 sequence is a CDR-L3 sequence comprising, consisting of, or consisting essentially of SEQ ID NOs: 305-307.

In some aspects, the CDR-H1 sequence is a Kabat CDR-H1 sequence comprising, consisting of, or consisting essentially of SEQ ID NOs: 63-121; the CDR-H2 sequence is a Kabat CDR-H2 sequence comprising, consisting of, or consisting essentially of SEQ ID NOs: 181-239; the CDR-H3 sequence is a CDR-H3 sequence comprising, consisting of, or consisting essentially of SEQ ID NOs: 240-298; the CDR-L1 sequence is a CDR-L1 sequence comprising, consisting of, or consisting essentially of SEQ ID NOs: 299-301; the CDR-L2 sequence is a CDR-L2 sequence comprising, consisting of, or consisting essentially of SEQ ID NOs: 302-304; and the CDR-L3 sequence is a CDR-L3 sequence comprising, consisting of, or consisting essentially of SEQ ID NOs: 305-307.

In some aspects, the CDR-H1 sequence is a Kabat CDR-H1 sequence comprising, consisting of, or consisting essentially of SEQ ID NO: 78; the CDR-H2 sequence is a Kabat CDR-H2 sequence comprising, consisting of, or consisting essentially of SEQ ID NO: 196; the CDR-H3 sequence is a CDR-H3 sequence comprising, consisting of, or consisting essentially of SEQ ID NO: 255; the CDR-L1 sequence is a CDR-L1 sequence comprising, consisting of, or consisting essentially of SEQ ID NOs: 299-301; the CDR-L2 sequence is a CDR-L2 sequence comprising, consisting of, or consisting essentially of SEQ ID NOs: 302-304; and the CDR-L3 sequence is a CDR-L3 sequence comprising, consisting of, or consisting essentially of SEQ ID NOs: 305-307.

In some aspects, the CDR-H1 sequence is a Kabat CDR-H1 sequence comprising, consisting of, or consisting essentially of SEQ ID NO: 117; the CDR-H2 sequence is a Kabat CDR-H2 sequence comprising, consisting of, or consisting essentially of SEQ ID NO: 235; the CDR-H3 sequence is a CDR-H3 sequence comprising, consisting of, or consisting essentially of SEQ ID NO: 294; the CDR-L1 sequence is a CDR-L1 sequence comprising, consisting of, or consisting essentially of SEQ ID NOs: 299-301; the CDR-L2 sequence is a CDR-L2 sequence comprising, consisting of, or consisting essentially of SEQ ID NOs: 302-304; and the CDR-L3 sequence is a CDR-L3 sequence comprising, consisting of, or consisting essentially of SEQ ID NOs: 305-307.

### 5.8.1. Variants of Antibodies Comprising All Six CDRs

In some embodiments, the CDR-H1, CDR-H2, CDR-H3, CDR-L1, CDR-L2, and CDR-L3 provided herein comprise a variant of an illustrative CDR-H1, CDR-H2, CDR-H3, CDR-L1, CDR-L2, and/or CDR-L3 sequence provided in this disclosure.

In some aspects, the CDR-H1 sequence comprises, consists of, or consists essentially of a variant of an illustrative Chothia or Kabat CDR-H1 sequence provided in this disclosure. In some aspects, the CDR-H1 sequence comprises, consists of, or consists essentially of a sequence having at least 70%, 75%, 80%, 85%, 90%, or 95% identity with any of the illustrative Chothia or Kabat CDR-H1 sequences provided in this disclosure. In some aspects, the CDR-H1 sequence comprises, consists of, or consists essentially of any of the illustrative Chothia or Kabat CDR-H1 sequences provided in this disclosure, with 1, 2, or 3 amino acid substitutions. In some aspects, the amino acid substitutions are conservative amino acid substitutions.

In some aspects, the CDR-H2 sequence comprises, consists of, or consists essentially of a variant of an illustrative Chothia or Kabat CDR-H2 sequence provided in this disclosure. In some aspects, the CDR-H2 sequence comprises, consists of, or consists essentially of a sequence having at least 70%, 75%, 80%, 85%, 90%, or 95% identity with any of the illustrative Chothia or Kabat CDR-H2 sequences provided in this disclosure. In some aspects, the CDR-H2 sequence comprises, consists of, or consists essentially of any of the illustrative Chothia or Kabat CDR-H2 sequences provided in this disclosure, with 1, 2, or 3 amino acid substitutions. In some aspects, the amino acid substitutions are conservative amino acid substitutions.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

### 7. Affinity

In some embodiments, the affinity of the antibody for folate receptor alpha as indicated by K_{D}, is less than about 10⁻⁵ M, less than about 10⁻⁶ M, less than about 10⁻⁷ M, less than about 10⁻⁸ M, less than about 10⁻⁹ M, less than about 10⁻¹⁰ M, less than about 10⁻¹¹ M, or less than about 10⁻¹ M. In some embodiments, the affinity of the antibody is between about 10⁻⁷ M and 10⁻¹¹ M. In some embodiments, the affinity of the antibody is between about 10⁻⁷ M and 10⁻¹⁰ M. In some embodiments, the affinity of the antibody is between about 10⁻⁷ M and 10⁻⁹ M. In some embodiments, the affinity of the antibody is between about 10⁻⁷ M and 10⁻⁸ M. In some embodiments, the affinity of the antibody is between about 10⁻⁸ M and 10⁻¹¹ M. In some embodiments, the affinity of the antibody is between about 10⁻⁸ M and 10⁻¹⁰ M. In some embodiments, the affinity of the antibody is between about 10⁻⁹ M and 10⁻¹¹ M. In some embodiments, the affinity of the antibody is between about 10⁻⁹ M and 10⁻¹⁰ M.

In some embodiments, the affinity of the antibody for human folate receptor alpha, as determined by surface plasmon resonance at 25° C., and as indicated by K_{D}, is from about 0.36×10⁻⁹ M to about 2.21×10⁻⁹ M. In some embodiments, the affinity of the antibody for human folate receptor alpha, as determined by surface plasmon resonance at 25° C., and as indicated by K_{D}, is from about 8.55×10⁻¹⁰ M to about 1.70×10⁻⁸ M. In some embodiments, the affinity of the antibody for human folate receptor alpha, as determined by surface plasmon resonance at 25° C., and as indicated by K_{D}, is from about 5.71×10⁻¹⁰ M to about 2.58×10⁻⁸ M. In some embodiments, the affinity of the antibody for human folate receptor alpha is about any of the K_{D} values reported for human folate receptor alpha in the examples below.

In some embodiments the antibody has a kₐ of at least about 104 M⁻¹×sec⁻¹. In some embodiments the antibody has a kₐ of at least about 10⁵ M⁻¹×sec⁻¹. In some embodiments the antibody has a kₐ of at least about 10⁶ M⁻¹×sec⁻¹. In some embodiments the antibody has a kₐ of at least about 10⁷ M⁻¹×sec⁻¹. In some embodiments the antibody has a kₐ of at least about 10⁸ M⁻¹×sec⁻¹. In some embodiments the antibody has a kₐ of at least about 109 M⁻¹×sec⁻¹. In some embodiments the antibody has a kₐ of between about 104 M⁻¹×sec⁻¹ and about 10¹⁰ M⁻¹×sec⁻¹. In some embodiments the antibody has a kₐ of between about 10⁵ M⁻¹×sec⁻¹ and about 10¹⁰ M⁻¹×sec⁻¹. In some embodiments the antibody has a kₐ of between about 10⁶ M⁻¹×sec⁻¹ and about 10¹⁰ M⁻¹×sec⁻¹. In some embodiments the antibody has a kₐ of between about 10⁷ M⁻¹×sec⁻¹ and about 10¹⁰ M⁻¹×sec⁻¹.

In some embodiments the antibody has a kₐ when associating with human folate receptor alpha, as determined by surface plasmon resonance at 25° C., of from about 4.44×10⁵ M⁻¹×sec⁻¹ to about 1.61×10⁵ M⁻¹×sec⁻¹. In some embodiments the antibody has a kₐ when associating with human folate receptor alpha, as determined by surface plasmon resonance at 25° C., of from about 2.90×10⁵ M⁻¹×sec⁻¹ to about 9.64×109 M⁻¹×sec⁻¹. In some embodiments the antibody has a kₐ when associating with human folate receptor alpha of about any of the kₐ values reported for human folate receptor alpha in the examples below.

In some embodiments the antibody has a kₐ of about 10⁻⁵ sec⁻¹ or less. In some embodiments the antibody has a kₐ of about 10⁻⁴ sec⁻¹ or less. In some embodiments the antibody has a kₐ of about 10⁻³ sec⁻¹ or less. In some embodiments the antibody has a kₐ of between about 10⁻² sec⁻¹ and about 10⁻⁵ sec⁻¹. In some embodiments the antibody has a kₐ of between about 10⁻² sec⁻¹ and about 10⁻⁴ sec⁻¹. In some embodiments the antibody has a kₐ of between about 10⁻³ sec⁻¹ and about 10⁻⁵ sec⁻¹.

In some embodiments the antibody has a kₐ when dissociating from human folate receptor alpha, as determined by surface plasmon resonance at 25° C., of from about 8.66×10⁻⁴ sec⁻¹ to about 1.08×10⁻² sec⁻¹. In some embodiments the antibody has a kₐ when dissociating from human folate receptor alpha, as determined by surface plasmon resonance at 25° C., of from about 2.28×10⁻⁴ sec⁻¹ to about 4.82×10¹ sec⁻¹. In some embodiments the antibody has a kₐ when dissociating from human folate receptor alpha of about any of the kₐ values reported for human folate receptor alpha in the examples below.

In some embodiments, the affinity of the antibody for cynomolgus folate receptor alpha, as determined by surface plasmon resonance at 25° C., and as indicated by KD, is from about 0.19×10⁻⁹ M to about 2.84×10⁻⁹ M. In some embodiments, the affinity of the antibody for cynomolgus folate receptor alpha is about any of the K_{D} values reported for cynomolgus folate receptor alpha in the examples below.

In some embodiments, the affinity of the antibody for mouse folate receptor alpha, as determined by surface plasmon resonance at 25° C., and as indicated by K_{D}, is from about 0.5×10⁻⁹ M to about 9.07×10⁻⁸ M. In some embodiments, the affinity of the antibody for mouse folate receptor alpha is about any of the KD values reported for mouse folate receptor alpha in the examples below.

In some aspects, the K_{D}, kₐ, and k_{d} are determined at 25° C. In some embodiments, the K_{D}, kₐ, and k_{d} are determined by surface plasmon resonance. In some embodiments, the K_{D}, kₐ, and kₐ are determined according to the methods described in the Examples provided herein.

### 8. Epitope Bins

In some embodiments, the antibody binds the same epitope as an antibody encompassing any of SEQ ID NOs: 308-366. In some embodiments, the antibody binds the same epitope as an antibody comprising any of the V_{H}-V_{L} pairs, above. In some embodiments, the antibody competes for epitope binding with an antibody encompassing any of SEQ ID NOs: 308-366. In some embodiments, the antibody competes for epitope binding with an antibody comprising any of the V_{H}-V_{L} pairs, above.

### 9. Glycosylation Variants

In certain embodiments, an antibody may be altered to increase, decrease or eliminate the extent to which it is glycosylated. Glycosylation of polypeptides is typically either “N-linked” or “O-linked.”

“N-linked” glycosylation refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site.

“O-linked” glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of N-linked glycosylation sites to the antibody may be accomplished by altering the amino acid sequence such that one or more of the above-described tripeptide sequences is created or removed. Addition or deletion of O-linked glycosylation sites may be accomplished by addition, deletion, or substitution of one or more serine or threonine residues in or to (as the case may be) the sequence of an antibody.

### 10. Fc Variants

In certain embodiments, amino acid modifications may be introduced into the Fc region of an antibody provided herein to generate an Fc region variant. In certain embodiments, the Fc region variant possesses some, but not all, effector functions. Such antibodies may be useful, for example, in applications in which the half-life of the antibody in vivo is important, yet certain effector functions are unnecessary or deleterious. Examples of effector functions include complement-dependent cytotoxicity (CDC) and antibody-directed complement-mediated cytotoxicity (ADCC). Numerous substitutions or substitutions or deletions with altered effector function are known in the art.

In some embodiments, the Fc comprises one or more modifications in at least one of the C_{H}3 sequences. In some embodiments, the Fc comprises one or more modifications in at least one of the C_{H}2 sequences. For example, the Fc can include one or modifications selected from the group consisting of: V262E, V262D, V262K, V262R, V262S, V264S, V303R, and V305R. In some embodiments, an Fc is a single polypeptide. In some embodiments, an Fc is multiple peptides, e.g., two polypeptides. Exemplary modifications in the Fc region are described, for example, in International Patent Application No. PCT/US2017/037545, filed Jun. 14, 2017.

An alteration in in CDC and/or ADCC activity can be confirmed using in vitro and/or in vivo assays. For example, Fc receptor (FcR) binding assays can be conducted to measure FcγR binding. The primary cells for mediating ADCC, NK cells, express FcγRIII only, whereas monocytes express FcγRI, FcγRII and FcγRIII. FcR expression on hematopoietic cells is summarized in Ravetch and Kinet, Ann. Rev. Immunol., 1991, 9:457-492, incorporated by reference in its entirety.

Non-limiting examples of in vitro assays to assess ADCC activity of a molecule of interest are provided in U.S. Pat. Nos. 5,500,362 and 5,821,337; Hellstrom et al., Proc. Natl. Acad. Sci. U.S.A., 1986, 83:7059-7063; Hellstrom et al., Proc. Natl. Acad. Sci. U.S.A., 1985, 82:1499-1502; and Bruggemann et al., J. Exp. Med., 1987, 166:1351-1361; each of which is incorporated by reference in its entirety. Useful effector cells for such assays include peripheral blood mononuclear cells (PBMC) and Natural Killer (NK) cells. Alternatively, or additionally, ADCC activity of the molecule of interest may be assessed in vivo, using an animal model such as that disclosed in Clynes et al. Proc. Natl. Acad. Sci. U.S.A., 1998, 95:652-656, incorporated by reference in its entirety.

C1q binding assays may also be carried out to confirm that the antibody is unable to bind C1q and hence lacks CDC activity. Examples of C1q binding assays include those described in WO 2006/029879 and WO 2005/100402, each of which is incorporated by reference in its entirety.

Complement activation assays include those described, for example, in Gazzano-Santoro et al., J. Immunol. Methods, 1996, 202:163-171; Cragg et al., Blood, 2003, 101:1045-1052; and Cragg and Glennie, Blood, 2004, 103:2738-2743; each of which is incorporated by reference in its entirety.

FcRn binding and in vivo clearance (half-life determination) can also be measured, for example, using the methods described in Petkova et al., Intl. Immunol., 2006, 18:1759-1769, incorporated by reference in its entirety.

### 12. Preparation of Antibody Conjugates

### 12.1. Antigen Preparation

The FOLR1 protein to be used for isolation of the antibodies may be intact FOLR1 or a fragment of FOLR1. The intact FOLR1 protein, or fragment of FOLR1, may be in the form of an isolated protein or protein expressed by a cell. Other forms of FOLR1 useful for generating antibodies will be apparent to those skilled in the art.

### 12.2. Monoclonal Antibodies

Monoclonal antibodies may be obtained, for example, using the hybridoma method first described by Kohler et al., Nature, 1975, 256:495-497 (incorporated by reference in its entirety), and/or by recombinant DNA methods (see e.g., U.S. Pat. No. 4,816,567, incorporated by reference in its entirety). Monoclonal antibodies may also be obtained, for example, using phage or yeast-based libraries. See e.g., U.S. Pat. Nos. 8,258,082 and 8,691,730, each of which is incorporated by reference in its entirety.

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal Antibodies: Principles and Practice 3^{rd} ed. (1986) Academic Press, San Diego, CA, incorporated by reference in its entirety.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, CA), and SP-2 or X63-Ag8-653 cells (available from the American Type Culture Collection, Rockville, MD). Human myeloma and mouse-human heteromyeloma cell lines also have been described for the production of human monoclonal antibodies. See e.g., Kozbor, J. Immunol., 1984, 133:3001, incorporated by reference in its entirety.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

### 12.3. Humanized Antibodies

Humanized antibodies may be generated by replacing most, or all, of the structural portions of a non-human monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad. Sci. U.S.A., 1998, 95:8910-8915; Steinberger et al., J. Biol. Chem., 2000, 275:36073-36078; Queen et al., Proc. Natd. Acad. Sci. U.S.A., 1989, 86:10029-10033; and U.S. Pat. Nos. 5,585,089, 5,693,761, 5,693,762, and 6,180,370; each of which is incorporated by reference in its entirety.

### 12.4. Human Antibodies

Human antibodies can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807; each of which is incorporated by reference in its entirety. Human antibodies can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905; each of which is incorporated by reference in its entirety). Human antibodies may also be generated by in vitro activated B cells (see e.g., U.S. Pat. Nos. 5,567,610 and 5,229,275, each of which is incorporated by reference in its entirety). Human antibodies may also be derived from yeast-based libraries (see e.g., U.S. Pat. No. 8,691,730, incorporated by reference in its entirety).

### 12.5. Conjugation

The antibody conjugates can be prepared by standard techniques. In certain embodiments, an antibody is contacted with a payload precursor under conditions suitable for forming a bond from the antibody to the payload to form an antibody-payload conjugate. In certain embodiments, an antibody is contacted with a linker precursor under conditions suitable for forming a bond from the antibody to the linker. The resulting antibody-linker is contacted with a payload precursor under conditions suitable for forming a bond from the antibody-linker to the payload to form an antibody-linker-payload conjugate. In certain embodiments, a payload precursor is contacted with a linker precursor under conditions suitable for forming a bond from the payload to the linker. The resulting payload-linker is contacted with an antibody under conditions suitable for forming a bond from the payload-linker to the antibody to form an antibody-linker-payload conjugate. Suitable linkers for preparing the antibody conjugates are disclosed herein, and exemplary conditions for conjugation are described in the Examples below.

In some embodiments, an anti-FOLR1 conjugate is prepared by contacting an anti-FOLR1 antibody as disclosed herein with a linker precursor having a structure of any of (A)-(L):

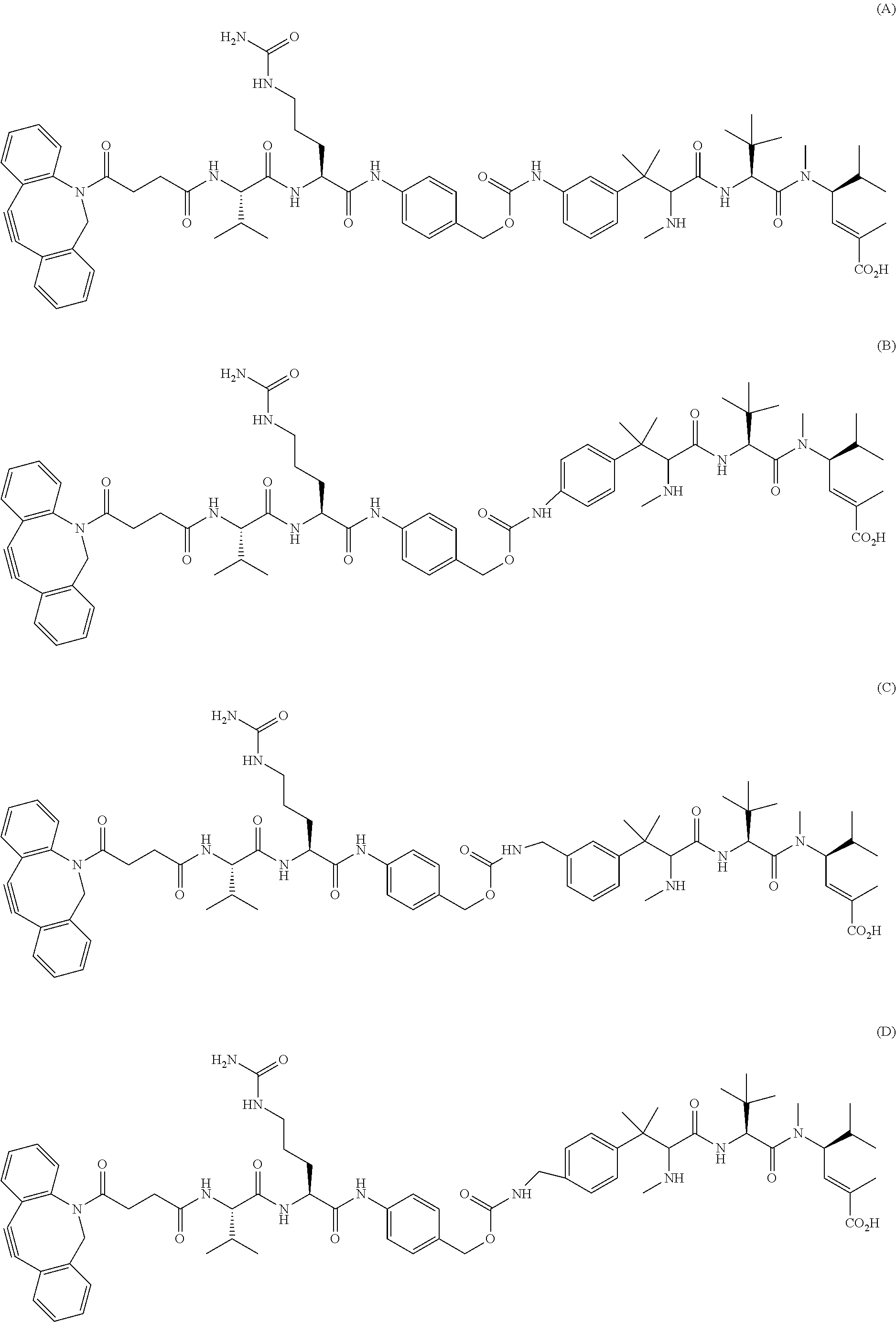

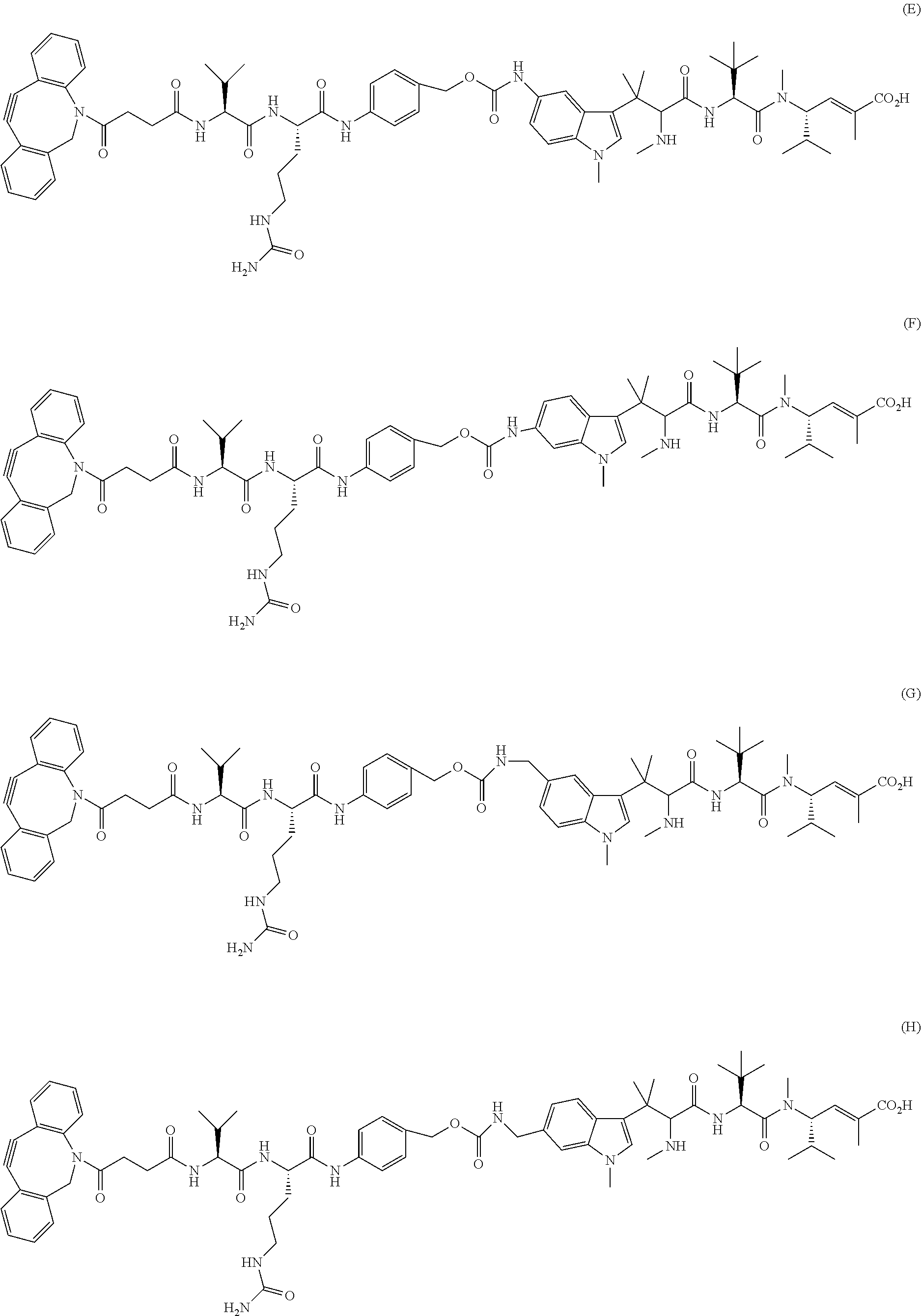

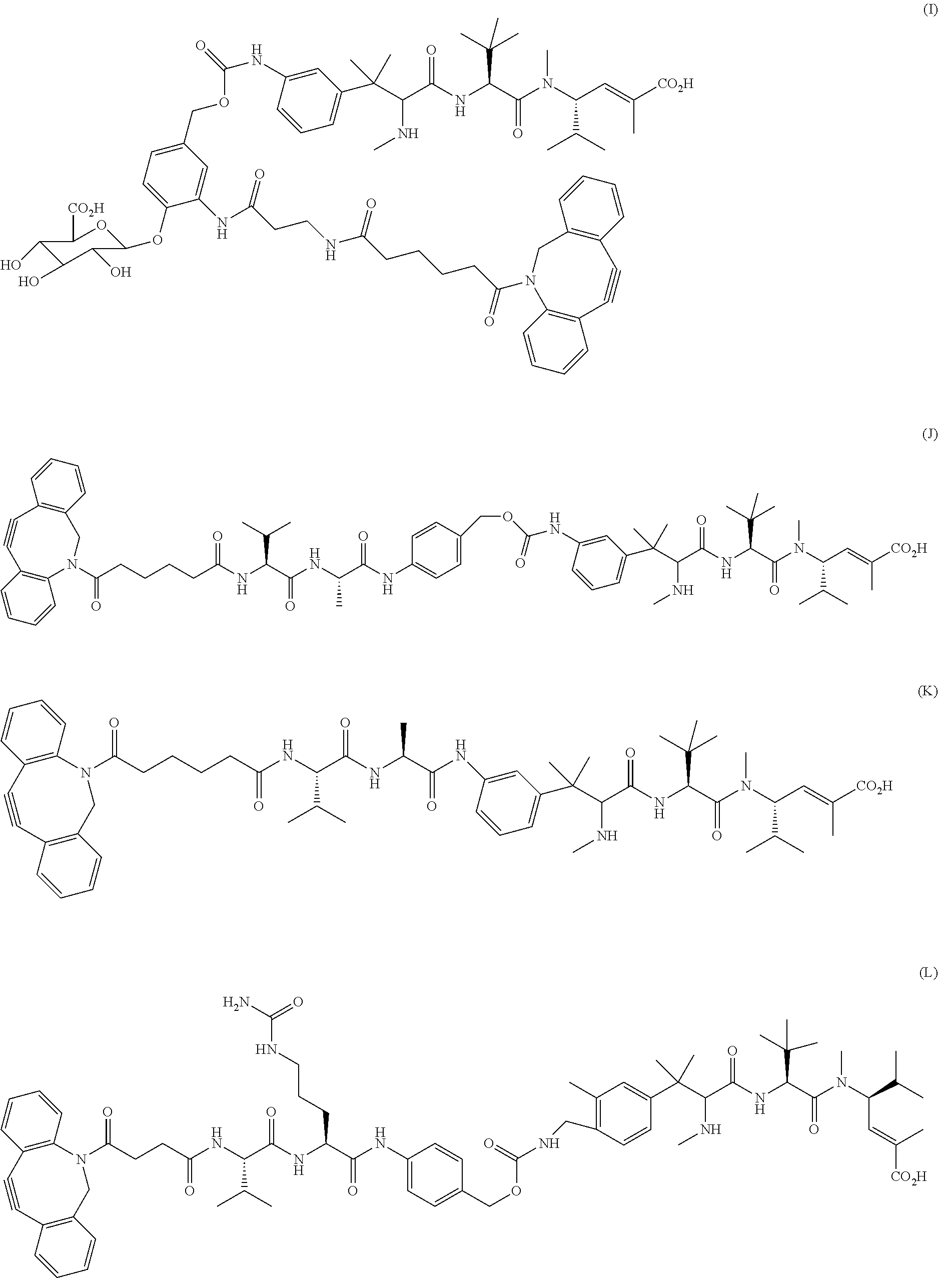

In some embodiments, the stereochemistry of the linker precursors identified as (A)-(L) is identified with R and S notation for each chiral center, from left to right as depicted in formulas (A1)-(L1) and (A2)-(L2) illustrated below:

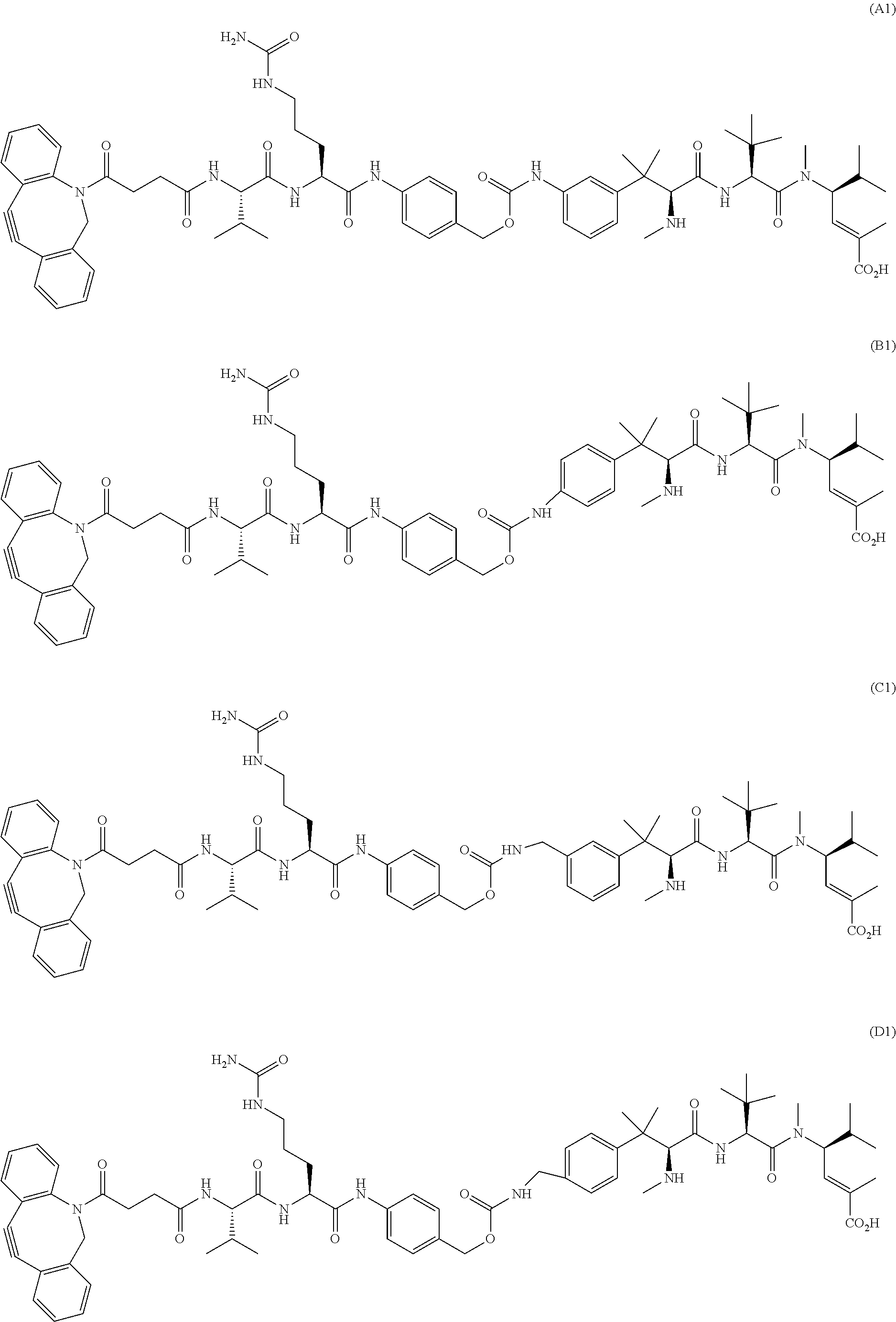

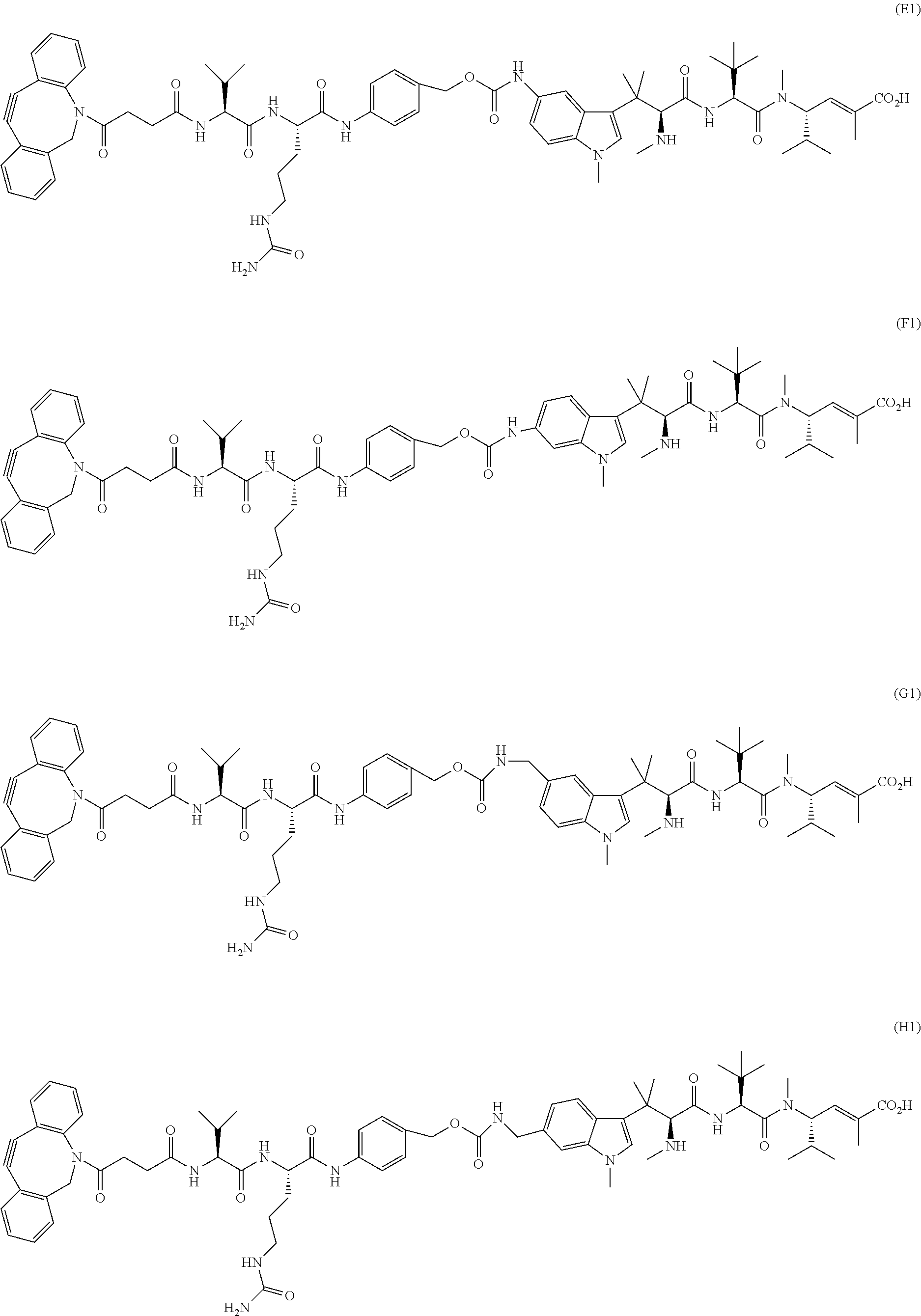

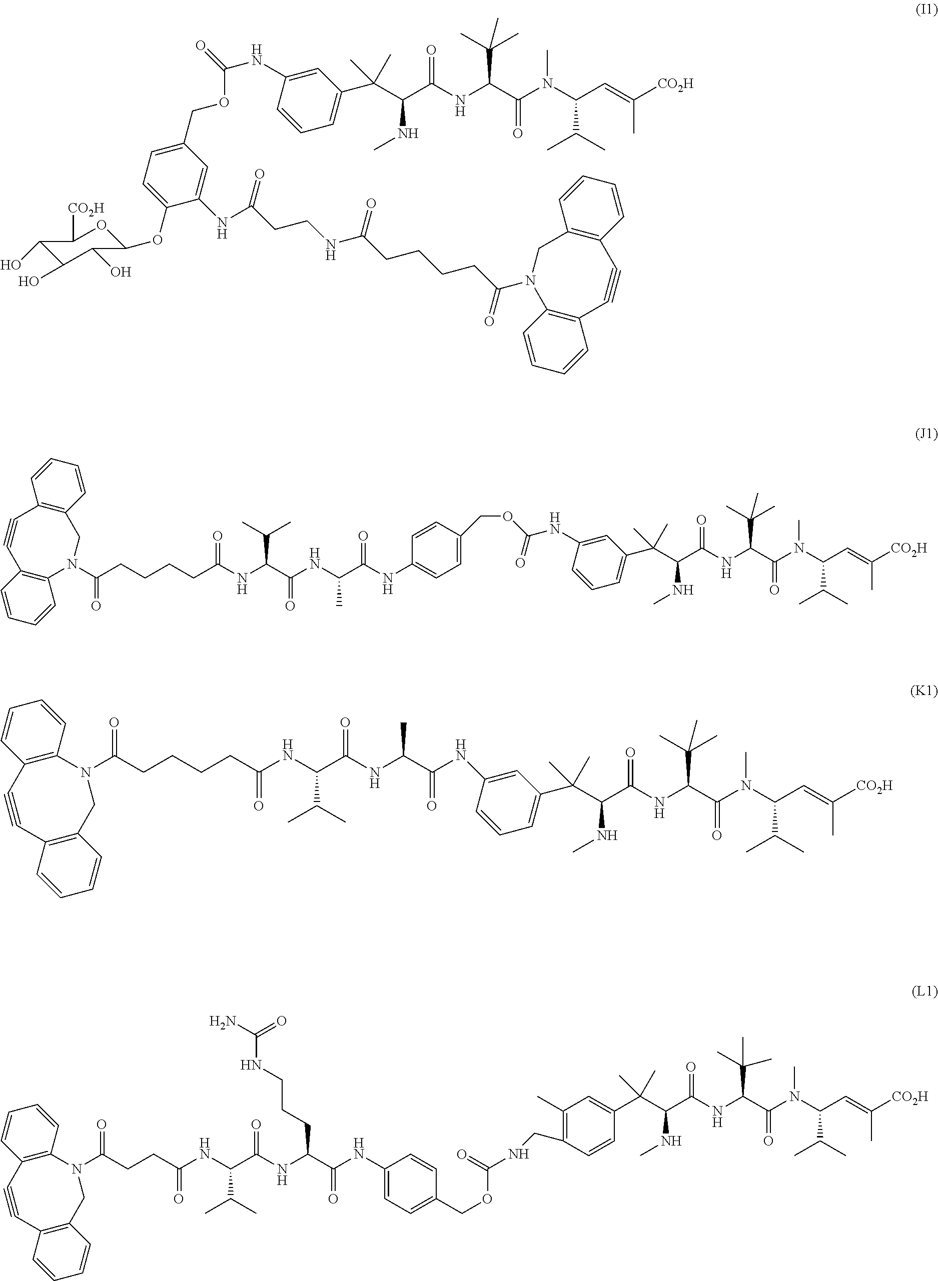

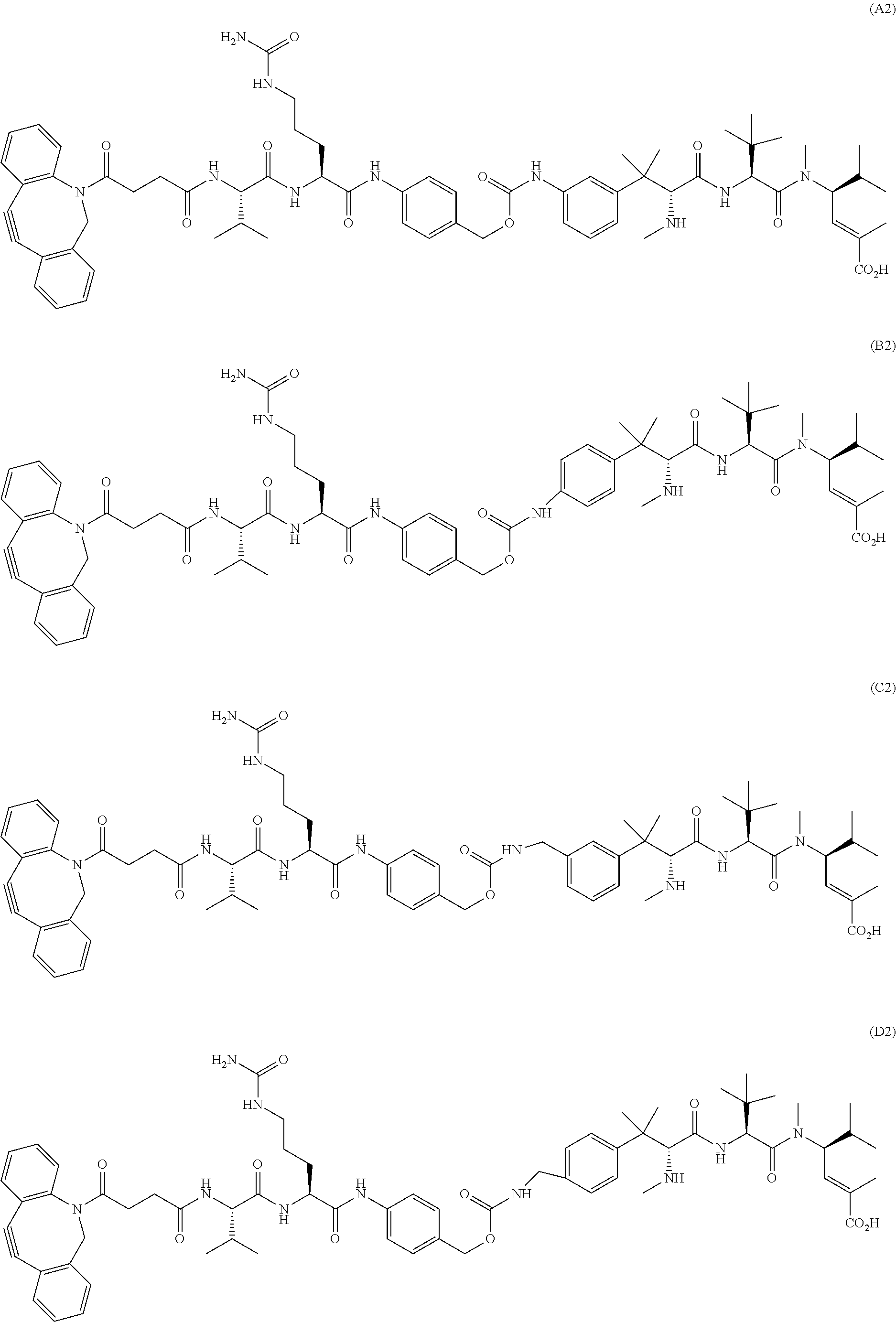

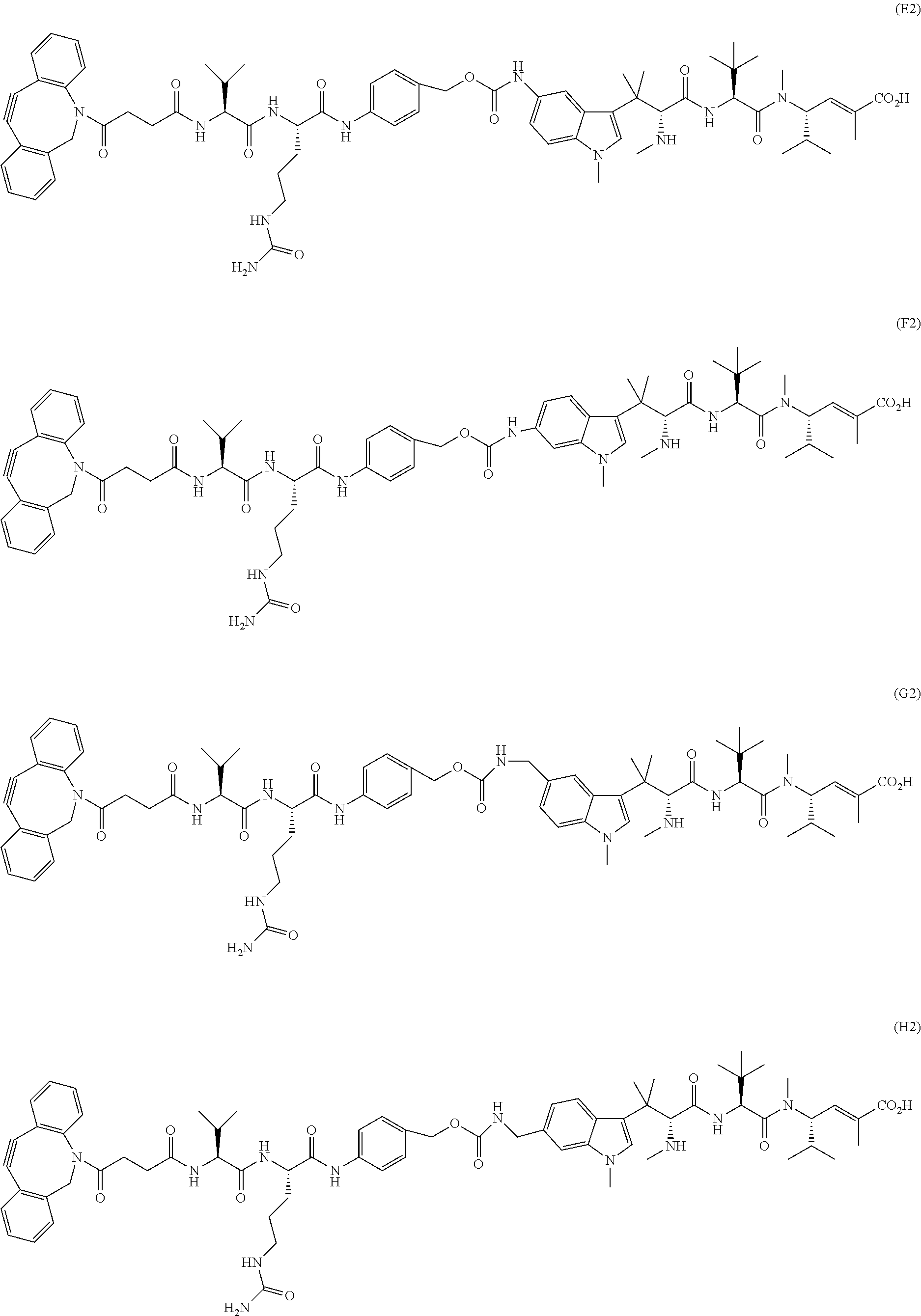

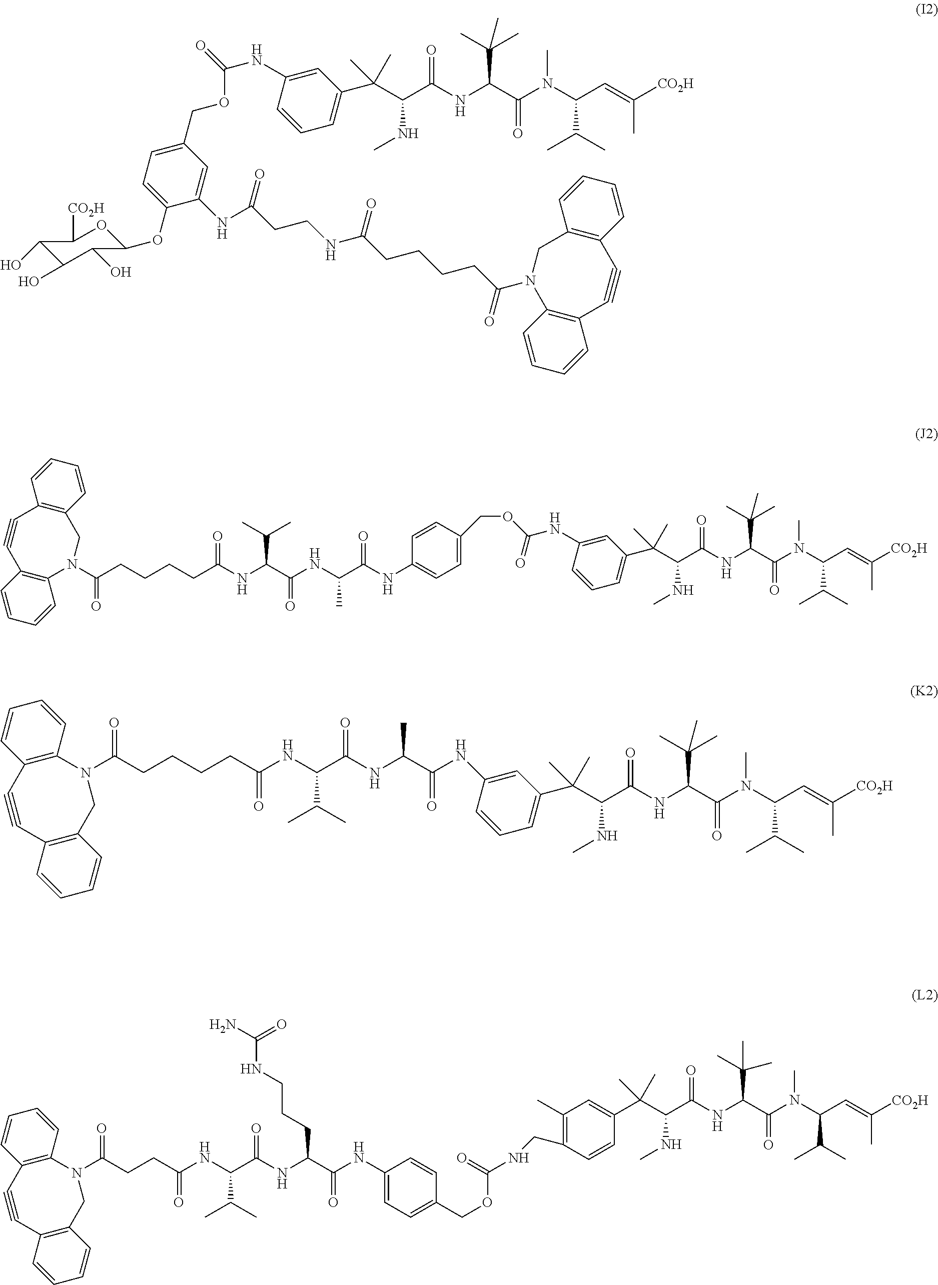

### 13. Vectors, Host Cells, and Recombinant Methods

Embodiments are also directed to the provision of isolated nucleic acids encoding anti-FOLR1 antibodies, vectors and host cells comprising the nucleic acids, and recombinant techniques for the production of the antibodies.

For recombinant production of the antibody, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include, but are not limited to, one or more of the following: a signal sequence, an origin of replication, one or more marker genes, an enhancer element, a promoter, and a transcription termination sequence, for example as described in U.S. Pat. No. 5,534,615, incorporated by reference in its entirety.

Illustrative examples of suitable host cells are provided below. These host cells are not meant to be limiting.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. licheniformis), Pseudomonas (P. aeruginosa), and Streptomyces. One useful E. coli cloning host is E. coli 294, although other strains such as E. coli B, E. coli x1776, and E. coli W3110 are suitable.

In addition to prokaryotes, eukaryotic microbes such as filamentous fungi or yeast are also suitable cloning or expression hosts for anti-FOLR1 antibody-encoding vectors. Saccharomyces cerevisiae, or common baker's yeast, is a commonly used lower eukaryotic host microorganism. However, a number of other genera, species, and strains are available and useful, such as Spodoptera frugiperda (e.g., SF9), Schizosaccharomyces pombe, Kluyveromyces (K lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the anti-FOLR1 antibody of this invention may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469, or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. For example, Carter et al. (Bio/Technology, 1992, 10:163-167) describes a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the antibody is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the antibody may be useful, for example, where the antibody accumulates in a cell as an insoluble aggregate, or where yields from periplasmic expression are low. The antibodies produced in a cell-free system may be aglycosylated depending on the source of the cells.

Where the antibody is secreted into the medium, supernatants from such expression systems are generally first concentrated using a commercially available protein concentration filter, for example, an Amicon® or Millipore® Pellicon® ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxyapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth., 1983, 62:1-13, incorporated by reference in its entirety). Protein G is useful for all mouse isotypes and for human γ3 (Guss et al., EMBO J, 1986, 5:1567-1575, incorporated by reference in its entirety).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the antibody of interest and contaminants may be subjected to low pH hydrophobic interaction chromatography using an elution buffer at a pH between about 2.5 to about 4.5, generally performed at low salt concentrations (e.g., from about 0 to about 0.25 M salt).

### 14. Pharmaceutical Compositions and Methods of Administration

The antibodies and antibody conjugates provided herein can be formulated into pharmaceutical compositions using methods available in the art and those disclosed herein. Any of the antibody conjugates provided herein can be provided in the appropriate pharmaceutical composition and be administered by a suitable route of administration.

The methods provided herein encompass administering pharmaceutical compositions comprising at least one antibody conjugate provided herein and one or more compatible and pharmaceutically acceptable carriers. In this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in Martin, E. W., Remington's Pharmaceutical Sciences.

In clinical practice the pharmaceutical compositions or antibody conjugates provided herein may be administered by any route known in the art. Exemplary routes of administration include, but are not limited to, the inhalation, intraarterial, intradermal, intramuscular, intraperitoneal, intravenous, nasal, parenteral, pulmonary, and subcutaneous routes. In some embodiments, a pharmaceutical composition or antibody conjugate provided herein is administered parenterally.

The compositions for parenteral administration can be emulsions or sterile solutions. Parenteral compositions may include, for example, propylene glycol, polyethylene glycol, vegetable oils, and injectable organic esters (e.g., ethyl oleate). These compositions can also contain wetting, isotonizing, emulsifying, dispersing and stabilizing agents. Sterilization can be carried out in several ways, for example using a bacteriological filter, by radiation or by heating. Parenteral compositions can also be prepared in the form of sterile solid compositions which can be dissolved at the time of use in sterile water or any other injectable sterile medium.

In some embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibody conjugates.

The pharmaceutical composition may comprise one or more pharmaceutical excipients. Any suitable pharmaceutical excipient may be used, and one of ordinary skill in the art is capable of selecting suitable pharmaceutical excipients. Non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific antibody in the dosage form. The composition or single unit dosage form, if desired, can also contain minor amounts of wetting or emulsifying agents, or pH buffering agents. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a co-solvent. Illustrative examples of co-solvents include ethanol, poly(ethylene) glycol, butylene glycol, dimethylacetamide, glycerin, and propylene glycol.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, and monosodium glutamate.

In some embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene(glycol) succinate.

In some embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6th Ed. (2009), The Pharmaceutical Press, incorporated by reference in its entirety.

In some embodiments, the pharmaceutical composition comprises a solvent. In some aspects, the solvent is saline solution, such as a sterile isotonic saline solution or dextrose solution. In some aspects, the solvent is water for injection.

In some embodiments, the pharmaceutical compositions are in a particulate form, such as a microparticle or a nanoparticle. Microparticles and nanoparticles may be formed from any suitable material, such as a polymer or a lipid. In some aspects, the microparticles or nanoparticles are micelles, liposomes, or polymersomes.

Further provided herein are anhydrous pharmaceutical compositions and dosage forms comprising an antibody conjugate, since, in some embodiments, water can facilitate the degradation of some antibodies.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Lactose-free compositions provided herein can comprise excipients that are well known in the art and are listed, for example, in the U.S. Pharmocopeia (USP) SP (XXI)/NF (XVI). In general, lactose-free compositions comprise an active ingredient, a binder/filler, and a lubricant in pharmaceutically compatible and pharmaceutically acceptable amounts. Exemplary lactose-free dosage forms comprise an active ingredient, microcrystalline cellulose, pre gelatinized starch, and magnesium stearate.

Also provided are pharmaceutical compositions and dosage forms that comprise one or more excipients that reduce the rate by which an antibody or antibody-conjugate will decompose. Such excipients, which are referred to herein as “stabilizers,” include, but are not limited to, antioxidants such as ascorbic acid, pH buffers, or salt buffers.

### 14.1. Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the antibodies disclosed herein can also be incorporated into the parenteral dosage forms.

### 15. Therapeutic Applications

For therapeutic applications, the conjugates provided herein can be administered to a mammal, generally a human, in a pharmaceutically acceptable dosage form such as those known in the art and those discussed above. For example, the antibody conjugates may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The antibody conjugates also are suitably administered by peritumoral, intralesional, or perilesional routes, to exert local as well as systemic therapeutic effects. The intravenous route may be particularly useful, for example, in the treatment of ovarian tumors.

The antibody conjugates provided herein may be useful for the treatment of any disease or condition involving folate receptor alpha (FOLR1). In some embodiments, the disease or condition is a disease or condition that can be diagnosed by overexpression of folate receptor alpha. In some embodiments, the disease or condition is a disease or condition that can benefit from treatment with an anti-folate receptor alpha antibody. In some embodiments, the disease or condition is a cancer.

Any suitable cancer may be treated with the antibody conjugates provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer (including triple-negative breast cancer, or TNBC), bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fallopian tube carcinoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer (NSCLC), oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, primary peritoneal carcinoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

In some embodiments, the disease to be treated with the antibody conjugates provided herein is gastric cancer, colorectal cancer, renal cell carcinoma, cervical cancer, non-small cell lung carcinoma, ovarian cancer, uterine cancer, fallopian tube carcinoma, primary peritoneal carcinoma, uterine corpus carcinoma, endometrial carcinoma, prostate cancer, breast cancer, head and neck cancer, brain carcinoma, liver cancer, pancreatic cancer, mesothelioma, and/or a cancer of epithelial origin. In particular embodiments, the disease is colorectal cancer. In some embodiments, the disease is ovarian cancer. In some embodiments, the disease is breast cancer. In some embodiments, the disease is triple-negative breast cancer (TNBC). In some embodiments, the disease is lung cancer. In some embodiments, the disease is non-small cell lung cancer (NSCLC). In some embodiments, the disease is head and neck cancer. In some embodiments, the disease is renal cell carcinoma. In some embodiments, the disease is brain carcinoma. In some embodiments, the disease is endometrial cancer.

In certain embodiments of this disclosure, the cancer is a solid tumor. For example, the cancer may be ovarian cancer, ovarian carcinoma, ovary cancer, endometrial cancer, endometrioid adenocarcinoma, fallopian tube cancer, or primary peritoneal carcinoma. In certain embodiments, the cancer is relapsed ovarian cancer. In certain embodiments, the cancer is refractory ovarian cancer. In certain embodiments, the cancer is relapsed/refractory ovarian cancer

In certain embodiments, the effectiveness of the methods herein (e.g., method of modulating an immune response in an individual) can be assessed by measuring the biological activity of cancer cells present in a sample isolated from the treated individual.

### 16. Combinations

In certain embodiments, the anti-FOLR1 antibody conjugates are used in combination with a second therapeutic agent that modulates angiogenesis. The anti-FOLR1 antibody conjugate can be any anti-FOLR1 antibody conjugate described herein. In particular embodiments, the second therapeutic agent is a VEGF inhibitor. Useful VEGF inhibitors are described herein. In particular embodiments, the second therapeutic agent is a GCSF. Useful GCSFs are described herein. In certain embodiments, the anti-FOLR1 antibody conjugates are used in combination with a VEGF inhibitor and with a GCSF.

Generally, the anti-FOLR1 antibody conjugate and the second therapeutic agent are administered according to their own doses and schedules. Thus, in certain embodiments, the anti-FOLR1 antibody conjugate is administered at a dose and schedule deemed useful by the practitioner of skill. In certain embodiments, the second therapeutic agent is administered at a dose and schedule deemed useful by the practitioner of skill. In particular embodiments, the second therapeutic agent is administered according to its labelled instruction.

In certain embodiments, the anti-FOLR1 antibody conjugate and the additional therapeutic agent are administered consecutively in either order. As used herein, the terms “consecutively,” “serially,” and “sequentially” refer to administration of an anti-FOLR1 antibody conjugate after an additional therapeutic agent, or administration of the additional therapeutic agent after the anti-FOLR1 antibody conjugate. For instance, consecutive administration may involve administration of the anti-FOLR1 antibody conjugate in the absence of the additional therapeutic agent during an induction phase (primary therapy), which is followed by a post-induction treatment phase comprising administration of the additional therapeutic agent. The methods may further comprise a maintenance phase comprising administration of the anti-FOLR1 antibody conjugate or the additional therapeutic agent, or both. Alternatively, consecutive administration may involve administration of the additional therapeutic agent in the absence of the anti-FOLR1 antibody conjugate during an induction phase (primary therapy), which is followed by a post-induction treatment phase comprising administration of the anti-FOLR1 antibody conjugate. The methods may further comprise a maintenance phase comprising administration of the anti-FOLR1 antibody conjugate or the additional therapeutic agent, or both.

In certain embodiments, the anti-FOLR1 antibody conjugate and the additional therapeutic agent are administered concurrently. As used herein, the terms “concurrently,” “simultaneously,” and “in parallel” refer to administration of an anti-FOLR1 antibody conjugate and an additional therapeutic agent during the same doctor visit or during the same phase of treatment. For instance, both the anti-FOLR1 antibody conjugate and the additional therapeutic agent may be administered during one or more of an induction phase, a treatment phase, and a maintenance phase. However, concurrent administration does not require that the anti-FOLR1 antibody conjugate and the additional therapeutic agent be present together in a single formulation or pharmaceutical composition, or that the anti-FOLR1 antibody conjugate and the additional therapeutic agent be administered at precisely the same time.

In certain embodiments, provided herein is a method of treating cancer responsive to inhibition of FOLR1 activity, the method comprising administering an effective amount of a combination provided herein to an individual to treat the cancer responsive to inhibition of FOLR1 activity. In certain embodiments, the cancer is an ovarian cancer such as one described herein.

In certain embodiments, provided herein is a method of treating cancer that is nonresponsive to the inhibition of FOLR1 activity alone, the method comprising administering an effective amount of a combination provided herein to such an individual to treat the cancer nonresponsive to inhibition of FOLR1 activity alone. In certain embodiments, the cancer is an ovarian cancer such as one described herein.

In certain embodiments, provided are compositions and therapeutic formulations comprising any of the antibody conjugates provided herein in combination with one or more second therapeutic agents, and methods of treatment comprising administering such combinations to subjects in need thereof. In some embodiments, the second agent is a VEGF inhibitor. In some embodiments, the one or more VEGF inhibitors comprise a an antibody that inhibits VEGF activity. In some embodiments, the one or more VEGF inhibitors are selected from bevacizumab (AVASTIN®) and bevacizumab biosimilars. In some embodiments, the bevacizumab biosimilar is selected from the group consisting of. MVASI (ABP 215, Amgen), Zirabev (Pfizer), Bevax (BEVZ92, mAbxience), Lumiere (Elea), Apotex (Apobiologix), Equidacent (FKB238, AstraZeneca/Centus Biotherapeutics), Avegra (BCD-021, Biocad), BP 01 (Aurobindo Pharma), BCD500 (BIOCND), Krabeva (Biocon), BAT1706 (Bio-Thera Solutions), BXT-2316 (BioXpress Therapeutics), Bevaro (CadilaPharmaceuticals), BI 695502 (Boehringer Ingelheim), CT-P16 (Celltrion), CHS-5217 (Coherus), DRZ_BZ (Dr Reddy's Laboratories), Cizumab (Hetero/Lupin), Byvasda (IB1I305, Innovent Biologics), MIL60 (Mabworks), MYL 14020 (Mylan), ONS-1045 (Oncobiologics/Viropro), HD204 (Prestige Biopharma), Ankeda (QL1101, Qilu Pharmaceutical), Bevacirel (Reliance Life Sciences), Aybintio (SB8, Samsung Bioepis), Onbevzi (Samsung Bioepis), HLX04 (Shanghai Henlius Biotech), TX16 (Tanvex BioPharma), MB02(mAbxience), BI 695502 (Boehringer Ingelheim), and Oyavas (STADA).

Examples of second agents also include, but are not limited to, Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially uncialamycin, calicheamicin gammall, and calicheamicin omegall (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pladienolide B, pteropterin, trimetrexate; purine analogs such as fludarabine, 6-mercaptopurine, thiamniprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (doxetaxel; Rhone-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

In some embodiments, the second therapeutic agent is a granulocyte colony-stimulating factor (GCSF). In some embodiments, the second therapeutic agent is second therapeutic agent is a pegylated GCSF. In some embodiments, the second therapeutic agent is GCSF is pegfilgrastim or filgrastim. In some embodiments, the second therapeutic agent is pegfilgrastim. Pegfilgrastim may be Neulasta® or a biosimilar. In some embodiments, the second therapeutic agent is filgrastim. Filgrastim may be Neupogen® or a biosimilar.

In some embodiments, the GCSF second therapeutic agent is administered concurrently with or sequentially to the first dose of the antibody conjugate. In some embodiments, the second therapeutic agent is second therapeutic agent is administered at least 7 days after administering the first dose of the antibody conjugate. In some embodiments, the second therapeutic agent is second therapeutic agent is administered at 7, 8, 9, 10 or 11 days after administering the first dose of the antibody conjugate. In some embodiments, the second therapeutic agent is second therapeutic agent is administered at 8±2 days after administering the first dose of the antibody conjugate. In some embodiments, the second therapeutic agent is second therapeutic agent is administered at around 8 days after administering the first dose of the antibody conjugate. In some embodiments, the second therapeutic agent is administered 8 days after administering the first dose of the antibody conjugate.

In some embodiments, the GCSF is pegfilgrastim, which is administered as directed or as deemed suitable by the practitioner of skill. In certain embodiments, the pegfilgrastim dose is 6 mg. In some embodiments, pegfilgrastim is administered once every 8-10 days for 1-5 cycles. In some embodiments, pegfilgrastim is administered once every 8-10 days for 1-3 cycles. In some embodiments, pegfilgrastim is administered once every 8-10 days for 1 cycle.

In some embodiments, the GCSF is filgrastim, which is administered as directed or as deemed suitable by the practitioner of skill. In certain embodiments, the first dose is administered at least 24 hours after administering the first dose of the antibody conjugate. In some embodiments, the GCSF is filgrastim and the dose is 5-10 μg/kg/day. In some embodiments, the GCSF is filgrastim and the dose is 5 μg/kg/day. In some embodiments, the GCSF is filgrastim, the dose is 5 μg/kg/day, and the patient is receiving myelosuppressive chemotherapy or induction and/or consolidation chemotherapy for AML. In some embodiments, the GCSF is intravenous filgrastim, the dose is 5 μg/kg/day, and the patient is receiving myelosuppressive chemotherapy or induction and/or consolidation chemotherapy for AML. In some embodiments, the GCSF is subcutaneous filgrastim, the dose is 5 μg/kg/day, and the patient is receiving myelosuppressive chemotherapy or induction and/or consolidation chemotherapy for AML. In some embodiments, the GCSF is filgrastim and the dose is 10 μg/kg/day. In some embodiments, the GCSF is filgrastim, the dose is 10 μg/kg/day, and the patient is undergoing bone marrow transplantation or undergoing autologous peripheral blood progenitor cell collection and therapy. In some embodiments, the GCSF is subcutaneous filgrastim, the dose is 10 μg/kg/day, and the patient is undergoing bone marrow transplantation. In some embodiments, the GCSF is intravenous filgrastim, the dose is 10 μg/kg/day, and the patient is undergoing autologous peripheral blood progenitor cell collection and therapy. In some embodiments, the GCSF is filgrastim and the dose is 6 μg/kg/day. In some embodiments, the GCSF is filgrastim, the dose is 6 μg/kg/day, and the patient has congenital neutropenia. In some embodiments, the GCSF is subcutaneous filgrastim, the dose is 6 μg/kg/day, and the patient has congenital neutropenia. In some embodiments, the GCSF is filgrastim, the dose is 5 μg/kg/day, and the patient has cyclic or idiopathic neutropenia. In some embodiments, the GCSF is subcutaneous filgrastim, the dose is 5 μg/kg/day, and the patient has cyclic or idiopathic neutropenia. In some embodiments, the GCSF is filgrastim, the dose is 10 μg/kg/day, and the patient has been acutely exposed to myelosuppressive doses of radiation. In some embodiments, the GCSF is subcutaneous filgrastim, the dose is 10 μg/kg/day, and the patient has been acutely exposed to myelosuppressive doses of radiation.

### 17. Kits

In some embodiments, the combination provided herein is provided in the form of a kit, i.e., a packaged combination of reagents in predetermined amounts with instructions for performing a procedure. In other embodiments, the procedure is a therapeutic procedure.

In some embodiments, the kit further comprises a solvent for the reconstitution of the anti-FOLR1 antibody conjugate. In some embodiments, the anti-FOLR1 antibody conjugate is provided in the form of a pharmaceutical composition.

In some embodiments, the kit further comprises a VEGF inhibitor, e.g., bevacizumab or a bevacizumab biosimilar, and instructions for use. In certain embodiments, the kit provides a GCSF, e.g., pegfilgrastim or filgrastim, and instructions for use. In certain embodiments, the kit provides pegfilgrastim and instructions for use. In certain embodiments, the kit provides filgrastim and instructions for use.

In some embodiments, the kit further comprises instructions for use with a VEGF inhibitor, e.g., bevacizumab or a bevacizumab biosimilar. In certain embodiments, the kit provides instructions for use with a GCSF, e.g., pegfilgrastim or filgrastim. In certain embodiments, the kit provides instructions for use with pegfilgrastim. In certain embodiments, the kit provides instructions for use with filgrastim.

In certain embodiments, the pharmaceutical package or kit comprises a container, a composition comprising a folate receptor alpha (FOLR1) antibody conjugate; and a package insert comprising instructions to administer the FOLR1 antibody conjugate according the methods described herein.

### EXAMPLES

### Example 1

Anti-Folr1 in Patients with Ovarian Cancer

In this study, the pharmacokinetics, safety and efficacy of Conjugate A were evaluated when given by intravenous (IV) administration for the treatment of advanced epithelial ovarian cancer (including fallopian tube or primary peritoneal cancers). Patients included individuals having relapsed platinum resistant ovarian cancer or other high grade serous epithelial ovarian, fallopian tube or primary peritoneal cancer.

Patients were excluded if they have any number of conditions including low grade ovarian carcinoma (grade 1); clear cell, mucinous, endometrioid, sarcomatous, and mixed histology ovarian carcinomas and sarcomatous ovarian carcinomas; prior treatment with FolRα targeting ADCs or ADCs that contain a tubulin inhibitor; prior anticancer therapy (prior to first dose of study drug): including chemotherapy within 3 weeks, PARP inhibitor within 2 weeks, other therapeutic anticancer antibodies within 3 weeks, radio- or toxin-immunoconjugates (e.g. ADCs) within 10 weeks, or radiation therapy/major surgery within 4 weeks of first dose of study drug; preexisting clinically significant ocular disorders; previous solid organ transplantation; bowel obstruction and/or signs/symptoms of bowel obstruction within the preceding 3 months; history of gastrointestinal perforation; residual CTCAE≥Grade 2 toxicity from prior anticancer therapy; history of significant congestive heart failure; Nephrotic syndrome; sensory or motor neuropathy grade >1; potentially fatal concurrent or recent malignancy; chronic or ongoing active infectious disease requiring systemic treatment; ongoing immunosuppressive therapy, including systemic corticosteroids; clinically significant cardiac disease; significant concurrent, uncontrolled medical condition including, but not limited to, renal, hepatic, hematological, gastrointestinal, endocrine, pulmonary, neurological, cerebral or psychiatric disease; history or clinical signs of meningeal or active central nervous system involvement; known severe chronic obstructive pulmonary disease or asthma (defined as FEV1<40% of expected) or active pneumonitis within 6 months of the first dose of study drug; history of significant cerebrovascular disease such as stroke or TIA within 6 months; known human immunodeficiency virus seropositivity; pregnancy or breastfeeding; positive serology for hepatitis B defined by a positive test for HBsAg; concurrent participation in another therapeutic treatment trial; evidence of recto-sigmoid involvement by pelvic examination or bowel involvement on CT scan; history of hemoptysis within 6 months; venous or arterial thromboembolic event within 3 months.

The initial dose of Conjugate A was 4.3 mg/kg or 5.2 mg/kg. Dosing was given every three weeks. Certain patients initiated with the 5.2 mg/kg dose then switched to the 4.3 mg/kg dose after two or three cycles of the initial dose.

Patients were monitored to evaluate adverse events, overall response rate (ORR), duration of response (DOR), overall survival (OS), and progression free survival (PFS) will be determined. Patients are monitored for potential drug-drug interactions.

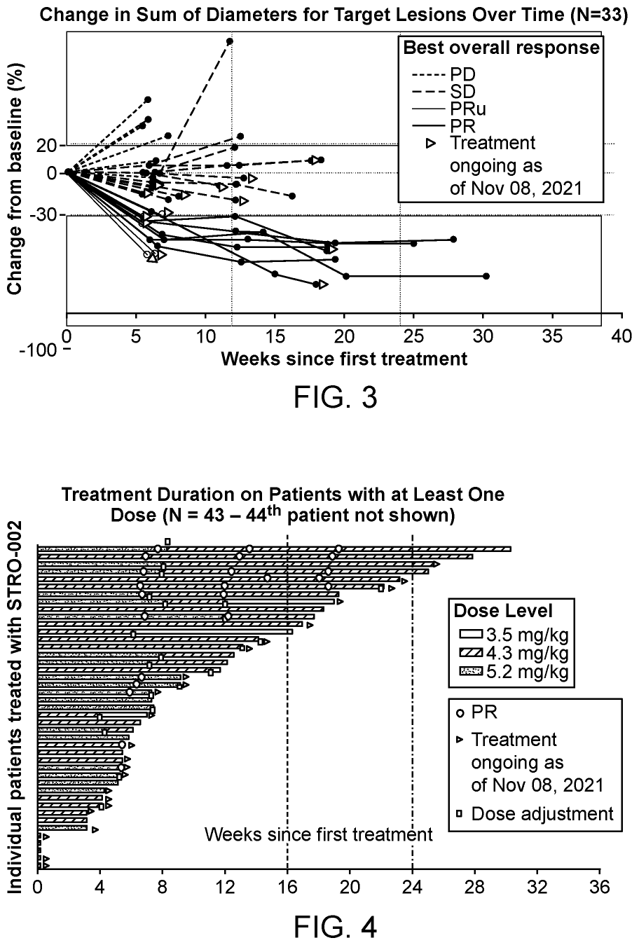

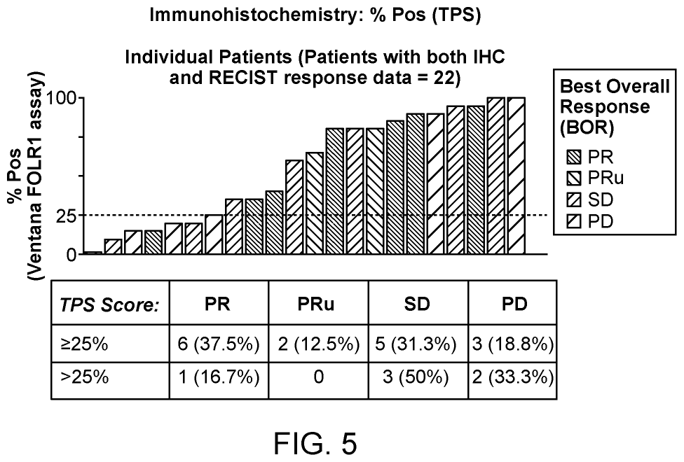

FIG. 1 provides the maximum change in tumor target lesions across 33 patients. Seven had confirmed partial responses, and five had unconfirmed partial responses as of the analysis date. FIG. 2 shows that about twice as many (48% vs. 25%) patients had a partial response (confirmed or unconfirmed) when comparing the initial doses of 5.2 mg/kg vs. 4.3 mg/kg. FIG. 3 provides the changes in size of target lesions in 33 patients. Good tumor control was achieved for many patients. FIG. 4 provides treatment durability across 43 patients. Patients were initially dosed with 4.3 mg/kg or 5.2 mg/kg of Conjugate A. Anti-tumor activity appeared to be robust after 2 cycles at 5.2 mg/kg. Patients with dose changes from 5.2 mg/kg to 4.3 mg/kg are indicated. Dose reductions did not seem to impact ability to maintain disease control. Patients with partial responses are also indicated. FIG. 5 provides immunohistochemistry positive scores across 22 patients. FolR1 The intensity of staining is scored from 0 (no staining) to 1+, 2+or 3+(the highest intensity). TPS is the percentage of cells with any intensity of staining. The proportion score is IHC positive tumor cells/total tumor cells x 100%.

In a dose expansion cohort of 28 patients with relapsed/recurrent platinum resistant ovarian cancer, the preliminary response rate (including both confirmed and unconfirmed responses per RECIST v1.1), was 32%.

### Example 2

Anti-Folr1 in Patients with Ovarian Cancer

In this study, the pharmacokinetics, safety, and efficacy of Conjugate A are evaluated when given by intravenous (IV) administration for the treatment of advanced epithelial ovarian cancer (including fallopian tube and primary peritoneal cancers). Patients include individuals having advanced relapsed and/or progressive disease.

The initial dose of Conjugate A is 5.2 mg/kg or 4.3 mg/kg. Dosing is given every three weeks. Pegfilgrastim is administered at 6 mg subcutaneously on day 8±2 days of a treatment cycle. Certain patients initiated with the 5.2 mg/kg dose are switched to the 4.3 mg/kg dose after one or two or three or four cycles of the initial dose. Certain patients on the 4.3 mg/kg dose are switched to the 3.5 mg/kg dose after one or two or three cycles of the prior dose.

Patients are monitored to evaluate adverse events, overall response rate (ORR), duration of response (DOR), overall survival (OS), and progression free survival (PFS). Patients are monitored for potential drug-drug interactions.

### Example 3

Table 5 provides sequences referred to herein.

TABLE 5
Sequences

| SEQ | | | | |
|---|---|---|---|---|
| ID | | | | |
| NO: | Molecule | Region | Scheme | Sequence |
| 1 | Human folate | | | MAQRMTTQLLLLLVWVAVVGEAQTRIAW |
| | receptor alpha | | | ARTELLNVCMNAKHHKEKPGPEDKLHEQ |
| | (hFOLR1) | | | CRPWRKNACCSTNTSQEAHKDVSYLYRF |
| | | | | NWNHCGEMAPACKRHFIQDTCLYECSPN |
| | | | | LGPWIQQVDQSWRKERVLNVPLCKEDCE |
| | | | | QWWEDCRTSYTCKSNWHKGWNWTSGENK |
| | | | | CAVGAACQPFHFYFPTPTVLCNEIWTHS |
| | | | | YKVSNYSRGSGRCIQMWFDPAQGNPNEE |
| | | | | VARFYAAAMSGAGPWAAWPFLLSLALML |
| | | | | LWLLS |
| 2 | Cynomolgus folate | | | MAQRMTTQLLLLLVWVAVVGEAQTRTAR |
| | receptor alpha | | | ARTELLNVCMNAKHHKEKPGPEDKLHEQ |
| | | | | CRPWKKNACCSTNTSQEAHKDVSYLYRF |
| | | | | NWNHCGEMAPACKRHFIQDTCLYECSPN |
| | | | | LGPWIQQVDQSWRKERVLNVPLCKEDCE |
| | | | | RWWEDCRTSYTCKSNWHKGWNWTSGENK |
| | | | | CPVGAACQPFHFYFPTPTVLCNEIWTYS |
| | | | | YKVSNYSRGSGRCIQMWFDPAQGNPNEE |
| | | | | VARFYAAAMSGAGPWAAWPLLLSLALTL |
| | | | | LWLLS |
| 3 | Murine folate | | | MAHLMTVQLLLLVMWMAECAQSRATRAR |
| | receptor alpha | | | TELLNVCMDAKHHKEKPGPEDNLHDQCS |
| | | | | PWKTNSCCSTNTSQEAHKDISYLYRENW |
| | | | | NHCGTMTSECKRHFIQDTCLYECSPNLG |
| | | | | PWIQQVDQSWRKERILDVPLCKEDCQQW |
| | | | | WEDCQSSFTCKSNWHKGWNWSSGHNECP |
| | | | | VGASCHPFTFYFPTSAALCEEIWSHSYK |
| | | | | LSNYSRGSGRCIQMWFDPAQGNPNEEVA |
| | | | | RFYAEAMSGAGFHGTWPLLCSLSLVLLW |
| | | | | VIS |
| 4 | SRP1848-A01 | CDR-H1 | Chothia | GFNITRY |
| 5 | SRP1848-A02 | CDR-H1 | Chothia | GFNISGF |
| 6 | SRP1848-A04 | CDR-H1 | Chothia | GENIDQS |
| 7 | SRP1848-A06 | CDR-H1 | Chothia | GENIGNS |
| 8 | SRP1848-A07 | CDR-H1 | Chothia | GFNIGYH |
| 9 | SRP1848-A08 | CDR-H1 | Chothia | GSNIRKH |
| 10 | SRP1848-A09 | CDR-H1 | Chothia | GFNIRKQ |
| 11 | SRP1848-A10 | CDR-H1 | Chothia | GFNIRKY |
| 12 | SRP1848-B01 | CDR-H1 | Chothia | GENIRNY |
| 13 | SRP1848-B03 | CDR-H1 | Chothia | GFNISMK |
| 14 | SRP1848-B04 | CDR-H1 | Chothia | SFNISNH |
| 15 | SRP1848-B05 | CDR-H1 | Chothia | GFNISNY |
| 16 | SRP1848-B06 | CDR-H1 | Chothia | GFNISNY |
| 17 | SRP1848-B07 | CDR-H1 | Chothia | GENISRE |
| 18 | SRP1848-B09 | CDR-H1 | Chothia | GFNITNY |
| 19 | SRP1848-B10 | CDR-H1 | Chothia | GENTTTK |
| 20 | SRP1848-B11 | CDR-H1 | Chothia | GFNIGNN |
| 21 | SRP1848-C01 | CDR-H1 | Chothia | GFNIGNS |
| 22 | SRP1848-C03 | CDR-H1 | Chothia | GFNIGVY |
| 23 | SRP1848-C04 | CDR-H1 | Chothia | GFNIRHY |
| 24 | SRP1848-C05 | CDR-H1 | Chothia | GENIRKY |
| 25 | SRP1848-C07 | CDR-H1 | Chothia | GFNIRKY |
| 26 | SRP1848-C10 | CDR-H1 | Chothia | GENIRTY |
| 27 | SRP1848-D02 | CDR-H1 | Chothia | GFNISHN |
| 28 | SRP1848-D03 | CDR-H1 | Chothia | GFNIRYF |
| 29 | SRP1848-D04 | CDR-H1 | Chothia | GFNISHY |
| 30 | SRP1848-D05 | CDR-H1 | Chothia | GENISIS |
| 31 | SRP1848-D07 | CDR-H1 | Chothia | GFNISKY |
| 32 | SRP1848-D09 | CDR-H1 | Chothia | GENISNY |
| 33 | SRP1848-D10 | CDR-H1 | Chothia | GFNISRN |
| 34 | SRP1848-E01 | CDR-H1 | Chothia | GFNITNK |
| 35 | SRP1848-E02 | CDR-H1 | Chothia | GFNIGKY |
| 36 | SRP1848-E03 | CDR-H1 | Chothia | GENIGNY |
| 37 | SRP1848-E05 | CDR-H1 | Chothia | GFNIGVY |
| 38 | SRP1848-E06 | CDR-H1 | Chothia | GENINRY |
| 39 | SRP1848-E07 | CDR-H1 | Chothia | GENIRKS |
| 40 | SRP1848-F01 | CDR-H1 | Chothia | GENIRTY |
| 41 | SRP1848-F02 | CDR-H1 | Chothia | GFNIRTY |
| 42 | SRP1848-F04 | CDR-H1 | Chothia | GENISNY |
| 43 | SRP1848-F05 | CDR-H1 | Chothia | GFNISKS |
| 44 | SRP1848-F06 | CDR-H1 | Chothia | GENISLS |
| 45 | SRP1848-F07 | CDR-H1 | Chothia | GFNISNH |
| 46 | SRP1848-F08 | CDR-H1 | Chothia | GFNISNH |
| 47 | SRP1848-F09 | CDR-H1 | Chothia | GFNISNH |
| 48 | SRP1848-F10 | CDR-H1 | Chothia | GFNISNN |
| 49 | SRP1848-F11 | CDR-H1 | Chothia | GFNISNN |
| 50 | SRP1848-G01 | CDR-H1 | Chothia | GFNISRH |
| 51 | SRP1848-G03 | CDR-H1 | Chothia | GFNISTY |
| 52 | SRP1848-G04 | CDR-H1 | Chothia | GFNIHST |
| 53 | SRP1848-G06 | CDR-H1 | Chothia | GENIRST |
| 54 | SRP1848-G07 | CDR-H1 | Chothia | GFNIHST |
| 55 | SRP1848-G09 | CDR-H1 | Chothia | GFNIRGT |
| 56 | SRP1848-G10 | CDR-H1 | Chothia | GFNIRST |
| 57 | SRP1848-G11 | CDR-H1 | Chothia | GFNISST |
| 58 | SRP1848-H01 | CDR-H1 | Chothia | GENIRTQ |
| 59 | SRP2060-E10 | CDR-H1 | Chothia | GFSLSTFGM |
| 60 | SRP2060-E05 | CDR-H1 | Chothia | GFSLSTFGM |
| 61 | SRP2060-B01 | CDR-H1 | Chothia | GFSLSTFGM |
| 62 | SRP2060-A06 | CDR-H1 | Chothia | GFSLSTFGM |
| 63 | SRP1848-A01 | CDR-H1 | Kabat | RYSIH |
| 64 | SRP1848-A02 | CDR-H1 | Kabat | GFRIH |
| 65 | SRP1848-A04 | CDR-H1 | Kabat | QSSIH |
| 66 | SRP1848-A06 | CDR-H1 | Kabat | NSYIH |
| 67 | SRP1848-A07 | CDR-H1 | Kabat | YHSIH |
| 68 | SRP1848-A08 | CDR-H1 | Kabat | KHSIH |
| 69 | SRP1848-A09 | CDR-H1 | Kabat | KQSIH |
| 70 | SRP1848-A10 | CDR-H1 | Kabat | KYSIH |
| 71 | SRP1848-B01 | CDR-H1 | Kabat | NYSIH |
| 72 | SRP1848-B03 | CDR-H1 | Kabat | MKYIH |
| 73 | SRP1848-B04 | CDR-H1 | Kabat | NHSIH |
| 74 | SRP1848-B05 | CDR-H1 | Kabat | NYYIH |
| 75 | SRP1848-B06 | CDR-H1 | Kabat | NYYIH |
| 76 | SRP1848-B07 | CDR-H1 | Kabat | RFYIH |
| 77 | SRP1848-B09 | CDR-H1 | Kabat | NYYIH |
| 78 | SRP1848-B10 | CDR-H1 | Kabat | TKSIH |
| 79 | SRP1848-B11 | CDR-H1 | Kabat | NNSIH |
| 80 | SRP1848-C01 | CDR-H1 | Kabat | NSYIH |
| 81 | SRP1848-C03 | CDR-H1 | Kabat | VYSIH |
| 82 | SRP1848-C04 | CDR-H1 | Kabat | HYSIH |
| 83 | SRP1848-C05 | CDR-H1 | Kabat | KYSIH |
| 84 | SRP1848-C07 | CDR-H1 | Kabat | KYSIH |
| 85 | SRP1848-C10 | CDR-H1 | Kabat | TYYIH |
| 86 | SRP1848-D02 | CDR-H1 | Kabat | HNYIH |
| 87 | SRP1848-D03 | CDR-H1 | Kabat | YFSIH |
| 88 | SRP1848-D04 | CDR-H1 | Kabat | HYSIH |
| 89 | SRP1848-D05 | CDR-H1 | Kabat | ISYIH |
| 90 | SRP1848-D07 | CDR-H1 | Kabat | KYYIH |
| 91 | SRP1848-D09 | CDR-H1 | Kabat | NYYIH |
| 92 | SRP1848-D10 | CDR-H1 | Kabat | RNSIH |
| 93 | SRP1848-E01 | CDR-H1 | Kabat | NKYIH |
| 94 | SRP1848-E02 | CDR-H1 | Kabat | KYSIH |
| 95 | SRP1848-E03 | CDR-H1 | Kabat | NYYIH |
| 96 | SRP1848-E05 | CDR-H1 | Kabat | VYYIH |
| 97 | SRP1848-E06 | CDR-H1 | Kabat | RYYIH |
| 98 | SRP1848-E07 | CDR-H1 | Kabat | KSSIH |
| 99 | SRP1848-F01 | CDR-H1 | Kabat | TYSIH |
| 100 | SRP1848-F02 | CDR-H1 | Kabat | TYSIH |
| 101 | SRP1848-F04 | CDR-H1 | Kabat | NYSIH |
| 102 | SRP1848-F05 | CDR-H1 | Kabat | KSSIH |
| 103 | SRP1848-F06 | CDR-H1 | Kabat | LSYIH |
| 104 | SRP1848-F07 | CDR-H1 | Kabat | NHSIH |
| 105 | SRP1848-F08 | CDR-H1 | Kabat | NHSIH |
| 106 | SRP1848-F09 | CDR-H1 | Kabat | NHYIH |
| 107 | SRP1848-F10 | CDR-H1 | Kabat | NNSIH |
| 108 | SRP1848-F11 | CDR-H1 | Kabat | NNYIH |
| 109 | SRP1848-G01 | CDR-H1 | Kabat | RHSIH |
| 110 | SRP1848-G03 | CDR-H1 | Kabat | TYYIH |
| 111 | SRP1848-G04 | CDR-H1 | Kabat | STDIH |
| 112 | SRP1848-G06 | CDR-H1 | Kabat | STDIH |
| 113 | SRP1848-G07 | CDR-H1 | Kabat | STDIH |
| 114 | SRP1848-G09 | CDR-H1 | Kabat | GTDIH |
| 115 | SRP1848-G10 | CDR-H1 | Kabat | STDIH |
| 116 | SRP1848-G11 | CDR-H1 | Kabat | STDIH |
| 117 | SRP1848-H01 | CDR-H1 | Kabat | TQSIH |
| 118 | SRP2060-E10 | CDR-H1 | Kabat | TFGMGVG |
| 119 | SRP2060-E05 | CDR-H1 | Kabat | TFGMGVG |
| 120 | SRP2060-B01 | CDR-H1 | Kabat | TFGMGVG |
| 121 | SRP2060-A06 | CDR-H1 | Kabat | TFGMGVG |
| 122 | SRP1848-A01 | CDR-H2 | Chothia | LPESGG |
| 123 | SRP1848-A02 | CDR-H2 | Chothia | YPESGA |
| 124 | SRP1848-A04 | CDR-H2 | Chothia | YPVDGT |
| 125 | SRP1848-A06 | CDR-H2 | Chothia | TPIDGN |
| 126 | SRP1848-A07 | CDR-H2 | Chothia | FPVDGT |
| 127 | SRP1848-A08 | CDR-H2 | Chothia | YPNDGT |
| 128 | SRP1848-A09 | CDR-H2 | Chothia | FPNDGT |
| 129 | SRP1848-A10 | CDR-H2 | Chothia | FPIDDI |
| 130 | SRP1848-B01 | CDR-H2 | Chothia | YPVDGI |
| 131 | SRP1848-B03 | CDR-H2 | Chothia | TPIDGM |
| 132 | SRP1848-B04 | CDR-H2 | Chothia | YPVDGI |
| 133 | SRP1848-B05 | CDR-H2 | Chothia | SPIDGY |
| 134 | SRP1848-B06 | CDR-H2 | Chothia | TPIDGY |
| 135 | SRP1848-B07 | CDR-H2 | Chothia | SPYDGF |
| 136 | SRP1848-B09 | CDR-H2 | Chothia | TPVDGY |
| 137 | SRP1848-B10 | CDR-H2 | Chothia | YPRDGI |
| 138 | SRP1848-B11 | CDR-H2 | Chothia | SPIDGF |
| 139 | SRP1848-C01 | CDR-H2 | Chothia | TPNDGY |
| 140 | SRP1848-C03 | CDR-H2 | Chothia | YPIDGN |
| 141 | SRP1848-C04 | CDR-H2 | Chothia | YPGPGN |
| 142 | SRP1848-C05 | CDR-H2 | Chothia | FPIDGI |
| 143 | SRP1848-C07 | CDR-H2 | Chothia | FPIDGI |
| 144 | SRP1848-C10 | CDR-H2 | Chothia | SPIDGY |
| 145 | SRP1848-D02 | CDR-H2 | Chothia | TPQDGY |
| 146 | SRP1848-D03 | CDR-H2 | Chothia | FPNDGS |
| 147 | SRP1848-D04 | CDR-H2 | Chothia | YPRDGI |
| 148 | SRP1848-D05 | CDR-H2 | Chothia | SPIDGY |
| 149 | SRP1848-D07 | CDR-H2 | Chothia | SPNDGY |
| 150 | SRP1848-D09 | CDR-H2 | Chothia | SPNDGY |
| 151 | SRP1848-D10 | CDR-H2 | Chothia | SPENDGT |
| 152 | SRP1848-E01 | CDR-H2 | Chothia | TPFDGF |
| 153 | SRP1848-E02 | CDR-H2 | Chothia | YPNDGN |
| 154 | SRP1848-E03 | CDR-H2 | Chothia | TPRDGF |
| 155 | SRP1848-E05 | CDR-H2 | Chothia | TPNDGY |
| 156 | SRP1848-E06 | CDR-H2 | Chothia | TPNDGY |
| 157 | SRP1848-E07 | CDR-H2 | Chothia | FPYDGS |
| 158 | SRP1848-F01 | CDR-H2 | Chothia | FPNDGT |
| 159 | SRP1848-F02 | CDR-H2 | Chothia | FPNDGT |
| 160 | SRP1848-F04 | CDR-H2 | Chothia | YPIDGI |
| 161 | SRP1848-F05 | CDR-H2 | Chothia | YPNDGS |
| 162 | SRP1848-F06 | CDR-H2 | Chothia | SPIDGN |
| 163 | SRP1848-F07 | CDR-H2 | Chothia | YPNDGI |
| 164 | SRP1848-F08 | CDR-H2 | Chothia | YPVDGI |
| 165 | SRP1848-F09 | CDR-H2 | Chothia | SPLDGY |
| 166 | SRP1848-F10 | CDR-H2 | Chothia | FPNDGY |
| 167 | SRP1848-F11 | CDR-H2 | Chothia | TPIDGN |
| 168 | SRP1848-G01 | CDR-H2 | Chothia | APNDGS |
| 169 | SRP1848-G03 | CDR-H2 | Chothia | TPSDGF |
| 170 | SRP1848-G04 | CDR-H2 | Chothia | TPAGGA |
| 171 | SRP1848-G06 | CDR-H2 | Chothia | TPAGGA |
| 172 | SRP1848-G07 | CDR-H2 | Chothia | TPAGGA |
| 173 | SRP1848-G09 | CDR-H2 | Chothia | TPAGGA |
| 174 | SRP1848-G10 | CDR-H2 | Chothia | TPAGGA |
| 175 | SRP1848-G11 | CDR-H2 | Chothia | TPAGGA |
| 176 | SRP1848-H01 | CDR-H2 | Chothia | FPIDGI |
| 177 | SRP2060-E10 | CDR-H2 | Chothia | WWDDD |
| 178 | SRP2060-E05 | CDR-H2 | Chothia | WWDDD |
| 179 | SRP2060-B01 | CDR-H2 | Chothia | WWDDD |
| 180 | SRP2060-A06 | CDR-H2 | Chothia | WWDDD |
| 181 | SRP1848-A01 | CDR-H2 | Kabat | GILPESGGTSYADSVKG |
| 182 | SRP1848-A02 | CDR-H2 | Kabat | GIYPESGATYYADSVKG |
| 183 | SRP1848-A04 | CDR-H2 | Kabat | VIYPVDGTTDYADSVKG |
| 184 | SRP1848-A06 | CDR-H2 | Kabat | GITPIDGNTDYADSVKG |
| 185 | SRP1848-A07 | CDR-H2 | Kabat | EIFPVDGTTDYADSVKG |
| 186 | SRP1848-A08 | CDR-H2 | Kabat | SIYPNDGTTDYADSVKG |
| 187 | SRP1848-A09 | CDR-H2 | Kabat | SIFPNDGTTDYADSVKG |
| 188 | SRP1848-A10 | CDR-H2 | Kabat | DIFPIDDITDYADSVKG |
| 189 | SRP1848-B01 | CDR-H2 | Kabat | EIYPVDGITDYADSVKG |
| 190 | SRP1848-B03 | CDR-H2 | Kabat | GITPIDGMTDYADSVKG |
| 191 | SRP1848-B04 | CDR-H2 | Kabat | EIYPVDGITDYADSVKG |
| 192 | SRP1848-B05 | CDR-H2 | Kabat | GISPIDGYTDYADSMKG |
| 193 | SRP1848-B06 | CDR-H2 | Kabat | GITPIDGYTDYADSVKG |
| 194 | SRP1848-B07 | CDR-H2 | Kabat | GISPYDGFTDYADSVKG |
| 195 | SRP1848-B09 | CDR-H2 | Kabat | GITPVDGYTDYADRVKG |
| 196 | SRP1848-B10 | CDR-H2 | Kabat | EIYPRDGITDYADSVKG |
| 197 | SRP1848-B11 | CDR-H2 | Kabat | DISPIDGFTDYADSVKG |
| 198 | SRP1848-C01 | CDR-H2 | Kabat | GVTPNDGYTDYADSVKG |
| 199 | SRP1848-C03 | CDR-H2 | Kabat | EIYPIDGNTDYADSVKG |
| 200 | SRP1848-C04 | CDR-H2 | Kabat | EIYPGPGNTDYADSVKG |
| 201 | SRP1848-C05 | CDR-H2 | Kabat | DIFPIDGINDYADSVKG |
| 202 | SRP1848-C07 | CDR-H2 | Kabat | DIFPIDGITDYADSVKG |
| 203 | SRP1848-C10 | CDR-H2 | Kabat | GISPIDGYTDYADSMKG |
| 204 | SRP1848-D02 | CDR-H2 | Kabat | GITPQDGYTDYADSVKG |
| 205 | SRP1848-D03 | CDR-H2 | Kabat | DIFPNDGSTDYADSVKG |
| 206 | SRP1848-D04 | CDR-H2 | Kabat | EIYPRDGITDYADSVKG |
| 207 | SRP1848-D05 | CDR-H2 | Kabat | GISPIDGYTDYADSVKG |
| 208 | SRP1848-D07 | CDR-H2 | Kabat | GISPNDGYTDYADSVKG |
| 209 | SRP1848-D09 | CDR-H2 | Kabat | GISPNDGYTDYADSVKG |
| 210 | SRP1848-D10 | CDR-H2 | Kabat | WISPNDGTTDYADSVKG |
| 211 | SRP1848-E01 | CDR-H2 | Kabat | GITPFDGFTDYADSVKG |
| 212 | SRP1848-E02 | CDR-H2 | Kabat | EIYPNDGNTDYADSVKG |
| 213 | SRP1848-E03 | CDR-H2 | Kabat | GITPRDGFTDYADSVKG |
| 214 | SRP1848-E05 | CDR-H2 | Kabat | GITPNDGYTDYADSVKG |
| 215 | SRP1848-E06 | CDR-H2 | Kabat | GITPNDGYTDYADSVEG |
| 216 | SRP1848-E07 | CDR-H2 | Kabat | EIFPYDGSTDYADNVKG |
| 217 | SRP1848-F01 | CDR-H2 | Kabat | SIFPNDGTTDYADSVKG |
| 218 | SRP1848-F02 | CDR-H2 | Kabat | SIFPNDGTTDYADSVKG |
| 219 | SRP1848-F04 | CDR-H2 | Kabat | EIYPIDGITDYADSVKG |
| 220 | SRP1848-F05 | CDR-H2 | Kabat | EIYPNDGSTDYADSVKG |
| 221 | SRP1848-F06 | CDR-H2 | Kabat | GISPIDGNTDYADSVKG |
| 222 | SRP1848-F07 | CDR-H2 | Kabat | EIYPNDGITDYADSVKG |
| 223 | SRP1848-F08 | CDR-H2 | Kabat | EIYPVDGITDYADSVKG |
| 224 | SRP1848-F09 | CDR-H2 | Kabat | GISPLDGYTDYADSVKG |
| 225 | SRP1848-F10 | CDR-H2 | Kabat | SIFPNDGYTDYADSVKG |
| 226 | SRP1848-F11 | CDR-H2 | Kabat | GITPIDGNTDYADSVKG |
| 227 | SRP1848-G01 | CDR-H2 | Kabat | WIAPNDGSTDYADSVKG |
| 228 | SRP1848-G03 | CDR-H2 | Kabat | GITPSDGFTDYADSVKG |
| 229 | SRP1848-G04 | CDR-H2 | Kabat | YITPAGGATFYADSVKG |
| 230 | SRP1848-G06 | CDR-H2 | Kabat | YITPAGGATYYADNVKG |
| 231 | SRP1848-G07 | CDR-H2 | Kabat | YITPAGGATWYADSVKG |
| 232 | SRP1848-G09 | CDR-H2 | Kabat | YITPAGGATFYADSVKG |
| 233 | SRP1848-G10 | CDR-H2 | Kabat | YITPAGGATYYADSVKG |
| 234 | SRP1848-G11 | CDR-H2 | Kabat | YITPAGGATWYADSVKG |
| 235 | SRP1848-H01 | CDR-H2 | Kabat | DIFPIDGITDYADSVKG |
| 236 | SRP2060-E10 | CDR-H2 | Kabat | HIWWDDDKYYHPALKG |
| 237 | SRP2060-E05 | CDR-H2 | Kabat | HIWWDDDKYYHPALKG |
| 238 | SRP2060-B01 | CDR-H2 | Kabat | HIWWDDDKYYHPALKG |
| 239 | SRP2060-A06 | CDR-H2 | Kabat | HIWWDDDKYYYPALKG |
| 240 | SRP1848-A01 | CDR-H3 | | HIYPWDWFSNYVLDY |
| 241 | SRP1848-A02 | CDR-H3 | | HLYVWDWVLDHVLDY |
| 242 | SRP1848-A04 | CDR-H3 | | GAWSWRSGYGYYIDY |
| 243 | SRP1848-A06 | CDR-H3 | | GAWSWRSGYGYYIDY |
| 244 | SRP1848-A07 | CDR-H3 | | GFWAWRSGYGYYLDY |
| 245 | SRP1848-A08 | CDR-H3 | | GSWFWRAGYGYYLDY |
| 246 | SRP1848-A09 | CDR-H3 | | GSWFWRSGYGYFLEY |
| 247 | SRP1848-A10 | CDR-H3 | | GSWSWPSGHSYYLDY |
| 248 | SRP1848-B01 | CDR-H3 | | GFWSWPSGYSYFLDY |
| 249 | SRP1848-B03 | CDR-H3 | | GSWSWPSGYSYYLDY |
| 250 | SRP1848-B04 | CDR-H3 | | GRYSWRAGYSYYLDY |
| 251 | SRP1848-B05 | CDR-H3 | | GSWFWQSGYGYYLDY |
| 252 | SRP1848-B06 | CDR-H3 | | GFWSWPSGYGYYQDY |
| 253 | SRP1848-B07 | CDR-H3 | | GSWSWPAGYGYYQDY |
| 254 | SRP1848-B09 | CDR-H3 | | GAWSWRSGYGYYMDY |
| 255 | SRP1848-B10 | CDR-H3 | | GGWHWRSGYSYYLDY |
| 256 | SRP1848-B11 | CDR-H3 | | GSWSWRAGYGYYLDY |
| 257 | SRP1848-C01 | CDR-H3 | | GSWFWRAGYGYYLDY |
| 258 | SRP1848-C03 | CDR-H3 | | GSWAWRSGYSYYLDY |
| 259 | SRP1848-C04 | CDR-H3 | | GSLSWRAGYGYYLDY |
| 260 | SRP1848-C05 | CDR-H3 | | GSWSWKAGYGYYLDY |
| 26 | SRP1848-C07 | CDR-H3 | | GSWSWPAGYGYYQDY |
| 262 | SRP1848-C10 | CDR-H3 | | GSWSWPAGYGYYLDY |
| 263 | SRP1848-D02 | CDR-H3 | | GAWSWRAGYGYYLDY |
| 264 | SRP1848-D03 | CDR-H3 | | GHWSWPSGYWYYLDY |
| 265 | SRP1848-D04 | CDR-H3 | | GYWFWRSGYGYYLDY |
| 266 | SRP1848-D05 | CDR-H3 | | GSWSWRAGYGYYLDY |
| 267 | SRP1848-D07 | CDR-H3 | | GFWAWRSGYGYYLDY |
| 268 | SRP1848-D09 | CDR-H3 | | GSWSWRHGYGYYLDY |
| 269 | SRP1848-D10 | CDR-H3 | | GAWSWRSGYGYYIDY |
| 270 | SRP1848-E01 | CDR-H3 | | GSWSWPAGYGYYQDY |
| 271 | SRP1848-E02 | CDR-H3 | | GSWSWRSGYGYYLDY |
| 272 | SRP1848-E03 | CDR-H3 | | GSWSWPAGHSYYLDY |
| 273 | SRP1848-E05 | CDR-H3 | | GFWAWRSGYGYYLDY |
| 274 | SRP1848-E06 | CDR-H3 | | GTWSWPSGHSYYLDY |
| 275 | SRP1848-E07 | CDR-H3 | | GAWSWRSGYGYYIDY |
| 276 | SRP1848-F01 | CDR-H3 | | GSWAWRAGYSYYLDY |
| 277 | SRP1848-F02 | CDR-H3 | | GSWSWQAGYGYYLDY |
| 278 | SRP1848-F04 | CDR-H3 | | GSWFWRSGYGYYLDY |
| 279 | SRP1848-F05 | CDR-H3 | | GSWAWRSGYSYFLDY |
| 280 | SRP1848-F06 | CDR-H3 | | GFWAWRSGYGYYLDY |
| 281 | SRP1848-F07 | CDR-H3 | | GSWDWRSGYSYYLDY |
| 282 | SRP1848-F08 | CDR-H3 | | GSWYWQSGYSYYLDY |
| 283 | SRP1848-F09 | CDR-H3 | | GAWSWRSGYGYYIDY |
| 284 | SRP1848-F10 | CDR-H3 | | GSWFWRSGYGYYLDY |
| 285 | SRP1848-F11 | CDR-H3 | | GSWYWRAGYGYYLDY |
| 286 | SRP1848-G01 | CDR-H3 | | GSWAWRSGYSYFLDY |
| 287 | SRP1848-G03 | CDR-H3 | | GSWSWPSGHGYFLDY |
| 288 | SRP1848-G04 | CDR-H3 | | YPYWFAGYMDY |
| 289 | SRP1848-G06 | CDR-H3 | | QPYWFAGYMDY |
| 290 | SRP1848-G07 | CDR-H3 | | YPFWFAGYMDY |
| 291 | SRP1848-G09 | CDR-H3 | | HEYWFSGYMDY |
| 292 | SRP1848-G10 | CDR-H3 | | YPYWFAGYIDY |
| 293 | SRP1848-G11 | CDR-H3 | | YPYWFSGYMDY |
| 294 | SRP1848-H01 | CDR-H3 | | GSWSWPSGMDYYLDY |
| 295 | SRP2060-E10 | CDR-H3 | | NHFPHYYGSSHWYFNV |
| 296 | SRP2060-E05 | CDR-H3 | | NHFPHYYGSSHWYFNV |
| 297 | SRP2060-B01 | CDR-H3 | | NHFPHYYGSSHWYFNV |
| 298 | SRP2060-A06 | CDR-H3 | | NHFPHYYGSSHWYFDV |
| 299 | trastuzumab | CDR-L1 | | RASQDVNTAVA |
| 300 | H6D1-LC4 | CDR-L1 | | KASQDINSYLS |
| 301 | H6D1-LC5 | CDR-L1 | | KASQDINSYLS |
| 302 | trastuzumab | CDR-L2 | | SASFLYS |
| 303 | H6D1-LC4 | CDR-L3 | | RANRLVD |
| 304 | H6D1-LC5 | CDR-L2 | | RANRLVD |
| 305 | trastuzumab | CDR-L3 | | QQHYTTPPT |
| 306 | H6D1-LC4 | CDR-L3 | | LQYDEFPYT |
| 307 | H6D1-LC5 | CDR-L3 | | LQYDEFPYT |
| 308 | SRP1848-A01 | VH | | EVQLVESGGGLVQPGGSLRLSCAASGEN |
| | | | | ITRYSIHWVRQAPGKGLEWVAGILPESG |
| | | | | GTSYADSVKGRFTISADTSKNTAYLQMN |
| | | | | SLRAEDTAVYYCARHIYPWDWFSNYVLD |
| | | | | YWGQGTLVTVSS |
| 309 | SRP1848-A02 | VH | | EVQLVESGGGLVQPGGSLRLSCAASGEN |
| | | | | ISGFRIHWVRQAPGKGLEWVAGIYPESG |
| | | | | ATYYADSVKGRFTISADTSKNTAYLQMN |
| | | | | SLRAEDTAVYYCARHLYVWDWVLDHVLD |
| | | | | YWGQGTLVTVSS |
| 310 | SRP1848-A04 | VH | | EVQLVESGGGLVQPGGSLRLSCAASGEN |
| | | | | IDQSSIHWVRQAPGKGLEWVGVIYPVDG |
| | | | | TTDYADSVKGRFTISADTSKNTAYLQMN |
| | | | | SLRAEDTAVYYCARGAWSWRSGYGYYID |
| | | | | YWGQGTLVTVSS |
| 311 | SRP1848-A06 | VH | | EVQLVESGGGLVQPGGSLRLSCAASGEN |
| | | | | IGNSYIHWVRQAPGKGLEWVGGITPIDG |
| | | | | NTDYADSVKGRFTISADTSKNTAYLQMN |
| | | | | SLRAEDTAVYYCARGAWSWRSGYGYYID |
| | | | | YWGQGTLVTVSS |
| 312 | SRP1848-A07 | VH | | EVQLVESGGGLVQPGGSLRLSCAASGEN |
| | | | | IGYHSIHWVRQAPGKGLEWVGEIFPVDG |
| | | | | TTDYADSVKGRFTISADTSKNTAYLHMN |
| | | | | SLRAEDTAVYYCARGFWAWRSGYGYYLD |
| | | | | YWGQGTLVTVSS |
| 313 | SRP1848-A08 | VH | | EVQLVESGGGLVQPGGSLRLSCAASGSN |
| | | | | IRKHSIHWVRQAPGKGLEWVGSIYPNDG |
| | | | | TTDYADSVKGRFTISADTSKNTAYLQMN |
| | | | | SLRAEDTAVYYCARGSWFWRAGYGYYLD |
| | | | | YWGQGTLVTVSS |
| 314 | SRP1848-A09 | VH | | EVQLVESGGGLVQPGGSLRLSCAASGEN |
| | | | | IRKQSIHWVRQAPGKGLEWVGSIFPNDG |
| | | | | TTDYADSVKGRFTISADTSKNTAYLQVN |
| | | | | SLRAEDTAVYYCARGSWFWRSGYGYFLE |
| | | | | YWGQGTLVTVSS |
| 315 | SRP1848-A10 | VH | | EVQLVESGGGLVQPGGSLRLSCAASGEN |
| | | | | IRKYSIHWARQAPGKGLEWVGDIFPIDD |
| | | | | ITDYADSVKGRFTISADTSKNTAYLQMN |
| | | | | SLRAEDTAVYYCARGSWSWPSGHSYYLD |
| | | | | YWGQGTLVTVSS |
| 316 | SRP1848-B01 | VH | | EVQLVESGGGLVQPGGSLRLSCAASGEN |
| | | | | IRNYSIHWVRQAPGKGLEWVGEIYPVDG |
| | | | | ITDYADSVKGRFTISADTSKNTAYLQMN |
| | | | | SLRAEDTAVYYCARGFWSWPSGYSYFLD |
| | | | | YWGQGTLVTVSS |
| 317 | SRP1848-B03 | VH | | EVQLVESGGGLVQPGGSLRLSCAASGEN |
| | | | | ISMKYIHWVRQAPGKGLEWVGGITPIDG |
| | | | | MTDYADSVKGRFTISADTSKNTAYLQMN |
| | | | | SLRAEDTAVYYCARGSWSWPSGYSYYLD |
| | | | | YWGQGTLVTVSS |
| 318 | SRP1848-B04 | VH | | EVQLVESGGGLVQPGGSLRLSCAASSEN |
| | | | | ISNHSIHWVRQAPGKGLEWVGEIYPVDG |
| | | | | ITDYADSVKGRFTISADTSKNTAYLQMN |
| | | | | SLRAEDTAVYYCARGRYSWRAGYSYYLD |
| | | | | YWGQGTLVTVSS |
| 319 | SRP1848-B05 | VH | | EVQLVESGGGLVQPGGSLRLSCAASGEN |
| | | | | ISNYYIHWVRQAPGKGLEWVGGISPIDG |
| | | | | YTDYADSMKGRFTISADTSKNTAYLQMS |
| | | | | SLRAEDTAVYYCARGSWFWQSGYGYYLD |
| | | | | YWGQGTLVTVSS |
| 320 | SRP1848-B06 | VH | | EVQLVESGGGLVQPGGSLRLSCAASGEN |
| | | | | ISNYYIHWVRQAPGKGLEWVGGITPIDG |
| | | | | YTDYADSVKGRFTISADTSKNTAYLQMN |
| | | | | SLRAEDTAVYYCARGFWSWPSGYGYYQD |
| | | | | YWGQGTLVTVSS |
| 321 | SRP1848-B07 | VH | | EVQLVESGGGLVQPGGSLRLSCAASGEN |
| | | | | ISRFYIHWVRQAPGKGLEWVGGISPYDG |
| | | | | FTDYADSVKGRFTISADTSKNTAYLQMN |
| | | | | SLRAEDTAVYYCARGSWSWPAGYGYYQD |
| | | | | YWGQGTLVTVSS |
| 322 | SRP1848-B09 | VH | | EVQLVESGGGLVQPGGSLRLSCAAGGEN |
| | | | | ITNYYIHWVRQAPGKGLEWVGGITPVDG |
| | | | | YTDYADRVKGRFTISADTSKNTAYLQMN |
| | | | | SLRAEDTAVYYCARGAWSWRSGYGYYMD |
| | | | | YWGQGTLVTVSS |
| 323 | SRP1848-B10 | VH | | EVQLVESGGGLVQPGGSLRLSCAASGEN |
| | | | | TTTKSIHWVRQAPGKGLEWVGEIYPRDG |
| | | | | ITDYADSVKGRFTISADTSKNTAYLQMN |
| | | | | SLRAEDTAVYYCARGGWHWRSGYSYYLD |
| | | | | YWGQGTLVTVSS |
| 324 | SRP1848-B11 | VH | | EVQLVESGGGLVQPGGSLRLSCAASGEN |
| | | | | IGNNSIHWVRQAPGKGLEWVGDISPIDG |
| | | | | FTDYADSVKGRFTISADTSKNTAYLQMN |
| | | | | SLRAEDTAVYYCARGSWSWRAGYGYYLD |
| | | | | YWGQGTLVTVSS |
| 325 | SRP1848-C01 | VH | | EVQLVESGGGLVQPGGSLRLSCAASGEN |
| | | | | IGNSYIHWVRQAPGKGLEWVGGVTPNDG |
| | | | | YTDYADSVKGRFTISADTSKNTTYLQMN |
| | | | | SLRAEDTAVYYCARGSWFWRAGYGYYLD |
| | | | | YWGQGALVTVSS |
| 326 | SRP1848-C03 | VH | | EVQLVESGGGLVQPGGSLRLSCAASGEN |
| | | | | IGVYSIHWVRQAPGKGLEWVGEIYPIDG |
| | | | | NTDYADSVKGRFTISADTSKNTAYLQMN |
| | | | | SLRAEDTAVYYCARGSWAWRSGYSYYLD |
| | | | | YWGQGTLVTVSS |
| 327 | SRP1848-C04 | VH | | EVQLVESGGGLVQPGGSLRLSCAASGEN |
| | | | | IRHYSIHWVRQAPGKGLEWVGEIYPGPG |
| | | | | NTDYADSVKGRFTISADTSKNTAYLQMN |
| | | | | SLRAEDTAVYYCARGSLSWRAGYGYYLD |
| | | | | YWGQGTLVTVSS |
| 328 | SRP1848-C05 | VH | | EVQLVESGGGLVQPGGSLRLSCAASGEN |
| | | | | IRKYSIHWVRQAPGKGLEWVGDIFPIDG |
| | | | | INDYADSVKGRFTISADTSKNTAYLQMN |
| | | | | SLRAEDTAVYYCARGSWSWKAGYGYYLD |
| | | | | YWGQGTLVTVSS |
| 329 | SRP1848-C07 | VH | | EVQLVESGGGLVQPGGSLRLSCAASGEN |
| | | | | IRKYSIHWVRQAPGKGLEWVGDIFPIDG |
| | | | | ITDYADSMKGRFTISADTSKNTAYLQMN |
| | | | | SLRAEDTAVYYCARGSWSWPAGYGYYQD |
| | | | | YWGQGTLVTVSS |
| 330 | SRP1848-C10 | VH | | EVQLVESGGGLVQPGGSLRLSCAASGEN |
| | | | | IRTYYIHWVRQAPGKGLEWVGGISPIDG |
| | | | | YTDYADSVKGRFTISADTSKNTAYLQMN |
| | | | | SLRAEDTAVYYCARGSWSWPAGYGYYLD |
| | | | | YWGQGTLVTVSS |
| 331 | SRP1848-D02 | VH | | EVQLVESGGGLVQPGGSLRLSCAASGEN |
| | | | | ISHNYIHWVRQAPGKGLEWVGGITPQDG |
| | | | | YTDYADSVKGRFTISADTSKNTAYLQMN |
| | | | | RLRAEDTAVYYCARGAWSWRAGYGYYLD |
| | | | | YWGQGTLVTVSS |
| 332 | SRP1848-D03 | VH | | EVQLVESGGGVVQPGGSLRLSCAASGEN |
| | | | | IRYFSIHWVRQAPGKGLEWVGDIFPNDG |
| | | | | STDYADSVKGRFTISADTSKNTAYLQMN |
| | | | | SLRAEETAVYYCARGHWSWPSGYWYYLD |
| | | | | YWGQGTLVTVSS |
| 333 | SRP1848-D04 | VH | | EVQLVESGGGLVQPGGSLRLSCAASGEN |
| | | | | ISHYSIHWVRQAPGKGLEWVGEIYPRDG |
| | | | | ITDYADSVKGRFTISADTSKNTAYLQMN |
| | | | | SLSAEDTAVYYCARGYWFWRSGYGYYLD |
| | | | | YWGQGTLVTVSS |
| 334 | SRP1848-D05 | VH | | EVQLVESGGGLVQPGGSLRLSCAASGEN |
| | | | | ISISYIHWVRQAPGKGLEWVGGISPIDG |
| | | | | YTDYADSVKGRFTISADTSKNTAYLQMN |
| | | | | SLRAEDTAVYYCARGSWSWRAGYGYYLD |
| | | | | YWGQGTLVTVSS |
| 335 | SRP1848-D07 | VH | | EVQLVESGGGLVQPGGSLRLSCAASGEN |
| | | | | ISKYYIHWVRQAPGKGLEWVGGISPNDG |
| | | | | YTDYADSVKGRFAISADTSKNTAYLQMN |
| | | | | SLRAEDTAVYYCARGFWAWRSGYGYYLD |
| | | | | YWGQGTLVTVSS |
| 336 | SRP1848-D09 | VH | | EVQLVESGGGLVQPGGSLRLSCAASGEN |
| | | | | ISNYYIHWVRQAPGKGLEWVGGISPNDG |
| | | | | YTDYADSVKGRFTISADTSKNTAYLQMN |
| | | | | SLRAEDTAVYYCARGSWSWRHGYGYYLD |
| | | | | YWGQGTLVTVSS |
| 337 | SRP1848-D10 | VH | | EVQLVESGGGLVQPGGSLRLSCAASGEN |
| | | | | ISRNSIHWVRQAPGKGLEWVGWISPNDG |
| | | | | TTDYADSVKGRFTISADGSKNTAYLQMN |
| | | | | SLRAEDTAVYYCARGAWSWRSGYGYYID |
| | | | | YWGQGTLVTVSS |
| 338 | SRP1848-E01 | VH | | EVQLVESGGGLVQPGGSLRLSCAASGEN |
| | | | | ITNKYIHWVRQAPGKGLEWVGGITPFDG |
| | | | | FTDYADSVKGRFTISADTSKNTAYLQMN |
| | | | | SLRAEDTAVYYCARGSWSWPAGYGYYQD |
| | | | | YWGQGTLVTVSS |
| 339 | SRP1848-E02 | VH | | EVQLVESGGGLVQPGGSLRLSCAASGEN |
| | | | | IGKYSIHWVRQAPGKGLEWVGEIYPNDG |
| | | | | NTDYADSVKGRFTISADTSKNTAYLQMN |
| | | | | SLRAEDTAVYYCARGSWSWRSGYGYYLD |
| | | | | YWGQGTLVTVSS |
| 340 | SRP1848-E03 | VH | | EVQLVESGGGLAQPGGSLRLSCAASGEN |
| | | | | IGNYYIHWVRQAPGKGLEWVGGITPRDG |
| | | | | FTDYADSVKGRFTISADTSKNTAYLQVN |
| | | | | SLRAEDTAVYYCARGSWSWPAGHSYYLD |
| | | | | YWGQGTLVTVSS |
| 341 | SRP1848-E05 | VH | | EVQLVESGGGLVQPGGSLRVSCAASGEN |
| | | | | IGVYYIHWVRQAPGKGLEWVGGITPNDG |
| | | | | YTDYADSVKGRFTISADTSKNTAYLQMN |
| | | | | SLRAEDTAVYYCARGFWAWRSGYGYYLD |
| | | | | YWGQGTLVTVSS |
| 342 | SRP1848-E06 | VH | | EVQLVESGGGLVQPSGSLRLSCAASGEN |
| | | | | INRYYIHWVRQAPGKGLEWVGGITPNDG |
| | | | | YTDYADSVEGRETTSADTSKNTAYLQMN |
| | | | | SLRAEDTAVYYCARGTWSWPSGHSYYLD |
| | | | | YWGQGTLVTVSS |
| 343 | SRP1848-E07 | VH | | EVQLVESGGGLVQPGGSLRLSCAASGEN |
| | | | | IRKSSIHWVRQAPGKGLEWVGEIFPYDG |
| | | | | STDYADNVKGRFTISADTSKNTAYLQMN |
| | | | | SLRAEDTAVYYCARGAWSWRSGYGYYID |
| | | | | YWGQGTLVTVSS |
| 344 | SRP1848-F01 | VH | | EVQLVESGGGLVQPGGSLRLSCAASGEN |
| | | | | IRTYSIHWVRQAPGKGLEWVGSIFPNDG |
| | | | | TTDYADSVKGRFTISADTSKNTAYLQMN |
| | | | | SLRAEDTAVYYCARGSWAWRAGYSYYLD |
| | | | | YWGQGTLVTVSS |
| 345 | SRP1848-F02 | VH | | EVQLVESGGGLVQPGGSLRLSCAASGEN |
| | | | | IRTYSIHWVRQAPGKGLEWVGSIFPNDG |
| | | | | TTDYADSVKGRLTISADTSKNTAYLQMN |
| | | | | SLRAEDTAVYYCARGSWSWQAGYGYYLD |
| | | | | YWGQGTLVTVSS |
| 346 | SRP1848-F04 | VH | | EVQLVESGGGLVQPGGSLRLSCAASGEN |
| | | | | ISNYSIHWVRQAPGKGLEWVGEIYPIDG |
| | | | | ITDYADSVKGRFTISADTSKNTAYLQMN |
| | | | | SLRAEDTAVYYCARGSWFWRSGYGYYLD |
| | | | | YWGQGTLVTVSS |
| 347 | SRP1848-F05 | VH | | EVQLVESGGGLVQPGGSLRLSCAASGEN |
| | | | | ISKSSIHWVRQAPGKGLEWVGEIYPNDG |
| | | | | STDYADSVKGRFTISADTSKNTAYLQMN |
| | | | | SLRAEDTAVYYCARGSWAWRSGYSYFLD |
| | | | | YWGQGTLVTVSS |
| 348 | SRP1848-F06 | VH | | EVQLVESGGGLVQPGGSLRLSCAASGEN |
| | | | | ISLSYIHWVRQAPGKGLEWVGGISPIDG |
| | | | | NTDYADSVKGRFTISADTSKNTAYLQMN |
| | | | | SLRAEDTAVYYCARGFWAWRSGYGYYLD |
| | | | | YWGQGTLVTVSS |
| 349 | SRP1848-F07 | VH | | EVQLVESGGGLVQPGGSLRLSCAASGEN |
| | | | | ISNHSIHWVRQAPGKGLEWVGEIYPNDG |
| | | | | ITDYADSVKGRFTISADTSKNTAYLQMN |
| | | | | SLSAEDTAVYYCARGSWDWRSGYSYYLD |
| | | | | YWGQGTLVTVSS |
| 350 | SRP1848-F08 | VH | | EVQLVESGGGLVQPGGSLRLSCAAGGEN |
| | | | | ISNHSIHWVRQAPGKGVEWVGEIYPVDG |
| | | | | ITDYADSVKGRFTISADTSKNTAYLRMN |
| | | | | SLRAEDTAVYYCARGSWYWQSGYSYYLD |
| | | | | YWGQGTLVTVSS |
| 351 | SRP1848-F09 | VH | | EVQLVESGGGLVQPGGSLRLSCAASGEN |
| | | | | ISNHYIHWVRQAPGKGLEWVGGISPLDG |
| | | | | YTDYADSVKGRFTISADTSKNTAYLQMN |
| | | | | SLRAEDTAVYYCARGAWSWRSGYGYYID |
| | | | | YWGQGTLVTVSS |
| 352 | SRP1848-F10 | VH | | EVQLVESGGGLVQPGGSLRLSCAASGEN |
| | | | | ISNNSIHWVRQAPGKGLEWVGSIFPNDG |
| | | | | YTDYADSVKGRFTISADTSKNTAYLQMN |
| | | | | SLRAEDTAVYYCARGSWFWRSGYGYYLD |
| | | | | YWGQGTLVTVSS |
| 353 | SRP1848-F11 | VH | | EVQLVESGGGLVQPGGSLRLSCAASGEN |
| | | | | ISNNYIHWVRQAPGKGLEWVGGITPIDG |
| | | | | NTDYADSVKGRFTISADTSMNTAYLQMN |
| | | | | SLRAEDTAVYYCARGSWYWRAGYGYYLD |
| | | | | YWGQGALVTVSS |
| 354 | SRP1848-G01 | VH | | EVQLVESGGGLVQPGGSLRLSCAASGEN |
| | | | | ISRHSIHWVRQAPGKGLEWVGWIAPNDG |
| | | | | STDYADSVKGRFTISADTSKNTAYLQMN |
| | | | | SLRAEDTAVYYCARGSWAWRSGYSYFLD |
| | | | | YWGQGTLVTVSS |
| 355 | SRP1848-G03 | VH | | EVQLVESGGGLVQPGGSLRLSCAASGEN |
| | | | | ISTYYIHWVRQAPGKGLEWVGGITPSDG |
| | | | | FTDYADSVKGRSTISADTSKNTAYLQMN |
| | | | | SLRAEDTAVYYCARGSWSWPSGHGYFLD |
| | | | | YWGQGTLVTVSS |
| 356 | SRP1848-G04 | VH | | EVQLVESGGGLVQPGGSLRLSCAASGEN |
| | | | | IHSTDIHWVRQAPGKGLEWVAYITPAGG |
| | | | | ATFYADSVKGRFTISADTSKNTAYLQMN |
| | | | | SLRAEDTAVYYCARYPYWFAGYMDYWGQ |
| | | | | GTLVTVSS |
| 357 | SRP1848-G06 | VH | | EVQLVESGGGLVQPGGSLRLSCAASGEN |
| | | | | IRSTDIHWVRQAPGKGLEWVAYITPAGG |
| | | | | ATYYADNVKGRFTISADTSKNTAYLQMN |
| | | | | SLRAEDTAVYYCARQPYWFAGYMDYWGQ |
| | | | | GTLVTVSS |
| 358 | SRP1848-G07 | VH | | EVQLVESGGGLVQPGGSLRLSCAASGEN |
| | | | | IHSTDIHWVRQAPGKGLEWVAYITPAGG |
| | | | | ATWYADSVKGRFTISADTSKNTAYLQMN |
| | | | | SLRAEDTAVYYCARYPFWFAGYMDYWGQ |
| | | | | GTLVTVSS |
| 359 | SRP1848-G09 | VH | | EVQLVESGGGLVQPGGSLRLSCAASGEN |
| | | | | IRGTDIHWVRQAPGKGLEWVAYITPAGG |
| | | | | ATFYADSVKGRFTISADTSKNTAYLQMN |
| | | | | SLRAEDTAVYYCARHEYWFSGYMDYWGQ |
| | | | | GTLVTVSS |
| 360 | SRP1848-G10 | VH | | EVQLVESGGGLVQPGSSLRLSCAASGEN |
| | | | | IRSTDIHWVRQAPGKGLEWVAYITPAGG |
| | | | | ATYYADSVKGRFTISADTSKNTAYLQMN |
| | | | | SLRAEDTAVYYCARYPYWFAGYIDYWGQ |
| | | | | GTLVTVSS |
| 361 | SRP1848-G11 | VH | | EVQLVESGGGLVQPGGSLRLSCAASGFN |
| | | | | ISSTDIHWVRQAPGKGLEWVAYITPAGG |
| | | | | ATWYADSVKGRFTISADTSKNTAYLQMN |
| | | | | SLRAEDTAVYYCARYPYWFSGYMDYWGQ |
| | | | | GTLVTVSS |
| 362 | SRP1848-H01 | VH | | EVQLVESGGGLVQPGGSLRLSCAASGEN |
| | | | | IRTQSIHWVRQAPGKGLEWIGDIFPIDG |
| | | | | ITDYADSVKGRFTISADTSKNTAYLQMN |
| | | | | SLRAEDTAVYYCARGSWSWPSGMDYYLD |
| | | | | YWGQGTLVTVSS |
| 363 | SRP2060-E10 | VH | | EVQLLESGGGLVQPGGSLRLSCAFSGES |
| | | | | LSTFGMGVGWVRQAPGKGLEWVSHIWWD |
| | | | | DDKYYHPALKGRFTISKDNSKNTVYLQM |
| | | | | NSLRAEDTAVYYCGRNHFPHYYGSSHWY |
| | | | | FNVWGQGTTVTVSS |
| 364 | SRP2060-E05 | VH | | EVQLLESGGGLVQPGGSLRLSCAFSGFS |
| | | | | LSTFGMGVGWVRQAPGKGLEWVSHIWWD |
| | | | | DDKYYHPALKGRFTVSKDNSKNTVYLQM |
| | | | | NSLRAEDTAVYYCGRNHFPHYYGSSHWY |
| | | | | FNVWGQGTTVTVSS |
| 365 | SRP2060-B01 | VH | | EVQLLESGGGLVQPGGSLRLSCALSGFS |
| | | | | LSTFGMGVGWVRQATGKGLEWVSHIWWD |
| | | | | DDKYYHPALKGRFTISKDNSKNTVHLQM |
| | | | | NSLRAEDTAVYYCGRNHFPHYYGSSHWY |
| | | | | FNVWGQGTTVTVSS |
| 366 | SRP2060-A06 | VH | | EVQLLESGGGLVQPGGSLRLSCAFSGFS |
| | | | | LSTFGMGVGWVRQAPGKGLEWVGHIWWD |
| | | | | DDKYYYPALKGRFTISKDNSKNTVYLQM |
| | | | | NSLRAEDTAVYYCGRNHFPHYYGSSHWY |
| | | | | FDVWGQGTTVTVSS |
| 367 | trastuzumab | VL | | DIQMTQSPSSLSASVGDRVTITCRASQD |
| | | | | VNTAVAWYQQKPGKAPKLLIYSASFLYS |
| | | | | GVPSRFSGSRSGTDFTLTISSLQPEDFA |
| | | | | TYYCQQHYTTPPTFGQGTKVEIK |
| 368 | H6D1-LC4 | VL | | EIVMTQSPATLSLSPGERATLSCKASQD |
| | | | | INSYLSWYQQKPGQAPRLLIYRANRLVD |
| | | | | GIPARFSGSGSGTDYTLTISSLEPEDFA |
| | | | | VYYCLQYDEFPYTFGGGTKVEIK |
| 369 | H6D1-LC5 | VL | | DIQMTQSPSTLSASVGDRVTITCKASQD |
| | | | | INSYLSWYQQKPGKAPKLLIYRANRLVD |
| | | | | GVPSRFSGSGSGTEFTLTISSLQPDDFA |
| | | | | TYYCLQYDEFPYTFGGGTKVEIK |
| 370 | Human IgG1 HC | | | ASTKGPSVFPLAPSSKSTSGGTAALGCL |
| | Constant | | | VKDYFPEPVTVSWNSGALTSGVHTFPAV |
| | | | | LQSSGLYSLSSVVTVPSSSLGTQTYICN |
| | | | | VNHKPSNTKVDKKVEPKSCDKTHTCPPC |
| | | | | PAPELLGGPSVFLFPPKPKDTLMISRTP |
| | | | | EVTCVVVDVSHEDPEVKFNWYVDGVEVH |
| | | | | NAKTKPREEQYNSTYRVVSVLTVLHQDW |
| | | | | LNGKEYKCKVSNKALPAPIEKTISKAKG |
| | | | | QPREPQVYTLPPSREEMTKNQVSLTCLV |
| | | | | KGFYPSDIAVEWESNGQPENNYKTTPPV |
| | | | | LDSDGSFFLYSKLTVDKSRWQQGNVFSC |
| | | | | SVMHEALHNHYTQKSLSLSPGK |
| 371 | Human IgG LC | | | RTVAAPSVFIFPPSDEQLKSGTASVVCL |
| | Constant Ckappa | | | LNNFYPREAKVQWKVDNALQSGNSQESV |
| | | | | TEQDSKDSTYSLSSTLTLSKADYEKHKV |
| | | | | YACEVTHQGLSSPVTKSENRGEC |
| 372 | Mouse IgG1 HC | | | AKTTPPSVYPLAPGSAAQTNSMVTLGCL |
| | Constant | | | VKGYFPEPVTVTWNSGSLSSGVHTFPAV |
| | | | | LQSDLYTLSSSVTVPSSTWPSETVTCNV |
| | | | | AHPASSTKVDKKIVPRDCGCKPCICTVP |
| | | | | EVSSVFIFPPKPKDVLTITLTPKVTCVV |
| | | | | VDISKDDPEVQFSWFVDDVEVHTAQTQP |
| | | | | REEQFNSTFRSVSELPIMHQDWLNGKEF |
| | | | | KCRVNSAAFPAPIEKTISKTKGRPKAPQ |
| | | | | VYTIPPPKEQMAKDKVSLTCMITDFFPE |
| | | | | DITVEWQWNGQPAENYKNTQPIMDTDGS |
| | | | | YFVYSKLNVQKSNWEAGNTFTCSVLHEG |
| | | | | LHNHHTEKSLSHSPG |
| 373 | Mouse IgG LC | | | RADAAPTVSIFPPSSEQLTSGGASVVCF |
| | Constant Ckappa | | | LNNFYPKDINVKWKIDGSERQNGVLNSW |
| | | | | TDQDSKDSTYSMSSTLTLTKDEYERHNS |
| | | | | YTCEATHKTSTSPIVKSENRNEC |
| 374 | Kappa LC | | | HMTVAAPSVFIFPPSDEQLKSGTASVVC |
| | | | | LLNNFYPREAKVQWKVDNALQSGNSQES |
| | | | | VTEQDSKDSTYSLSSTLTLSKADYEKHK |
| | | | | VYACEVTHQGLSSPVTKSENRGEC |
| 375 | Lambda LD | | | GQPKAAPSVTLFPPSSEELQANKATLVC |
| | | | | LISDFYPGAVTVAWKADSSPVKAGVETT |
| | | | | TPSKQSNNKYAASSYLSLTPEQWKSHRS |
| | | | | YSCQVTHEGSTVEKTVAPTECS |
| 376 | FlagHis Tag | | | GSGDYKDDDDKGSGHHHHHH |
| 377 | Linker | | | GGGGSGGGGSGGGGS |
| 378 | Linker | | | AAGSDQEPKSS |
| 379 | 1848-B10-VH- | scFv | | MEVQLVESGGGLVQPGGSLRLSCAASGF |
| | (G4S)3-VL | | | NTTTKSIHWVRQAPGKGLEWVGEIYPRD |
| | | | | GITDYADSVKGRFTISADTSKNTAYLQM |
| | | | | NSLRAEDTAVYYCARGGWHWRSGYSYYL |
| | | | | DYWGQGTLVTVSSGGGGSGGGGSGGGGS |
| | | | | DIQMTQSPSSLSASVGDRVTITCRASQD |
| | | | | VNTAVAWYQQKPGKAPKLLIYSASFLYS |
| | | | | GVPSRFSGSRSGTDFTLTISSLQPEDFA |
| | | | | TYYCQQHYTTPPTFGQGTKVEIK |
| 380 | 1848-B10-VL- | scFv | | MDIQMTQSPSSLSASVGDRVTITCRASQ |
| | (G4S)3-VH | | | DVNTAVAWYQQKPGKAPKLLIYSASFLY |
| | | | | SGVPSRFSGSRSGTDFTLTISSLQPEDF |
| | | | | ATYYCQQHYTTPPTFGQGTKVEIKGGGG |
| | | | | SGGGGSGGGGSEVQLVESGGGLVQPGGS |
| | | | | LRLSCAASGFNTTTKSIHWVRQAPGKGL |
| | | | | EWVGEIYPRDGITDYADSVKGRFTISAD |
| | | | | TSKNTAYLQMNSLRAEDTAVYYCARGGW |
| | | | | HWRSGYSYYLDYWGQGTLVTVSS |
| 381 | 1848-B10-VH- | scFv-Fc | | MEVQLVESGGGLVQPGGSLRLSCAASGF |
| | (G4S)3-VL | | | NTTTKSIHWVRQAPGKGLEWVGEIYPRD |
| | | | | GITDYADSVKGRFTISADTSKNTAYLQM |
| | | | | NSLRAEDTAVYYCARGGWHWRSGYSYYL |
| | | | | DYWGQGTLVTVSSGGGGSGGGGSGGGGS |
| | | | | DIQMTQSPSSLSASVGDRVTITCRASQD |
| | | | | VNTAVAWYQQKPGKAPKLLIYSASFLYS |
| | | | | GVPSRFSGSRSGTDFTLTISSLQPEDFA |
| | | | | TYYCQQHYTTPPTFGQGTKVEIKAAGSD |
| | | | | QEPKSSDKTHTCPPCPAPELLGGPSVEL |
| | | | | FPPKPKDTLMISRTPEVTCVVVDVSHED |
| | | | | PEVKFNWYVDGVEVHNAKTKPREEQYNS |
| | | | | TYRVVSVLTVLHQDWLNGKEYKCKVSNK |
| | | | | ALPAPIEKTISKAKGQPREPQVYTLPPS |
| | | | | REEMTKNQVSLTCLVKGFYPSDIAVEWE |
| | | | | SNGQPENNYKTTPPVLDSDGSFFLYSKL |
| | | | | TVDKSRWQQGNVFSCSVMHEALHNHYTQ |
| | | | | KSLSLSPGK |
| 382 | 1848-B10-VL- | scFv-Fc | | MDIQMTQSPSSLSASVGDRVTITCRASQ |
| | (G4S)3-VH | | | DVNTAVAWYQQKPGKAPKLLIYSASFLY |
| | | | | SGVPSRFSGSRSGTDFTLTISSLQPEDF |
| | | | | ATYYCQQHYTTPPTFGQGTKVEIKGGGG |
| | | | | SGGGGSGGGGSEVQLVESGGGLVQPGGS |
| | | | | LRLSCAASGENTTTKSIHWVRQAPGKGL |
| | | | | EWVGEIYPRDGITDYADSVKGRFTISAD |
| | | | | TSKNTAYLQMNSLRAEDTAVYYCARGGW |
| | | | | HWRSGYSYYLDYWGQGTLVTVSSAAGSD |
| | | | | QEPKSSDKTHTCPPCPAPELLGGPSVEL |
| | | | | FPPKPKDTLMISRTPEVTCVVVDVSHED |
| | | | | PEVKFNWYVDGVEVHNAKTKPREEQYNS |
| | | | | TYRVVSVLTVLHQDWLNGKEYKCKVSNK |
| | | | | ALPAPIEKTISKAKGQPREPQVYTLPPS |
| | | | | REEMTKNQVSLTCLVKGFYPSDIAVEWE |
| | | | | SNGQPENNYKTTPPVLDSDGSFFLYSKL |
| | | | | TVDKSRWQQGNVFSCSVMHEALHNHYTQ |
| | | | | KSLSLSPGK |

### EQUIVALENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject mailer of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

One or more features from any embodiments described herein or in the figures may be combined with one or more features of any other embodiments described herein or in the figures without departing from the scope of the invention.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of treating or preventing cancer in a subject in need thereof, comprising administering to the subject:
(a) an antibody conjugate comprising an antibody that specifically binds to folate receptor alpha (FOLR1) linked site-specifically to at least one payload moiety, at a first dose one to three times; and
(b) then the antibody conjugate at a second dose at least one to three times, wherein the second dose is lower than the first dose.

2. A method of treating cancer in a subject in need thereof, comprising:
administering to the subject a first dose of a folate receptor alpha (FOLR1) antibody conjugate, wherein the FOLR1 antibody conjugate comprises an antibody linked to at least one payload moiety, and wherein the first dose is about 5.2 mg/kg or 4.3 mg/kg; and
administering to the subject a second dose of the FOLR1 antibody conjugate,
wherein the second dose is administered after the first dose, wherein the amount of the second dose is less than the amount of the first dose, and wherein the first dose and second dose are administered at least about three weeks apart.

3. A method of treating cancer in a subject in need thereof, comprising:
administering to the subject a first dose of a folate receptor alpha (FOLR1) antibody conjugate, wherein the FOLR1 antibody conjugate comprises an antibody linked to at least one payload moiety; and
administering to the subject a second dose of the FOLR1 antibody conjugate,
wherein the second dose is about 4.3 mg/kg or less, wherein the second dose is administered after the first dose, wherein the amount of the first dose is greater than the amount of the second dose, and wherein the first dose is administered once every three weeks or longer for one to six cycles.

4. A method of treating cancer in a subject in need thereof, comprising:
administering to the subject a first dose of a folate receptor alpha (FOLR1) antibody conjugate, wherein the FOLR1 antibody conjugate comprises an antibody linked to at least one payload moiety; and
administering to the subject a second dose of the FOLR1 antibody conjugate,
wherein the second dose is administered after the first dose, wherein the amount of the first dose is the same or greater than the amount of the second dose, wherein a tumor tissue sample from the subject has a tumor proportion score (TPS) of about 25% or more, and wherein the subject has a tumor reduction of at least 30% by volume after three weeks from administering the first dose.

5. The method of any one of the previous claims, wherein the first dose is about 5.2 mg/kg.

6. The method of any one of the previous claims, wherein the second dose is selected from about 4.3 mg/kg or less, about 3.5 mg/kg or less, and about 2.9 mg/kg or less.

7. The method of any one of the previous claims, wherein the second dose is about 4.3 mg/kg, about 3.5 mg/kg, or about 2.9 mg/kg.

8. The method of any one of the previous claims, wherein the first dose is about 5.2 mg/kg and the second dose is about 4.3 mg/kg; or wherein the first dose is about 5.2 mg/kg and the second dose is about 3.5 mg/kg; or wherein the first dose is about 5.2 mg/kg and the second dose is about 2.9 mg/kg; or wherein the first dose is about 4.3 mg/kg and the second dose is about 3.5 mg/kg; or wherein the first dose is about 4.3 mg/kg and the second dose is about 2.9 mg/kg.

9. The method of any one of the previous claims, wherein the first dose is administered once every three weeks or longer for one to five cycles.

10. The method of claim 9, wherein the first dose is administered for one to three cycles.

11. The method of claim 9, wherein the first dose is administered for two to four cycles.

12. The method of any one of the previous claims, wherein the second dose is administered at least about three weeks after the first dose is administered.

13. The method of any one of the previous claims, wherein the second dose is administered at least about six weeks after the first dose is administered.

14. The method of any one of the previous claims, wherein the second dose is administered at least about six weeks after the first dose is administered.

15. The method of any one of the previous claims, wherein the second dose is administered once every three weeks.

16. The method of any one of the previous claims, wherein the second dose is administered once every three weeks for at least about 3 weeks.

17. The method of any one of claims 1-15, wherein the second dose is administered once every three weeks for at least about 2 months.

18. The method of any one of claims 1-15, wherein the second dose is administered once every three weeks for at least about 3 months.

19. The method of any one of claims 1-15, wherein the second dose is administered once every three weeks for at least about 4 months.

20. The method of any one of claims 1-15, wherein the second dose is administered once every three weeks for the remainder of the treating.

21. The method of any one of claims 1-4, wherein the first dose and the second dose are administered at least about three weeks apart.

22. The method of any one of the previous claims, further comprising administering a third dose of the FOLR1 antibody conjugate.

23. The method of claim 22, wherein the amount of the third dose is less than the amount of the second dose.

24. The method of claim 22 or 23, wherein the amount of the third dose is about 3.5 mg/kg or less or about 2.9 mg/kg or less.

25. The method of claim 23, wherein the amount of the third dose is about 3.5 mg/kg or about 2.9 mg/kg.

26. The method of any one of the previous claims, wherein the FOLR1 antibody conjugate is administered intravenously.

27. The method of any one of the previous claims, wherein the antibody is linked site-specifically to at least one payload.

28. The method of any one of the previous claims, wherein the antibody comprises one or more non-natural amino acids at sites selected from the group consisting of: HC F404, HC-K121, HC-Y180, HC-F241, HC-221, LC-T22, LC-S7, LC-N152, LC-K42, LC-E161, LC-D170, HC-S136, HC S25, HC-A40, HC-S119, HC-S190, HC-K222, HC-R19, HC-Y52, or HC-S70, according to the Kabat, Chothia, or EU numbering scheme.

29. The method of any one of the previous claims, wherein the antibody comprises one or more non-natural amino acids at sites selected from the group consisting of: HC F404, HC-Y180, and LC-K42, according to the Kabat, Chothia, or EU numbering scheme.

30. The method of claim 29, wherein the antibody comprises a non-natural amino acid at site HC-F404.

31. The method of claim 29, wherein the antibody comprises a non-natural amino acid at site HC-F404.

32. The method of claim 29, wherein the antibody comprises non-natural amino acids at sites HC-F404 and HC-Y180.

33. The method of any one of claims 28-32, wherein a residue of the one or more non-natural amino acids is linked to the payload moiety via a linker that is hydrolytically stable.

34. The method of any one of claims 28-32, wherein a residue of the one or more non-natural amino acids is linked to the payload moiety via a linker that is cleavable.

35. The method of any one of claims 28-34, wherein the one or more non-natural amino acids is selected from the group consisting of p-acetyl-L-phenylalanine, O-methyl-L-tyrosine, an -3-(2-naphthyl)alanine, 3-methyl-phenylalanine, O-4-allyl-L-tyrosine, 4-propyl-L-tyrosine, a tri-O-acetyl-GlcNAcP-serine, L-Dopa, fluorinated phenylalanine, isopropyl-L-phenylalanine, p-azido-L-phenylalanine, p-azido-methyl-L-phenylalanine, compound 56, p-acyl-L-phenylalanine, p-benzoyl-L-phenylalanine, L-phosphoserine, phosphonoserine, phosphonotyrosine, p-iodo-phenylalanine, p-bromophenylalanine, p-amino-L-phenylalanine, isopropyl-L-phenylalanine, and p-propargyloxy-phenylalanine.

36. The method of any one of claims 28-34, wherein the non-natural amino acid residue is a residue of compound (30) or compound (56).

37. The method of any one of the previous claims, wherein the at least one payload moiety is selected from the group consisting of maytansines, hemiasterlins, amanitins, and auristatins.

38. The method of any one of the previous claims, wherein the at least one payload moiety is selected from the group consisting of DM1, hemiasterlin, amanitin, MMAF, and MMAE.

39. The method of any one of the previous claims, wherein the at least one payload moiety is a hemiasterlin derivative.

40. The method of any one of the previous claims, wherein the at least one payload moiety is 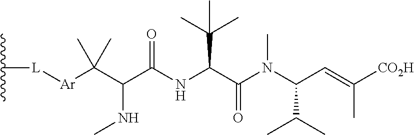
wherein Ar is aryl or heteroaryl, L is a linker, and the wiggly line indicates a bond to the antibody.

41. The method of any one of the previous claims, wherein the antibody comprises:
(i) three heavy chain CDRs of the V_{H} region SEQ ID NO: 362, and three light chain CDRs of the V_{L} region SEQ ID NO: 367;
(ii) three heavy chain CDRs of the V_{H} region SEQ ID NO: 323, and three light chain CDRs of the V_{L} region SEQ ID NO: 367;
(iii) three heavy chain CDRs of the V_{H} region SEQ ID NO: 308, and three light chain CDRs of V_{L} region SEQ ID NO: 367;
(iv) three heavy chain CDRs the V_{H} region SEQ ID NO: 309, and three light chain CDRs of V_{L} region SEQ ID NO: 367;
(v) three heavy chain CDRs of the V_{H} region SEQ ID NO: 310, and three light chain CDRs of V_{L} region SEQ ID NO: 367;
(vi) three heavy chain CDRs and three light chain CDRs of the V_{H} region SEQ ID NO: 311, and three light chain CDRs of V_{L} region SEQ ID NO: 367;
(vii) three heavy chain CDRs of the V_{H} region SEQ ID NO: 312, and three light chain CDRs of V_{L} region SEQ ID NO: 367;
(viii) three heavy chain CDRs of the V_{H} region SEQ ID NO: 313, and three light chain CDRs of V_{L} region SEQ ID NO: 367;
(ix) three heavy chain CDRs of the V_{H} region SEQ ID NO: 314, and three light chain CDRs of V_{L} region SEQ ID NO: 367;
(x) three heavy chain CDRs of the V_{H} region SEQ ID NO: 315, and three light chain CDRs of V_{L} region SEQ ID NO: 367;
(xi) three heavy chain CDRs of the V_{H} region SEQ ID NO: 316, and three light chain CDRs of V_{L} region SEQ ID NO: 367;
(xii) three heavy chain CDRs of the V_{H} region SEQ ID NO: 317, and three light chain CDRs of V_{L} region SEQ ID NO: 367;
(xiii) three heavy chain CDRs of the V_{H} region SEQ ID NO: 318, and three light chain CDRs of V_{L} region SEQ ID NO: 367;
(xiv) three heavy chain CDRs of the V_{H} region SEQ ID NO: 319, and three light chain CDRs of V_{L} region SEQ ID NO: 367;
(xv) three heavy chain CDRs of the V_{H} region SEQ ID NO: 320, and three light chain CDRs of V_{L} region SEQ ID NO: 367;
(xvi) three heavy chain CDRs of the V_{H} region SEQ ID NO: 321, and three light chain CDRs of V_{L} region SEQ ID NO: 367;
(xvii) three heavy chain CDRs of the V_{H} region SEQ ID NO: 322, and three light chain CDRs of V_{L} region SEQ ID NO: 367;
(xviii) three heavy chain CDRs of the V_{H} region SEQ ID NO: 324, and three light chain CDRs of V_{L} region SEQ ID NO: 367;
(xix) three heavy chain CDRs of the V_{H} region SEQ ID NO: 325, and three light chain CDRs of V_{L} region SEQ ID NO: 367;
(xx) three heavy chain CDRs of the V_{H} region SEQ ID NO: 326, and three light chain CDRs of V_{L} region SEQ ID NO: 367; or
(xxi) three heavy chain CDRs of the V_{H} region SEQ ID NO: 327, and three light chain CDRs of V_{L} region SEQ ID NO: 367;
(xxii) three heavy chain CDRs of the V_{H} region SEQ ID NO: 328, and three light chain CDRs of V_{L} region SEQ ID NO: 367;
(xxiii) three heavy chain CDRs of the V_{H} region SEQ ID NO: 329, and three light chain CDRs of V_{L} region SEQ ID NO: 367;
(xxiv) three heavy chain CDRs of the V_{H} region SEQ ID NO: 330, and three light chain CDRs of V_{L} region SEQ ID NO: 367;
(xxv) three heavy chain CDRs of the V_{H} region SEQ ID NO: 331, and three light chain CDRs of V_{L} region SEQ ID NO: 367;
(xxvi) three heavy chain CDRs of the V_{H} region SEQ ID NO: 332, and three light chain CDRs of V_{L} region SEQ ID NO: 367;
(xxvii) three heavy chain CDRs of the V_{H} region SEQ ID NO: 333, and three light chain CDRs of V_{L} region SEQ ID NO: 367;
(xxviii) three heavy chain CDRs of the V_{H} region SEQ ID NO: 334, and three light chain CDRs of V_{L} region SEQ ID NO: 367;
(xxix) three heavy chain CDRs of the V_{H} region SEQ ID NO: 335, and three light chain CDRs of V_{L} region SEQ ID NO: 367;
(xxx) three heavy chain CDRs of the V_{H} region SEQ ID NO: 336, and three light chain CDRs of V_{L} region SEQ ID NO: 367;
(xxxi) three heavy chain CDRs of the V_{H} region SEQ ID NO: 337, and three light chain CDRs of V_{L} region SEQ ID NO: 367;
(xxxii) three heavy chain CDRs of the V_{H} region SEQ ID NO: 338, and three light chain CDRs of V_{L} region SEQ ID NO: 367;
(xxxiii) three heavy chain CDRs of the V_{H} region SEQ ID NO: 339, and three light chain CDRs of V_{L} region SEQ ID NO: 367;
(xxxiv) three heavy chain CDRs of the V_{H} region SEQ ID NO: 340, and three light chain CDRs of V_{L} region SEQ ID NO: 367;
(xxxv) three heavy chain CDRs of the V_{H} region SEQ ID NO: 341, and three light chain CDRs of V_{L} region SEQ ID NO: 367;
(xxxvi) three heavy chain CDRs of the V_{H} region SEQ ID NO: 342, and three light chain CDRs of V_{L} region SEQ ID NO: 367;
(xxxvii) three heavy chain CDRs of the V_{H} region SEQ ID NO: 343, and three light chain CDRs of V_{L} region SEQ ID NO: 367;
(xxxviii) three heavy chain CDRs of the V_{H} region SEQ ID NO: 344, and three light chain CDRs of V_{L} region SEQ ID NO: 367;
(xxxix) three heavy chain CDRs of the V_{H} region SEQ ID NO: 345, and three light chain CDRs of V_{L} region SEQ ID NO: 367;
(xl) three heavy chain CDRs of the V_{H} region SEQ ID NO: 346, and three light chain CDRs of V_{L} region SEQ ID NO: 367;
(xli) three heavy chain CDRs of the V_{H} region SEQ ID NO: 347, and three light chain CDRs of V_{L} region SEQ ID NO: 367;
(xlii) three heavy chain CDRs of the V_{H} region SEQ ID NO: 348, and three light chain CDRs of V_{L} region SEQ ID NO: 367;
(xliii) three heavy chain CDRs of the V_{H} region SEQ ID NO: 349, and three light chain CDRs of V_{L} region SEQ ID NO: 367;
(xliv) three heavy chain CDRs of the V_{H} region SEQ ID NO: 350, and three light chain CDRs of V_{L} region SEQ ID NO: 367;
(xlv) three heavy chain CDRs of the V_{H} region SEQ ID NO: 351, and three light chain CDRs of V_{L} region SEQ ID NO: 367;
(xlvi) three heavy chain CDRs of the V_{H} region SEQ ID NO: 352, and three light chain CDRs of V_{L} region SEQ ID NO: 367;
(xlvii) three heavy chain CDRs of the V_{H} region SEQ ID NO: 353, and three light chain CDRs of V_{L} region SEQ ID NO: 367;
(xlviii) three heavy chain CDRs of the V_{H} region SEQ ID NO: 354, and three light chain CDRs of V_{L} region SEQ ID NO: 367;
(xlix) three heavy chain CDRs of the V_{H} region SEQ ID NO: 355, and three light chain CDRs of V_{L} region SEQ ID NO: 367;
(1) three heavy chain CDRs of the V_{H} region SEQ ID NO: 356, and three light chain CDRs of V_{L} region SEQ ID NO: 367;
(li) three heavy chain CDRs of the V_{H} region SEQ ID NO: 357, and three light chain CDRs of V_{L} region SEQ ID NO: 367;
(lii) three heavy chain CDRs of the V_{H} region SEQ ID NO: 358, and three light chain CDRs of V_{L} region SEQ ID NO: 367;
(liii) three heavy chain CDRs of the V_{H} region SEQ ID NO: 359, and three light chain CDRs of V_{L} region SEQ ID NO: 367;
(liv) three heavy chain CDRs of the V_{H} region SEQ ID NO: 360, and three light chain CDRs of V_{L} region SEQ ID NO: 367;
(lv) three heavy chain CDRs of the V_{H} region SEQ ID NO: 361, and three light chain CDRs of V_{L} region SEQ ID NO: 367;
(lvi) three heavy chain CDRs of the V_{H} region SEQ ID NO: 363, and three light chain CDRs of V_{L} region SEQ ID NO: 368;
(lvii) three heavy chain CDRs of the V_{H} region SEQ ID NO: 364, and three light chain CDRs of V_{L} region SEQ ID NO: 368;
(lviii) three heavy chain CDRs of the V_{H} region SEQ ID NO: 365, and three light chain CDRs of V_{L} region SEQ ID NO: 369; or
(lix) three heavy chain CDRs of the V_{H} region SEQ ID NO: 366, and three light chain CDRs of V_{L} region SEQ ID NO: 369.

42. The method of any of the previous claims, wherein the antibody comprises:
(i) a VH comprising: a CDR-H1 comprising one of SEQ ID NOs: 58 and 117; a CDR-H2 comprising one of SEQ ID NOs: 176 and 235; and a CDR-H3 comprising SEQ ID NO: 294;
(ii) a VH comprising: a CDR-H1 comprising one of SEQ ID NOs: 19 and 78; a CDR-H2 comprising one of SEQ ID NOs: 137 and 196; and a CDR-H3 comprising SEQ ID NO: 255;
(iii) a VH comprising: a CDR-H1 comprising one of SEQ ID NOs: 4 and 63; a CDR-H2 comprising one of SEQ ID NOs: 122 and 181; and a CDR-H3 comprising SEQ ID NO: 240;
(iv) a VH comprising: a CDR-H1 comprising one of SEQ ID NOs: 5 and 64; a CDR-H2 comprising one of SEQ ID NOs: 123 and 182; and a CDR-H3 comprising SEQ ID NO: 241;
(v) a V_{H} comprising: a CDR-H1 comprising one of SEQ ID NOs: 6 and 65; a CDR-H2 comprising one of SEQ ID NOs: 124 and 183; and a CDR-H3 comprising SEQ ID NO: 242;
(vi) a VH comprising: a CDR-H1 comprising one of SEQ ID NOs: 7 and 66; a CDR-H2 comprising one of SEQ ID NOs: 125 and 184; and a CDR-H3 comprising SEQ ID NO: 243;
(vii) a VH comprising: a CDR-H1 comprising one of SEQ ID NOs: 8 and 67; a CDR-H2 comprising one of SEQ ID NOs: 126 and 185; and a CDR-H3 comprising SEQ ID NO: 244;
(viii) a VH comprising: a CDR-H1 comprising one of SEQ ID NOs: 9 and 68; a CDR-H2 comprising one of SEQ ID NOs: 127 and 186; and a CDR-H3 comprising SEQ ID NO: 245;
(ix) a VH comprising: a CDR-H1 comprising one of SEQ ID NOs: 10 and 69; a CDR-H2 comprising one of SEQ ID NOs: 128 and 187; and a CDR-H3 comprising SEQ ID NO: 246;
(x) a VH comprising: a CDR-H1 comprising one of SEQ ID NOs: 11 and 70; a CDR-H2 comprising one of SEQ ID NOs: 129 and 188; and a CDR-H3 comprising SEQ ID NO: 247;
(xi) a VH comprising: a CDR-H1 comprising one of SEQ ID NOs: 12 and 71; a CDR-H2 comprising one of SEQ ID NOs: 130 and 189; and a CDR-H3 comprising SEQ ID NO: 248;
(xii) a VH comprising: a CDR-H1 comprising one of SEQ ID NOs: 13 and 72; a CDR-H2 comprising one of SEQ ID NOs: 131 and 190; and a CDR-H3 comprising SEQ ID NO: 249;
(xiii) a VH comprising: a CDR-H1 comprising one of SEQ ID NOs: 14 and 73; a CDR-H2 comprising one of SEQ ID NOs: 132 and 191; and a CDR-H3 comprising SEQ ID NO: 250;
(xiv) a VH comprising: a CDR-H1 comprising one of SEQ ID NOs: 15 and 74; a CDR-H2 comprising one of SEQ ID NOs: 133 and 192; and a CDR-H3 comprising SEQ ID NO: 251;
(xv) a VH comprising: a CDR-H1 comprising one of SEQ ID NOs: 16 and 75; a CDR-H2 comprising one of SEQ ID NOs: 134 and 193; and a CDR-H3 comprising SEQ ID NO: 252;
(xvi) a VH comprising: a CDR-H1 comprising one of SEQ ID NOs: 17 and 76; a CDR-H2 comprising one of SEQ ID NOs: 135 and 194; and a CDR-H3 comprising SEQ ID NO: 253;
(xvii) a VH comprising: a CDR-H1 comprising one of SEQ ID NOs: 18 and 77; a CDR-H2 comprising one of SEQ ID NOs: 136 and 195; and a CDR-H3 comprising SEQ ID NO: 254;
(xviii) a VH comprising: a CDR-H1 comprising one of SEQ ID NOs: 20 and 79; a CDR-H2 comprising one of SEQ ID NOs: 138 and 197; and a CDR-H3 comprising SEQ ID NO: 256;
(xix) a VH comprising: a CDR-H1 comprising one of SEQ ID NOs: 21 and 80; a CDR-H2 comprising one of SEQ ID NOs: 139 and 198; and a CDR-H3 comprising SEQ ID NO: 257;
(xx) a VH comprising: a CDR-H1 comprising one of SEQ ID NOs: 22 and 81; a CDR-H2 comprising one of SEQ ID NOs: 140 and 199; and a CDR-H3 comprising SEQ ID NO: 258;
(xxi) a VH comprising: a CDR-H1 comprising one of SEQ ID NOs: 23 and 82; a CDR-H2 comprising one of SEQ ID NOs: 141 and 200; and a CDR-H3 comprising SEQ ID NO: 259;
(xxii) a VH comprising: a CDR-H1 comprising one of SEQ ID NOs: 24 and 83; a CDR-H2 comprising one of SEQ ID NOs: 142 and 201; and a CDR-H3 comprising SEQ ID NO: 260;
(xxiii) a VH comprising: a CDR-H1 comprising one of SEQ ID NOs: 25 and 84; a CDR-H2 comprising one of SEQ ID NOs: 143 and 202; and a CDR-H3 comprising SEQ ID NO: 261;
(xxiv) a VH comprising: a CDR-H1 comprising one of SEQ ID NOs: 26 and 85; a CDR-H2 comprising one of SEQ ID NOs: 144 and 203; and a CDR-H3 comprising SEQ ID NO: 262;
(xxv) a VH comprising: a CDR-H1 comprising one of SEQ ID NOs: 27 and 86; a CDR-H2 comprising one of SEQ ID NOs: 145 and 204; and a CDR-H3 comprising SEQ ID NO: 263;
(xxvi) a VH comprising: a CDR-H1 comprising one of SEQ ID NOs: 28 and 87; a CDR-H2 comprising one of SEQ ID NOs: 146 and 205; and a CDR-H3 comprising SEQ ID NO: 264;
(xxvii) a VH comprising: a CDR-H1 comprising one of SEQ ID NOs: 29 and 88; a CDR-H2 comprising one of SEQ ID NOs: 147 and 206; and a CDR-H3 comprising SEQ ID NO: 265;
(xxviii) a VH comprising: a CDR-H1 comprising one of SEQ ID NOs: 30 and 89; a CDR-H2 comprising one of SEQ ID NOs: 148 and 207; and a CDR-H3 comprising SEQ ID NO: 266;
(xxix) a VH comprising: a CDR-H1 comprising one of SEQ ID NOs: 31 and 90; a CDR-H2 comprising one of SEQ ID NOs: 149 and 208; and a CDR-H3 comprising SEQ ID NO: 267;
(xxx) a VH comprising: a CDR-H1 comprising one of SEQ ID NOs: 32 and 91; a CDR-H2 comprising one of SEQ ID NOs: 150 and 209; and a CDR-H3 comprising SEQ ID NO: 268;
(xxxi) a VH comprising: a CDR-H1 comprising one of SEQ ID NOs: 33 and 92; a CDR-H2 comprising one of SEQ ID NOs: 151 and 210; and a CDR-H3 comprising SEQ ID NO: 269;
(xxxii) a VH comprising: a CDR-H1 comprising one of SEQ ID NOs: 34 and 93; a CDR-H2 comprising one of SEQ ID NOs: 152 and 211; and a CDR-H3 comprising SEQ ID NO: 270;
(xxxiii) a VH comprising: a CDR-H1 comprising one of SEQ ID NOs: 35 and 94; a CDR-H2 comprising one of SEQ ID NOs: 153 and 212; and a CDR-H3 comprising SEQ ID NO: 271;
(xxxiv) a VH comprising: a CDR-H1 comprising one of SEQ ID NOs: 36 and 95; a CDR-H2 comprising one of SEQ ID NOs: 154 and 213; and a CDR-H3 comprising SEQ ID NO: 272;
(xxxv) a VH comprising: a CDR-H1 comprising one of SEQ ID NOs: 37 and 96; a CDR-H2 comprising one of SEQ ID NOs: 155 and 214; and a CDR-H3 comprising SEQ ID NO: 273;
(xxxvi)a VH comprising: a CDR-H1 comprising one of SEQ ID NOs: 38 and 97; a CDR-H2 comprising one of SEQ ID NOs: 156 and 215; and a CDR-H3 comprising SEQ ID NO: 274;
(xxxvii) a VH comprising: a CDR-H1 comprising one of SEQ ID NOs: 39 and 98; a CDR-H2 comprising one of SEQ ID NOs: 157 and 216; and a CDR-H3 comprising SEQ ID NO: 275;
(xxxviii) a VH comprising: a CDR-H1 comprising one of SEQ ID NOs: 40 and 99; a CDR-H2 comprising one of SEQ ID NOs: 158 and 217; and a CDR-H3 comprising SEQ ID NO: 276;
(xxxix) a VH comprising: a CDR-H1 comprising one of SEQ ID NOs: 41 and 100; a CDR-H2 comprising one of SEQ ID NOs: 159 and 218; and a CDR-H3 comprising SEQ ID NO: 277;
(xl) a VH comprising: a CDR-H1 comprising one of SEQ ID NOs: 42 and 101; a CDR-H2 comprising one of SEQ ID NOs: 160 and 219; and a CDR-H3 comprising SEQ ID NO: 278;
(xli) a VH comprising: a CDR-H1 comprising one of SEQ ID NOs: 43 and 102; a CDR-H2 comprising one of SEQ ID NOs: 161 and 220; and a CDR-H3 comprising SEQ ID NO: 279;
(xlii) a VH comprising: a CDR-H1 comprising one of SEQ ID NOs: 44 and 103; a CDR-H2 comprising one of SEQ ID NOs: 162 and 221; and a CDR-H3 comprising SEQ ID NO: 280;
(xliii) a VH comprising: a CDR-H1 comprising one of SEQ ID NOs: 45 and 104; a CDR-H2 comprising one of SEQ ID NOs: 163 and 222; and a CDR-H3 comprising SEQ ID NO: 281;
(xliv) a VH comprising: a CDR-H1 comprising one of SEQ ID NOs: 46 and 10⁵; a CDR-H2 comprising one of SEQ ID NOs: 164 and 223; and a CDR-H3 comprising SEQ ID NO: 282;
(xlv) a VH comprising: a CDR-H1 comprising one of SEQ ID NOs: 47 and 106; a CDR-H2 comprising one of SEQ ID NOs: 165 and 224; and a CDR-H3 comprising SEQ ID NO: 283;
(xlvi) a VH comprising: a CDR-H1 comprising one of SEQ ID NOs: 48 and 10⁷; a CDR-H2 comprising one of SEQ ID NOs: 166 and 225; and a CDR-H3 comprising SEQ ID NO: 284;
(xlvii) a VH comprising: a CDR-H1 comprising one of SEQ ID NOs: 49 and 108; a CDR-H2 comprising one of SEQ ID NOs: 167 and 226; and a CDR-H3 comprising SEQ ID NO: 285;
(xlviii) a VH comprising: a CDR-H1 comprising one of SEQ ID NOs: 50 and 109; a CDR-H2 comprising one of SEQ ID NOs: 168 and 227; and a CDR-H3 comprising SEQ ID NO: 286;
(xlix) a VH comprising: a CDR-H1 comprising one of SEQ ID NOs: 51 and 110; a CDR-H2 comprising one of SEQ ID NOs: 169 and 228; and a CDR-H3 comprising SEQ ID NO: 287;
(1) a VH comprising: a CDR-H1 comprising one of SEQ ID NOs: 52 and 111; a CDR-H2 comprising one of SEQ ID NOs: 170 and 229; and a CDR-H3 comprising SEQ ID NO: 288;
(li) a VH comprising: a CDR-H1 comprising one of SEQ ID NOs: 53 and 112; a CDR-H2 comprising one of SEQ ID NOs: 171 and 230; and a CDR-H3 comprising SEQ ID NO: 289;
(lii) a VH comprising: a CDR-H1 comprising one of SEQ ID NOs: 54 and 113; a CDR-H2 comprising one of SEQ ID NOs: 172 and 231; and a CDR-H3 comprising SEQ ID NO: 290;
(liii) a VH comprising: a CDR-H1 comprising one of SEQ ID NOs: 55 and 114; a CDR-H2 comprising one of SEQ ID NOs: 173 and 232; and a CDR-H3 comprising SEQ ID NO: 291;
(liv) a VH comprising: a CDR-H1 comprising one of SEQ ID NOs: 56 and 115; a CDR-H2 comprising one of SEQ ID NOs: 174 and 233; and a CDR-H3 comprising SEQ ID NO: 292;
(lv) a VH comprising: a CDR-H1 comprising one of SEQ ID NOs: 57 and 116; a CDR-H2 comprising one of SEQ ID NOs: 175 and 234; and a CDR-H3 comprising SEQ ID NO: 293;
(lvi) a VH comprising: a CDR-H1 comprising one of SEQ ID NOs: 59 and 118; a CDR-H2 comprising one of SEQ ID NOs: 177 and 236; and a CDR-H3 comprising SEQ ID NO: 295;
(lvii) a VH comprising: a CDR-H1 comprising one of SEQ ID NOs: 60 and 119; a CDR-H2 comprising one of SEQ ID NOs: 178 and 237; and a CDR-H3 comprising SEQ ID NO: 296;
(lviii) a VH comprising: a CDR-H1 comprising one of SEQ ID NOs: 61 and 120; a CDR-H2 comprising one of SEQ ID NOs: 179 and 238; and a CDR-H3 comprising SEQ ID NO: 297; or
(lix) a VH comprising: a CDR-H1 comprising one of SEQ ID NOs: 62 and 121; a CDR-H2 comprising one of SEQ ID NOs: 180 and 239; and a CDR-H3 comprising SEQ ID NO: 298.

43. The method of any of the previous claims, wherein the antibody comprises:
(a) a VL comprising: a CDR-L1 comprising SEQ ID NO: 300; a CDR-L2 comprising SEQ ID NO: 303; and a CDR-L3 comprising SEQ ID NO: 306; or
(b) a VL comprising: a CDR-L1 comprising SEQ ID NO: 301; a CDR-L2 comprising SEQ ID NO: 304; and a CDR-L3 comprising SEQ ID NO: 307.

44. The method of any of the previous claims, wherein the antibody comprises:
(i) the VH region SEQ ID NO: 362, or a variant thereof, and the VL region SEQ ID NO: 367, or a variant thereof;
(ii) the VH region SEQ ID NO: 323, or a variant thereof, and the VL region SEQ ID NO: 367, or a variant thereof;
(iii) the VH region SEQ ID NO: 308, or a variant thereof, and the VL region SEQ ID NO: 367, or a variant thereof;
(iv) the VH region SEQ ID NO: 309, or a variant thereof, and the VL region SEQ ID NO: 367, or a variant thereof;
(v) the VH region SEQ ID NO: 310, or a variant thereof, and the VL region SEQ ID NO: 367, or a variant thereof;
(vi) the VH region SEQ ID NO: 311, or a variant thereof, and the VL region SEQ ID NO: 367, or a variant thereof;
(vii) the VH region SEQ ID NO: 312, or a variant thereof, and the VL region SEQ ID NO: 367, or a variant thereof;
(viii) the VH region SEQ ID NO: 313, or a variant thereof, and the VL region SEQ ID NO: 367, or a variant thereof;
(ix) the VH region SEQ ID NO: 314, or a variant thereof, and the VL region SEQ ID NO: 367, or a variant thereof;
(x) the VH region SEQ ID NO: 315, or a variant thereof, and the VL region SEQ ID NO: 367, or a variant thereof;
(xi) the VH region SEQ ID NO: 316, or a variant thereof, and the VL region SEQ ID NO: 367, or a variant thereof;
(xii) the VH region SEQ ID NO: 317, or a variant thereof, and the VL region SEQ ID NO: 367, or a variant thereof;
(xiii) the VH region SEQ ID NO: 318, or a variant thereof, and the VL region SEQ ID NO: 367, or a variant thereof;
(xiv) the VH region SEQ ID NO: 319, or a variant thereof, and the VL region SEQ ID NO: 367, or a variant thereof;
(xv) the VH region SEQ ID NO: 320, or a variant thereof, and the VL region SEQ ID NO: 367, or a variant thereof;
(xvi) the VH region SEQ ID NO: 321, or a variant thereof, and the VL region SEQ ID NO: 367, or a variant thereof;
(xvii) the VH region SEQ ID NO: 322, or a variant thereof, and the VL region SEQ ID NO: 367, or a variant thereof;
(xviii) the VH region SEQ ID NO: 324, or a variant thereof, and the VL region SEQ ID NO: 367, or a variant thereof;
(xix) the VH region SEQ ID NO: 325, or a variant thereof, and the VL region SEQ ID NO: 367, or a variant thereof;
(xx) the VH region SEQ ID NO: 326, or a variant thereof, and the VL region SEQ ID NO: 367, or a variant thereof; or
(xxi) the VH region SEQ ID NO: 327, or a variant thereof, and the VL region SEQ ID NO: 367, or a variant thereof;
(xxii) the VH region SEQ ID NO: 328, or a variant thereof, and the VL region SEQ ID NO: 367, or a variant thereof;
(xxiii) the VH region SEQ ID NO: 329, or a variant thereof, and the VL region SEQ ID NO: 367, or a variant thereof,
(xxiv) the VH region SEQ ID NO: 330, or a variant thereof, and the VL region SEQ ID NO: 367, or a variant thereof;
(xxv) the VH region SEQ ID NO: 331, or a variant thereof, and the VL region SEQ ID NO: 367, or a variant thereof;
(xxvi) the VH region SEQ ID NO: 332, or a variant thereof, and the VL region SEQ ID NO: 367, or a variant thereof;
(xxvii) the VH region SEQ ID NO: 333, or a variant thereof, and the VL region SEQ ID NO: 367, or a variant thereof,
(xxviii) the VH region SEQ ID NO: 334, or a variant thereof, and the VL region SEQ ID NO: 367, or a variant thereof,
(xxix) the VH region SEQ ID NO: 335, or a variant thereof, and the VL region SEQ ID NO: 367, or a variant thereof,
(xxx) the VH region SEQ ID NO: 336, or a variant thereof, and the VL region SEQ ID NO: 367, or a variant thereof;
(xxxi) the VH region SEQ ID NO: 337, or a variant thereof, and the VL region SEQ ID NO: 367, or a variant thereof;
(xxxii) the VH region SEQ ID NO: 338, or a variant thereof, and the VL region SEQ ID NO: 367, or a variant thereof,
(xxxiii) the VH region SEQ ID NO: 339, or a variant thereof, and the VL region SEQ ID NO: 367, or a variant thereof,
(xxxiv) the VH region SEQ ID NO: 340, or a variant thereof, and the VL region SEQ ID NO: 367, or a variant thereof,
(xxxv) the VH region SEQ ID NO: 341, or a variant thereof, and the VL region SEQ ID NO: 367, or a variant thereof,
(xxxvi) the VH region SEQ ID NO: 342, or a variant thereof, and the VL region SEQ ID NO: 367, or a variant thereof,
(xxxvii) the VH region SEQ ID NO: 343, or a variant thereof, and the VL region SEQ ID NO: 367, or a variant thereof,
(xxxviii) the VH region SEQ ID NO: 344, or a variant thereof, and the VL region SEQ ID NO: 367, or a variant thereof,
(xxxix) the VH region SEQ ID NO: 345, or a variant thereof, and the VL region SEQ ID NO: 367, or a variant thereof,
(xl) the VH region SEQ ID NO: 346, or a variant thereof, and the VL region SEQ ID NO: 367, or a variant thereof;
(xli) the VH region SEQ ID NO: 347, or a variant thereof, and the VL region SEQ ID NO: 367, or a variant thereof;
(xlii) the VH region SEQ ID NO: 348, or a variant thereof, and the VL region SEQ ID NO: 367, or a variant thereof;
(xliii) the VH region SEQ ID NO: 349, or a variant thereof, and the VL region SEQ ID NO: 367, or a variant thereof;
(xliv) the VH region SEQ ID NO: 350, or a variant thereof, and the VL region SEQ ID NO: 367, or a variant thereof;
(xlv) the VH region SEQ ID NO: 351, or a variant thereof, and the VL region SEQ ID NO: 367, or a variant thereof;
(xlvi) the VH region SEQ ID NO: 352, or a variant thereof, and the VL region SEQ ID NO: 367, or a variant thereof;
(xlvii) the VH region SEQ ID NO: 353, or a variant thereof, and the VL region SEQ ID NO: 367, or a variant thereof,
(xlviii) the VH region SEQ ID NO: 354, or a variant thereof, and the VL region SEQ ID NO: 367, or a variant thereof,
(xlix) the VH region SEQ ID NO: 355, or a variant thereof, and the VL region SEQ ID NO: 367, or a variant thereof;
(1) the VH region SEQ ID NO: 356, or a variant thereof, and the VL region SEQ ID NO: 367, or a variant thereof;
(li) the VH region SEQ ID NO: 357, or a variant thereof, and the VL region SEQ ID NO: 367, or a variant thereof;
(lii) the VH region SEQ ID NO: 358, or a variant thereof, and the VL region SEQ ID NO: 367, or a variant thereof;
(liii) the VH region SEQ ID NO: 359, or a variant thereof, and the VL region SEQ ID NO: 367, or a variant thereof;
(liv) the VH region SEQ ID NO: 360, or a variant thereof, and the VL region SEQ ID NO: 367, or a variant thereof;
(lv) the VH region SEQ ID NO: 361, or a variant thereof, and the VL region SEQ ID NO: 367, or a variant thereof;
(lvi) the VH region SEQ ID NO: 363, or a variant thereof, and the VL region SEQ ID NO: 368, or a variant thereof;
(lvii) the VH region SEQ ID NO: 364, or a variant thereof, and the VL region SEQ ID NO: 368, or a variant thereof;
(lviii) the VH region SEQ ID NO: 365, or a variant thereof, and the VL region SEQ ID NO: 369, or a variant thereof; or
(lix) the VH region SEQ ID NO: 366, or a variant thereof, and the VL region SEQ ID NO: 369, or a variant thereof.

45. The method of any one of the previous claims, wherein the antibody conjugate has the structure of Conjugate P: 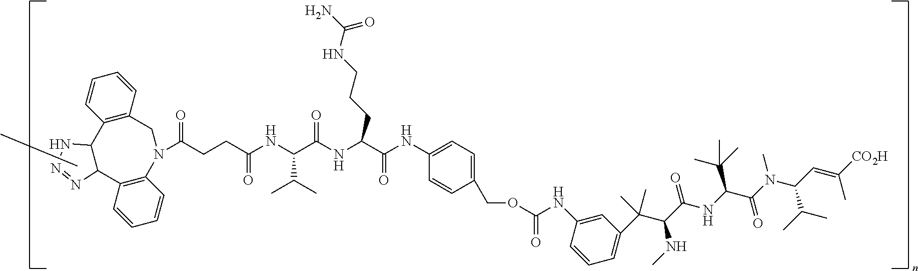
wherein n is an integer from 1 to 6.

46. The method of any of the previous claims, wherein the antibody conjugate has the structure of Conjugate M: 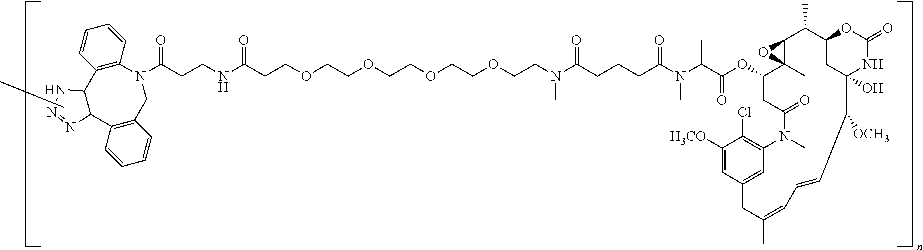
wherein n is an integer from 1 to 6.

47. The method of any of the previous claims, wherein the antibody conjugate has the structure of Conjugate Q: 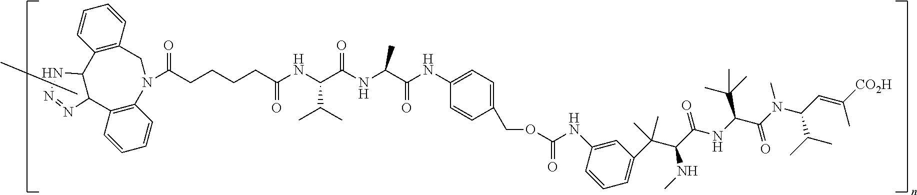
wherein n is an integer from 1 to 6.

48. The method of any one of the previous claims, wherein the antibody comprises a VH region of SEQ ID NO: 362, or a variant thereof 7 or fewer amino acid substitutions, and a VL region of SEQ ID NO: 367, or a variant thereof having 7 or fewer amino acid substitutions.

49. The method of any one of the previous claims, wherein the antibody comprises a VH region of SEQ ID NO: 323, or a variant thereof 7 or fewer amino acid substitutions, and a VL region of SEQ ID NO: 367, or a variant thereof having 7 or fewer amino acid substitutions.

50. The method of any of the previous claims, wherein the amino acid substitutions are conservative amino acid substitutions.

51. The method of any one of the previous claims, wherein the antibody further comprises at least one constant region domain.

52. The method of claim 51, wherein the constant region comprises a sequence selected from SEQ ID NOs: 370, 371, and 372.

53. The method of claim 52, wherein the antibody constant regions are according to SEQ ID NO:370.

54. The method of claim 52, wherein the antibody constant regions are according to SEQ ID NO:371.

55. The method of claim 52, wherein the antibody constant regions are according to SEQ ID NO:372.

56. The method of any of the previous claims, wherein the antibody is a monoclonal antibody.

57. The method of any of the previous claims, wherein the antibody is an IgA, an IgD, an IgE, an IgG, or an IgM.

58. The method of any of the previous claims, wherein the antibody is humanized or human.

59. The method of any of the previous claims, wherein the antibody is aglycosylated.

60. The method of any of the previous claims, wherein the antibody is an antibody fragment.

61. The method of claim 60, wherein the antibody fragment is selected from an Fv fragment, a Fab fragment, a F(ab′)₂ fragment, a Fab′ fragment, an scFv (sFv) fragment, and an scFv-Fc fragment.

62. The method of claim 61, wherein the antibody fragment is an scFv fragment.

63. The method of claim 61, wherein the antibody fragment is an scFv-Fc fragment.

64. The method of any one of the previous claims, wherein the subject previously received cancer treatment.

65. The method of any one of the previous claims, wherein the subject did not previously receive cancer treatment.

66. The method of any one of the previous claims, wherein the subject was diagnosed with cancer.

67. The method of any one of the previous claims, wherein the cancer is ovarian cancer.

68. The method of any one of the previous claims, wherein the cancer is endometrial cancer.

69. The method of any of the previous claims wherein the antibody conjugate has the structure of Conjugate P: 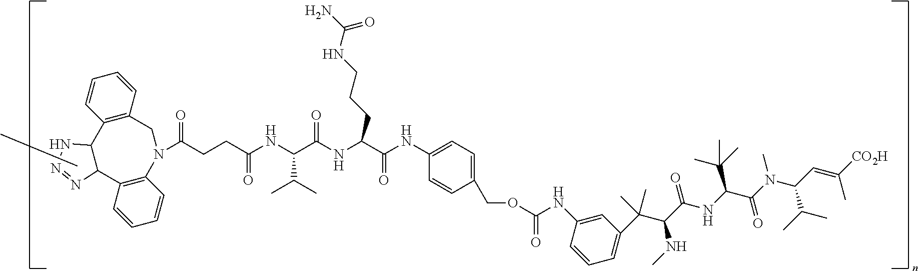
wherein
n is 4;
the antibody is an IgG antibody comprising VH regions according to SEQ ID NO: 362, and VL regions according SEQ ID NO: 367;
the antibody further comprises para-azidomethylphenylalanine residues substituting for Y180 and F404 of each VH region; and
each bond in the structure to the antibody is to the side chain of one of the para-azidomethylphenylalanine residues.

70. The method of any of the previous claims, further comprising administering a second therapeutic agent.

71. The method of claim 70, wherein the second therapeutic agent is a granulocyte colony-stimulating factor (GCSF).

72. The method of claim 70, wherein the second therapeutic agent is a pegylated GCSF.

73. The method of claim 71, wherein the GCSF is pegfilgrastim or filgrastim.

74. The method of claim 71, wherein the GCSF is pegfilgrastim.

75. The method of any one of claims 70-74, wherein the second therapeutic agent is administered concurrently with or sequentially to the first dose of the antibody conjugate.

76. The method of any one of claims 70-74, wherein the second therapeutic agent is administered at least 7 days after administering the first dose of the antibody conjugate.

77. The method of any one of claims 70-74, wherein the second therapeutic agent is administered at 7, 8, 9, 10 or 11 days after administering the first dose of the antibody conjugate.

78. The method of any one of claims 70-74, wherein the second therapeutic agent is administered at 8±2 days after administering the first dose.

79. The method of any one of claims 70-74, wherein the second therapeutic agent is administered at around 8 days after administering the first dose.

80. The method of any one of claims 71-74, wherein the GCSF is pegfilgrastim and the dose is 6 mg.

81. The method of any one of claims 70-74, wherein the second therapeutic agent is administered once every 8-10 days for 1-5 cycles.

82. The method of any one of claims 70-74, wherein the second therapeutic agent is administered once every 8-10 days for 1-3 cycles.

83. The method of any one of claims 70-74, wherein the second therapeutic agent is administered at least 24 hours after administering the first dose of the antibody conjugate.

84. The method of any one of claims 70-73, wherein the GCSF is filgrastim and the dose is 5-10 μg/kg/day.

85. A pharmaceutical package or kit, comprising:
a container;
a composition comprising a folate receptor alpha (FOLR1) antibody conjugate; and
a package insert comprising instructions to administer the FOLR1 antibody conjugate according the method of any of the previous claims.

86. The pharmaceutical package or kit further comprising instructions to administer the FOLR1 antibody conjugate in combination with a second therapeutic agent.

87. The pharmaceutical package or kit further comprising instructions to administer the FOLR1 antibody conjugate in combination with pegfilgrastim.

88. The pharmaceutical package or kit further comprising instructions to administer the FOLR1 antibody conjugate in combination with filgrastim.
